(19) United States
(12) Patent Application Publication
(10) Pub. No.: US 2015/0183802 A1
(43) Pub. Date: Jul. 2, 2015

(54) TRICYCLIC SULFONAMIDE DERIVATIVES

(71) Applicants: Shuhui CHEN, Shanghai (CN); Haiyang HE, Shanghai (CN); Bharat LAGU, Acton, MA (US); Hua QIN, Shanghai (CN); Chengde WU, Shanghai (CN); Yisong XIAO, Shanghai (CN)

(72) Inventors: Shuhui CHEN, Shanghai (CN); Haiyang HE, Shanghai (CN); Bharat LAGU, Acton, MA (US); Hua QIN, Shanghai (CN); Chengde WU, Shanghai (CN); Yisong XIAO, Shanghai (CN)

(73) Assignee: NOVARTIS AG, Basel (CH)

(21) Appl. No.: 14/580,192

(22) Filed: Dec. 22, 2014

(30) Foreign Application Priority Data
Dec. 30, 2013 (CN) PCT/CN2013/090854

(51) Int. Cl. C07D 498/10; A61K 31/424; C07D 498/04; A61K 45/06; C07D 413/04; A61K 31/519; A61K 31/5386; A61K 31/5383; A61K 31/4439; A61K 31/4745

(52) U.S. Cl. CPC C07D 498/10; A61K 31/4439; A61K 31/424; C07D 498/04; A61K 31/4745; C07D 413/04; A61K 31/519; A61K 31/5386; A61K 31/5383; A61K 45/06

(57) ABSTRACT

The invention relates to derivatives of formula (I),

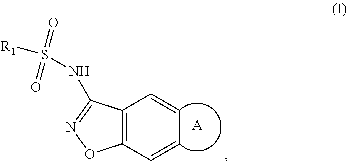

wherein the substituents are as defined in the specification; to processes for the preparation of such derivatives; pharmaceutical compositions comprising such derivatives; such derivatives as a medicament; such derivatives for the treatment of pain.

### FIELD OF THE INVENTION

The present invention relates to novel tricyclic sulfonamide derivatives that are sodium channel blockers, in particular selective inhibitors of the voltage-gated sodium channel 1.7 (Nav1.7), processes for their preparation, pharmaceutical compositions and medicaments containing them and to their use in diseases and disorders which respond to inhibition of Nav1.7.

### BACKGROUND OF THE INVENTION

The compounds of the present invention are sodium channel blockers, in particular selective inhibitors of the voltage-gated sodium channel 1.7 (Nav1.7) which is involved in pain. Since other sodium channel subtypes are involved in different essential physiological processes such as heart activity (Nav1.5), muscle contraction (Nav1.4) and CNS neurotransmission (Nav1.1, 1.2 and 1.6), selectivity for Nav1.7 is believed to be associated with the potential elimination of side effects.

Several Nav1.7 blockers are described: The tarantula venom peptide Pro-TX-II is a potent inhibitor of Nav1.7 (Schmalhofer et al, Molecular Pharmacology 2008, 74, 1476-1484). A series of Benzazepinone Nav1.7 blockers are described to show activity in pre-clinical pharmacological models of pain (Williams et al, Biochemistry, 2007, 46(50), 14693-14703; McGowan et al., Anesth Analg, 2009, 109, 951-958). Amino-thiazoles and amino-pyridines are described as Nav1.7 inhibitors (WO2007109324) and isoxazoles are described as Nav1.7 inhibitors (WO2009010784).

Nonsense mutations in SCN9A, the gene coding Nav1.7, appear to be linked to Congenital Indifference to Pain (CIP) (Cox et al, Nature, 2006, 444(7121), 894-898). Patients with CIP are essentially completely indifferent to sensations that would cause pain in most individuals e.g. bone fractures, burns, dental abscesses, appendicitis and childbirth. Concurrently, they are able to distinguish between other sensations, such as thermal (hot/cold) and tactile (sharp/dull) stimuli (Goldberg et al, Clinical Genetics, 2007, 71(4), 311-319).

Recent clinical reports indicate that gain of function mutations in human Nav1.7 are typically associated with severe pathological conditions. Primary Erythermalgia has been associated with mutations T2573A and T2543C in Nav1.7 (Yang et al, Journal of Medical Genetics, 2004, 41(3), 171-4). Paroxysmal Extreme Pain Disorder is described in association with mutations M1627K, T14641 and I1461T located in the inactivation gate area of Nav1.7 (Fertleman et al, Neuron, 2006, 52(5), 767-774).

Thus selective inhibition of Nav1.7 channels may provide comprehensive analgesia.

Hence, there is a continuing need for compounds which may be useful for treating and preventing disorders or diseases which respond to inhibition of Nav1.7, particularly for compounds with improved efficacy, tolerability and/or selectivity.

### SUMMARY OF THE INVENTION

In a first aspect, there is provided a compound of formula (I)

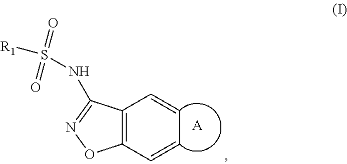

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

A is selected from the group consisting of

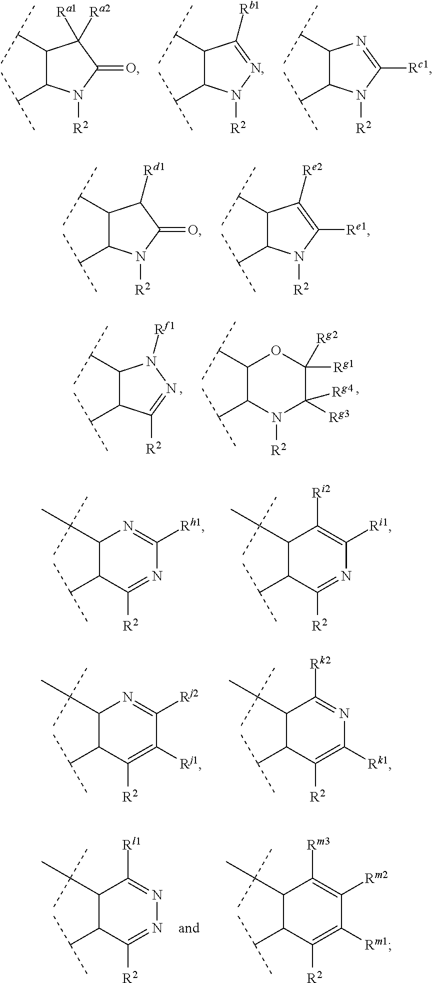

R^{a1} and R^{a2} are independently selected from the group consisting of H, C₁₋₄ alkyl, C₁₋₄ haloalkyl, halogen and CN;

or R^{a1} and R^{a2} form together with the carbon atom to which they are attached a C₃₋₆ cycloalkyl ring;

R^{b1} is selected from the group consisting of H, C₁₋₄ alkyl, C₁₋₄ haloalkyl, halogen and CN;

R^{c1} is selected from the group consisting of H, C₁₋₄ alkyl, C₁₋₄ haloalkyl, halogen and CN;

R^{d1} is selected from the group consisting of H, C₁₋₄ alkyl and C₁₋₄ haloalkyl;

R^{e1} is selected from the group consisting of H, C₁₋₄ alkyl, C₁₋₄ haloalkyl, halogen and CN;

R^{e2} is selected from the group consisting of H, C₁₋₄ alkyl, C₁₋₄ haloalkyl, halogen, CN and —C(═O)NR^{e21}R^{e22};

R^{e21} and R^{e22} are independently selected from H and C₁₋₄ alkyl;

R^{f1} selected from the group consisting of H, C₁₋₄ alkyl and C₁₋₄ haloalkyl;

R^{g1} and R^{g2} are independently selected from the group consisting of H, C₁₋₄ alkyl and C₁₋₄ haloalkyl;

or R^{g1} and R^{g2} form together with the carbon atom to which they are attached a C₃₋₆ cycloalkyl ring;

R^{g3} and R^{g4} are independently selected from the group consisting of H, C₁₋₄ alkyl and C₁₋₄ haloalkyl;

or R^{g3} and R^{g4} form together an oxo group;

R^{h1}, Rⁱ¹, Rⁱ², R^{j1}, R^{j2}, R^{k1}, R^{k2}, R^{l1}, R^{m1}, R^{m2} and R^{m3} are independently selected from the group consisting of H, C₁₋₄ alkyl, C₁₋₄ haloalkyl, halogen and CN;

R² is selected from the group consisting of

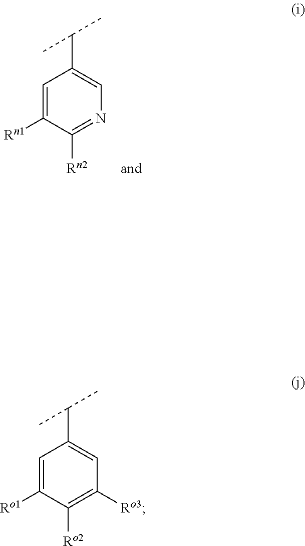

Rⁿ¹ is selected from the group consisting of H, halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

In another aspect, there is provided a compound as defined in the first aspect for use as a medicine, in particular for the treatment of pain.

In a further aspect, there is provided a method of treating pain, comprising administering to the subject in need thereof a therapeutically effective amount of a compound as defined in the first aspect.

### DETAILED DESCRIPTION OF THE INVENTION

### Embodiment 1

A compound of formula (I)

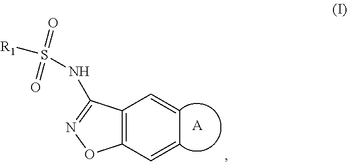

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

A is selected from the group consisting of

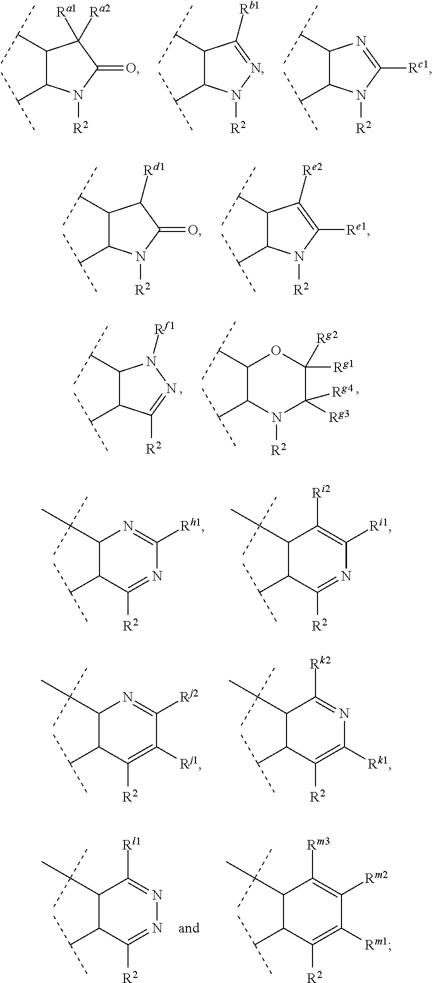

R^{a1} and R^{a2} are independently selected from the group consisting of H, C₁₋₄ alkyl, C₁₋₄ haloalkyl, halogen and CN;

or R^{a1} and R^{a2} form together with the carbon atom to which they are attached a C₃₋₆ cycloalkyl ring;

R^{b1} is selected from the group consisting of H, C₁₋₄ alkyl, C₁₋₄ haloalkyl, halogen and CN;

R^{c1} is selected from the group consisting of H, C₁₋₄ alkyl, C₁₋₄ haloalkyl, halogen and CN;

R^{d1} is selected from the group consisting of H, C₁₋₄ alkyl and C₁₋₄ haloalkyl;

R^{e1} is selected from the group consisting of H, C₁₋₄ alkyl, C₁₋₄ haloalkyl, halogen and CN;

R^{e2} is selected from the group consisting of H, C₁₋₄ alkyl, C₁₋₄ haloalkyl, halogen, CN and —C(═O)NR^{e21}R^{e22};

R^{e21} and R^{e22} are independently selected from H and C₁₋₄ alkyl;

R^{f1} selected from the group consisting of H, C₁₋₄ alkyl and C₁₋₄ haloalkyl;

R^{g1} and R^{g2} are independently selected from the group consisting of H, C₁₋₄ alkyl and C₁₋₄ haloalkyl;

or R^{g1} and R^{g2} form together with the carbon atom to which they are attached a C₃₋₆ cycloalkyl ring;

R^{g3} and R^{g4} are independently selected from the group consisting of H, C₁₋₄ alkyl and C₁₋₄ haloalkyl;

or R^{g3} and R^{g4} form together an oxo group;

R^{h1}, Rⁱ¹, Rⁱ², R^{j1}, R^{j2}, R^{k1}, R^{k2}, R^{l1}, R^{m1}, R^{m2} and R^{m3} are independently selected from the group consisting of H, C₁₋₄ alkyl, C₁₋₄ haloalkyl, halogen and CN;

R² is selected from the group consisting of

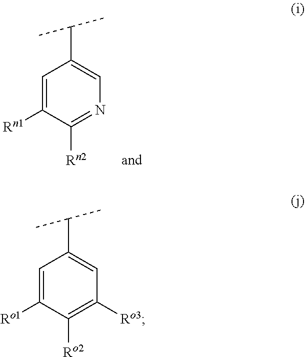

Rⁿ¹ is selected from the group consisting of H, halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### DEFINITIONS

The term ‘C₁₋₄alkyl’ as used herein as a group or a part of the group refers to a linear or branched saturated hydrocarbon group containing from 1 to 4 carbon atoms. Examples of such groups include methyl, ethyl, n-propyl, isopropyl, n-butyl, isobutyl, sec-butyl, tert-butyl, and the like. Unless a particular structure is specified, the terms propyl, butyl etc. include all straight and branched chain forms having the appropriate number of carbon atoms e.g. propyl includes n-propyl and isopropyl.

The term ‘C₁₋₄alkoxy’ as used herein refers to an —O—C₁₋₄alkyl group wherein C₁₋₄alkyl is as defined herein. Examples of such groups include methoxy, ethoxy, propoxy, butoxy. As for alkyl unless a particular structure is specified the terms propoxy, butoxy etc. include all straight and branched chain forms having the appropriate number of carbon atoms e.g. propoxy includes n-propoxy and isopropoxy.

The term ‘halo’ or ‘halogen’, as used herein, may be fluoro, chloro, bromo or iodo.

The term ‘C₁₋₄haloalkyl’ as used herein refers to a C₁₋₄alkyl group as defined herein substituted with one or more halogen groups which halogen groups may be the same or different, e.g. —CF₃, —CF₂H or —CH₂CF₃.

The term ‘C₁₋₄haloalkoxy’ as used herein refers to a C₁₋₄alkoxy group as defined herein substituted with one or more halogen groups which halogen groups may be the same or different.

The term ‘C₃₋₆cycloalkyl’ as used herein refers to a saturated monocyclic hydrocarbon ring of 3 to 6 carbon atoms. Examples of such groups include cyclopropyl, cyclobutyl, cyclopentyl and cyclohexyl.

The term ‘4 to 6 membered heterocyclic ring’ or ‘4 to 6 membered heterocyclyl’ refers to a 4 to 6 membered saturated or partially unsaturated aliphatic monocyclic ring which contains 1 to 2 heteroatoms selected from oxygen, nitrogen and sulphur. Suitable examples of such groups include azetidinyl, pyrrolidinyl, piperidinyl, piperazinyl, morpholinyl and thiomorpholinyl.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by NaV1.7 or (ii) associated with NaV1.7 activity, or (iii) characterized by activity (normal or abnormal) of NaV1.7; or (2) reduce or inhibit the activity of NaV1.7; or (3) reduce or inhibit the expression of NaV1.7. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of NaV1.7; or at least partially reducing or inhibiting the expression of NaV1.7.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

As used herein, when one embodiment refers to several other embodiments by using the term “according to any one of”, for example “according to any one of embodiments 1 to 5”, then said embodiment refers not only to embodiments indicated by the integers such as 1 and 2 but also to embodiments indicated by numbers with a decimal component such as 1.1, 1.2 or 2.1, 2.2, 2.3. For example, “according to any one of embodiments 1 to 2” means according to any one of embodiments 1, 1.1, 1.2, 1.3, 2.

Various embodiments of the invention are described herein. It will be recognized that features specified in each embodiment may be combined with other specified features to provide further embodiments.

### Embodiment 1.1

A compound of formula (I)

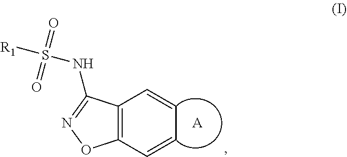

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

A is selected from the group consisting of

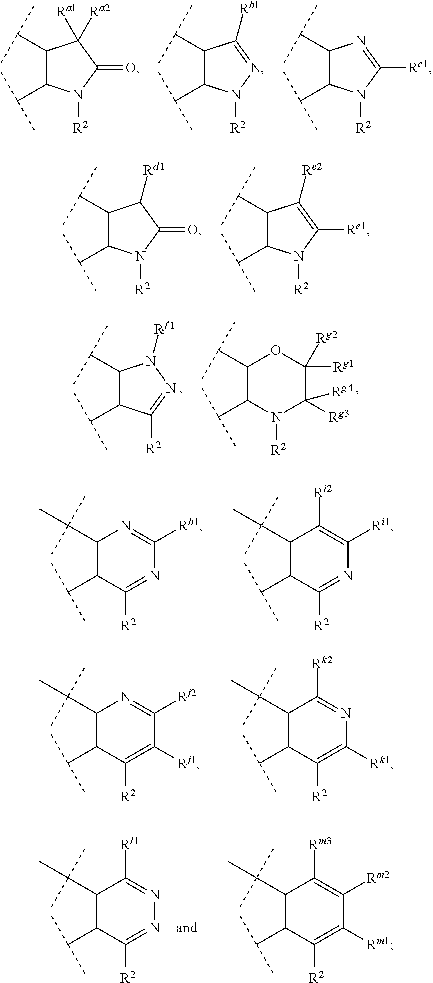

R^{a1} and R^{a2} are independently selected from the group consisting of H, C₁₋₄ alkyl, C₁₋₄ haloalkyl, halogen and CN;

or R^{a1} and R^{a2} form together with the carbon atom to which they are attached a C₃₋₆ cycloalkyl ring;

R^{b1} is selected from the group consisting of H, C₁₋₄ alkyl, C₁₋₄ haloalkyl, halogen and CN;

R^{c1} is selected from the group consisting of H, C₁₋₄ alkyl, C₁₋₄ haloalkyl, halogen and CN;

R^{d1} is selected from the group consisting of H, C₁₋₄ alkyl and C₁₋₄ haloalkyl;

R^{e1} is selected from the group consisting of H, C₁₋₄ alkyl, C₁₋₄ haloalkyl, halogen and CN;

R^{e2} is selected from the group consisting of H, C₁₋₄ alkyl, C₁₋₄ haloalkyl, halogen, CN and —C(═O)NR^{e21}R^{e22};

R^{e21} and R^{e22} are independently selected from H and C₁₋₄ alkyl;

R^{f1} selected from the group consisting of H, C₁₋₄ alkyl and C₁₋₄ haloalkyl;

R^{g1} and R^{g2} are independently selected from the group consisting of H, C₁₋₄ alkyl and C₁₋₄ haloalkyl;

or R^{g1} and R^{g2} form together with the carbon atom to which they are attached a C₃₋₆ cycloalkyl ring;

R^{g3} and R^{g4} are independently selected from the group consisting of H, C₁₋₄ alkyl, C₁₋₄ haloalkyl, halogen and CN;

or R^{g3} and R^{g4} form together an oxo group or together with the carbon atom to which they are attached a C₃₋₆ cycloalkyl ring;

R^{h1}, Rⁱ¹, Rⁱ², R^{j1}, R^{j2}, R^{k1}, R^{k2}, R^{l1}, R^{m1}, R^{m2} and R^{m3} are independently selected from the group consisting of H, C₁₋₄ alkyl, C₁₋₄ haloalkyl, halogen and CN;

R² is selected from the group consisting of

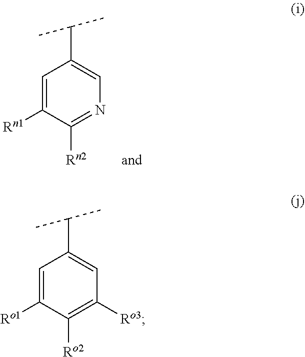

Rⁿ¹ is selected from the group consisting of H, halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen; n is independently selected from the group consisting of 0, 1 and 2;

R^{o3} is selected from the group consisting of halogen and CN;

with the proviso that N-(7′-(5-chloro-6-isobutoxypyridin-3-yl)-6′-oxo-6′,7′-dihydrospiro[cyclopropane-1,5′-isoxazolo[4,5-f]indol]-3′-yl)azetidine-1-sulfonamide is excluded;

or a pharmaceutically acceptable salt thereof.

### Embodiment 1.2

A compound of formula (I)

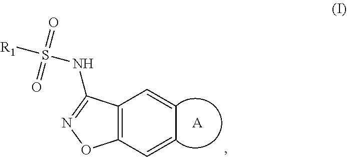

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl; A is selected from the group consisting of

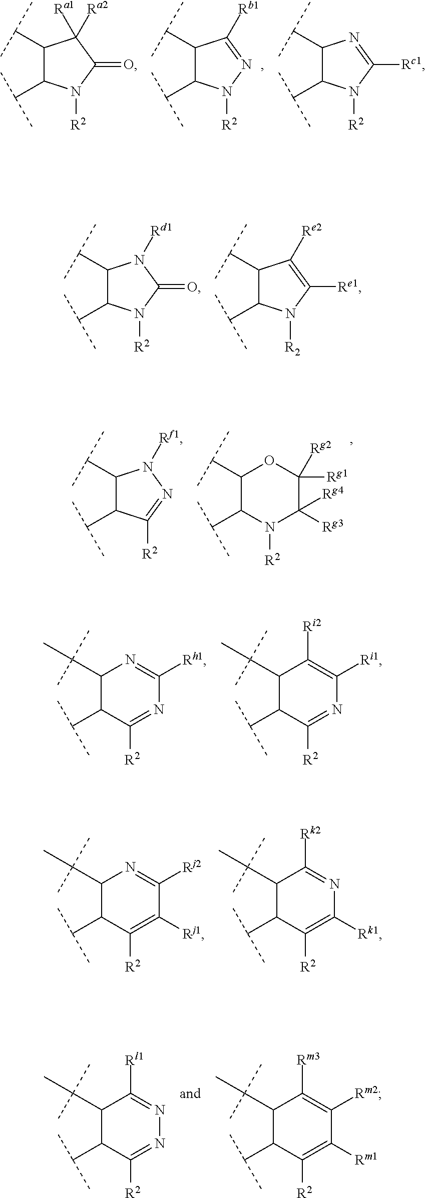

R^{a1} and R^{a2} are independently selected from the group consisting of H, C₁₋₄ alkyl, C₁₋₄ haloalkyl, halogen and CN;

or R^{a1} and R^{a2} form together with the carbon atom to which they are attached a C₃₋₆ cycloalkyl ring;

R^{b1} selected from the group consisting of H, C₁₋₄ alkyl, C₁₋₄ haloalkyl, halogen and CN;

R^{c1} selected from the group consisting of H, C₁₋₄ alkyl, C₁₋₄ haloalkyl, halogen and CN;

R^{d1} is selected from the group consisting of H, C₁₋₄ alkyl and C₁₋₄ haloalkyl;

R^{e1} is selected from the group consisting of H, C₁₋₄ alkyl, C₁₋₄ haloalkyl, halogen and CN;

R^{e2} is selected from the group consisting of H, C₁₋₄ alkyl, C₁₋₄ haloalkyl, halogen, CN and —C(═O)NR^{e21}R^{e22};

R^{e21} and R^{e22} are independently selected from H and C₁₋₄ alkyl;

R^{f1} selected from the group consisting of H, C₁₋₄ alkyl and C₁₋₄ haloalkyl;

R^{g1} and R^{g2} are independently selected from the group consisting of H, C₁₋₄ alkyl and C₁₋₄ haloalkyl;

or R^{g1} and R^{g2} form together with the carbon atom to which they are attached a C₃₋₆ cycloalkyl ring;

R^{g3} and R^{g4} are independently selected from the group consisting of H, C₁₋₄ alkyl, C₁₋₄ haloalkyl, halogen and CN;

or R^{g3} and R^{g4} form together an oxo group or together with the carbon atom to which they are attached a C₃₋₆ cycloalkyl ring;

R^{h1}, Rⁱ¹, Rⁱ², R^{j1}, R^{j2}, R^{k1}, R^{k2}, R^{l1}, R^{m1}, R^{m2} and R^{m3} are independently selected from the group consisting of H, C₁₋₄ alkyl, C₁₋₄ haloalkyl, halogen and CN;

R² is selected from the group consisting of

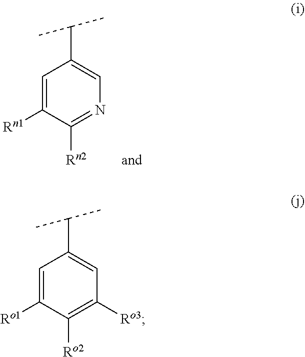

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 1.3

A compound of formula (I)

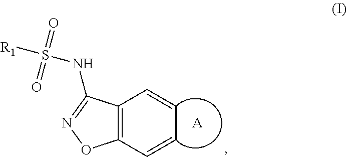

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

A is selected from the group consisting of

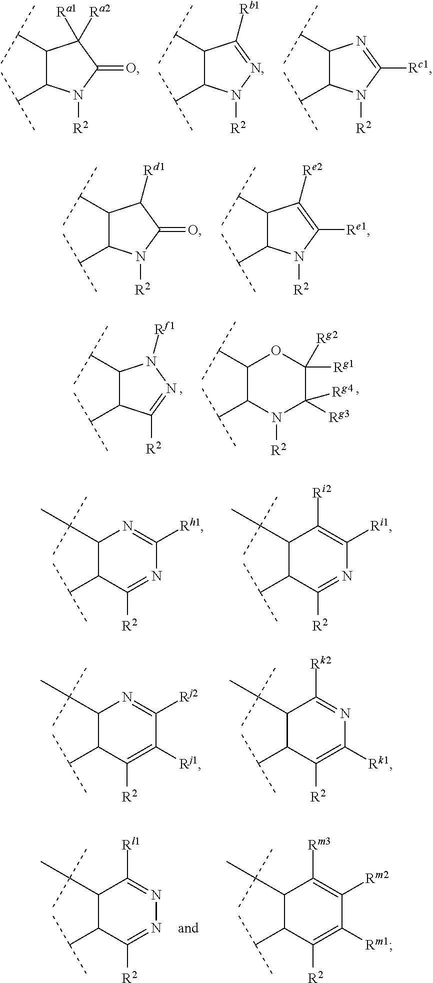

R^{a1} and R^{a2} are independently selected from the group consisting of H, C₁₋₄ alkyl, C₁₋₄ haloalkyl, halogen and CN;

or R^{a1} and R^{a2} form together with the carbon atom to which they are attached a C₃₋₆ cycloalkyl ring;

R^{b1} selected from the group consisting of H, C₁₋₄ alkyl, C₁₋₄ haloalkyl, halogen and CN;

R^{c1} selected from the group consisting of H, C₁₋₄ alkyl, C₁₋₄ haloalkyl, halogen and CN;

R^{d1} is selected from the group consisting of H, C₁₋₄ alkyl and C₁₋₄ haloalkyl;

R^{e1} is selected from the group consisting of H, C₁₋₄ alkyl, C₁₋₄ haloalkyl, halogen and CN;

R^{e2} is selected from the group consisting of H, C₁₋₄ alkyl, C₁₋₄ haloalkyl, halogen, CN and —C(═O)NR^{e21}R^{e22};

R^{e21} and R^{e22} are independently selected from H and C₁₋₄ alkyl;

R^{f1} selected from the group consisting of H, C₁₋₄ alkyl and C₁₋₄ haloalkyl;

R^{g1} and R^{g2} are independently selected from the group consisting of H, C₁₋₄ alkyl and C₁₋₄ haloalkyl;

or R^{g1} and R^{g2} form together with the carbon atom to which they are attached a C₃₋₆ cycloalkyl ring;

R^{g3} and R^{g4} are independently selected from the group consisting of H, C₁₋₄ alkyl, C₁₋₄ haloalkyl, halogen and CN;

or R^{g3} and R^{g4} form together an oxo group or together with the carbon atom to which they are attached a C₃₋₆ cycloalkyl ring;

R^{h1}, Rⁱ¹, Rⁱ², R^{j1}, R^{j2}, R^{k1}, R^{k2}, R^{l1}, R^{m1}, R^{m2} and R^{m3} are independently selected from the group consisting of H, C₁₋₄ alkyl, C₁₋₄ haloalkyl, halogen and CN;

R² is selected from the group consisting of

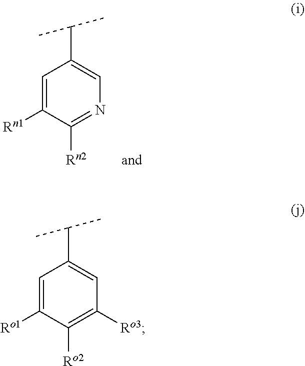

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

with the proviso that N-(7′-(5-chloro-6-isobutoxypyridin-3-yl)-6′-oxo-6′,7′-dihydrospiro[cyclopropane-1,5′-isoxazolo[4,5-f]indol]-3′-yl)azetidine-1-sulfonamide is excluded;

or a pharmaceutically acceptable salt thereof.

### Embodiment 2

A compound of formula (I)

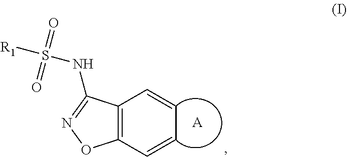

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

A is selected from the group consisting of

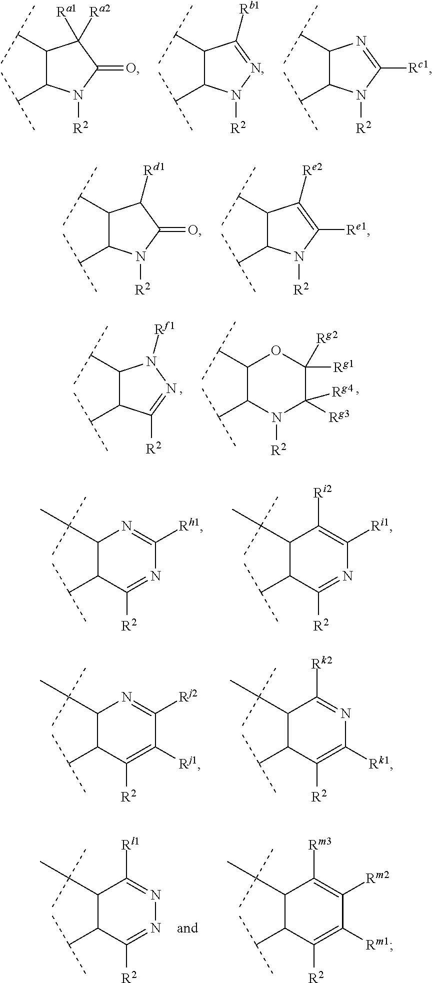

R^{a1} and R^{a2} are independently selected from the group consisting of H and C₁₋₄ alkyl;

or R^{a1} and R^{a2} form together with the carbon atom to which they are attached a C₃₋₆ cycloalkyl ring;

R^{b1} selected from the group consisting of H and C₁₋₄ alkyl;

R^{c1} selected from the group consisting of H and C₁₋₄ haloalkyl;

R^{d1} is selected from the group consisting of H and C₁₋₄ alkyl;

R^{e1} is selected from the group consisting of H, C₁₋₄ alkyl, C₁₋₄ haloalkyl and CN;

R^{e2} is selected from the group consisting of H, C₁₋₄ alkyl, C₁₋₄ haloalkyl, CN and —C(═O)NR^{e21}R^{e22};

R^{e21} and R^{e22} are independently selected from H and C₁₋₄ alkyl;

R^{f1} selected from the group consisting of H and C₁₋₄ alkyl;

R^{g1} and R^{g2} are independently selected from the group consisting of H and C₁₋₄ alkyl;

or R^{g1} and R^{g2} form together with the carbon atom to which they are attached a C₃₋₆ cycloalkyl ring;

R^{g3} and R^{g4} are independently selected from the group consisting of H, C₁₋₄ alkyl and C₁₋₄ haloalkyl;

or R^{g3} and R^{g4} form together an oxo group or together with the carbon atom to which they are attached a C₃₋₆ cycloalkyl ring;

R^{h1}, Rⁱ¹, Rⁱ², R^{j1}, R^{j2}, R^{k1}, R^{k2}, R^{l1}, R^{m1}, R^{m2} and R^{m3} are independently selected from the group consisting of H and C₁₋₄ alkyl;

R² is selected from the group consisting of

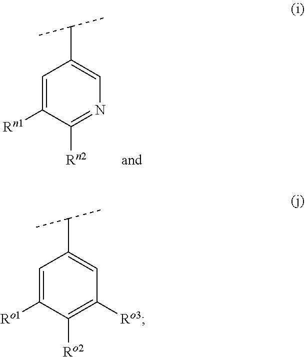

Rⁿ¹ is halogen;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 3

A compound or salt according to embodiment 1 or 2, wherein

R² is

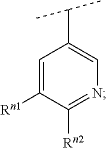

Rⁿ¹ is halogen;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

n is independently selected from the group consisting of 0, 1 and 2;

Rⁿ²¹ and Rⁿ²² are independently selected from the group consisting of H, C₁₋₄ alkyl and halogen;

or a pharmaceutically acceptable salt thereof.

### Embodiment 4

A compound or salt according to embodiment 3, wherein

Rⁿ¹ is chloro or fluoro and Rⁿ² is selected from the group consisting of

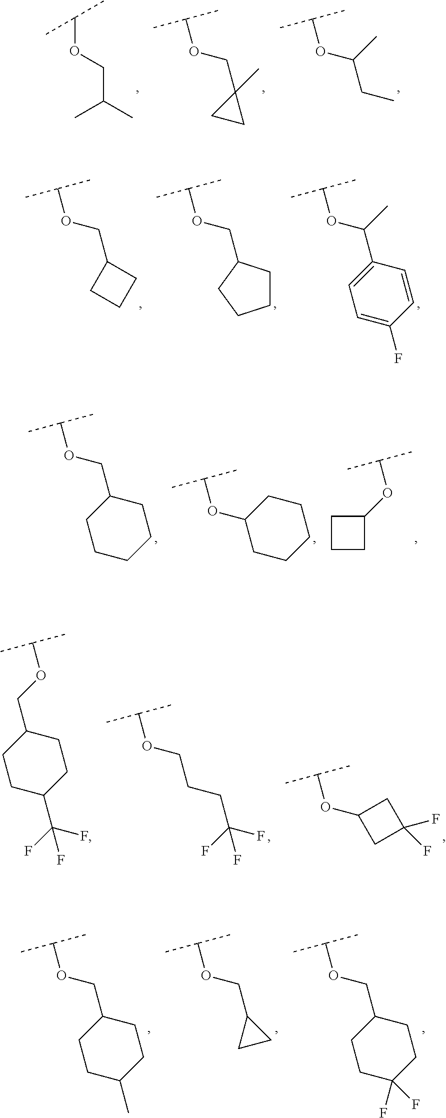

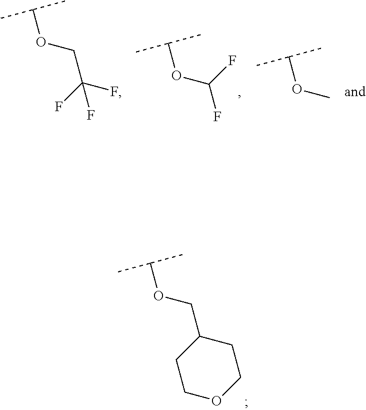

or a pharmaceutically acceptable salt thereof.

### Embodiment 5

A compound or salt according to embodiment 1 or 2, wherein

R² is

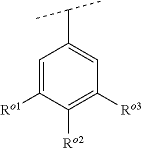

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹Rⁿ²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 6

A compound or salt according to embodiment 1 or 2, wherein

R² is

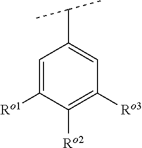

R^{o1} is selected from the group consisting of H and chloro;

R^{o2} is selected from the group consisting of chloro and C₁₋₄ alkoxy;

R^{o3} is selected from the group consisting of chloro, fluoro and CN;

or a pharmaceutically acceptable salt thereof.

### Embodiment 7

A compound or salt according to embodiment 5, wherein

R^{o1} is H or chloro, R^{o3} is chloro, fluoro or CN and R^{o2} is selected from the group consisting of chloro and

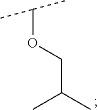

or a pharmaceutically acceptable salt thereof.

### Embodiment 8

A compound according to any one of embodiments 1 to 7 of formula (Ia)

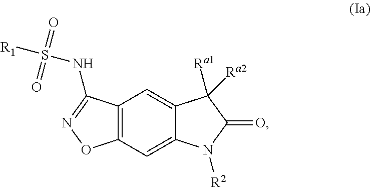

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{a1} and R^{a2} are independently selected from the group consisting of H, C₁₋₄ alkyl, C₁₋₄ haloalkyl, halogen and CN;

or R^{a1} and R^{a2} form together with the carbon atom to which they are attached a C₃₋₆ cycloalkyl ring;

R² is selected from the group consisting of

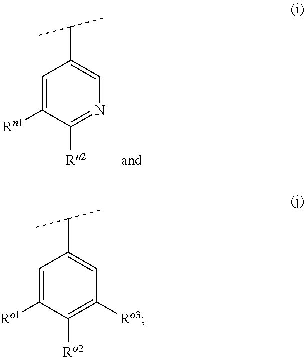

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}CR^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 9

A compound according to any one of embodiments 1 to 7 of formula (Ib)

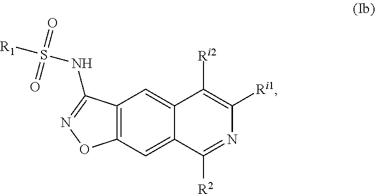

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

Rⁱ¹ and Rⁱ² are independently selected from the group consisting of H, C₁₋₄ alkyl, C₁₋₄ haloalkyl, halogen and CN;

R² is selected from the group consisting of

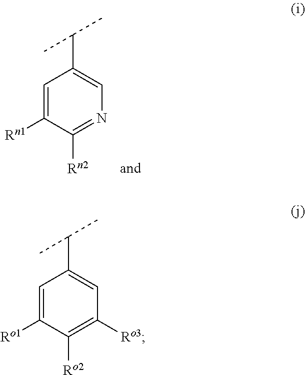

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 10

A compound according to any one of embodiments 1 to 7 of formula (Ic)

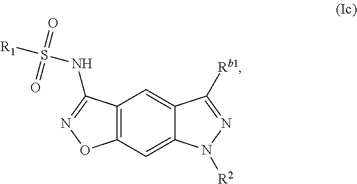

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{b1} selected from the group consisting of H, C₁₋₄ alkyl, C₁₋₄ haloalkyl, halogen and CN;

R² is selected from the group consisting of

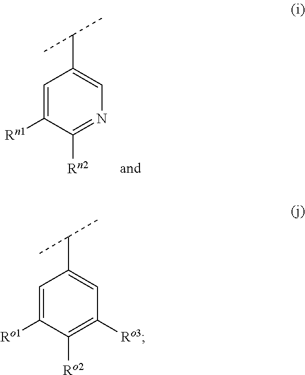

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 11

A compound according to any one of embodiments 1 to 7 of formula (Id)

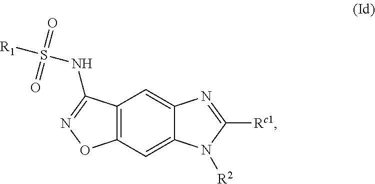

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{c1} selected from the group consisting of H, C₁₋₄ alkyl, C₁₋₄ haloalkyl, halogen and CN;

R² is selected from the group consisting of

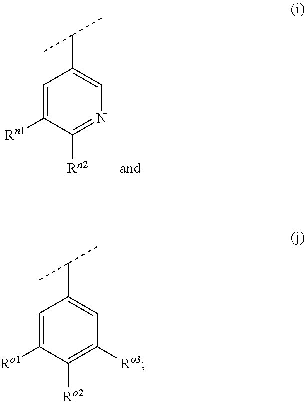

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 12

A compound according to any one of embodiments 1 to 7 of formula (Ie)

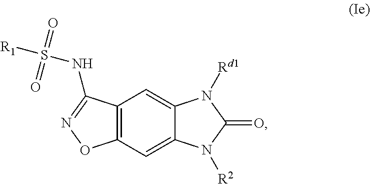

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{d1} is selected from the group consisting of H, C₁₋₄ alkyl and C₁₋₄ haloalkyl;

R² is selected from the group consisting of

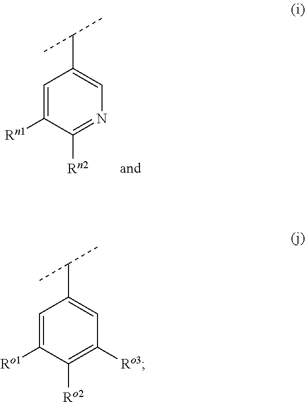

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹Rⁿ²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 13

A compound according to any one of embodiments 1 to 7 of formula (If)

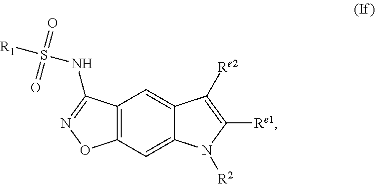

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{e1} is selected from the group consisting of H, C₁₋₄ alkyl, C₁₋₄ haloalkyl, halogen and CN;

R^{e2} is selected from the group consisting of H, C₁₋₄ alkyl, C₁₋₄ haloalkyl, halogen, CN and —C(═O)NR^{e21}R^{e22};

R^{e21} and R^{e22} are independently selected from H and C₁₋₄ alkyl;

R² is selected from the group consisting of

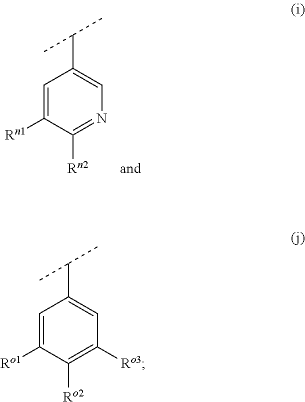

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹Rⁿ²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 14

A compound according to any one of embodiments 1 to 7 of formula (Ig)

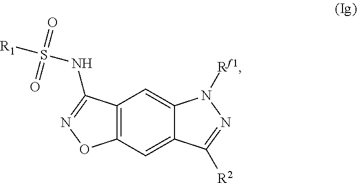

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{f1} selected from the group consisting of H, C₁₋₄ alkyl and C₁₋₄ haloalkyl;

R² is selected from the group consisting of

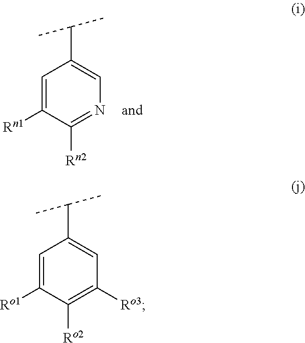

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 15

A compound according to any one of embodiments 1 to 7 of formula (Ih)

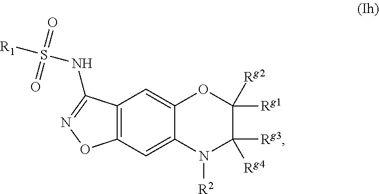

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{g1} and R^{g2} are independently selected from the group consisting of H, C₁₋₄ alkyl and C₁₋₄ haloalkyl;

or R^{g1} and R^{g2} form together with the carbon atom to which they are attached a C₃₋₆ cycloalkyl ring;

R^{g3} and R^{g4} are independently selected from the group consisting of H, C₁₋₄ alkyl, C₁₋₄ haloalkyl, halogen and CN;

or R^{g3} and R^{g4} form together an oxo group;

R² is selected from the group consisting of

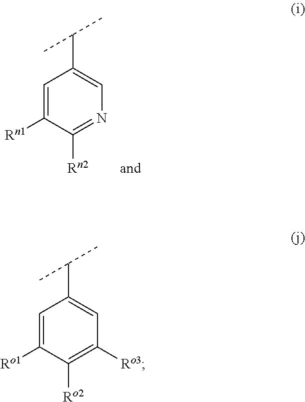

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹Rⁿ²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 16

A compound according to any one of embodiments 1 to 7 of formula (Ii)

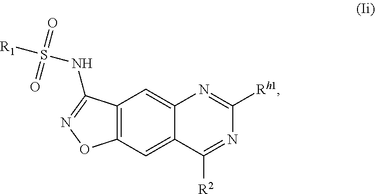

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{h1} is selected from the group consisting of H, C₁₋₄ alkyl, C₁₋₄ haloalkyl, halogen and CN;

R² is selected from the group consisting of

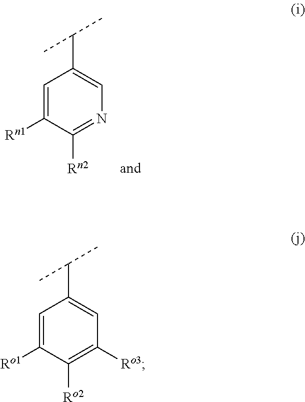

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹Rⁿ²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 17

A compound according to any one of embodiments 1 to 7 of formula (Ij)

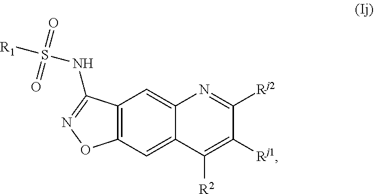

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{j1} and R^{j2} are independently selected from the group consisting of H, C₁₋₄ alkyl, C₁₋₄ haloalkyl, halogen and CN;

R² is selected from the group consisting of

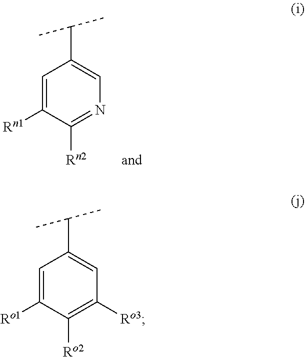

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 18

A compound according to any one of embodiments 1 to 7 of formula (Ik)

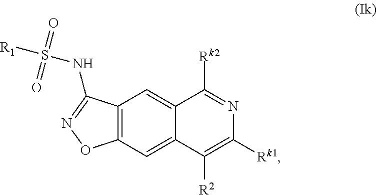

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{k1} and R^{k2} are independently selected from the group consisting of H, C₁₋₄ alkyl, C₁₋₄ haloalkyl, halogen and CN;

R² is selected from the group consisting of

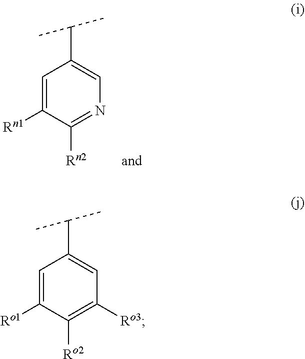

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 19

A compound according to any one of embodiments 1 to 7 of formula (In)

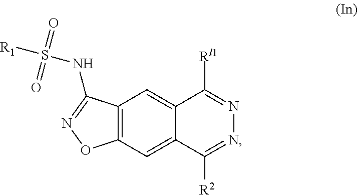

wherein

R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;

R¹¹ and R¹² are independently selected from the group consisting H and C₁₋₄ alkyl;

R^{l1} is selected from the group consisting of H, C₁₋₄ alkyl, C₁₋₄ haloalkyl, halogen and CN;

R² is selected from the group consisting of

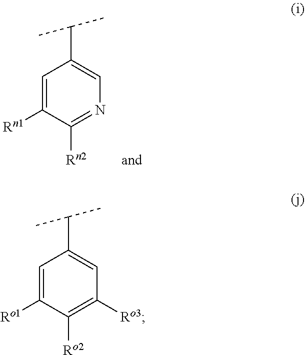

Rⁿ¹ is selected from the group consisting of halogen and CN;

Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;

Rⁿ²¹ and Rⁿ²² are independently selected from the group consisting of H, C₁₋₄ alkyl and halogen;

R^{o1} is selected from the group consisting of H and halogen;

R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹Rⁿ²²)ₙ-4 to 6-membered heterocyclyl and phenyl, wherein the cycloalkyl, heterocyclyl and phenyl are unsubstituted or substituted by 1 to 3 substituents independently selected from C₁₋₄ alkyl, halogen and C₁₋₄ haloalkyl;

R^{o21} and R^{o22} are independently selected from the group consisting of H, C₁₋₄ alkyl and halogen;

n is independently selected from the group consisting of 0, 1 and 2;

R^{o3} is selected from the group consisting of halogen and CN;

or a pharmaceutically acceptable salt thereof.

### Embodiment 20

A compound or salt according to embodiment 1 selected from the group consisting of
N-(8-(5-chloro-6-isobutoxypyridin-3-yl)naphtho[2,3-d]isoxazol-3-yl)cyclopropanesulfonamide;
N-(7-(5-chloro-6-isobutoxypyridin-3-yl)-5-(trifluoromethyl)-7H-isoxazolo[4,5-f]indol-3-yl)methanesulfonamide;
N-(7-(5-chloro-6-isobutoxypyridin-3-yl)-5-methyl-7H-isoxazolo[4,5-f]indazol-3-yl)cyclopropanesulfonamide;
N-(7-(5-chloro-6-isobutoxypyridin-3-yl)-6-(trifluoromethyl)-7H-isoxazolo[4,5-f]indol-3-yl)methanesulfonamide;
N-(7′-(5-chloro-6-isobutoxypyridin-3-yl)-6′-oxo-6′,7′-dihydrospiro[cyclobutane-1,5′-isoxazolo[4,5-f]indole]-3′-yl)cyclopropanesulfonamide;
N-(7-(5-chloro-6-isobutoxypyridin-3-yl)-5-(trifluoromethyl)-7H-isoxazolo[4,5-f]indol-3-yl)cyclopropanesulfonamide;
N-(7′-(5-chloro-6-((1-methylcyclopropyl)methoxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-((1-methylcyclopropyl)methoxy)pyridin-3-yl)-6′-oxo-6′,7′-dihydrospiro[cyclopropane-1,5′-isoxazolo[4,5-f]indole]-3′-yl)methanesulfonamide;
N-(7-(5-chloro-6-isobutoxypyridin-3-yl)-5,5-dimethyl-6-oxo-6,7-dihydro-5H-isoxazolo[4,5-f]indol-3-yl)cyclopropanesulfonamide;
N-(7-(5-chloro-6-isobutoxypyridin-3-yl)-5-methyl-7H-isoxazolo[4,5-f]indol-3-yl)cyclopropanesulfonamide;
N-(7′-(6-sec-butoxy-5-chloropyridin-3-yl)-6′-oxo-6′,7′-dihydrospiro[cyclopropane-1,5′-isoxazolo[4,5-f]indole]-3′-yl)cyclopropanesulfonamide;
N-(7-(5-chloro-6-isobutoxypyridin-3-yl)-6′-oxo-6′,7′-dihydrospiro[cyclopropane-1,5-isoxazolo[4,5-f]indole]-3′-yl)cyclopropanesulfonamide;
N-(7′-(5-chloro-6-(cyclobutylmethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-(cyclopentylmethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7-(5-chloro-6-isobutoxypyridin-3-yl)-5-methyl-6-oxo-6,7-dihydro-5H-imidazo[4′,5′:4,5]benzo[1,2-d]isoxazol-3-yl)methanesulfonamide;
N-(7-(5-chloro-6-isobutoxypyridin-3-yl)-5-methyl-7H-isoxazolo[4,5-f]indazol-3-yl)methanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)propane-2-sulfonamide;
N-(7′-(5-chloro-6-isobutoxypyridin-3-yl)-6′-oxo-6′,7′-dihydrospiro[cyclopropane-1,5-isoxazolo[4,5-f]indole]-3′-yl)ethanesulfonamide;
N-(7-(5-chloro-6-isobutoxypyridin-3-yl)-5-methyl-6-oxo-6,7-dihydro-5H-imidazo[4′,5′:4,5]benzo[1,2-d]isoxazol-3-yl)cyclopropanesulfonamide;
N-(7′-(3-chloro-5-fluoro-4-isobutoxyphenyl)-6′-oxo-6′,7′-dihydrospiro[cyclopropane-1,5′-isoxazolo[4,5-f]indole]-3′-yl)cyclopropanesulfonamide;
(S)—N-(7′-(5-chloro-6-(1-(4-fluorophenyl)ethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7-(5-chloro-6-isobutoxypyridin-3-yl)-6-(trifluoromethyl)-7H-imidazo[4′,5′:4,5]benzo[1,2-d]isoxazol-3-yl)cyclopropanesulfonamide;
N-(7′-(3,4-dichlorophenyl)-5-methyl-7H-isoxazolo[4,5-f]indazol-3-yl)cyclopropanesulfonamide;
N-(7′-(5-chloro-6-(cyclohexylmethoxy)pyridin-3-yl)-6′-oxo-6′,7′-dihydrospiro[cyclopropane-1,5′-isoxazolo[4,5-f]indole]-3′-yl)methanesulfonamide;
N-(8′-(5-chloro-6-isobutoxypyridin-3-yl)-7-oxo-7′,8′-dihydrospiro[cyclopropane-1,6′-isoxazolo[5′,4′:4,5]benzo[1,2-b][1,4]oxazin]-3′-yl)cyclopropanesulfonamide;
N-(8-(5-chloro-6-isobutoxypyridin-3-yl)isoxazolo[4,5-g]isoquinolin-3-yl)cyclopropanesulfonamide;
N-(7′-(5-chloro-6-(cyclohexyloxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)methanesulfonamide;
N-(7′-(5-chloro-6-cyclobutoxypyridin-3-yl)-6′-oxo-6′,7′-dihydrospiro[cyclopropane-1,5-isoxazolo[4,5-f]indole]-3′-yl)cyclopropanesulfonamide;
N-(7′-(5-chloro-6-((4-(trifluoromethyl)cyclohexyl)methoxy)pyridin-3-yl)-6′-oxo-6′,7′-dihydrospiro[cyclopropane-1,5′-isoxazolo[4,5-f]indole]-3′-yl)methanesulfonamide;
N-(7′-(5-chloro-6-(4,4,4-trifluorobutoxyl)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-(3,3-difluorocyclobutoxyl)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-((4-methylcyclohexyl)methoxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-(cyclopropylmethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7-(5-chloro-6-isobutoxypyridin-3-yl)-5-cyano-7H-isoxazolo[4,5-f]indol-3-yl)cyclopropanesulfonamide;
N-(7′-(5-chloro-6-((4,4-difluorocyclohexyl)methoxy)pyridin-3-yl)-6′-oxo-6′,7′-dihydrospiro[cyclopropane-1,5′-isoxazolo[4,5-f]indole]-3′-yl)methanesulfonamide;
N-(8-(5-chloro-6-isobutoxypyridin-3-yl)isoxazolo[4,5-g]isoquinolin-3-yl)methanesulfonamide;
N-(7′-(5-fluoro-6-isobutoxypyridin-3-yl)-6′-oxo-6′,7′-dihydrospiro[cyclopropane-1,5′-isoxazolo[4,5-f]indole]-3′-yl)cyclopropanesulfonamide;
N-(7-(5-chloro-6-isobutoxypyridin-3-yl)-5-methyl-5H-isoxazolo[5,4-f]indazol-3-yl)cyclopropanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)-1,1,1-trifluoromethanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)pyrrolidine-1-sulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)thiophene-2-sulfonamide;
N-(7′-(5-chloro-6-isobutoxypyridin-3-yl)-6′-oxo-6′,7′-dihydrospiro[cyclopentane-1,5′-isoxazolo[4,5-f]indole]-3′-yl)cyclopropanesulfonamide;
N-(7′-(5-chloro-6-(2,2,2-trifluoroethoxyl)pyridin-3-yl)-6′-oxo-6′,7′-dihydrospiro[cyclopropane-1,5′-isoxazolo[4,5-f]indole]-3′-yl)cyclopropanesulfonamide;
N-(8-(5-chloro-6-isobutoxypyridin-3-yl)-6-methylisoxazolo[4,5-g]isoquinolin-3-yl)cyclopropanesulfonamide;
N-(8-(5-chloro-6-isobutoxypyridin-3-yl)-7,8-dihydro-6H-isoxazolo[5′,4′:4,5]benzo[1,2-b][1,4]oxazin-3-yl)methanesulfonamide;
N-(8-(5-chloro-6-isobutoxypyridin-3-yl)isoxazolo[5,4-g]isoquinolin-3-yl)cyclopropanesulfonamide;
N-(7′-(5-chloro-6-(difluoromethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-isobutoxypyridin-3-yl)-6′-oxo-6′,7′-dihydrospiro[cyclohexane-1,5′-isoxazolo[4,5-f]indole]-3′-yl)cyclopropanesulfonamide;
N-(7′-(5-chloro-6-isobutoxypyridin-3-yl)-6′-oxo-6′,7′-dihydrospiro[cyclopropane-1,5′-isoxazolo[4,5-f]indol]-3′-yl)pyrrolidine-1-sulfonamide;
N-(7′-(3,4-dichlorophenyl)-6′-oxo-6′,7′-dihydrospiro[cyclopropane-1,5′-isoxazolo[4,5-f]indole]-3′-yl)methanesulfonamide;
N-(8-(3,4-dichlorophenyl)isoxazolo[4,5-g]isoquinolin-3-yl)cyclopropanesulfonamide;
N-(7′-(5-chloro-6-methoxypyridin-3-yl)-6′-oxo-6′,7′-dihydrospiro[cyclopropane-1,5′-isoxazolo[4,5-f]indole]-3′-yl)cyclopropanesulfonamide;
N-(8-(5-chloro-6-isobutoxypyridin-3-yl)isoxazolo[5,4-g]quinolin-3-yl)cyclopropanesulfonamide;
N-(7′-(3-chloro-5-cyano-4-isobutoxyphenyl)-6′-oxo-6′,7′-dihydrospiro[cyclopropane-1,5′-isoxazolo[4,5-f]indole]-3′-yl)methanesulfonamide;
N-(8-(5-chloro-6-isobutoxypyridin-3-yl)isoxazolo[4,5-g]isoquinolin-3-yl)N—N-dimethylsulfamide;
7-(5-chloro-6-isobutoxypyridin-3-yl)-3-(cyclopropanesulfonamido)-7H-isoxazolo[4,5-f]indole-5-carboxamide;
N-(6-(5-chloro-6-isobutoxypyridin-3-ylamino)benzo[d]isoxazol-3-yl)cyclopropanesulfonamide;
N-(8-(5-chloro-6-isobutoxypyridin-3-yl)-5-methylisoxazolo[4,5-g]isoquinolin-3-yl)cyclopropanesulfonamide;
N-(7′-(5-chloro-6-isobutoxypyridin-3-yl)-6′-oxo-6′,7′-dihydrospiro[cyclopropane-1,5′-isoxazolo[4,5-f]indol]-3′-yl)dimethylamino-1-sulfonamide;
N-(7′-(5-chloro-6-((tetrahydro-2H-pyran-4-yl)methoxy)pyridin-3-yl)-6′-oxo-6′,7′-dihydrospiro[cyclopropane-1,5′-isoxazolo[4,5-f]indole]-3′-yl)methanesulfonamide;
N-(8-(5-chloro-6-isobutoxypyridin-3-yl)isoxazolo[5,4-g]isoquinolin-3-yl)methanesulfonamide;
N-(6′-(5-chloro-6-isobutoxypyridin-3-yl)-7′-oxo-6′,7′-dihydrospiro[cyclopropane-1,8′-isoxazolo[5,4-e]indole]-3′-yl)methanesulfonamide; and
N-(7′-(5-chloro-6-isobutoxypyridin-3-yl)-6′-oxo-6′,7′-dihydrospiro[cyclopropane-1,5′-isoxazolo[4,5-f]indole]-3′-yl)benzenesulfonamide;

or a pharmaceutically acceptable salt thereof.

### Embodiment 21

A compound or salt according to embodiment 1 selected from the group consisting of
N-(8-(5-chloro-6-isobutoxypyridin-3-yl)naphtho[2,3-d]isoxazol-3-yl)cyclopropanesulfonamide;
N-(7-(5-chloro-6-isobutoxypyridin-3-yl)-5-(trifluoromethyl)-7H-isoxazolo[4,5-f]indol-3-yl)methanesulfonamide;
N-(7-(5-chloro-6-isobutoxypyridin-3-yl)-5-methyl-7H-isoxazolo[4,5-f]indazol-3-yl)cyclopropanesulfonamide;
N-(7-(5-chloro-6-isobutoxypyridin-3-yl)-6-(trifluoromethyl)-7H-isoxazolo[4,5-f]indol-3-yl)methanesulfonamide;
N-(7′-(5-chloro-6-isobutoxypyridin-3-yl)-6′-oxo-6′,7′-dihydrospiro[cyclobutane-1,5′-isoxazolo[4,5-f]indole]-3′-yl)cyclopropanesulfonamide;
N-(7-(5-chloro-6-isobutoxypyridin-3-yl)-5-(trifluoromethyl)-7H-isoxazolo[4,5-f]indol-3-yl)cyclopropanesulfonamide;
N-(7′-(5-chloro-6-((1-methylcyclopropyl)methoxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-((1-methylcyclopropyl)methoxy)pyridin-3-yl)-6′-oxo-6′,7′-dihydrospiro[cyclopropane-1,5′-isoxazolo[4,5-f]indole]-3′-yl)methanesulfonamide;
N-(7-(5-chloro-6-isobutoxypyridin-3-yl)-5,5-dimethyl-6-oxo-6,7-dihydro-5H-isoxazolo[4,5-f]indol-3-yl)cyclopropanesulfonamide;
N-(7-(5-chloro-6-isobutoxypyridin-3-yl)-5-methyl-7H-isoxazolo[4,5-f]indol-3-yl)cyclopropanesulfonamide;
N-(7′-(6-sec-butoxy-5-chloropyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-(cyclobutylmethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-(cyclopentylmethoxy)pyridin-3-yl)-6′-oxo-6′,7′-dihydrospiro[cyclopropane-1,5-isoxazolo[4,5-f]indole]-3′-yl)cyclopropanesulfonamide;
N-(7-(5-chloro-6-isobutoxypyridin-3-yl)-5-methyl-7H-isoxazolo[4,5-f]indazol-3-yl)methanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)methanesulfonamide;
N-(7′-(5-chloro-6-isobutoxypyridin-3-yl)-6′-oxo-6′,7′-dihydrospiro[cyclopropane-1,5-isoxazolo[4,5-f]indole]-3′-yl)propane-2-sulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)ethanesulfonamide;
N-(7′-(3-chloro-5-fluoro-4-isobutoxyphenyl)-6′-oxo-6′,7′-dihydrospiro[cyclopropane-1,5-isoxazolo[4,5-f]indole]-3′-yl)cyclopropanesulfonamide;
(S)—N-(7′-(5-chloro-6-(1-(4-fluorophenyl)ethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7-(3,4-dichlorophenyl)-5-methyl-7H-isoxazolo[4,5-f]indazol-3-yl)cyclopropanesulfonamide;
N-(8-(5-chloro-6-isobutoxypyridin-3-yl)isoxazolo[4,5-g]isoquinolin-3-yl)cyclopropanesulfonamide;
N-(7′-(5-chloro-6-(cyclohexylmethoxy)pyridin-3-yl)-6′-oxo-6′,7′-dihydrospiro[cyclopropane-1,5′-isoxazolo[4,5-f]indole]-3′-yl)methanesulfonamide;

or a pharmaceutically acceptable salt thereof.

### Embodiment 22

A compound or salt according to embodiment 1 selected from the group consisting of
N-(8-(5-chloro-6-isobutoxypyridin-3-yl)naphtho[2,3-d]isoxazol-3-yl)cyclopropanesulfonamide;
N-(7-(5-chloro-6-isobutoxypyridin-3-yl)-5-(trifluoromethyl)-7H-isoxazolo[4,5-f]indol-3-yl)methanesulfonamide;
N-(7′-(5-chloro-6-isobutoxypyridin-3-yl)-6′-oxo-6′,7′-dihydrospiro[cyclobutane-1,5′-isoxazolo[4,5-f]indole]-3′-yl)cyclopropanesulfonamide;
N-(7′-(5-chloro-6-((1-methylcyclopropyl)methoxy)pyridin-3-yl)-6′-oxo-6′,7′-dihydrospiro[cyclopropane-1,5′-isoxazolo[4,5-f]indole]-3′-yl)methanesulfonamide;
N-(7-(5-chloro-6-isobutoxypyridin-3-yl)-5,5-dimethyl-6-oxo-6,7-dihydro-5H-isoxazolo[4,5-f]indol-3-yl)cyclopropanesulfonamide;
N-(7-(5-chloro-6-isobutoxypyridin-3-yl)-5-methyl-7H-isoxazolo[4,5-f]indol-3-yl)cyclopropanesulfonamide;
N-(7′-(6-sec-butoxy-5-chloropyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-(cyclobutylmethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-(cyclopentylmethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-isobutoxypyridin-3-yl)-5-methyl-7H-isoxazolo[4,5-f]indazol-3-yl)methanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)methanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)propane-2-sulfonamide;
N-(7-(3,4-dichlorophenyl)-5-methyl-7H-isoxazolo[4,5-f]indazol-3-yl)cyclopropanesulfonamide;
N-(8-(5-chloro-6-isobutoxypyridin-3-yl)isoxazolo[4,5-g]isoquinolin-3-yl)cyclopropanesulfonamide;
N-(7′-(5-chloro-6-(cyclohexylmethoxy)pyridin-3-yl)-6′-oxo-6′,7′-dihydrospiro[cyclopropane-1,5′-isoxazolo[4,5-f]indole]-3′-yl)methanesulfonamide;

or a pharmaceutically acceptable salt thereof.

### Embodiment 23

A compound according to embodiment 1 which is
N-(7′-(5-chloro-6-isobutoxypyridin-3-yl)-6′-oxo-6′,7′-dihydrospiro[cyclopropane-1,5′-isoxazolo[4,5-f]indol]-3′-yl)methanesulfonamide

of formula

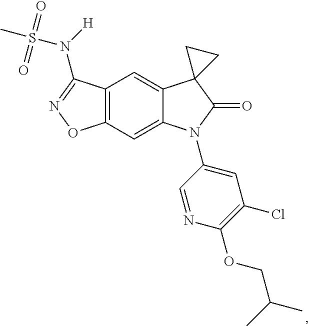 [SMILES: CC(C)COc1ncc(N2C(=O)C3(CC3)c3cc4c(NS(C)(=O)=O)noc4cc32)cc1Cl]

or a pharmaceutically acceptable salt thereof.

### Embodiment 24

A compound according to embodiment 1 which is
N-(7′-(5-chloro-6-isobutoxypyridin-3-yl)-6′-oxo-6′,7′-dihydrospiro[cyclopropane-1,5′-isoxazolo[4,5-f]indol]-3′-yl)cyclopropanesulfonamide of formula

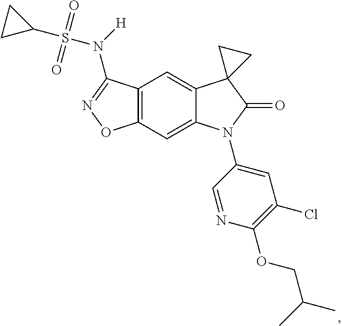 [SMILES: CC(C)COc1ncc(N2C(=O)C3(CC3)c3cc4c(NS(=O)(=O)C5CC5)noc4cc32)cc1Cl]

or a pharmaceutically acceptable salt thereof.

### Embodiment 25

A compound according to embodiment 1 which is N-(8-(5-chloro-6-isobutoxypyridin-3-yl)isoxazolo[4,5-g]isoquinolin-3-yl)cyclopropanesulfonamide of formula

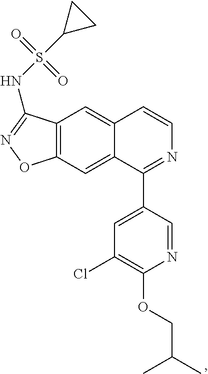 [SMILES: CC(C)COc1ncc(-c2nccc3cc4c(NS(=O)(=O)C5CC5)noc4cc23)cc1Cl]

or a pharmaceutically acceptable salt thereof.

### Embodiment 26

A compound according to embodiment 1 which is N-(7′-(5-chloro-6-(cyclopentylmethoxy)pyridin-3-yl)-6′-oxo-6′,7′-dihydrospiro[cyclopropane-1,5′-isoxazolo[4,5-f]indole]-3′-yl)cyclopropanesulfonamide of formula

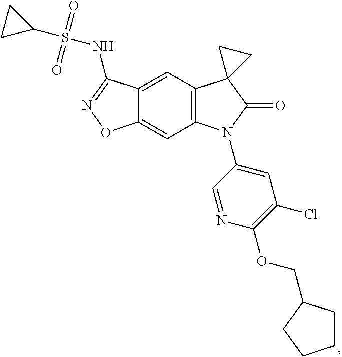 [SMILES: O=C1N(c2cnc(OCC3CCCC3)c(Cl)c2)c2cc3onc(NS(=O)(=O)C4CC4)c3cc2C12CC2]

or a pharmaceutically acceptable salt thereof.

### Embodiment 27

A compound according to embodiment 1 which is N-(7′-(5-chloro-6-isobutoxypyridin-3-yl)-6′-oxo-6′,7′-dihydrospiro[cyclobutane-1,5′-isoxazolo[4,5-f]indole]-3′-yl)cyclopropanesulfonamide of formula

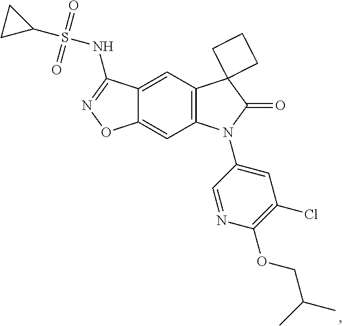 [SMILES: CC(C)COc1ncc(N2C(=O)C3(CCC3)c3cc4c(NS(=O)(=O)C5CC5)noc4cc32)cc1Cl]

or a pharmaceutically acceptable salt thereof.

### Embodiment 28

A compound according to embodiment 1 which is N-(7′-(5-chloro-6-(cyclopentylmethoxy)pyridin-3-yl)-6′-oxo-6′,7′-dihydrospiro[cyclopropane-1,5′-isoxazolo[4,5-f]indole]-3′-yl)cyclopropanesulfonamide of formula

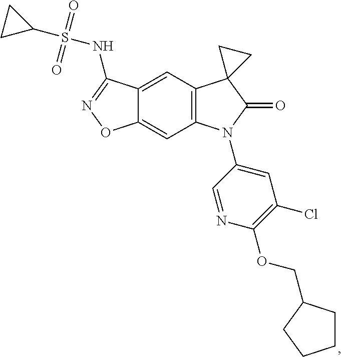 [SMILES: O=C1N(c2cnc(OCC3CCCC3)c(Cl)c2)c2cc3onc(NS(=O)(=O)C4CC4)c3cc2C12CC2]

or a pharmaceutically acceptable salt thereof.

### Embodiment 29

A pharmaceutical composition comprising a therapeutically effective amount of a compound according to any one of embodiments 1 to 28 or a pharmaceutically acceptable salt thereof and one or more pharmaceutically acceptable carriers.

### Embodiment 30

A combination comprising a therapeutically effective amount of a compound according to any one of embodiments 1 to 28 or a pharmaceutically acceptable salt thereof and one or more therapeutically active co-agents.

### Embodiment 31

A combination comprising a therapeutically effective amount of a compound according to any one of embodiments 1 to 28 or a pharmaceutically acceptable salt thereof and one or more pain-relieving agent.

### Embodiment 32

A combination according to embodiment 31 wherein the pain-relieving agents selected from the group consisting of

a) opioid analgesics, for example morphine, ketobemidone or fentanyl; b) analgesics of the NSAID or COX-1/2 class, for example ibuprofen, naproxen, celecoxib or acetylsalicylic acid, and their analogues containing nitric oxide-donating groups; c) analgesic adjuvants such as amitriptyline, imipramine, duloxetine or mexiletine; d) NMDA antagonists for example ketamine or dextrometorfan; e) sodium channel blocking agents, for example lidocaine; f) anticonvulsants, for example carbamazepine, topiramate or lamotrigine; g) anticonvulsant/analgesic amino acids such as gabapentin or pregabalin; h) cannabinoids.

### Embodiment 33

A compound or salt according to any one of embodiments 1 to 28 for use as a medicament.

### Embodiment 34

A compound or salt according to any one of embodiments 1 to 28 for use in the treatment of a disorder or disease mediated by NaV1.7.

### Embodiment 35

A compound or salt according to any one of embodiments 1 to 28 for use in the treatment of pain, particularly chronic pain, more particularly neuropathic, nociceptive and inflammatory pain, even more particularly dental pain, pain associated with osteoarthritis, erythromelalgia, diabetic neuropathy, peroxymal extreme pain disorder (PEPD) and ocular pain.

### Embodiment 36

Use of a compound according to any one of embodiments 1 to 28 or a pharmaceutically acceptable salt thereof in the manufacture of a medicament for the treatment of a disorder or disease mediated by NaV1.7.

### Embodiment 37

Use of a compound according to any one of embodiments 1 to 28 or a pharmaceutically acceptable salt thereof in the manufacture of a medicament for the treatment of pain, particularly chronic pain, more particularly neuropathic, nociceptive and inflammatory pain, even more particularly dental pain, pain associated with osteoarthritis, erythromelalgia, diabetic neuropathy, peroxymal extreme pain disorder (PEPD) and ocular pain.

### Embodiment 38

A method of treating a disorder or disease mediated by NaV1.7, comprising administering to the subject a therapeutically effective amount of a compound according to any one of embodiments 1 to 28 or a pharmaceutically acceptable salt thereof.

### Embodiment 39

A method of treatment of pain, particularly chronic pain, more particularly neuropathic, nociceptive and inflammatory pain, even more particularly dental pain, pain associated with osteoarthritis, erythromelalgia, diabetic neuropathy, peroxymal extreme pain disorder (PEPD) and ocular pain, comprising administering to the subject a therapeutically effective amount of a compound according to any one of embodiments 1 to 28 or a pharmaceutically acceptable salt thereof.

The term “compounds of the (present) invention” or “a compound of the (present) invention” refers to a compound as defined in any one of embodiments 1 to 28.

In a further aspect, there are provided processes for preparing compounds according to any one of embodiment 1 to 28.

Compounds of formula (I) wherein A is

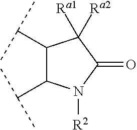

may be prepared according to Scheme 1 or 2.

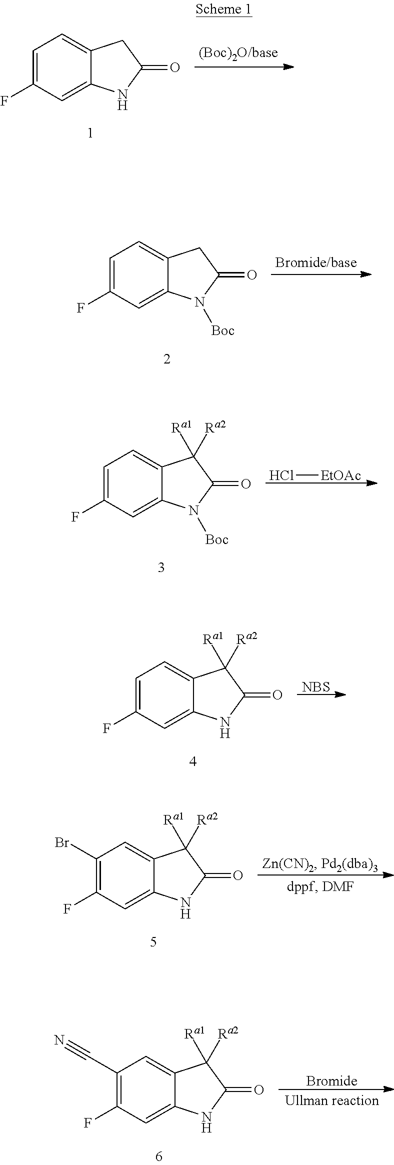

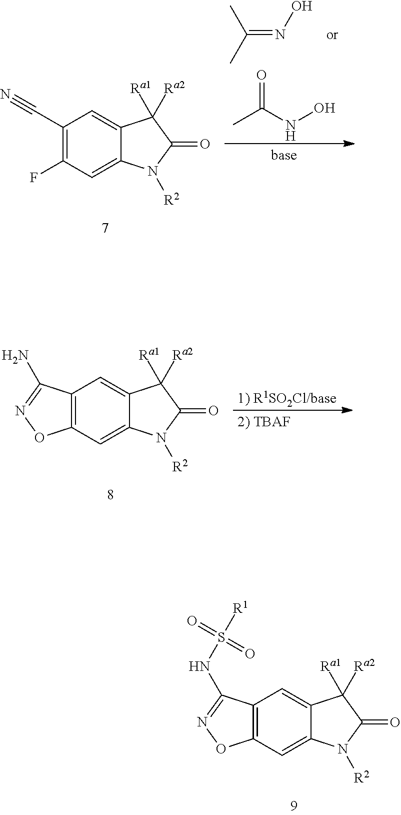

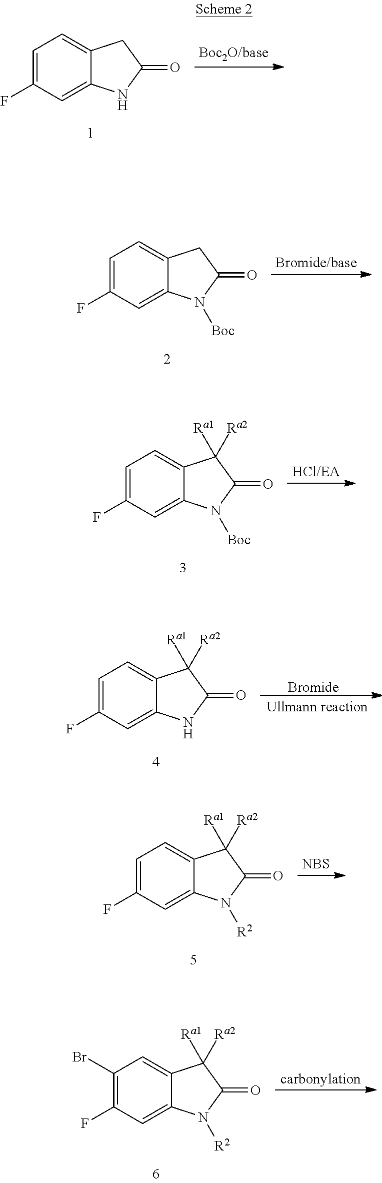

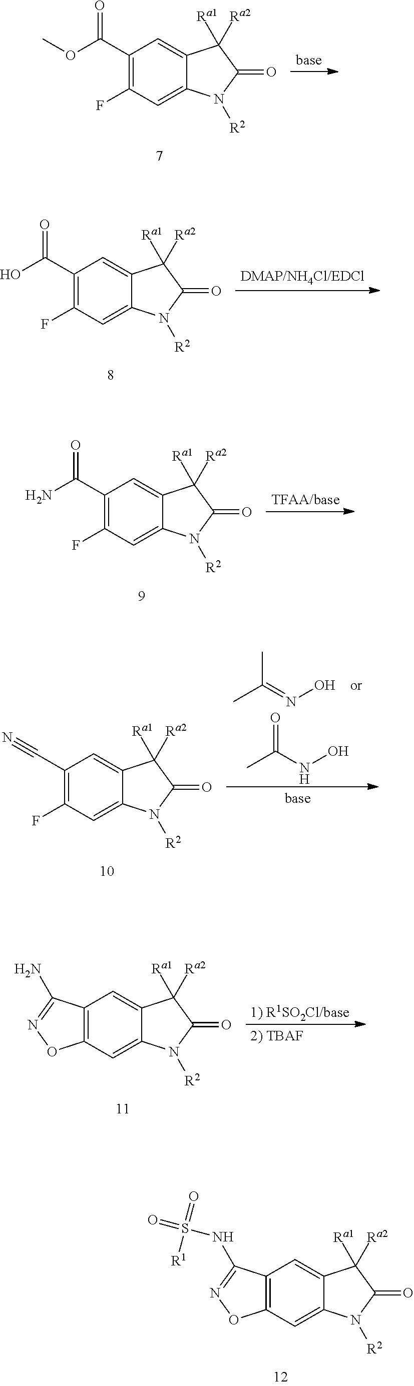

Compounds of formula (I) wherein A is

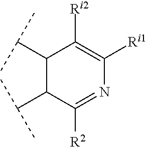

may be prepared according to Scheme 3 or 4.

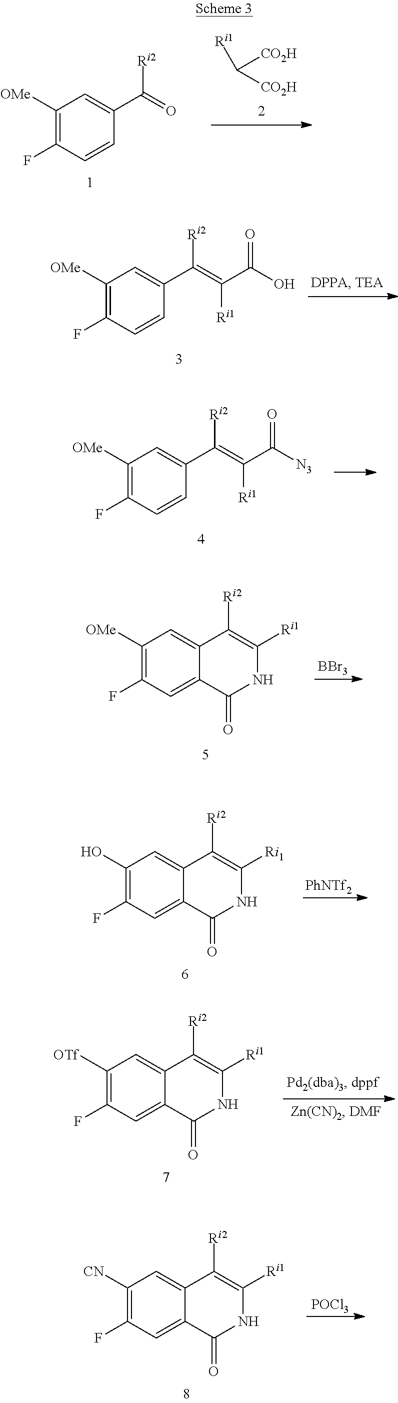

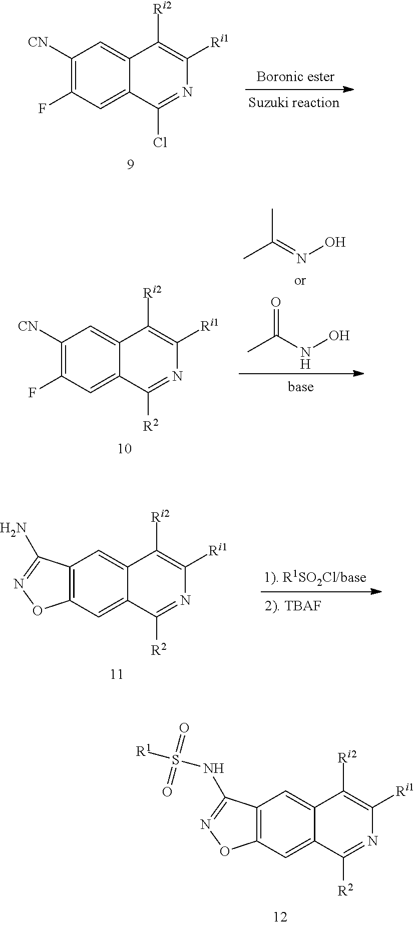

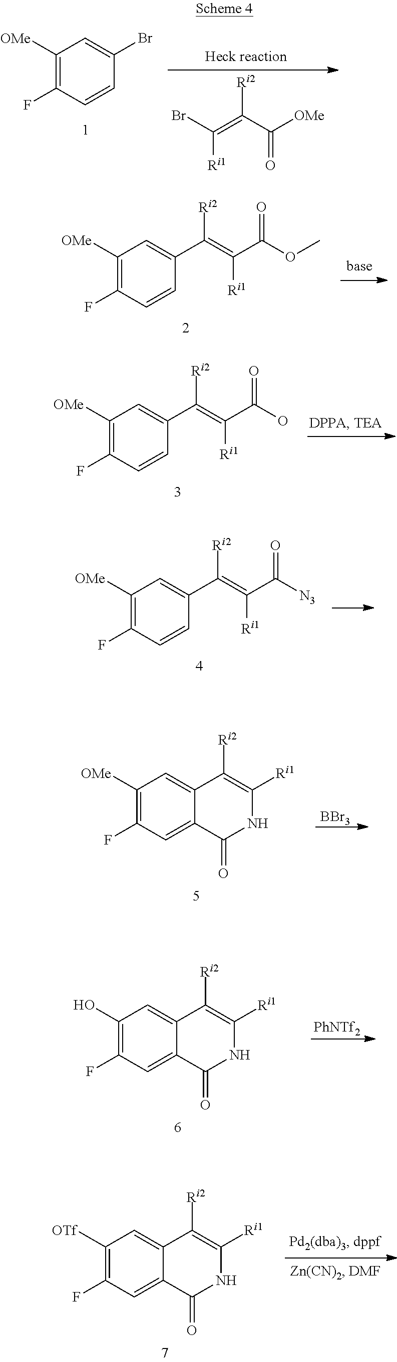

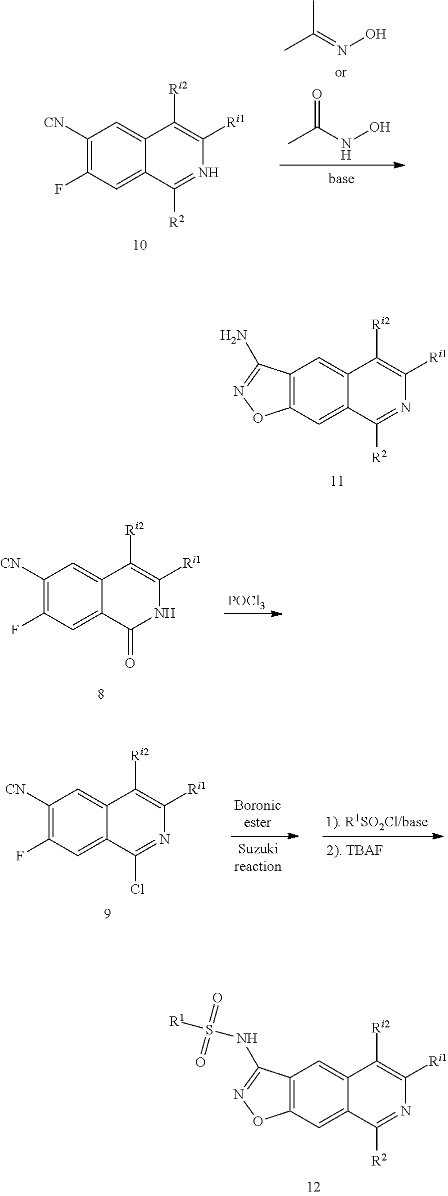

Compounds of formula (I) wherein A is

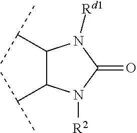

may be prepared according to Scheme 5.

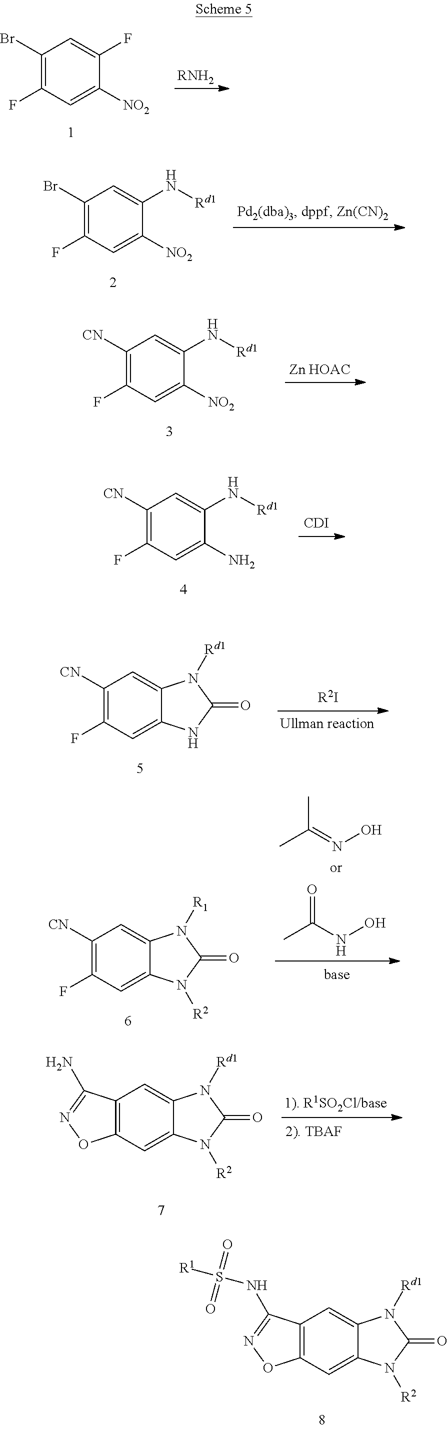

Compounds of formula (I) wherein A is

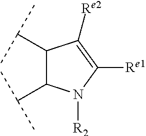

may be prepared according to Scheme 6a and 6b.

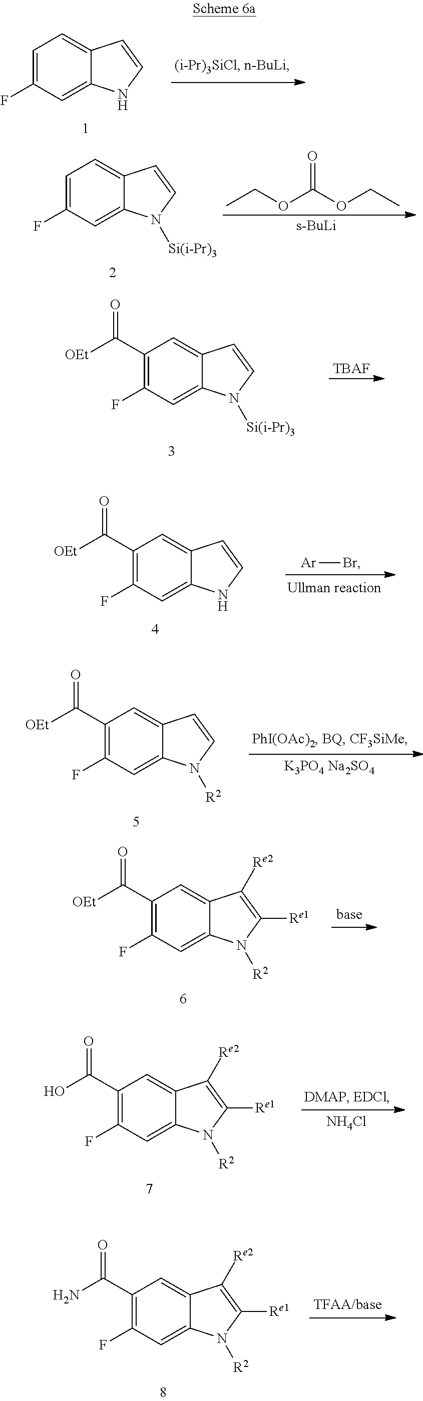

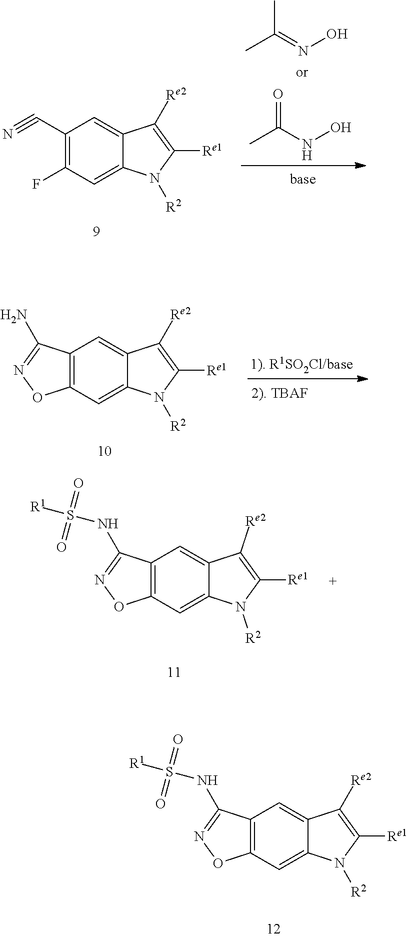

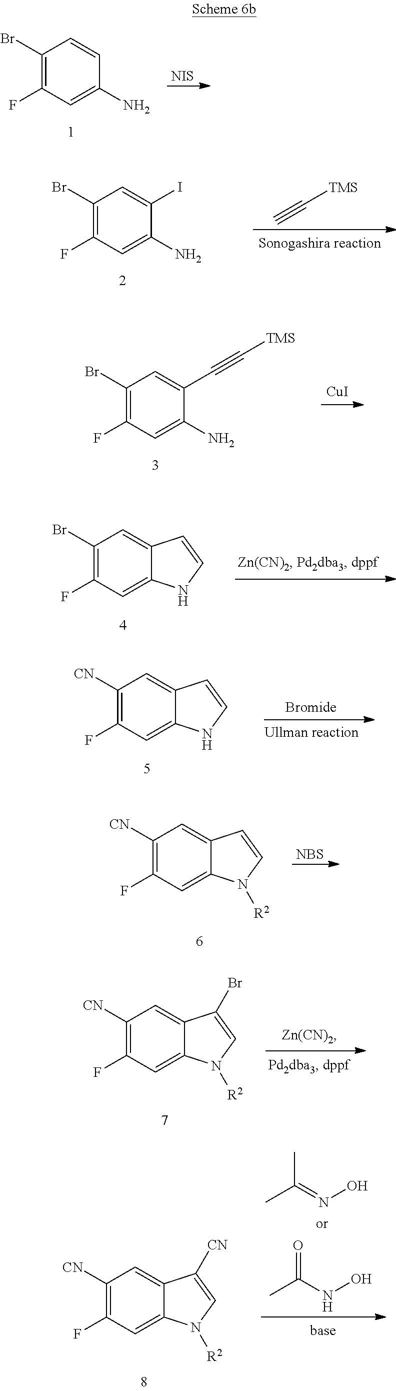

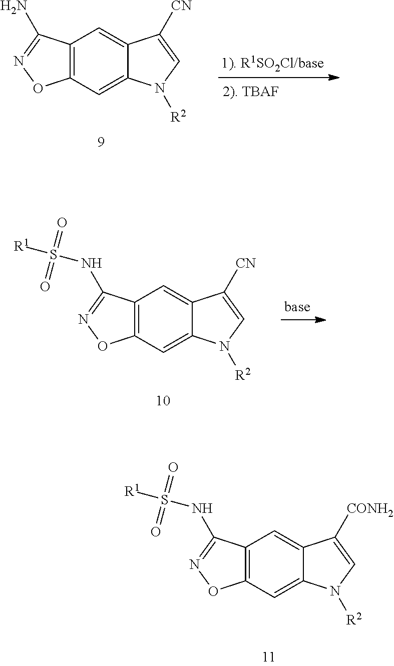

Compounds of formula (I) wherein A is

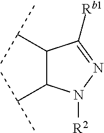

may be prepared according to Scheme 7.

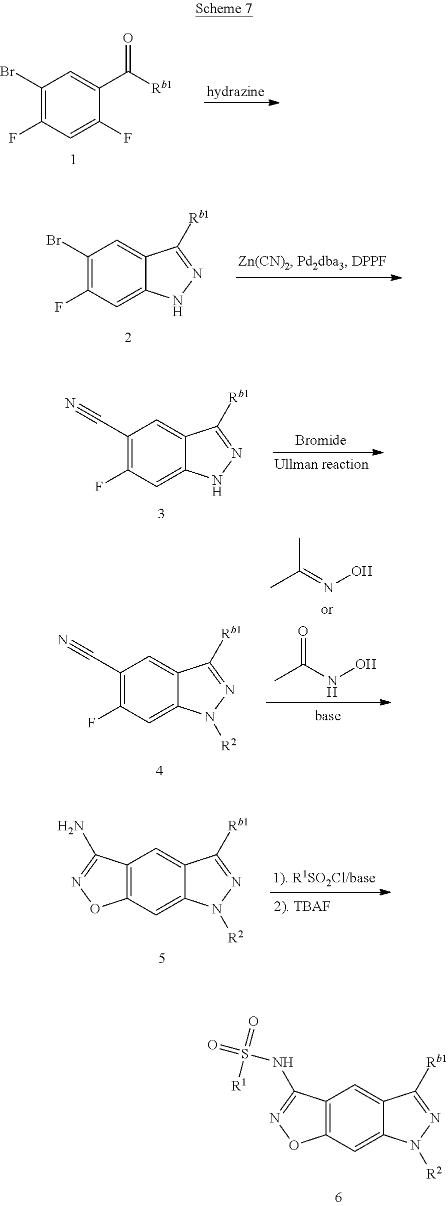

Compounds of formula (I) wherein A is

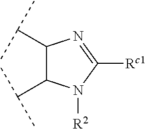

may be prepared according to Scheme 8.

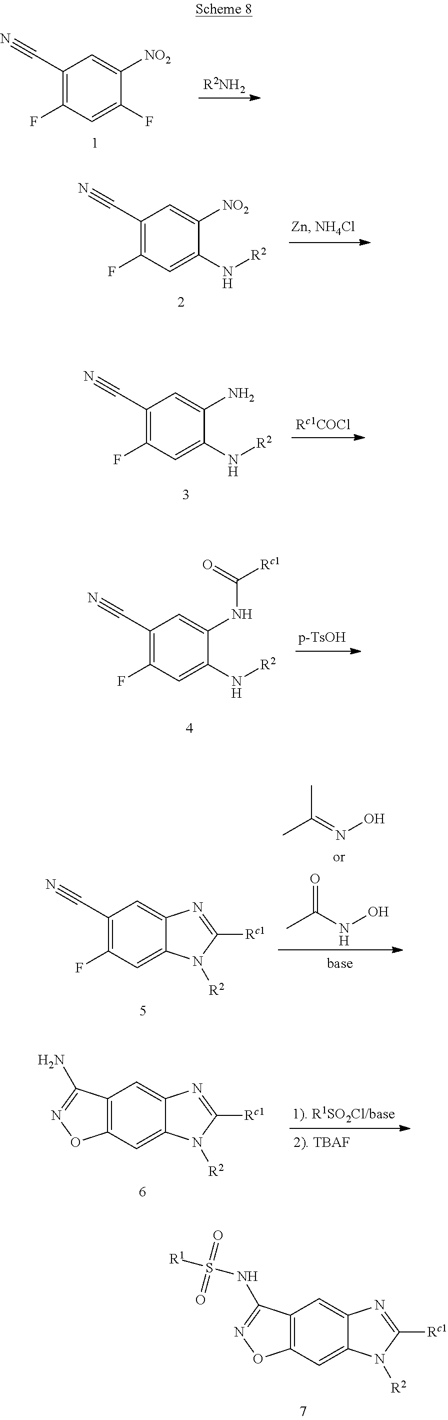

Compounds of formula (I) wherein A is

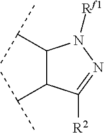

may be prepared according to Scheme 9.

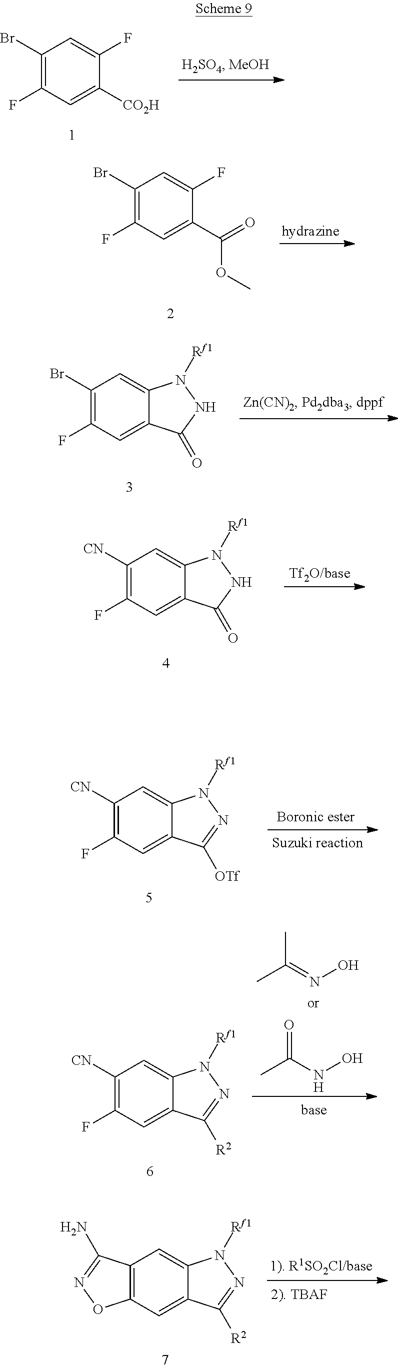

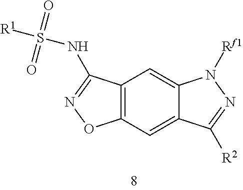

Compounds of formula (I) wherein A is

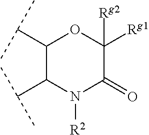

may be prepared according to Scheme 10.

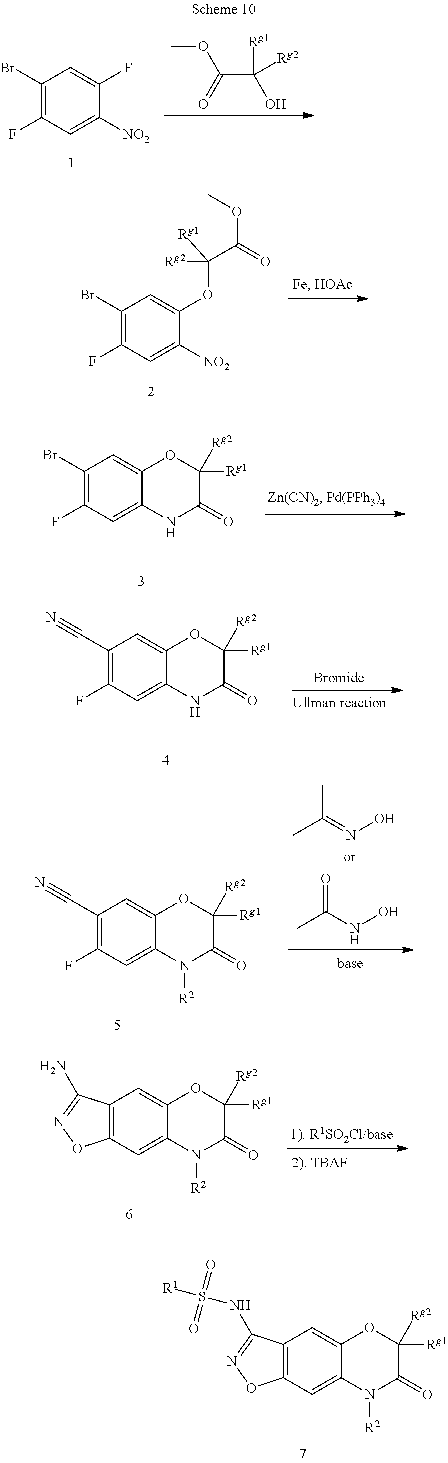

Compounds of formula (I) wherein A is

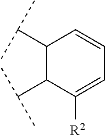

may be prepared according to Scheme 11.

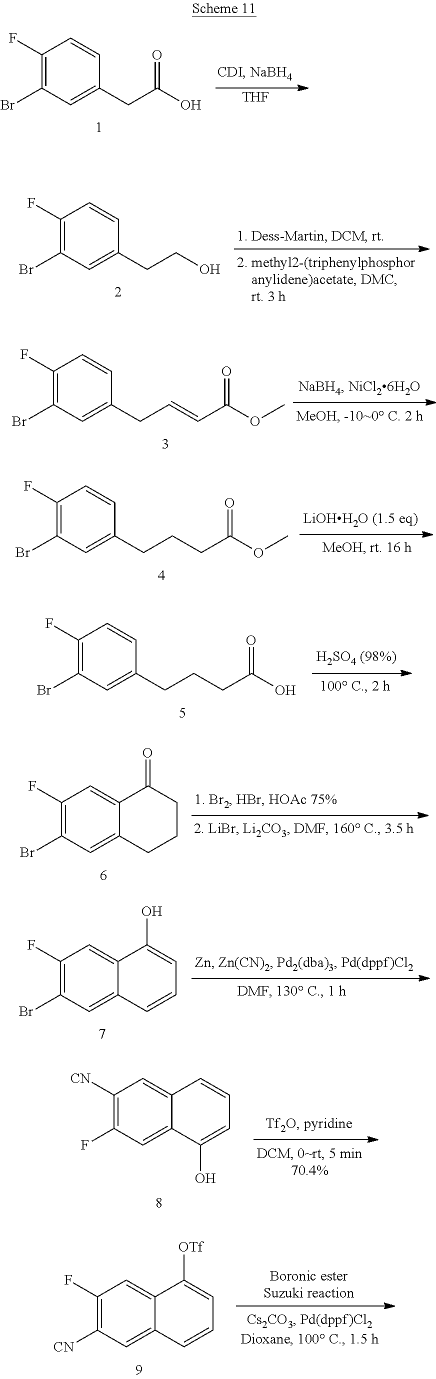

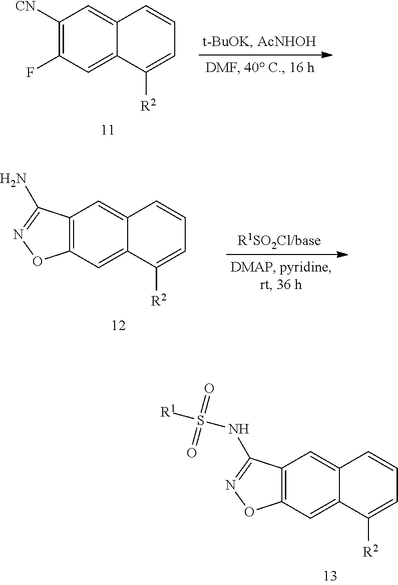

Compounds of formula (I) wherein A is

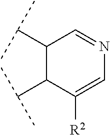

may be prepared according to Scheme 12.

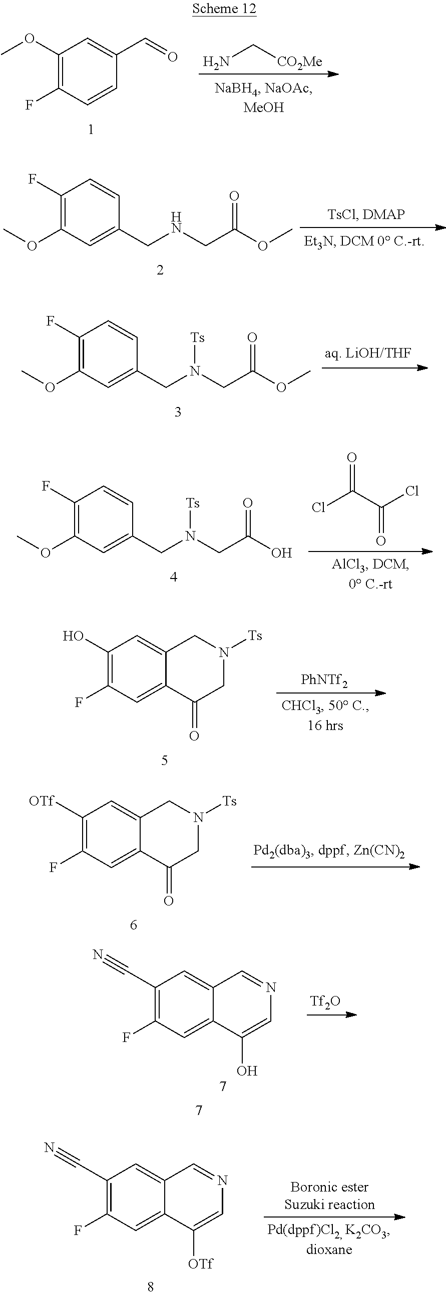

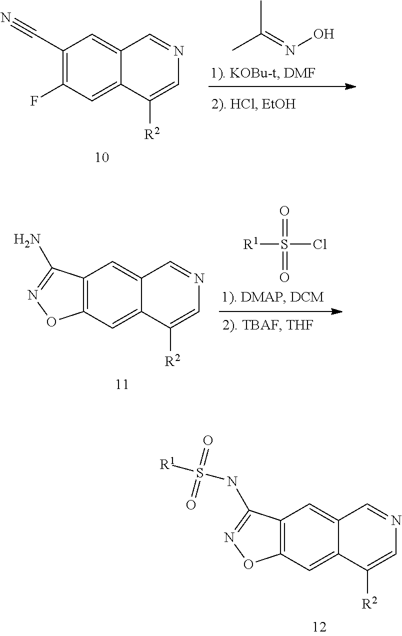

Compounds of formula (I) wherein A is

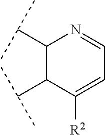

may be prepared according to Scheme 13.

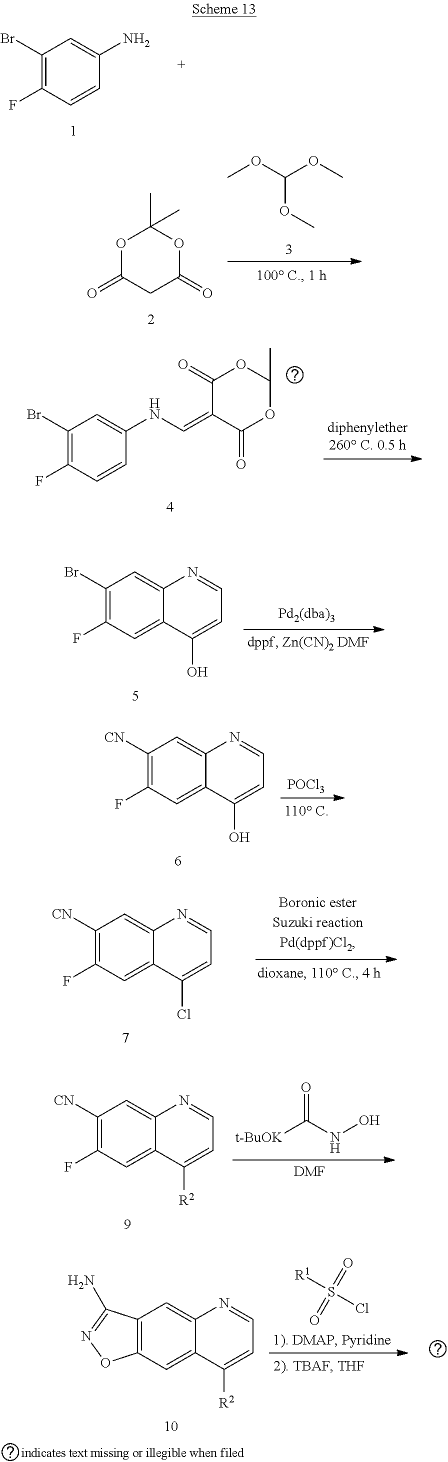

Compounds of formula (I) wherein A is

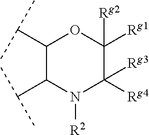

may be prepared according to Scheme 14.

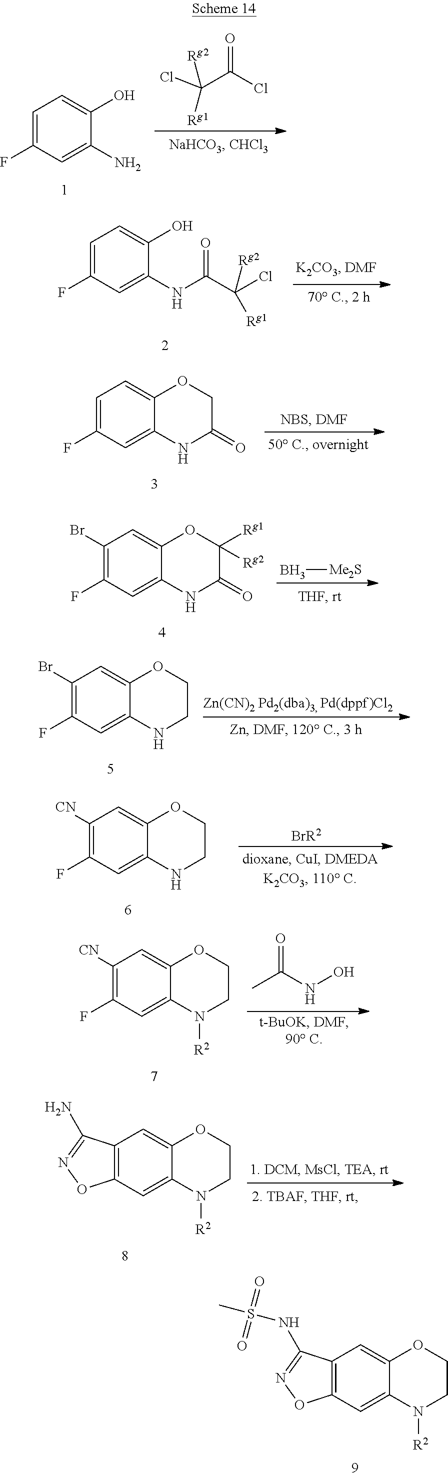

Compounds of the present invention and intermediates can also be converted into each other according to methods generally known to those skilled in the art.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers. Salts can be converted into the free compounds in accordance with methods known to those skilled in the art. Metal and ammonium salts can be converted, for example, by treatment with suitable acids, and acid addition salts, for example, by treatment with a suitable basic agent.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter. All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere. At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds of the present invention, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art.

As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compounds of the present invention. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds of the present invention described herein may contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds of the present invention may be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound of the present invention contains a double bond, the substituent may be E or Z configuration. If the compound of the present invention contains a disubstituted cycloalkyl, the cycloalkyl substituent may have a cis- or trans-configuration.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of the present invention and, which typically are not biologically or otherwise undesirable. In many cases, the compounds of the present invention are capable of forming acid and/or base salts by virtue of the presence of amino and/or carboxyl/sulfonamide groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the compounds of the present invention can be synthesized from a basic or acidic moiety, by conventional chemical methods. Generally, such salts can be prepared by reacting free acid forms of the compounds of the present invention with a stoichiometric amount of the appropriate base (such as Na, Ca, Mg, or K hydroxide, carbonate, bicarbonate or the like), or by reacting free base forms of the compounds of the present invention with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds of the present invention. Isotopically labeled compounds of the present invention have structures depicted by the formulas given herein except that one or more atoms are replaced by an atom having a selected atomic mass or mass number. Examples of isotopes that can be incorporated into compounds of the present invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl and ¹²⁵I. The invention includes various isotopically labeled compounds of the present invention, for example those into which radioactive isotopes, such as ³H and ¹⁴C, or those into which non-radioactive isotopes, such as ²H and ¹³C are present. Such isotopically labelled compounds of the present invention are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound of the present invention may be particularly desirable for PET or SPECT studies. Isotopically-labeled compounds of the present invention can generally be prepared by conventional techniques known to those skilled in the art or by processes analogous to those described in the accompanying Generic Schemes, Examples and Preparations using an appropriate isotopically-labeled reagent in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of the present invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of the present invention that contain groups capable of acting as donors and/or acceptors for hydrogen bonds may be capable of forming co-crystals with suitable co-crystal formers. These co-crystals may be prepared from compounds of the present invention by known co-crystal forming procedures. Such procedures include grinding, heating, co-subliming, co-melting, or contacting in solution compounds of the present invention with the co-crystal former under crystallization conditions and isolating co-crystals thereby formed. Suitable co-crystal formers include those described in WO 2004/078163. Hence the invention further provides co-crystals comprising a compound of the present invention.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The compounds of the present invention, including salts, hydrates and solvates thereof, may inherently or by design form polymorphs.

The compounds of the present invention in free form or in salt form, exhibit valuable pharmacological properties, e.g. as indicated in in vitro tests as provided herein, and are therefore indicated for therapy or for use as research chemicals, e.g. as tool compounds.

Thus, there is provided a compound according to any one of embodiments 1 to 28 for use in medicine.

The compounds according to any one of embodiments 1 to 28 are potent inhibitors of NaV1.7 (see IC₅₀ data disclosed herein). The compound of the present invention are hence useful in the treatment of an NaV1.7-dependent or NaV1.7-mediated disease or condition. The compounds according to any one of embodiments 1 to 28 have favourable pharmacokinetic properties, particularly following oral administration, more particularly at higher doses. The compounds according to any one of embodiments 1 to 28 have particularly favourable solubility and absorption profiles.

Thus, there is provided a compound according to any one of embodiments 1 to 28 for use in the treatment of a NaV1.7-dependent or NaV1.7-mediated disease or condition. There is also provided the use of a compound according to any one of embodiments 1 to 28 in the treatment of an NaV1.7-dependent or NaV1.7-mediated disease or condition. There is further provided the use of a compound according to any one of embodiments 1 to 28 in the manufacture of a medicament for the treatment of a NaV1.7-dependent or NaV1.7-mediated disease or condition. There is provided a method of treating a NaV1.7-dependent or NaV1.7-mediated disease or condition comprising administering to the subject a therapeutically effective amount of a compound according to any one of embodiments 1 to 28.

Disorders for which a Nav1.7 inhibitor is indicated include pain, i.e. chronic and acute pain, particularly chronic pain, more particularly neuropathic, nociceptive and inflammatory pain, even more particularly dental pain, pain associated with osteoarthritis, erythromelalgia, diabetic neuropathy, peroxymal extreme pain disorder (PEPD) and ocular pain.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf and Salter, 2000, Science, 288, 1765-1768). Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating. Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf and Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus). The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan and Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge and Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (I BD). These GI disorders include a wide range of disease states that are currently only moderately controlled, including, in respect of FBD, gastro-esophageal reflux, dyspepsia, irritable bowel syndrome (I BS) and functional abdominal pain syndrome (FAPS), and, in respect of I BD, Crohn's disease, ileitis and ulcerative colitis, all of which regularly produce visceral pain. Other types of visceral pain include the pain associated with dysmenorrhea, cystitis and pancreatitis and pelvic pain. It should be noted that some types of pain have multiple aetiologies and thus can be classified in more than one area, e.g. back pain and cancer pain have both nociceptive and neuropathic components.

Other types of pain include:
pain resulting from musculoskeletal disorders, including myalgia, fibromyalgia, spondylitis, sero-negative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders; erythermalgia;
orofacial pain, including dental pain, otic pain, burning mouth syndrome and temporomandibular myofascial pain;
eye pain (ophthalmalgia): ocular pain, orbital pain.

Hence, the compounds of the invention are useful for the treatment of pain.

Furthermore, the compounds of the invention may also be useful for the treatment of cough. They may be useful for the treatment of respiratory hypersensitivity such as cough and more particularly, can be used for the treatment of non-productive cough.

In particular embodiments, the compounds and pharmaceutical formulations described herein are used for the treatment of non-productive cough in a subject suffering from idiopathic pulmonary fibrosis (IPF). IPF is a condition characterized by fibroproliferation and modest mononuclear inflammation of the pulmonary interstitium. A subject suffering from IPF typically experience worsening shortness of breath, with non-productive cough as an additional distressing feature in more than 50% of cases. (Hope-Gill et al., American Journal of Respiratory and Critical Care Medicine (2003), 168:995-1002). As used herein, a non-productive cough is a dry cough in contrast to a productive cough (when sputum is coughed up).

The compounds of the invention will be typically formulated as pharmaceutical compositions.

Thus, the present invention provides a pharmaceutical composition comprising a compound according to any one of embodiments 1 to 30, or a pharmaceutically acceptable salt thereof, and a pharmaceutically acceptable carrier.

The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc. Typically, the pharmaceutical compositions are tablets or gelatin capsules comprising the active ingredient together with

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and/or

e) absorbents, colorants, flavors and sweeteners.

Tablets may be either film coated or enteric coated according to methods known in the art.

Suitable compositions for oral administration include an effective amount of a compound of the present invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by blockade of the epithelial sodium channel. Products provided as a combined preparation include a composition comprising the compound of the present invention and the other therapeutic agent(s) together in the same pharmaceutical composition, or the compound of the present invention and the other therapeutic agent(s) in separate form, e.g. in the form of a kit.

Thus, the invention provides a pharmaceutical composition comprising a compound according to any one of embodiments 1 to 28 and one or more therapeutically active co-agent. Optionally, the pharmaceutical composition may comprise a pharmaceutically acceptable excipient, as described above.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In another embodiment of the invention, there is provided a pharmaceutical combination, comprising a therapeutically effective amount of the compound according to any one of embodiments 1 to 28, or a pharmaceutically acceptable salt thereof, and one or more therapeutically active co-agent.

In another embodiment of the invention, there is provided a pharmaceutical combination, comprising a therapeutically effective amount of the compound according to any one of embodiments 1 to 28, or a pharmaceutically acceptable salt thereof, and one or more therapeutically active co-agent, wherein the therapeutically active co-agent is selected from one or more pain-relieving agent.

In another embodiment of the invention, there is provided a pharmaceutical combination, comprising a therapeutically effective amount of the compound according to any one of embodiments 1 to 28, or a pharmaceutically acceptable salt thereof, and one or more therapeutically active co-agent, wherein the therapeutically active co-agent is selected from a) opioid analgesics, for example morphine, ketobemidone or fentanyl; b) analgesics of the NSAID or COX-1/2 class, for example ibuprofen, naproxen, celecoxib or acetylsalicylic acid, and their analogues containing nitric oxide-donating groups; c) analgesic adjuvants such as amitriptyline, imipramine, duloxetine or mexiletine; d) NMDA antagonists for example ketamine or dextrometorfan; e) sodium channel blocking agents, for example lidocaine; f) anticonvulsants, for example carbamazepine, topiramate or lamotrigine; g) anticonvulsant/analgesic amino acids such as gabapentin or pregabalin; h) cannabinoids.

The activity of a compound according to the present invention can be assessed by the following in vitro methods.

### EXAMPLES

### General Conditions

Mass spectra were acquired on LC-MS, SFC-MS, or GC-MS systems using electrospray, chemical and electron impact ionization methods from a range of instruments of the following configurations: Shimadzu LC20 HPLC systems with an Shimadzu2010 Mass Spectrometer [M+H]+ refers to protonated molecular ion of the chemical species.

NMR spectra were run on Bruker AVANCE 400 MHz NMR spectrometers using ICON-NMR (under TopSpin program control) or on Varian 400 MHz NMR spectrometers (under VmrJ program control). Spectra were measured at 298 K, unless indicated otherwise, and were referenced relative to the solvent resonance.

Instrumentation

MS Methods:

Using Shimadzu LC20 HPLC systems with an Shimadzu2010 Mass Spectrometer

Method 10-80AB 7minLC_v001

| | |
|---|---|
| Column | Xtimate C18 2.1 × 30 mm, 3.0 μm |
| Column | 50° C. |
| Temperature | |
| Eluents | A: H₂O (4 L) + TFA (1.5 ml), B: acetonitrile (4 L) + TFA |
| | (0.75 ml) |
| Flow Rate | 0.8 ml/min |
| Gradient | 10% to 80% B in 6.0 min, 80% to 10% B in 0.5 min, |
| | 10% B 0.5 min |

Method 0-60AB 7minLC_v002

| | |
|---|---|
| Column | Xtimate C18 2.1 × 30 mm, 3.0 μm |
| Column | 50° C. |
| Temperature | |
| Eluents | A: H₂O (4 L) + TFA (1.5 ml), B: acetonitrile (4 L) + TFA |
| | (0.75 ml) |
| Flow Rate | 0.8 ml/min |
| Gradient | 0% to 60% B in 6.0 min, 60% to 0% B in 0.5 min, |
| | 0% B 0.5 min |

Method 5-95AB 1.5minLC_v003

| | |
|---|---|
| Column | Merck, RP-18e, 25-2 mm |
| Column | 50° C. |
| Temperature | |
| Eluents | A: H₂O (4 L) + TFA (1.5 ml), B: acetonitrile (4 L) + TFA |
| | (0.75 ml) |
| Flow Rate | 1.5 ml/min |
| Gradient | 5% to 95% B in 0.7 min, 95% B 0.4 min, 5% B 0.4 min |

Abbreviations:

app apparent

ATP adenosine 5′-triphosphate

BINAP racemic 2,2′-bis(diphenylphosphino)-1,1′-binaphthyl

BOC tertiary butyl carboxy

(Boc)₂O di-tert-butyl dicarbonate

br broad

BSA bovine serum albumin

CDI 1,1′-carbonyldiimidazole

d doublet

dd doublet of doublets

DCM dichloromethane

DIEA diethylisopropylamine

DMAP 4-dimethylaminopyridine

DME 1,4-dimethoxyethane

DMEDA N,N′-dimethylethylenediamine

DMF N,N-dimethylformamide

DMSO dimethylsulfoxide

DPPF 1,1′-bis(diphenylphosphino)ferrocene

DPPA diphenyl phosphoryl azide

EDTA ethylenediamine tetraacetic acid

EDC.HCl 1-(3-dimethylaminopropyl)-3-ethylcarbodiimide hydrochloride

ESI electrospray ionization

EtOAc ethyl acetate

EtOH ethanol

h hour(s)

HPLC high pressure liquid chromatography

LCMS liquid chromatography and mass spectrometry

MS mass spectrometry

m multiplet

mg milligram

min minutes

ml milliliter

mmol millimol

m/z mass to charge ratio

NMR nuclear magnetic resonance

ppm parts per million

rac racemic

Rt retention time

s singlet

t triplet

TBAF tetrabutylammonium fluoride

TMEDA N¹,N¹,N²,N²-tetramethylethane-1,2-diamine

t-BuOK potassium 2-methylpropan-2-olate

t-BuLi tert-butyllithium

TEA triethylamine

TFA trifluoroacetic acid

THF tetrahydrofuran

TLC thin layer chromatography

TOP tri-o-tolylphosphine

### Example 1

### N-(7′-(5-chloro-6-isobutoxypyridin-3-yl)-6′-oxo-6′,7′-dihydrospiro[cyclopropane-1,5′-isoxazolo[4,5-f]indol]-3′-yl)methanesulfonamide

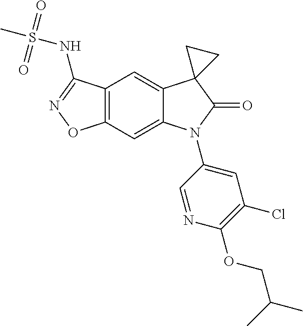 [SMILES: CC(C)COc1ncc(N2C(=O)C3(CC3)c3cc4c(NS(C)(=O)=O)noc4cc32)cc1Cl]

### Step 1: tert-butyl 6-fluoro-2-oxoindoline-1-carboxylate

To a mixture of 6-fluoroindolin-2-one (Aldrich) (151 g, 1 mol) and DMAP (24 g, 0.2 mol) in DCM (1000 ml) was added (Boc)₂O (240 g, 1.1 mol). The resulting mixture was stirred at room temperature for 16 hrs. When TLC indicated the starting material was consumed, the reaction mixture was washed with water (×2), dried over anhydrous magnesium sulfate and filtered. The filtration was concentrated under reduced pressure to give crude product, which was purified by column chromatography on silica-gel eluting with 0-5% ethyl acetate in hexanes to afford the title compound as a yellow solid.

LC-MS: Rt=1.48 mins; MS m/z [M+H]+ 252.2; Method 5-95AB 1.5minLC_v003

¹H NMR (400 MHz, CDCl₃) δ 7.57 (1H, dd), 7.16 (1H, dd), 6.84 (1H, m,), 3.60 (2H, s), 1.63 (9H, s).

### Step 2: tert-butyl 6′-fluoro-2′-oxospiro[cyclopropane-1,3′-indoline]-1′-carboxylate

To a mixture of tert-butyl 6-fluoro-2-oxoindoline-1-carboxylate (step 1) (176 g, 0.7 mol) and K₂CO₃ (483 g, 3.5 mol) in DMSO (1000 ml) was added 1,2-dibromoethane (326 g, 1.7 mol). The resulting mixture was stirred at room temperature for 16 hrs. When TLC indicated the starting material was consumed, the reaction mixture was poured into water (3000 ml). The mixture was extracted with EtOAc (×3). The combined organic phases were washed with 1N HCl aqueous solution (×3) and brine (×3). Then the mixture was dried over anhydrous magnesium sulfate, filtered and concentrated to give the crude product, which was purified by column chromatography on silica-geleluting with 0˜5% EtOAc in hexane to afford the title compound as a yellow solid.

LC-MS: Rt=1.41 mins; MS m/z [M+H]+ 278.2; Method 5-95AB 1.5minLC_v003

¹H NMR (400 MHz, CDCl₃) δ 7.70 (1H, dd), 6.82 (1H, dd), 6.73 (1H, dd), 1.81 (2H, m), 1.65 (9H, s), 1.52 (2H, m).

### Step 3: 6′-fluorospiro[cyclopropane-1,3′-indolin]-2′-one

To a mixture of tert-butyl 6′-fluoro-2′-oxospiro[cyclopropane-1,3′-indoline]-1′-carboxylate (step 2) (83 g, 0.3 mol) in EtOAc (300 ml) was added a solution of HCl in EtOAc (2N, 100 ml) in ice-bath. The resulting mixture was stirred at room temperature for 5 hrs. When TLC indicated the starting material was consumed, the reaction mixture was concentrated under reduced pressure to give the title compound as a white solid.

LC-MS: Rt=1.34 mins; MS m/z [M+H]+ 178.1; Method 5-95AB 1.5minLC_v003

¹H NMR (400 MHz, CDCl₃) δ 8.99 (1H, br s), 6.68 (3H, m), 1.74 (2H, m), 1.52 (2H, m).

### Step 4: 5′-bromo-6′-fluorospiro[cyclopropane-1,3′-indolin]-2′-one

To a mixture of 6′-fluorospiro[cyclopropane-1,3′-indolin]-2′-one (step 3) (44 g, 0.25 mol) in CH₃CN (500 ml) was added NBS (53 g, 0.3 mol) by portion. The resulting mixture was stirred at room temperature overnight. When TLC indicated the starting material was consumed, the solid was collected and washed with hot water (×2) and dried to afford the title compound as a whit solid.

LC-MS: Rt=1.37 mins; MS m/z [M+H]+ 255.9; Method 5-95AB 1.5minLC_v003

¹H NMR (400 MHz, DMSO-d₆) δ 10.8 (1H, br s), 7.33 (1H, d), 6.88 (1H, d), 1.63 (2H, m), 1.46 (2H, m).

### Step 5: 6′-fluoro-2′-oxospiro[cyclopropane-1,3′-indoline]-5′-carbonitrile

To a mixture of 5′-bromo-6′-fluorospiro[cyclopropane-1,3′-indolin]-2′-one (step 4) (50 g, 0.2 mol), DPPF (44 g, 0.08 mol) and Zn(CN)₂ (46 g, 0.60 mmol) in DMF (500 ml) was added Pd₂(dba)₃ (37 g, 0.04 mol) at room temperature. The resulting mixture was degassed and charged with nitrogen three times. After heating to 120° C., the reaction mixture was stirred at that temperature under nitrogen atmosphere for 4 hrs. When LC/MS indicated the starting material was consumed, the reaction mixture was cooled down and diluted with ethyl acetate (1000 ml). The mixture was filtered through a pad of celite and the filtration was then washed with brine (×3), dried over anhydrous magnesium sulfate and filtered. The filtration was evaporated under reduced pressure to give the crude product, which was purified by column chromatography on silica gel eluting with 0-30% ethyl acetate in hexanes to afford the title compound as a white solid.

LC-MS: Rt=1.32 mins; MS m/z [M+H]+ 203.0; Method 5-95AB 1.5minLC_v003

¹H NMR (400 MHz, DMSO-d₆) δ 11.2 (1H, br s), 7.350 (1H, d), 6.94 (1H, d), 1.68 (2H, m), 1.50 (2H, m).

### Step 6: I-(5-chloro-6-isobutoxypyridin-3-yl)-6′-fluoro-2′-oxospiro[cyclopropane-1,3′-indoline]-5′-carbonitrile

To a mixture of 6′-fluoro-2′-oxospiro[cyclopropane-1,3′-indoline]-5′-carbonitrile (step 5) (21 g, 0.1 mol) in CH₃CN (300 ml) was added 5-bromo-3-chloro-2-isobutoxypyridine (intermediate A) (79 g, 0.3 mmol), CuI (38 g, 0.2 mol), K₂CO₃ (28 g, 0.2 mol) and DMEDA (35 g, 0.4 mol) at room temperature. The resulting mixture was degassed and charged with nitrogen three times. After heating to 100° C., the reaction mixture was stirred at that temperature for 16 hrs. When LC/MS indicated the starting material was consumed, the reaction mixture was cooled down and diluted with ethyl acetate (1000 ml). The mixture was filtered through a pad of celite and the filtration was then washed with brine (×3), dried over anhydrous magnesium sulfate and filtered. The filtration was evaporated under reduced pressure to give the crude product, which was purified by column chromatography on silica gel eluting with 0-20% ethyl acetate in hexanes to afford the title compound as a white solid.

LC-MS: Rt=1.49 mins; MS m/z [M+H]+ 386.1; Method 5-95AB 1.5minLC_v003

¹H NMR (400 MHz, CDCl₃) δ 8.10 (1H, d), 7.70 (1H, d), 7.08 (1H, d), 6.69 (1H, d), 4.21 (2H, d), 2.19 (1H, m), 1.95 (2H, m), 1.73 (2H, m), 1.07 (6H, d).

### Step 7: 3′-amino-7′-(5-chloro-6-isobutoxypyridin-3-yl)spiro[cyclopropane-1,5′-isoxazolo[4,5-f]indol]-6′(7′H)-one

To a mixture of propan-2-one oxime (4.4 g, 60 mmol) in anhydrous DMF (50 ml) was added t-BuOK (6.8 g, 60 mmol) by portion at room temperature. The resulting mixture was stirred at room temperature for 30 min and then added a solution of 1′-(5-chloro-6-isobutoxypyridin-3-yl)-6′-fluoro-2′-oxospiro[cyclopropane-1,3′-indoline]-5′-carbonitrile (step 6) (15 g, 40 mmol) in DMF (50 ml). After stirring at room temperature for 5 hrs, the reaction was quenched with saturated ammonium chloride solution. The reaction mixture was extracted with ethyl acetate (×3). The combined organic phases were washed with brine (×2), dried over anhydrous magnesium sulfate and filtered. The filtration was concentrated under reduced pressure to give the crude product, which was treated with 400 ml of ethanol and 400 ml of 5% HCl aqueous solution. The resulting mixture was heated to reflux for 45 min. After removal of the organic solvent, the remaining aqueous phase was basified with saturated Na₂CO₃ aqueous solution. The mixture was extracted with ethyl acetate (×3), washed with brine (×2), dried over anhydrous magnesium sulfate and filtered. The filtration was concentrated under reduced pressure to give the crude product, which was purified by flash chromatography on silica gel eluting with 0-20% ethyl acetate in hexanes to afford the title compound as a white solid.

LC-MS: Rt=0.88 mins; MS m/z [M+H]+ 398.9; Method 5-95AB 1.5minLC_v003

¹H NMR (400 MHz, CDCl₃) δ 8.16 (1H, d), 7.76 (1H, d), 6.98 (1H, s), 6.85 (1H, s), 4.36 (2H, br s), 4.21 (2H, d), 2.19 (1H, m), 1.93 (2H, m), 1.67 (2H, m), 1.08 (6H, d).

### Step 8: N-(7′-(5-chloro-6-isobutoxypyridin-3-yl)-6′-oxo-6′,7′-dihydrospiro[cyclopropane-1,5′-isoxazolo[4,5-f]indol]-3′-yl)methanesulfonamide

To a mixture of 3′-amino-7′-(5-chloro-6-isobutoxypyridin-3-yl)spiro[cyclopropane-1,5′-isoxazolo[4,5-f]indol]-6′(7′H)-one (step 7) (1 g, 2.5 mmol) in dry DCM (10 ml) was added TEA (1.5 g, 15 mmol) and methanesulfonyl chloride (1.2 g, 10 mmol) at room temperature. The resulting mixture was stirred at room temperature for 2 hrs and quenched with saturated ammonium chloride aqueous solution. The reaction mixture was washed with brine (×3), dried over anhydrous magnesium sulfate and filtered. The filtration was concentrated under reduced pressure to afford the crude product, which was dissovled in THF (10 ml). To the above mixture was added TBAF (1.3 g, 5 mmol) and the resulting mixture was then stirred at room temperature for 2 hrs. After removal of the solvent, the residue was treated with EtOAc and water. The separated organic phase was washed with water (×2), dried over anhydrous magnesium sulfate and filtered. The filtration was concentrated under reduced pressure to give the crude product, which was purified by column chromatograph on silica gel eluting with 0˜20% EtOAc in hexanes to afford the title compound as a white solid.

LC-MS: Rt=3.67 mins; MS m/z [M+H]+ 477.0; Method 10-80AB 7minLC_v001

¹H NMR (400 MHz, DMSO-d₆) δ 11.31 (1H, s), 8.31 (1H, d), 8.20 (1H, d), 7.55 (1H, s), 7.17 (1H, s), 4.21 (2H, d), 3.31 (3H, br. s), 2.12 (1H, m), 1.75 (4H, s), 1.02 (6H, d).

### Example 2

### N-(7′-(5-chloro-6-isobutoxypyridin-3-yl)-6′-oxo-6′,7′-dihydrospiro[cyclopropane-1,5′-isoxazolo[4,5-f]indol]-3′-yl)cyclopropanesulfonamide

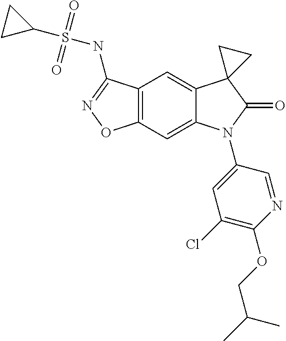 [SMILES: CC(C)COc1ncc(N2C(=O)C3(CC3)c3cc4c(NS(=O)(=O)C5CC5)noc4cc32)cc1Cl]

The title compound was prepared by a method similar to that of Example 1 by replacing methanesulfonyl chloride (Example 1 step 8) with cyclopropanesulfonyl chloride;

LC-MS: Rt=4.85 mins; MS m/z [M+H]+ 503.0; Method 10-80AB 7minLC_v002

¹H NMR (400 MHz, CDCl₃) δ 11.31 (1H, s), 8.16 (1H, d), 7.76 (1H, d), 7.41 (1H, s), 6.90 (1H, s), 4.22 (2H, d), 2.68 (1H, m), 2.20 (1H, m), 1.94 (2H, m), 1.71 (2H, m), 1.23 (2H, m), 1.09 (2H, m), 1.08 (6H, d).

### Example 3

### N-(7′-(5-chloro-6-isobutoxypyridin-3-yl)-6′-oxo-6′,7′-dihydrospiro[cyclopropane-1,5′-isoxazolo[4,5-f]indol]-3′-yl)propane-2-sulfonamide

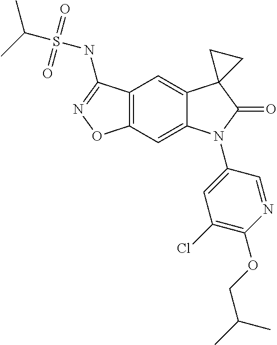 [SMILES: CC(C)COc1ncc(N2C(=O)C3(CC3)c3cc4c(NS(=O)(=O)C(C)C)noc4cc32)cc1Cl]

The title compound was prepared by a method similar to that of Example 1 by replacing methanesulfonyl chloride (Example 1 step 8) with propane-2-sulfonyl chloride;

LC-MS: Rt=4.48 mins; MS m/z [M+H]+ 505.1; Method 10-80AB 7minLC_v002

1H NMR (400 MHz, CDCl₃) δ 8.16 (1H, d), 7.75 (1H, d), 7.44 (1H, s), 7.29 (1H, s), 6.93 (1H, s), 4.22 (2H, d), 3.57 (1H, m), 2.20 (1H, m), 1.745 (2H, m), 1.59 (2H, m), 1.49 (6H, d), 1.09 (6H, d).

### Example 4

### N-(7′-(5-chloro-6-isobutoxypyridin-3-yl)-6′-oxo-6′,7′-dihydrospiro[cyclopropane-1,5′-isoxazolo[4,5-f]indol]-3′-yl)ethanesulfonamide

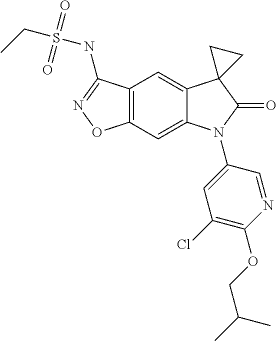 [SMILES: CCS(=O)(=O)Nc1noc2cc3c(cc12)C1(CC1)C(=O)N3c1cnc(OCC(C)C)c(Cl)c1]

The title compound was prepared by a method similar to that of Example 1 by replacing methanesulfonyl chloride (Example 1 step 8) with ethanesulfonyl chloride;

LC-MS: Rt=5.12 mins; MS m/z [M+H]+ 491.1; Method 10-80AB 7minLC_v002

1H NMR (400 MHz, CDCl₃) δ 8.15 (1H, d), 7.75 (1H, d), 7.38 (2H, s), 6.93 (1H, s), 4.22 (2H, d), 3.38 (2H, q), 2.17 (1H, m), 1.94 (2H, m), 1.73 (2H, m), 1.48 (3H, t), 1.09 (6H, d).

### Example 5

### N-(7′-(5-chloro-6-isobutoxypyridin-3-yl)-6′-oxo-6′,7′-dihydrospiro[cyclopropane-1,5′-isoxazolo[4,5-f]indol]-3′-yl)-1,1,1-trifluoromethanesulfonamide

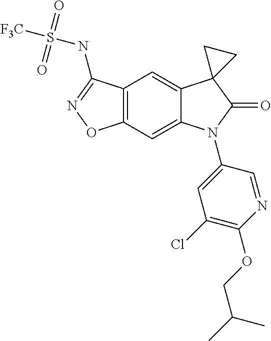 [SMILES: CC(C)COc1ncc(N2C(=O)C3(CC3)c3cc4c(NS(=O)(=O)C(F)(F)F)noc4cc32)cc1Cl]

The title compound was prepared by a method similar to that of Example 1 by replacing methanesulfonyl chloride (Example 1 step 8) with trifluoromethanesulfonyl chloride;

LC-MS: Rt=4.02 mins; MS m/z [M+H]+ 531.3; Method 10-80AB 7minLC_v002

1H NMR (400 MHz, CDCl₃) δ 8.31 (1H, s), 8.01 (1H, s), 7.63 (1H, s), 7.02 (1H, s), 6.60 (1H, s), 4.13 (2H, d), 2.13 (1H, m), 1.72 (2H, m), 1.03 (2H, m), 1.02 (6H, d).

### Example 6

### N-(7′-(5-chloro-6-isobutoxypyridin-3-yl)-6′-oxo-6′,7′-dihydrospiro[cyclopropane-1,5′-isoxazolo[4,5-f]indol]-3′-yl)benzenesulfonamide

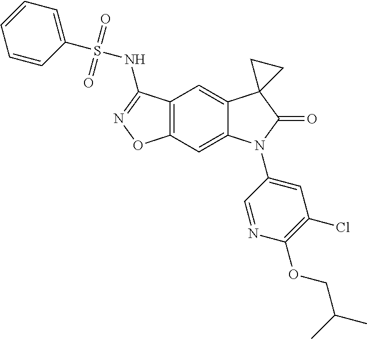 [SMILES: CC(C)COc1ncc(N2C(=O)C3(CC3)c3cc4c(NS(=O)(=O)c5ccccc5)noc4cc32)cc1Cl]

The title compound was prepared by a method similar to that of Example 1 by replacing methanesulfonyl chloride (Example 1 step 8) with benzenesulfonyl chloride;

LC-MS: Rt=5.77 mins; MS m/z [M+H]+ 539.0; Method 10-80AB 7minLC_v002

¹H NMR (400 MHz, CDCl₃) δ 8.16 (1H, d), 7.81 (1H, s), 7.79 (1H, d), 7.75 (1H, d), 7.60 (2H, m), 7.52 (1H, s), 7.48 (2H, t), 6.89 (1H, s), 4.22 (2H, d), 2.18 (1H, m), 1.97 (2H, m), 1.76 (2H, m), 1.08 (6H, d).

### Example 7

### N-(7′-(5-chloro-6-isobutoxypyridin-3-yl)-6′-oxo-6′,7′-dihydrospiro[cyclopropane-1,5′-isoxazolo[4,5-f]indol]-3′-yl)thiophene-2-sulfonamide

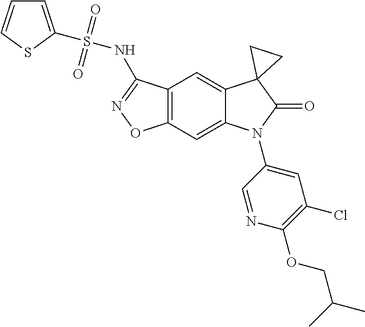 [SMILES: CC(C)COc1ncc(N2C(=O)C3(CC3)c3cc4c(NS(=O)(=O)c5cccs5)noc4cc32)cc1Cl]

The title compound was prepared by a method similar to that of Example 1 by replacing methanesulfonyl chloride (Example 1 step 8) with thiophene-2-sulfonyl chloride;

LCMS: Rt=5.53 mins; MS m/z: [M+H]+ 545.1; Method 10-80AB 7minLC_v002

¹H NMR (400 MHz, DMSO-d₆) δ 8.29 (1H, d), 8.18 (1H, d), 7.95 (1H, m), 7.74 (1H, d), 7.49 (1H, s), 7.18 (1H, t), 7.13 (1H, s), 4.20 (2H, d), 2.11 (1H, m), 1.74 (4H, m), 1.02 (6H, d).

### Example 8

### N-(7′-(5-chloro-6-isobutoxypyridin-3-yl)-6′-oxo-6′,7′-dihydrospiro[cyclopropane-1,5′-isoxazolo[4,5-f]indol]-3′-yl)aminosulfonamide

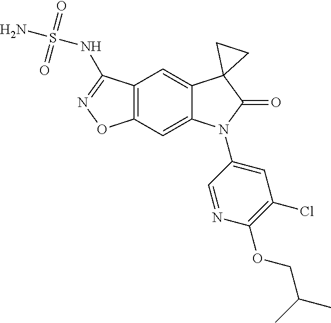 [SMILES: CC(C)COc1ncc(N2C(=O)C3(CC3)c3cc4c(NS(N)(=O)=O)noc4cc32)cc1Cl]

The title compound was prepared by a method similar to that of Example 1 by replacing methanesulfonyl chloride (Example 1 step 8) with sulfamoyl chloride;

LC-MS: Rt=3.87 mins; MS m/z [M+H]+ 478.0; Method 10-80AB 7minLC_v002

¹H NMR (400 MHz, CDCl₃) δ 8.31 (1H, s), 8.13 (1H, d), 7.74 (1H, d), 7.28 (1H, s), 7.08 (2H, s), 6.84 (1H, s), 4.20 (2H, d), 2.13 (1H, m), 1.90 (2H, m), 1.68 (2H, m), 1.06 (6H, d).

### Example 9

### N-(7′-(5-chloro-6-isobutoxypyridin-3-yl)-6′-oxo-6′,7′-dihydrospiro[cyclopropane-1,5′-isoxazolo[4,5-f]indol]-3′-yl)dimethylamino-1-sulfonamide

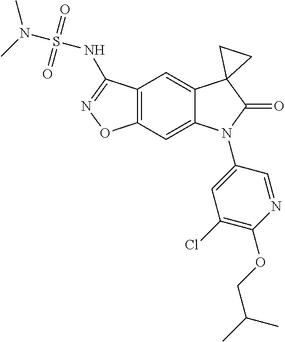 [SMILES: CC(C)COc1ncc(N2C(=O)C3(CC3)c3cc4c(NS(=O)(=O)N(C)C)noc4cc32)cc1Cl]

The title compound was prepared by a method similar to that of Example 1 by replacing methanesulfonyl chloride (Example 1 step 8) with dimethylsulfamoyl chloride;

LC-MS: Rt=5.01 mins; MS m/z [M+H]+ 506.2; Method 10-80AB 7minLC_v002

¹H NMR (400 MHz, CDCl₃) δ 8.16 (1H, d), 7.76 (1H, d), 7.41 (1H, s), 7.34 (1H, s), 6.92 (1H, s), 4.23 (2H, d), 2.93 (6H, s), 2.20 (1H, m), 1.96 (2H, q), 1.74 (2H, m), 1.08 (6H, d).

### Example 10

### N-(7′-(5-chloro-6-isobutoxypyridin-3-yl)-6′-oxo-6′,7′-dihydrospiro[cyclopropane-1,5′-isoxazolo[4,5-f]indol]-3′-yl)pyrrolidine-1-sulfonamide

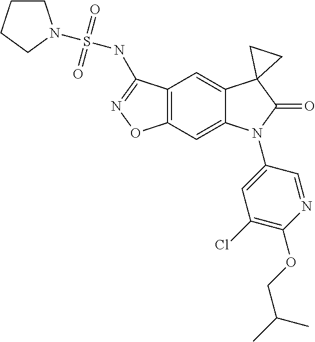 [SMILES: CC(C)COc1ncc(N2C(=O)C3(CC3)c3cc4c(NS(=O)(=O)N5CCCC5)noc4cc32)cc1Cl]

The title compound was prepared by a method similar to that of Example 1 by replacing methanesulfonyl chloride (Example 1 step 8) with pyrrolidine-1-sulfonyl chloride;

LC-MS: Rt=5.82 mins; MS m/z [M+H]+ 532.0; Method 10-80AB 7minLC_v002

¹H NMR (400 MHz, CDCl₃) δ 8.16 (1H, d), 7.76 (1H, d), 7.43 (1H, s), 7.33 (1H, s), 6.91 (1H, s), 4.23 (2H, d), 3.45 (4H, m), 2.20 (1H, m), 1.90 (6H, m), 1.72 (2H, m), 1.08 (6H, m).

### Example 11

### N-(7′-(5-chloro-6-(cyclohexylmethoxy)pyridin-3-yl)-6′-oxo-6′,7′-dihydrospiro[cyclopropane-1,5′-isoxazolo[4,5-f]indol]-3′-yl)methanesulfonamide

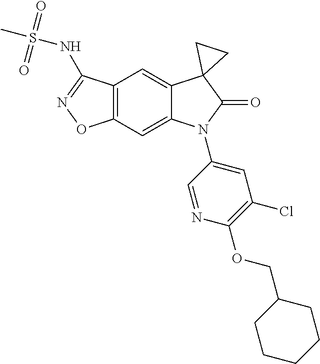 [SMILES: CS(=O)(=O)Nc1noc2cc3c(cc12)C1(CC1)C(=O)N3c1cnc(OCC2CCCCC2)c(Cl)c1]

The title compound was prepared by a method similar to that of Example 1 by replacing 5-bromo-3-chloro-2-isobutoxypyridine (intermediate A Example 1 step 6) with 5-bromo-3-chloro-2-(cyclohexylmethoxy)pyridine (intermediate B);

LC-MS: Rt=4.57 mins; MS m/z [M+H]+ 517.0; Method 10-80AB 7minLC_v002

1HNMR (400 MHz, CDCl₃) δ 8.16 (1H, d), 7.75 (1H, d), 7.36 (1H, s), 7.25 (1H, s), 6.93 (1H, s), 4.25 (2H, d), 3.25 (3H, s), 1.93 (5H, m), 1.79 (2H, m), 1.73 (2H, m), 1.28 (4H, m), 1.11 (2H, m).

### Example 12

### N-(7′-(5-chloro-6-((tetrahydro-2H-pyran-4-yl)methoxy)pyridin-3-yl)-6′-oxo-6′,7′-dihydrospiro[cyclopropane-1,5′-isoxazolo[4,5-f]indol]-3′-yl)methanesulfonamide

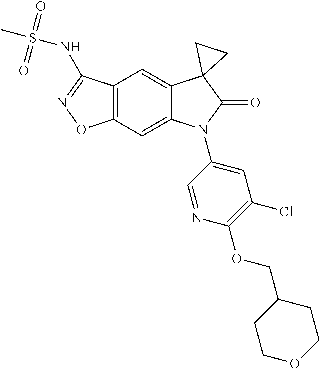 [SMILES: CS(=O)(=O)Nc1noc2cc3c(cc12)C1(CC1)C(=O)N3c1cnc(OCC2CCOCC2)c(Cl)c1]

The title compound was prepared by a method similar to that of Example 1 by replacing 5-bromo-3-chloro-2-isobutoxypyridine (intermediate A Example 1 step 6) with 5-bromo-3-chloro-2-((tetrahydro-2H-pyran-4-yl)methoxy)pyridine (intermediate C);

LC-MS: Rt=4.30 mins; MS m/z [M+H]+ 519.0; Method 10-80AB 7minLC_v002

¹H NMR (400 MHz, CDCl₃) δ 8.16 (1H, d), 7.76 (1H, s), 7.52 (1H, s), 7.35 (1H, s), 6.94 (1H, s), 4.31 (2H, d), 4.04 (2H, m), 3.48 (2H, t), 3.26 (3H, s), 2.18 (1H, br.s), 1.96 (2H, m), 1.762 (4H, m), 1.53 (2H, m).

### Example 13

### N-(7′-(5-chloro-6-((4,4-difluorocyclohexyl)methoxy)pyridin-3-yl)-6′-oxo-6′,7′-dihydrospiro[cyclopropane-1,5′-isoxazolo[4,5-f]indol]-3′-yl)methanesulfonamide

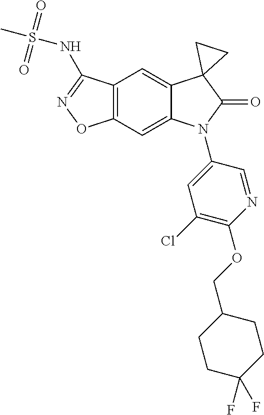 [SMILES: CS(=O)(=O)Nc1noc2cc3c(cc12)C1(CC1)C(=O)N3c1cnc(OCC2CCC(F)(F)CC2)c(Cl)c1]

The title compound was prepared by a method similar to that of Example 1 by replacing 5-bromo-3-chloro-2-isobutoxypyridine (intermediate A Example 1 step 6) with 5-bromo-3-chloro-2-((4,4-difluorocyclohexyl)methoxy)pyridine (intermediate D);

LC-MS: Rt=5.10 mins; MS m/z [M+H]+ 553.0; Method 10-80AB 7minLC_v002

¹H NMR (400 MHz, DMSO-d₆) δ 11.30 (1H, s), 8.32 (1H, d), 8.21 (1H, d), 7.56 (1H, s), 7.17 (1H, s), 4.32 (2H, d), 3.34 (3H, m), 1.99 (3H, m), 1.91 (3H, br.s), 1.81 (1H, m), 1.75 (4H, s), 1.34 (2H, m).

### Example 14

### N-(7′-(5-chloro-6-((4-(trifluoromethyl)cyclohexyl)methoxy)pyridin-3-yl)-6′-oxo-6′,7′-dihydrospiro[cyclopropane-1,5′-isoxazolo[4,5-f]indol]-3′-yl)methanesulfonamide

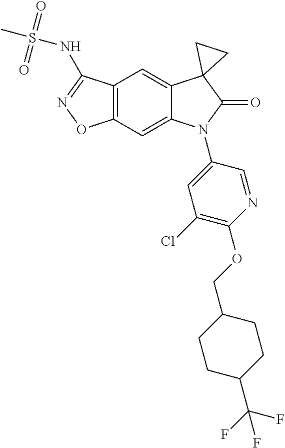 [SMILES: CS(=O)(=O)Nc1noc2cc3c(cc12)C1(CC1)C(=O)N3c1cnc(OCC2CCC(C(F)(F)F)CC2)c(Cl)c1]

The title compound was prepared by a method similar to that of Example 1 by replacing 5-bromo-3-chloro-2-isobutoxypyridine (intermediate A Example 1 step 6) with 5-bromo-3-chloro-2-((4-(trifluoromethyl)cyclohexyl)methoxy)pyridine (intermediate E);

LC-MS: Rt=4.67 mins; MS m/z [M+H]+ 585.1; Method 10-80AB 7minLC_v002

¹H NMR (400 MHz, CDCl₃) δ 8.17 (1H, d), 7.76 (1H, d), 7.71 (1H, s), 7.35 (1H, s), 6.94 (1H, s), 4.42 (2H, d), 3.28 (3H, s), 2.20 (2H, m), 1.95 (2H, m), 1.86 (2H, m), 1.72 (8H, m).

### Example 15

### N-(7′-(5-chloro-6-((1-methylcyclopropyl)methoxy)pyridin-3-yl)-6′-oxo-6′,7′-dihydrospiro[cyclopropane-1,5′-isoxazolo[4,5-f]indol]-3′-yl) methanesulfonamide

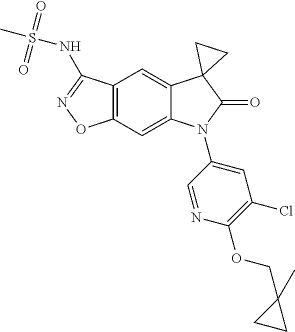

The title compound was prepared by a method similar to that of Example 1 by replacing 5-bromo-3-chloro-2-isobutoxypyridine (intermediate A Example 1 step 6) with 5-bromo-3-chloro-2-((1-methylcyclopropyl)methoxy)pyridine (intermediate I);

LC-MS: Rt=5.16 mins; MS m/z [M−68+H]+ 421.0; Method 10-80AB 7minLC_v002

1H NMR (400 MHz, DMSO-d₆) δ11.28 (1H, s), 8.28 (1H, d), 8.18 (1H, d), 7.48 (1H, s), 7.07 (1H, s), 4.23 (2H, s), 3.21 (3H, s), 1.75 (4H, m), 1.23 (3H, s), 0.58 (2H, m), 0.43 (2H, m).

### Example 16

### N-(7′-(5-chloro-6-((1-methylcyclopropyl)methoxy)pyridin-3-yl)-6′-oxo-6′,7′-dihydrospiro[cyclopropane-1,5′-isoxazolo[4,5-f]indol]-3′-yl)cyclopropanesulfonamide

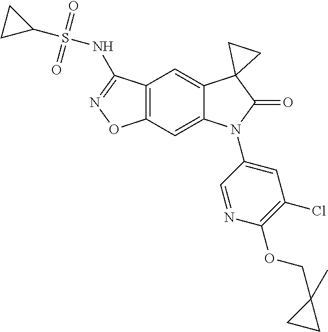 [SMILES: CC1(COc2ncc(N3C(=O)C4(CC4)c4cc5c(NS(=O)(=O)C6CC6)noc5cc43)cc2Cl)CC1]

The title compound was prepared by a method similar to that of Example 1 by replacing 5-bromo-3-chloro-2-isobutoxypyridine (intermediate A Example 1 step 6) with 5-bromo-3-chloro-2-((1-methylcyclopropyl)methoxy)pyridine (intermediate I) and replacing methanesulfonyl chloride (Example 1 step 8) with cyclopropanesulfonyl chloride;

LC-MS: Rt=5.261 mins; MS m/z [M+H]+ 515.1; Method 10-80AB 7minLC_v002

¹H NMR (400 MHz, DMSO-d₆) δ 11.21 (1H, s), 8.29 (1H, d), 8.20 (1H, d), 7.56 (1H, s), 7.16 (1H, s), 4.23 (2H, s), 2.95 (1H, m), 1.75 (4H, m), 1.23 (3H, s), 1.08 (4H, m), 0.59 (2H, m), 0.42 (2H, m).

### Example 17

### N-(7′-(5-chloro-6-(4,4,4-trifluorobutoxyl)pyridin-3-yl)-6′-oxo-6′,7′-dihydrospiro[cyclopropane-1,5′-isoxazolo[4,5-f]indol]-3′-yl)cyclopropanesulfonamide

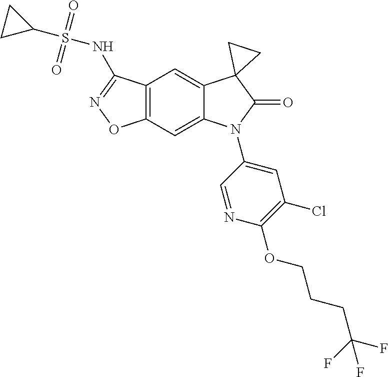 [SMILES: O=C1N(c2cnc(OCCCC(F)(F)F)c(Cl)c2)c2cc3onc(NS(=O)(=O)C4CC4)c3cc2C12CC2]

The title compound was prepared by a method similar to that of Example 1 by replacing 5-bromo-3-chloro-2-isobutoxypyridine (intermediate A Example 1 step 6) with 5-bromo-3-chloro-2-(4,4,4-trifluorobutoxyl)pyridine (intermediate F) and replacing methanesulfonyl chloride (Example 1 step 8) with cyclopropanesulfonyl chloride;

LCMS: Rt=5.06 mins; MS m/z: [M+H]+ 557.0; Method 10-80AB 7minLC_v002

¹H NMR (400 MHz, CDCl₃) δ 8.16 (1H, d), 7.77 (1H, d), 7.42 (2H, s), 6.92 (1H, s), 4.50 (2H, t), 2.6 (1H, m), 2.3 (2H, m), 2.1 (2H, m), 1.93 (2H, q), 1.72 (2H, q), 1.2 (2H, m), 1.1 (2H, m).

### Example 18

### (S)—N-(7′-(5-chloro-6-(1-(4-fluorophenyl)ethoxy)pyridin-3-yl)-6′-oxo-6′,7′-dihydrospiro[cyclopropane-1,5′-isoxazolo[4,5-f]indol]-3′-yl)cyclopropanesulfonamide

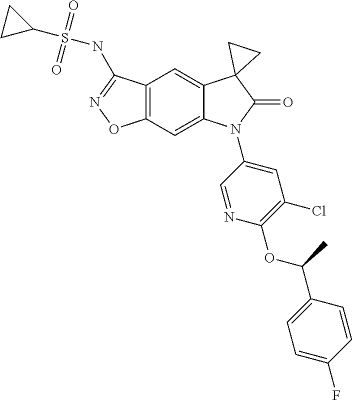 [SMILES: C[C@H](Oc1ncc(N2C(=O)C3(CC3)c3cc4c(NS(=O)(=O)C5CC5)noc4cc32)cc1Cl)c1ccc(F)cc1]

The title compound was prepared by a method similar to that of Example 1 by replacing 5-bromo-3-chloro-2-isobutoxypyridine (intermediate A Example 1 step 6) with (S)-5-bromo-3-chloro-2-(1-(4-fluorophenyl)ethoxy)pyridine (intermediate H) and replacing methanesulfonyl chloride (Example 1 step 8) with cyclopropanesulfonyl chloride;

LCMS: Rt=4.43 mins; MS m/z: [M+H]+ 569.0; Method 10-80AB 7minLC_v002

¹H NMR (400 MHz, CDCl₃) δ 8.10 (1H, d), 7.74 (1H, d), 7.46 (2H, dd), 7.39 (1H, s), 7.29 (1H, s), 7.04 (2H, t), 6.89 (1H, s), 6.29 (1H, q), 2.68 (1H, m), 1.91 (2H, q), 1.69 (5H, d), 1.23 (2H, m), 1.05 (2H, m).

### Example 19

### N-(7′-(5-chloro-6-(3,3-difluorocyclobutoxyl)pyridin-3-yl)-6′-oxo-6′,7′-dihydrospiro[cyclopropane-1,5′-isoxazolo[4,5-f]indol]-3′-yl)cyclopropanesulfonamide

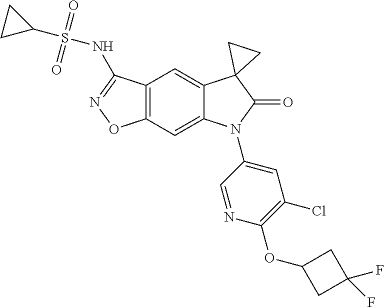 [SMILES: O=C1N(c2cnc(OC3CC(F)(F)C3)c(Cl)c2)c2cc3onc(NS(=O)(=O)C4CC4)c3cc2C12CC2]

The title compound was prepared by a method similar to that of Example 1 by replacing 5-bromo-3-chloro-2-isobutoxypyridine (intermediate A Example 1 step 6) with 5-bromo-3-chloro-2-(3,3-difluorocyclobutoxyl)pyridine (intermediate G) and replacing methanesulfonyl chloride (Example 1 step 8) with cyclopropanesulfonyl chloride;

LC-MS: Rt=4.83 mins; MS m/z [M+H]+ 537.1; Method 10-80AB 7minLC_v002

1HNMR (400 MHz, CDCl₃) δ 8.17 (1H, d), 7.81 (1H, d), 7.44 (1H, s), 7.32 (1H, s), 6.95 (1H, s), 5.24 (1H, m), 3.21 (2H, m), 2.85 (2H, m), 2.68 (1H, m), 1.95 (2H, m), 1.75 (2H, m), 1.23 (2H, m), 1.09 (2H, m).

### Example 20

### N-(7′-(5-chloro-6-(2,2,2-trifluoroethoxyl)pyridin-3-yl)-6′-oxo-6′,7′-dihydrospiro[cyclopropane-1,5′-isoxazolo[4,5-f]indol]-3′-yl)cyclopropanesulfonamide

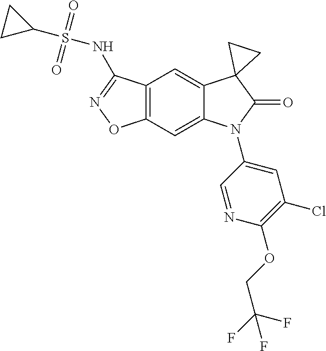 [SMILES: O=C1N(c2cnc(OCC(F)(F)F)c(Cl)c2)c2cc3onc(NS(=O)(=O)C4CC4)c3cc2C12CC2]

The title compound was prepared by a method similar to that of Example 1 by replacing 5-bromo-3-chloro-2-isobutoxypyridine (intermediate A Example 1 step 6) with 5-bromo-3-chloro-2-(2,2,2-trifluoroethoxyl)pyridine (intermediate J) and replacing methanesulfonyl chloride (Example 1 step 8) with cyclopropanesulfonyl chloride;

LC-MS: Rt=3.71 mins; MS m/z 528.9 [M+H]+; Method 10-80AB 1.5minLC_v002

1HNMR (400 MHz, CDCl₃) δ 8.19 (1H, s), 7.85 (1H, s), 7.42 (1H, s), 7.22 (1H, s), 6.93 (1H, s), 4.87 (2H, q), 2.66 (1H, m), 1.94 (2H, m), 1.73 (2H, m), 1.22 (2H, m), 1.07 (2H, m).

### Example 21

### N-(7′-(5-chloro-6-cyclobutoxypyridin-3-yl)-6′-oxo-6′,7′-dihydrospiro[cyclopropane-1,5′-isoxazolo[4,5-f]indol]-3′-yl)cyclopropanesulfonamide

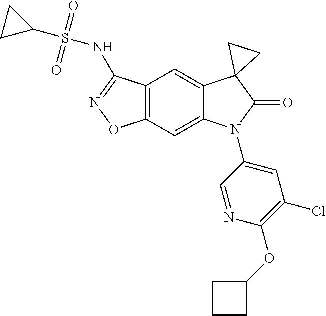 [SMILES: O=C1N(c2cnc(OC3CCC3)c(Cl)c2)c2cc3onc(NS(=O)(=O)C4CC4)c3cc2C12CC2]

The title compound was prepared by a method similar to that of Example 1 by replacing 5-bromo-3-chloro-2-isobutoxypyridine (intermediate A Example 1 step 6) with 5-bromo-3-chloro-2-cyclobutoxypyridine (intermediate K) and replacing methanesulfonyl chloride (Example 1 step 8) with cyclopropanesulfonyl chloride;

LC-MS: Rt=4.68 mins; MS m/z 523.1 [M+Na]+; Method 10-80AB 1.5minLC_v002

¹H NMR (400 MHz, DMSO-d₆) δ: 11.22 (1H, s), 8.29 (1H, d), 8.19 (1H, d), 7.55 (1H, s), 7.16 (1H, s), 5.26 (1H, m), 2.98 (1H, m), 2.44 (2H, m), 2.16 (2H, m), 1.85 (1H, m), 1.75 (3H, d), 1.66 (1H, m), 1.23 (1H, s), 1.06 (4H, m);

### Example 22

### N-(7′-(5-fluoro-6-isobutoxypyridin-3-yl)-6′-oxo-6′,7′-dihydrospiro[cyclopropane-1,5′-isoxazolo[4,5-f]indol]-3′-yl)cyclopropanesulfonamide

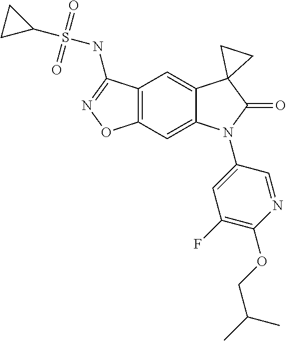 [SMILES: CC(C)COc1ncc(N2C(=O)C3(CC3)c3cc4c(NS(=O)(=O)C5CC5)noc4cc32)cc1F]

The title compound was prepared by a method similar to that of Example 1 by replacing 5-bromo-3-chloro-2-isobutoxypyridine (intermediate A Example 1 step 6) with 5-bromo-3-fluoro-2-isobutoxypyridine (intermediate L) and replacing methanesulfonyl chloride (Example 1 step 8) with cyclopropanesulfonyl chloride;

LC-MS: Rt=3.77 mins; MS m/z 487.1 [M+H]+; Method 10-80AB 1.5minLC_v002

1HNMR (400 MHz, CDCl₃) δ 10.58 (1H, s), 8.04 (1H, s), 7.46 (1H, d), 7.41 (1H, s), 6.93 (1H, s), 4.22 (2H, d), 2.67 (1H, m), 2.21 (1H, m), 1.92 (2H, m), 1.71 (2H, m), 1.21 (2H, m), 1.05 (8H, m).

### Example 23

### N-(7′-(5-chloro-6-((4-methylcyclohexyl)methoxy)pyridin-3-yl)-6′-oxo-6′,7′-dihydrospiro[cyclopropane-1,5′-isoxazolo[4,5-f]indol]-3′-yl)cyclopropanesulfonamide

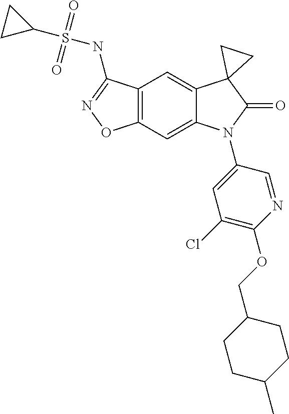 [SMILES: CC1CCC(COc2ncc(N3C(=O)C4(CC4)c4cc5c(NS(=O)(=O)C6CC6)noc5cc43)cc2Cl)CC1]

The title compound was prepared by a method similar to that of Example 1 by replacing 5-bromo-3-chloro-2-isobutoxypyridine (intermediate A Example 1 step 6) with 5-bromo-3-chloro-2-((4-methylcyclohexyl)methoxy)pyridine (intermediate M) and replacing methanesulfonyl chloride (Example 1 step 8) with cyclopropanesulfonyl chloride;

LC-MS: Rt=5.71 mins; MS m/z 557.4 [M+H]+; Method 10-80AB 1.5minLC_v002

¹H NMR (400 MHz, DMSO-d₆) δ 11.21 (1H, s), 8.29 (1H, m), 8.19 (1H, d), 7.54 (1H, s), 7.15 (1H, s), 4.29 (2H, m), 2.96 (1H, m), 1.99 (1H, m), 1.84 (1H, d), 1.67 (5H, m), 1.49 (4H, m), 1.27 (2H, m), 1.13 (5H, m), 0.89 (3H, m);

### Example 24

### N-(7′-(6-(sec-butoxy)-5-chloropyridin-3-yl)-6′-oxo-6′,7′-dihydrospiro[cyclopropane-1,5′-isoxazolo[4,5-f]indol]-3′-yl)cyclopropanesulfonamide

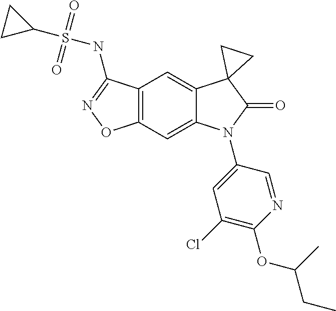 [SMILES: CCC(C)Oc1ncc(N2C(=O)C3(CC3)c3cc4c(NS(=O)(=O)C5CC5)noc4cc32)cc1Cl]

The title compound was prepared by a method similar to that of Example 1 by replacing 5-bromo-3-chloro-2-isobutoxypyridine (intermediate A Example 1 step 6) with 5-bromo-2-(sec-butoxy)-3-chloropyridine (intermediate N) and replacing methanesulfonyl chloride (Example 1 step 8) with cyclopropanesulfonyl chloride;

LC-MS: Rt=4.76 mins; MS m/z 503.2 [M+H]+; Method 10-80AB 1.5minLC_v002

1HNMR (400 MHz, CDCl₃) δ 8.14 (1H, d), 7.73 (1H, d), 7.41 (1H, s), 7.21 (1H, s), 6.93 (1H, s), 5.20 (1H, m), 2.69 (1H, m), 1.94 (2H, m), 1.73 (4H, m), 1.40 (3H, d), 1.21 (2H, m), 1.10 (5H, m).

### Example 25

### N-(7′-(5-chloro-6-(cyclobutylmethoxy)pyridin-3-yl)-6′-oxo-6′,7′-dihydrospiro[cyclopropane-1,5′-isoxazolo[4,5-f]indol]-3′-yl)cyclopropanesulfonamide

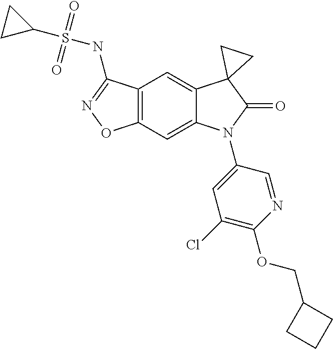 [SMILES: O=C1N(c2cnc(OCC3CCC3)c(Cl)c2)c2cc3onc(NS(=O)(=O)C4CC4)c3cc2C12CC2]

The title compound was prepared by a method similar to that of Example 1 by replacing 5-bromo-3-chloro-2-isobutoxypyridine (intermediate A Example 1 step 6) with 5-bromo-3-chloro-2-(cyclobutylmethoxy)pyridine (intermediate 0) and replacing methanesulfonyl chloride (Example 1 step 8) with cyclopropanesulfonyl chloride;

LC-MS: Rt=4.94 mins; MS m/z 515.2 [M+H]+; Method 10-80AB 1.5minLC_v002

1HNMR (400 MHz, CDCl₃) δ 8.15 (1H, d), 7.74 (1H, d), 7.41 (1H, s), 7.21 (1H, s), 6.92 (1H, s), 4.41 (2H, d), 2.84 (1H, m), 2.65 (1H, m), 2.15 (2H, m), 1.90 (6H, m), 1.72 (2H, q), 1.20 (2H, m), 1.08 (2H, m).

### Example 26

### N-(7′-(5-chloro-6-(cyclohexyloxy)pyridin-3-yl)-6′-oxo-6′,7′-dihydrospiro[cyclopropane-1,5′-isoxazolo[4,5-f]indol]-3′-yl)cyclopropanesulfonamide

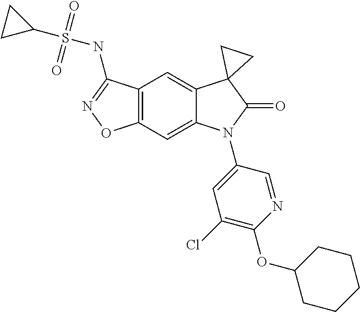 [SMILES: O=C1N(c2cnc(OC3CCCCC3)c(Cl)c2)c2cc3onc(NS(=O)(=O)C4CC4)c3cc2C12CC2]

The title compound was prepared by a method similar to that of Example 1 by replacing 5-bromo-3-chloro-2-isobutoxypyridine (intermediate A Example 1 step 6) with 5-bromo-3-chloro-2-(cyclohexyloxy)pyridine (intermediate P) and replacing methanesulfonyl chloride (Example 1 step 8) with cyclopropanesulfonyl chloride;

LC-MS: Rt=5.16 mins; MS m/z 529.1 [M+H]+; Method 10-80AB 1.5minLC_v002

1HNMR (400 MHz, CDCl₃) δ 8.30 (1H, d), 8.17 (1H, d), 7.49 (1H, s), 7.29 (1H, s), 7.10 (1H, s), 5.17 (1H, m), 2.90 (1H, m), 1.99 (2H, m), 1.73 (6H, m), 1.57 (3H, m), 1.36 (3H, m), 0.99 (4H, m).

### Example 27

### N-(7′-(5-chloro-6-(cyclopentylmethoxy)pyridin-3-yl)-6′-oxo-6′,7′-dihydrospiro[cyclopropane-1,5′-isoxazolo[4,5-f]indol]-3′-yl)cyclopropanesulfonamide

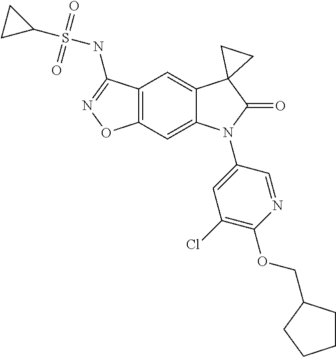 [SMILES: O=C1N(c2cnc(OCC3CCCC3)c(Cl)c2)c2cc3onc(NS(=O)(=O)C4CC4)c3cc2C12CC2]

The title compound was prepared by a method similar to that of Example 1 by replacing 5-bromo-3-chloro-2-isobutoxypyridine (intermediate A Example 1 step 6) with 5-bromo-3-chloro-2-(cyclopentylmethoxy)pyridine (intermediate Q) and replacing methanesulfonyl chloride (Example 1 step 8) with cyclopropanesulfonyl chloride;

LC-MS: Rt=5.19 mins; MS m/z 529.1 [M+H]+; Method 10-80AB 1.5minLC_v002

1HNMR (400 MHz, CDCl₃) δ 8.16 (1H, d), 7.76 (1H, d), 7.48 (1H, s), 7.43 (1H, s), 6.31 (1H, s), 4.34 (2H, q), 2.72 (1H, m), 2.47 (1H, m), 1.95 (4H, m), 1.73 (6H, m), 1.40 (2H, m), 1.22 (2H, m), 1.08 (2H, m).

### Example 28

### N-(7′-(5-chloro-6-(cyclopropylmethoxy)pyridin-3-yl)-6′-oxo-6′,7′-dihydrospiro[cyclopropane-1,5′-isoxazolo[4,5-f]indol]-3′-yl)cyclopropanesulfonamide

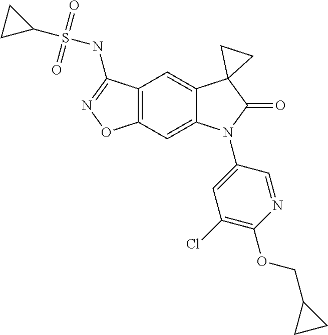 [SMILES: O=C1N(c2cnc(OCC3CC3)c(Cl)c2)c2cc3onc(NS(=O)(=O)C4CC4)c3cc2C12CC2]

The title compound was prepared by a method similar to that of Example 1 by replacing 5-bromo-3-chloro-2-isobutoxypyridine (intermediate A Example 1 step 6) with 5-bromo-3-chloro-2-(cyclopropylmethoxy)pyridine (intermediate R) and replacing methanesulfonyl chloride (Example 1 step 8) with cyclopropanesulfonyl chloride;

LC-MS: Rt=4.47 mins; MS m/z 501.1 [M+H]+; Method 10-80AB 1.5minLC_v002

1HNMR (400 MHz, CDCl₃) δ 8.15 (1H, s), 7.76 (1H, s), 7.43 (1H, s), 7.23 (1H, s), 6.92 (1H, s), 4.31 (2H, d), 2.70 (1H, m), 1.94 (2H, m), 1.73 (2H, m), 1.24 (1H, m), 1.09 (2H, m), 1.02 (2H, m), 0.65 (2H, m), 0.45 (2H, m).

### Example 29

### N-(7′-(5-chloro-6-methoxypyridin-3-yl)-6′-oxo-6′,7′-dihydrospiro[cyclopropane-1,5′-isoxazolo[4,5-f]indol]-3′-yl)cyclopropanesulfonamide

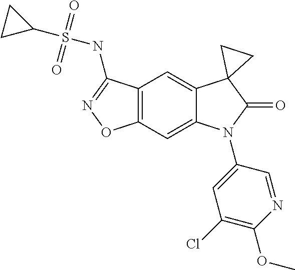 [SMILES: COc1ncc(N2C(=O)C3(CC3)c3cc4c(NS(=O)(=O)C5CC5)noc4cc32)cc1Cl]

The title compound was prepared by a method similar to that of Example 1 by replacing 5-bromo-3-chloro-2-isobutoxypyridine (intermediate A Example 1 step 6) with 5-bromo-3-chloro-2-methoxypyridine (intermediate S) and replacing methanesulfonyl chloride (Example 1 step 8) with cyclopropanesulfonyl chloride;

LC-MS: Rt=3.76 mins; MS m/z 461.1 [M+H]+; Method 10-80AB 1.5minLC_v002

1HNMR (400 MHz, CDCl₃) δ 8.19 (1H, s), 7.77 (1H, d), 7.41 (1H, s), 7.21 (1H, s), 6.91 (1H, s), 4.10 (3H, s), 2.69 (1H, m), 1.94 (2H, m), 1.73 (2H, m), 1.23 (2H, m), 1.07 (2H, m).

### Example 30

### N-(7′-(5-chloro-6-(difluoromethoxy)pyridin-3-yl)-6′-oxo-6′,7′-dihydrospiro[cyclopropane-1,5′-isoxazolo[4,5-f]indol]-3′-yl)cyclopropanesulfonamide

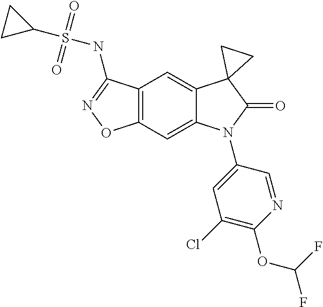 [SMILES: O=C1N(c2cnc(OC(F)F)c(Cl)c2)c2cc3onc(NS(=O)(=O)C4CC4)c3cc2C12CC2]

The title compound was prepared by a method similar to that of Example 1 by replacing 5-bromo-3-chloro-2-isobutoxypyridine (intermediate A Example 1 step 6) with 5-bromo-3-chloro-2-(difluoromethoxy)pyridine (intermediate T) and replacing methanesulfonyl chloride (Example 1 step 8) with cyclopropanesulfonyl chloride;

LC-MS: Rt=4.05 mins; MS m/z 497.1 [M+H]+; Method 10-80AB 1.5minLC_v002

1HNMR (400 MHz, DMSO-d₆) δ 11.26 (1H, s), 8.46 (2H, s), 7.83 (1H, t), 7.49 (1H, s), 7.15 (1H, s), 2.90 (1H, m), 1.74 (4H, m), 1.00 (4H, m).

### Example 31

### N-(7′-(3,4-dichlorophenyl)-6′-oxo-6′,7′-dihydrospiro[cyclopropane-1,5′-isoxazolo[4,5-f]indol]-3′-yl)methanesulfonamide

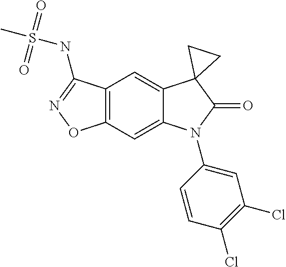 [SMILES: CS(=O)(=O)Nc1noc2cc3c(cc12)C1(CC1)C(=O)N3c1ccc(Cl)c(Cl)c1]

The title compound was prepared by a method similar to that of Example 1 by replacing 5-bromo-3-chloro-2-isobutoxypyridine (intermediate A Example 1 step 6) with 4-bromo-1,2-dichlorobenzene;

LC-MS: Rt=4.78 mins; MS m/z [M+H]+ 438.0; Method 10-80AB 7minLC_v002

1H NMR (400 MHz, DMSO-d₆) δ 11.31 (1H, s), 7.85 (2H, m), 7.55 (2H, m), 7.18 (1H, s), 3.30 (3H, s), 1.75 (4H, m).

### Example 32

### N-(7′-(3-chloro-5-cyano-4-isobutoxyphenyl)-6′-oxo-6′,7′-dihydro spiro[cyclopropane-1,5′-isoxazolo[4,5-f]indol]-3′-yl)methanesulfonamide

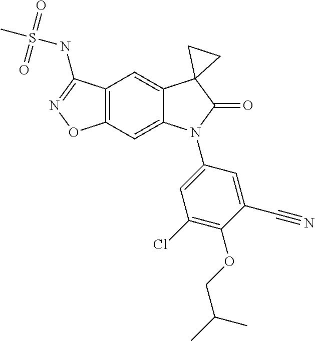 [SMILES: CC(C)COc1c(Cl)cc(N2C(=O)C3(CC3)c3cc4c(NS(C)(=O)=O)noc4cc32)cc1C#N]

The title compound was prepared by a method similar to that of Example 1 by replacing 5-bromo-3-chloro-2-isobutoxypyridine (intermediate A Example 1 step 6) with 5-bromo-3-chloro-2-isobutoxybenzonitrile (intermediate X).

LC-MS: Rt=4.38 mins; MS m/z [M+H]+ 501.0; Method 10-80AB 7minLC_v002

1H NMR (400 MHz, CDCl₃) δ 7.68 (1H, d), 7.56 (1H, d), 7.29 (1H, s), 7.25 (1H, s), 6.92 (1H, s), 4.03 (2H, d), 3.19 (3H, s), 2.17 (1H, m), 1.89 (2H, m), 1.70 (2H, m), 1.07 (6H, d).

### Example 33

### N-(7′-(3-chloro-5-fluoro-4-isobutoxyphenyl)-6′-oxo-6′,7′-dihydro spiro[cyclopropane-1,5′-isoxazolo[4,5-f]indol]-3′-yl)cyclopropanesulfonamide

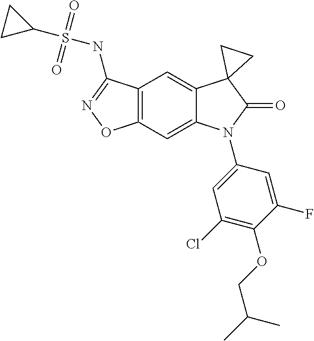 [SMILES: CC(C)COc1c(F)cc(N2C(=O)C3(CC3)c3cc4c(NS(=O)(=O)C5CC5)noc4cc32)cc1Cl]

The title compound was prepared by a method similar to that of Example 1 by replacing 5-bromo-3-chloro-2-isobutoxypyridine (intermediate A Example 1 step 6) with 5-bromo-1-chloro-3-fluoro-2-isobutoxybenzene (intermediate w) and replacing methanesulfonyl chloride (Example 1 step 8) with cyclopropanesulfonyl chloride;

LCMS: Rt=4.50 mins; MS m/z: [M+H]+ 520.0; Method 10-80AB 7minLC_v002

¹HNMR (400 MHz, MeOD) δ 7.42 (1H, m), 7.35 (2H, m), 6.95 (1H, s), 3.97 (2H, d), 2.90 (1H, m), 2.13 (1H, m), 1.80 (4H, m), 1.14 (2H, m), 1.09 (6H, d), 0.96 (2H, m).

### Example 34

### N-(7-(5-chloro-6-isobutoxypyridin-3-yl)-5-methyl-6-oxo-6,7-dihydro-5H-imidazo[4′,5′:4,5]benzo[1,2-d]isoxazol-3-yl)methanesulfonamide

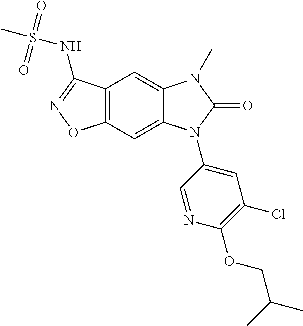 [SMILES: CC(C)COc1ncc(-n2c(=O)n(C)c3cc4c(NS(C)(=O)=O)noc4cc32)cc1Cl]

### Step 1: 5-bromo-4-fluoro-N-methyl-2-nitroaniline

To a solution of 1-bromo-2,5-difluoro-4-nitrobenzene (Aldrich) (15 g, 60 mmol) in DMF (50 ml) was added methanamine (2 M in THF, 47 ml, 90 mmol) and DIPEA (28 ml, 150 mmol). The resulting mixture was stirred at 25° C. for 16 hrs. When TLC indicated the starting material was consumed, the mixture was evaporated under reduced pressure to give the residue, which was treated with hexanes and ethyl acetate. The solid was collected and dried to give the title compound.

LC-MS: Rt=1.23 mins; MS m/z [M+H]+ 248.9; Method 5-95AB 1.5minLC_v003

¹H NMR (400 MHz, CDCl₃) δ 7.94 (2H, m), 7.07 (1H, d), 3.03 (3H, d).

### Step 2: 2-fluoro-5-(methylamino)-4-nitrobenzonitrile

To a mixture of 5-bromo-4-fluoro-N-methyl-2-nitroaniline (step 1) (13 g, 52 mmol), DPPF (3.4 g, 6.2 mmol) and Zn(CN)₂ (3.6 g, 29 mmol) in DMF (50 ml) was added Pd₂(dba)₃ (2.3 g, 2.6 mmol) at room temperature. The resulting mixture was degassed and charged with nitrogen three times. After heating to 120° C., the reaction mixture was stirred at that temperature under nitrogen atmosphere for 16 hrs. When LC/MS indicated the starting material was consumed, the reaction mixture was cooled down and diluted with ethyl acetate (1000 ml). The mixture was filtered through a pad of celite and the filtration was then washed with brine (×3), dried over anhydrous magnesium sulfate and filtered. The filtration was evaporated under reduced pressure to give the crude product, which was purified by column chromatography on silica gel eluting with 0-35% ethyl acetate in hexanes to afford the title compound as a white solid.

LC-MS: Rt=1.25 mins; MS m/z [M+H]+ 196.1; Method 5-95AB 1.5minLC_v003

¹H NMR (400 MHz, CDCl₃) δ 8.06 (1H, d), 7.88 (1H, br.s), 7.12 (1H, d), 3.06 (3H, d).

### Step 3: 4-amino-2-fluoro-5-(methylamino)benzonitrile

To a mixture of 2-fluoro-5-(methylamino)-4-nitrobenzonitrile (step 2) (3 g, 15 mmol) in DCM (300 ml) was added Zn (5 g, 75 mmol) and HOAc (4.6 g, 75 mmol) at 0° C. The resulting mixture was stirred at 25° C. for 0.5 h. After filtered, the filtrate was concentrated under reduced pressure to give the residue, which was treated with ethyl acetate and saturated NaHCO₃ aqueous solution. The separated organic phase was washed with brine (×2), dried over anhydrousmagnesium sulfate and filtered. The filtration solution was concentrated under reduced pressure to give the crude product, which was purified by flash column chromatography on silica gel eluting with 0˜50% ethyl acetate in hexanes to afford the title compound.

LC-MS: Rt=1.09 mins; MS m/z [M+H]+ 166.1; Method 5-95AB 1.5minLC_v003

¹H NMR (400 MHz, CDCl₃) δ 6.72 (1H, d), 6.47 (1H, d), 5.62 (2H, br, s), 4.02 (1H, m), 2.84 (3H, d).

### Step 4: 6-fluoro-3-methyl-2-oxo-2,3-dihydro-1H-benzo[d]imidazole-5-carbonitrile

To a mixture of 4-amino-2-fluoro-5-(methylamino)benzonitrile (step 3) (1.5 g, 8.8 mmol) in toluene (50 ml) was added CDI (4.3 g, 26 mmol). The resulting mixture was stirred at 80° C. for 1 h. When LC/MS indicated the reaction was completed, the reaction mixture was evaporated under reduced pressure to give the residue, which was treated with ethyl acetate and water. The separated organic phase was washed with brine (×2), dried over anhydrousmagnesium sulfate and filtered. The filtration solution was concentrated under reduced pressure to give the crude product, which was purified by flash column chromatography on silica gel eluting with 0˜30% ethyl acetate in hexanes to afford the title compound.

LC-MS: Rt=1.18 mins; MS m/z [M+H]+ 192.2; Method 5-95AB 1.5minLC_v003

¹H NMR (400 MHz, DMSO-d₆) δ 10.64 (1H, s), 7.64 (1H, d), 7.13 (1H, d), 3.29 (3H, s).

### Step 5: 1-(5-chloro-6-isobutoxypyridin-3-yl)-6-fluoro-3-methyl-2-oxo-2,3-dihydro-1H-benzo[d]imidazole-5-carbonitrile

To a mixture of 6-fluoro-3-methyl-2-oxo-2,3-dihydro-1H-benzo[d]imidazole-5-carbonitrile (step 4) (600 mg, 3.16 mmol) in CH₃CN (30 ml) was added 5-bromo-3-chloro-2-isobutoxypyridine (intermediate A) (2.0 g, 7.9 mmol), CuI (1.2 g, 6.3 mmol), K₂CO₃ (880 g, 6.3 mmol) and DMEDA (560 mg, 6.3 mmol) at room temperature. The resulting mixture was degassed and charged with nitrogen three times. After heating to 100° C., the reaction mixture was stirred at that temperature for 16 hrs. When LC/MS indicated the starting material was consumed, the reaction mixture was cooled down and diluted with ethyl acetate (100 ml). The mixture was filtered through a pad of celite and the filtration was then washed with brine (×3), dried over anhydrous magnesium sulfate and filtered. The filtration was evaporated under reduced pressure to give the crude product, which was purified by column chromatography on silica gel eluting with 0˜20% ethyl acetate in hexanes to afford the title compound as a white solid.

LC-MS: Rt=1.23 mins; MS m/z [M+H]+ 374.8; Method 5-95AB 1.5minLC_v003

¹H NMR (400 MHz, CDCl₃) δ 8.13 (1H, d), 7.75 (1H, d), 7.17 (1H, d), 6.82 (1H, d), 4.22 (2H, m), 3.20 (3H, s), 2.17 (1H, m), 1.05 (6H, d).

### Step 6: 3-amino-7-(5-chloro-6-isobutoxypyridin-3-yl)-5-methyl-5H-imidazo[4′,5′:4,5]benzo[1,2-d]isoxazol-6(7H)-one

To a mixture of propan-2-one oxime (30 mg, 0.40 mmol) in anhydrous DMF (2 ml) was added t-BuOK (450 mg, 0.40 mmol) at room temperature. The resulting mixture was stirred at room temperature for 30 min and then a solution of 1-(5-chloro-6-isobutoxypyridin-3-yl)-6-fluoro-3-methyl-2-oxo-2,3-dihydro-1H-benzo[d]imidazole-5-carbonitrile (step 5) (100 mg, 0.27 mmol) in DMF (1 ml) added. After stirring at room temperature for 3 hrs, the reaction was quenched with saturated ammonium chloride solution. The reaction mixture was extracted with ethyl acetate (×3). The combined organic phases were washed with brine (×2), dried over anhydrous magnesium sulfate and filtered. The filtration was concentrated under reduced pressure to give the crude product, which was treated with 10 ml of ethanol and 10 ml of 5% HCl aqueous solution. The resulting mixture was heated to reflux for 45 min. After removal of the organic solvent, the remaining aqueous phase was basified with saturated Na₂CO₃ aqueous solution. The mixture was extracted with ethyl acetate (×3), washed with brine (×2), dried over anhydrous magnesium sulfate and filtered. The filtration was concentrated under reduced pressure to give the crude product, which was purified by flash chromatography on silica gel eluting with 0˜20% ethyl acetate in hexanes to afford the title compound as a white solid.

LC-MS: Rt=1.03 mins; MS m/z [M+H]+ 387.8; Method 5-95AB 1.5minLC_v003

¹H NMR (400 MHz, CDCl₃) δ 8.20 (1H, d), 7.83 (1H, d), 7.29 (1H, s), 7.25 (1H, s), 4.30 (2H, s), 4.20 (2H, d), 3.29 (3H, s), 2.18 (1H, m), 1.07 (6H, d).

### Step 7: N-(7-(5-chloro-6-isobutoxypyridin-3-yl)-5-methyl-6-oxo-6,7-dihydro-5H-imidazo[4′,5′:4,5]benzo[1,2-d]isoxazol-3-yl)-N-(methylsulfonyl)methanesulfonamide

To a mixture of 3-amino-7-(5-chloro-6-isobutoxypyridin-3-yl)-5-methyl-5H-imidazo[4′,5′:4,5]benzo[1,2-d]isoxazol-6(7H)-one (step 6) (100 mg, 0.26 mmol) in dry DCM (10 ml) was added TEA (78 mg, 0.78 mmol) and methanesulfonyl chloride (88 mg, 0.78 mmol) at room temperature. The resulting mixture was stirred at room temperature for 2 hrs and quenched with saturated ammonium chloride aqueous solution. The reaction mixture was washed with brine (×3), dried over anhydrous magnesium sulfate and filtered. The filtration was concentrated under reduced pressure to afford the crude product, which was dissovled in THF (10 ml). To the above mixture was added TBAF (144 mg, 0.55 mmol) and the resulting mixture was then stirred at room temperature for 2 hrs. After removal of the solvent, the residue was treated with ethyl acetate and water. The separated organic phase was washed with water (×2), dried over anhydrous magnesium sulfate and filtered. The filtration was concentrated under reduced pressure to give the crude product, which was purified by preparative HPLC to afford the title compound as a white solid.

LC-MS: Rt=5.44 mins; MS m/z [M+H]+ 387.8; Method 0-60AB 7minLC_v002

¹H NMR (400 MHz, CDCl₃) δ 8.21 (1H, d), 7.81 (1H, d), 7.59 (1H, s), 7.45 (1H, s), 7.10 (1H, s), 4.22 (2H, d), 3.55 (3H, s), 3.29 (3H, s), 2.19 (1H, m), 1.08 (6H, d).

### Example 35

### N-(7-(5-chloro-6-isobutoxypyridin-3-yl)-5-methyl-6-oxo-6,7-dihydro-5H-imidazo[4′,5′:4,5]benzo[1,2-d]isoxazol-3-yl)cyclopropanesulfonamide

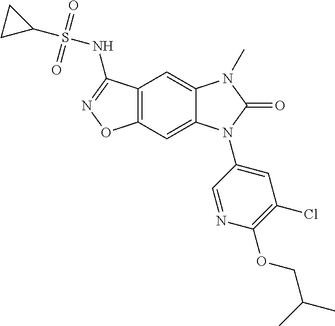 [SMILES: CC(C)COc1ncc(-n2c(=O)n(C)c3cc4c(NS(=O)(=O)C5CC5)noc4cc32)cc1Cl]

The title compound was prepared by a method similar to that of Example 35 by replacing methanesulfonyl chloride (Example 35 step 7) with cyclopropanesulfonyl chloride;

LCMS: Rt 4.97 mins; MS m/z: [M+H]+ 492.0; Method 10-80AB 7minLC_v002

¹H NMR (400 MHz, DMSO-d₆) δ 11.26 (1H, s), 8.34 (1H, d), 8.19 (1H, d), 7.61 (1H, s), 7.37 (1H, s), 4.20 (2H, d), 3.43 (3H, s), 3.00 (1H, m), 2.10 (1H, m), 1.08 (4H, m), 1.01 (6H, d).

### Example 36

### N-(7-(5-chloro-6-isobutoxypyridin-3-yl)-5,5-dimethyl-6-oxo-6,7-dihydro-5H-isoxazolo[4,5-f]indol-3-yl)cyclopropanesulfonamide

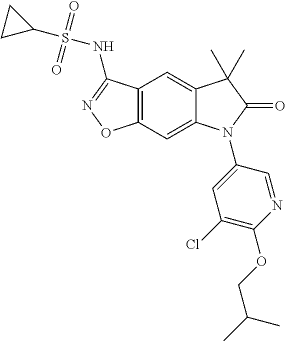 [SMILES: CC(C)COc1ncc(N2C(=O)C(C)(C)c3cc4c(NS(=O)(=O)C5CC5)noc4cc32)cc1Cl]

### Step 1: tert-butyl 6-fluoro-3,3-dimethyl-2-oxoindoline-1-carboxylate

To a mixture of tert-butyl 6-fluoro-2-oxoindoline-1-carboxylate (intermediate from example 1, step 1) (4.5 g 18 mmol) in 100 ml of THF was added NaH (5 g, 99 mmol) by portion at 0° C. When the addition was completed, the resulting mixture was stirred at room temperature for 30 min. Then iodomethane (25 g, 179 mmol) was added to the reaction mixture. After stirring at room temperature for additional 3 hrs, the reaction mixture was poured into saturated NH₄Cl aqueous solution. The mixture was extracted with DCM (×3). The combined organic layers were washed with brine, dried over anhydrous magnesium sulfate and filtered. The filtration was evaporated under reduced pressure to afford crude product, which was purified by flash column chromatography on silica gel using a gradient 0-10% ethyl acetate in hexanes to afford the title compound as yellow oil.

LC-MS: Rt=1.42 mins; MS m/z [M+H]+ 280.1; Method 5-95AB 1.5minLC_v003

¹H NMR (400 MHz, CDCl₃) δ 7.63 (1H, dd), 7.13 (1H, dd), 6.86 (1H, dt), 1.64 (9H, s), 1.4 (6H, s).

### Step 2: 6-fluoro-3,3-dimethylindolin-2-one

To a mixture of tert-butyl 6-fluoro-3,3-dimethyl-2-oxoindoline-1-carboxylate (step 1) (1.2 g, 4.3 mmol) in 10 ml of ethyl acetate was added 4N HCl/ethyl acetate (10 ml). The resulting mixture was stirred at room temperature for 1 h and then evaporated under reduced pressure to afford the title compound as a off-white solid.

LC-MS: Rt=1.26 mins; MS m/z [M+H]+ 180.1; Method 5-95AB 1.5minLC_v003

¹H NMR (400 MHz, CDCl₃) δ 8.11 (1H, br. s), 7.10 (1H, dd), 6.67 (1H, m), 6.64 (1H, dd), 1.37 (6H, s).

### Step 3: 5-bromo-6-fluoro-3,3-dimethylindolin-2-one

To a mixture of 6-fluoro-3,3-dimethylindolin-2-one (step 2) (750 mg, 4.2 mmol) in 10 ml of MeCN was added NBS (819 mg, 4.6 mmol). The resulting reaction was stirred at room temperature for 16 hrs. After removal of the solvent, the residue was treated with hot water (>90° C.). The solid was collected and dried to give the title compound as off-white solid.

LC-MS: Rt=1.48 mins; MS m/z [M+H]+ 258.0; Method 5-95AB 1.5minLC_v003

¹H NMR (400 MHz, CDCl₃) δ 8.08 (1H, br. s), 7.30 (1H, dd), 6.27 (1H, m), 1.37 (6H, s).

### Step 4: 6-fluoro-3,3-dimethyl-2-oxoindoline-5-carbonitrile

To a mixture of 5-bromo-6-fluoro-3,3-dimethylindolin-2-one (step 3) (980 mg, 3.8 mmol), Zn(CN)₂ (892 mg, 7.6 mmol) and dppf (842 mg, 1.5 mmol) in DMF (30 ml) was added Pd₂dba₃ (695 mg, 0.76 mmol) at 20° C. The resulting mixture was degassed and charged with N₂ three times, and then stirred at 120° C. under N₂ atmosphere for 5 hrs. When LC/MS indicated the starting material was consumed, the reaction mixture was diluted with ethyl acetate. The mixture was filtered over celite. The filtration was washed with water (×3), dried over anhydrous magnesium sulfate and filtered. The filtration was evaporated under reduced pressure to afford the crude product, which was purified by flash column chromatography on silica gel using a gradient 0-25% ethyl acetate in hexanes to afford the title compound as yellow solid.

LC-MS: Rt=1.40 mins; MS m/z [M+H]+ 205.2; Method 5-95AB 1.5minLC_v003

¹H NMR (400 MHz, CDCl₃) δ: 8.60 (1H, br. s), 7.37 (1H, d), 6.80 (1H, d), 1.42 (6H, s).

### Step 5: 1-(5-chloro-6-isobutoxypyridin-3-yl)-6-fluoro-3,3-dimethyl-2-oxoindoline-5-carbonitrile

To a mixture of 6-fluoro-3,3-dimethyl-2-oxoindoline-5-carbonitrile (step 4) (350 mg, 1.7 mmol), 5-bromo-3-chloro-2-isobutoxypyridine (intermediate A) (1.36 g, 5.1 mmol), DMEDA (0.37 ml, 3.4 mmol) and K₂CO₃ (472 mg, 3.4 mmol) in 30 ml of 1,4-dioxane was added CuI (325 mg, 3.4 mmol) at room temperature. The resulting mixture was degassed and charged with N₂ three times, and then stirred at 100° C. under N₂ atmosphere for 3 hrs. When LC/MS indicated the starting material was consumed, the reaction mixture was diluted with ethyl acetate. The mixture was filtered over celite. The filtration was washed with water (×3), dried over anhydrous magnesium sulfate and filtered. The filtration was evaporated under reduced pressure to afford the crude product, which was purified by flash column chromatography on silica gel using a gradient 0-25% ethyl acetate in hexanes to afford the title compound as white foam.

LC-MS: Rt=1.49 mins; MS m/z [M+H]+ 388.0; Method 5-95AB 1.5minLC_v003

¹H NMR (400 MHz, CDCl₃) δ: 8.04 (1H, d), 7.64 (1H, d), 7.6 (1H, d), 6.61 (1H, d), 4.19 (2H, d), 2.16 (1H, qd), 1.49 (6H, s), 1.05 (6H, d).

### Step 6: 3-amino-7-(5-chloro-6-isobutoxypyridin-3-yl)-5,5-dimethyl-5H-isoxazolo[4,5-f]indol-6(7H)-one

To a mixture of 1-(5-chloro-6-isobutoxypyridin-3-yl)-6-fluoro-3,3-dimethyl-2-oxoindoline-5-carbonitrile (step 5) (700 mg, 1.8 mmol) and N-hydroxyacetamide (406 mg, 5.4 mmol) in 30 ml of DMF was added t-BuOK (5.4 ml, 1 M in THF, 5.4 mmol). This reaction mixture was stirred at room temperature for 4 hrs. After removal of the solvent under reduced pressure, the residue was treated with water and ethyl acetate. The separated organic phase was washed with brine (×3), dried over anhydrous Magnesium sulfate and filtered. The filtration was evaporated under reduced pressure to afford the crude product, which was purified by flash column chromatography on silica gel using a gradient 0-30% ethyl acetate in hexanes to afford the title compound as yellow solid.

LC-MS: Rt=1.04 mins; MS m/z [M+H]+ 401.3; Method 5-95AB 1.5minLC_v003

¹H NMR (400 MHz, CDCl₃) δ: 8.10 (1H, d), 7.71 (1H, d), 7.35 (1H, s), 6.8 (1H, s), 4.32 (2H, s), 4.20 (2H, d), 2.17 (1H, dt), 1.52 (6H, s), 1.06 (6H, d).

### Step 7: N-(7-(5-chloro-6-isobutoxypyridin-3-yl)-5,5-dimethyl-6-oxo-6,7-dihydro-5H-isoxazolo[4,5-f]indol-3-yl)cyclopropanesulfonamide

To a mixture of 3-amino-7-(5-chloro-6-isobutoxypyridin-3-yl)-5,5-dimethyl-5H-isoxazolo[4,5-f]indol-6(7H)-one (step 6) (100 mg, 0.25 mmol), DMAP (31 mg, 0.25 mmol) in 3 ml of pyridine was added cyclopropanesulfonyl chloride (0.25 ml, 2.5 mmol). The resulting reaction was stirred at room temperature for 18 hrs. When LC/MS indicated the reaction was completed, the mixture was diluted with DCM (10 ml) and washed with water (×2). The organic layer was evaporated under reduced pressure to afford crude product, which was purified by preparative HPLC to give the title compound as white solid.

LC-MS: Rt=3.77 mins; MS m/z [M+H]+ 505.0; Method 10-80AB 7minLC_v001

¹H NMR (400 MHz, CDCl₃) δ: 8.11 (1H, d), 7.80 (1H, s), 7.71 (1H, d), 7.21 (1H, d), 6.85 (1H, s), 4.21 (2H, d), 2.67 (1H, m), 2.18 (1H, m), 1.54 (6H, s), 1.22 (2H, m), 1.01 (8H, m).

### Example 37

### N-(7′-(5-chloro-6-isobutoxypyridin-3-yl)-6′-oxo-6′,7′-dihydrospiro[cyclobutane-1,5′-isoxazolo[4,5-f]indol]-3′-yl)cyclopropanesulfonamide

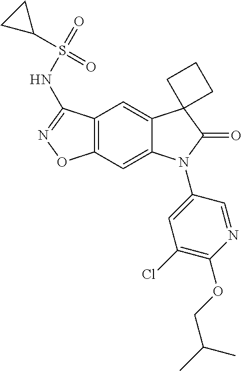 [SMILES: CC(C)COc1ncc(N2C(=O)C3(CCC3)c3cc4c(NS(=O)(=O)C5CC5)noc4cc32)cc1Cl]

The title compound was prepared by a method similar to that of Example 36 by replacing iodomethane (Example 36 step 1) with 1,3-diiodopropane;

LC-MS: Rt=5.95 mins; MS m/z [M+H]+ 517.1; Method 0-60AB 7minLC_v002

¹H NMR (400 MHz, CDCl₃) δ 8.08 (2H, m), 7.71 (1H, d), 7.21 (1H, s), 6.77 (1H, m), 4.20 (2H, d), 2.78 (3H, m), 2.48 (3H, m), 2.13 (1H, m), 2.18 (1H, m), 1.27 (2H, m), 1.06 (8H, m).

### Example 38

### N-(7′-(5-chloro-6-isobutoxypyridin-3-yl)-6′-oxo-6′,7′-dihydrospiro[cyclopentane-1,5′-isoxazolo[4,5-f]indol]-3′-yl)cyclopropanesulfonamide

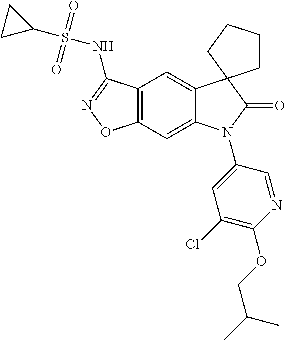 [SMILES: CC(C)COc1ncc(N2C(=O)C3(CCCC3)c3cc4c(NS(=O)(=O)C5CC5)noc4cc32)cc1Cl]

The title compound was prepared by a method similar to that of Example 36 by replacing iodomethane (Example 36 step 1) with 1,4-diiodobutane;

LCMS: Rt=5.77 mins; MS m/z: [M+H]+ 531.1; Method 10-80AB 7minLC_v002

¹H NMR (400 MHz, DMSO-d₆) δ 11.28 (1H, s), 8.28 (1H, d), 8.18 (1H, d), 7.92 (1H, s), 7.06 (1H, s), 4.20 (2H, d), 3.00 (1H, m), 2.15 (3H, m), 2.05 (4H, m), 1.92 (2H, m), 1.10 (4H, m), 1.02 (6H, d).

### Example 39

### N-(7′-(5-chloro-6-isobutoxypyridin-3-yl)-6′-oxo-6′,7′-dihydrospiro[cyclohexane-1,5′-isoxazolo[4,5-f]indol]-3′-yl)cyclopropanesulfonamide

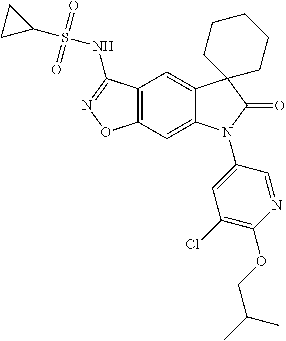 [SMILES: CC(C)COc1ncc(N2C(=O)C3(CCCCC3)c3cc4c(NS(=O)(=O)C5CC5)noc4cc32)cc1Cl]

The title compound was prepared by a method similar to that of Example 36 by replacing iodomethane (Example 36 step 1) with 1,5-diiodopentane;

LC-MS: Rt=6.10 mins; MS m/z [M+H]+ 545.1; Method 10-80AB 7minLC_v002

¹H NMR (400 MHz, DMSO-d₆) δ 11.33 (1H, s), 8.26 (1H, d), 8.20 (1H, s), 8.16 (1H, d), 7.05 (1H, s), 4.20 (2H, d), 3.05 (1H, m), 2.12 (1H, m), 1.82 (7H, m), 1.68 (2H, m), 1.59 (1H, br. s), 1.13 (2H, m), 1.08 (2H, m), 1.02 (6H, d).

### Example 40a

### N-(7-(5-chloro-6-isobutoxypyridin-3-yl)-5-(trifluoromethyl)-7H-isoxazolo[4,5-f]indol-3-yl)methanesulfonamid and Example 40b N-(7-(5-chloro-6-isobutoxypyridin-3-yl)-6-(trifluoromethyl)-7H-isoxazolo[4,5-f]indol-3-yl)methanesulfonamide

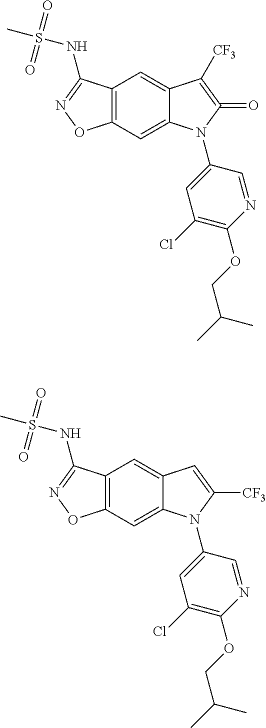

### Step 1: 6-fluoro-1-(triisopropylsilyl)-1H-indole

To a mixture of 6-fluoro-1H-indole (Aldrich) (14 g, 100 mmol) in 200 ml of THF was added n-BuLi (2.5 N in hexane, 40 ml, 100 mmol) at −78° C. dropwise under N₂ atmosphere. After stirring at −78° C. for 15 min, the mixture was added chlorotriisobutylsilane (19 g, 100 mmol) by one portion. The resulting mixture was stirred at −78° C. for another 15 min then warmed to 25° C. for additional 1 h. The reaction mixture was quenched with NH₄Cl aqueous solution. After removal of the solvents, the residue was treated with ethyl acetate and water. The separated organic phases were washed with brine (×3), and dried overmagnesium sulfate and filtered. The filtration was concentrated under reduced pressure to give crude product, which was purified by flash column chromatography on silica gel eluting hexanes to afford the title compound as yellow oil.

LC-MS: Rt=1.42 mins; MS m/z [M+H]+ 291.9; Method 5-95AB 1.5minLC_v003

¹H NMR (400 MHz, CDCl₃) δ 7.54 (1H, dd), 7.24 (2H, d), 6.90 (1H, m), 6.62 (1H, d), 1.70 (3H, m), 1.17 (18H, d).

### Step 2: ethyl 6-fluoro-1-(triisopropylsilyl)-1H-indole-5-carboxylate

To a mixture of 6-fluoro-1-(triisopropylsilyl)-1H-indole (step 1) (15 g, 52 mmol) in 150 ml of THF was added s-BuLi (40 ml, 52 mmol) slowly at −78° C. under N₂ atmosphere. The mixture was stirred at −78° C. for 2 hrs and then added diethyl carbonate (10 ml, 63 mmol). After stirring for additional 5 hrs, the reaction mixture was quenched with NH₄Cl aqueous solution. After removal of the solvents, the residue was treated with ethyl acetate and water. The separated organic phases were washed with brine (×3), and dried overmagnesium sulfate and filtered. The filtration was concentrated under reduced pressure to give a crude product, which was purified by flash column chromatography on silica gel eluting a gradient 0-5% ethyl acetate in hexanes to afford the title compound as white solid.

LC-MS: Rt=1.31 mins; MS m/z [M+H]+ 364.2; Method 5-95AB 1.5minLC_v003

¹H NMR (400 MHz, CDCl₃) 68.15 (1H, d), 8.10 (2H, m), 7.93 (1H, dd), 4.26 (2H, q), 1.33 (3H, t), 1.78 (3H, m), 1.16 (18H, d).

### Step 3: ethyl 6-fluoro-1H-indole-5-carboxylate

To a mixture of ethyl 6-fluoro-1-(triisopropylsilyl)-1H-indole-5-carboxylate (step 2) (21 g, 79 mmol) in THF (250 ml) was added TBAF (21 g, 79 mmol). The mixture was stirred at room temperature for 0.5 h. When TLC indicated the reaction was completed, the reaction mixture was evaporated under reduced pressure to give the residue, which was treated with water and ethyl acetate. The separated organic extracts were washed with brine (×2), and dried over anhydrous magnesium sulfate, and filtered. The filtrate was concentrated under reduced pressure to give crude product, which was purified by flash column chromatography on silica gel eluting a gradient from 0-20% ethyl acetate in hexanes to afford the title compound as white solid.

LC-MS: Rt=1.09 mins; MS m/z [M+H]+ 207.9; Method 5-95AB 1.5minLC_v003

¹H NMR (400 MHz, DMSO-d₆) δ 12.44 (1H, m), 8.20 (1H, d), 8.13 (2H, m), 7.97 (1H, dd), 4.29 (2H, q), 1.30 (3H, t).

### Step 4: ethyl 1-(5-chloro-6-isobutoxypyridin-3-yl)-6-fluoro-1H-indole-5-carboxylate

To a mixture of ethyl 6-fluoro-1H-indole-5-carboxylate (step 3) (8.3 g, 40 mmol) in CH₃CN (100 ml) was added 5-bromo-3-chloro-2-isobutoxypyridine (intermediate A) (15.8 g, 60 mmol), CuI (23 g, 120 mmol), K₂CO₃ (17 g, 120 mmol) and DMEDA (5.3 g, 60 mmol) at room temperature. The resulting mixture was degassed and charged with nitrogen three times. After heating to 100° C., the reaction mixture was stirred at that temperature for 16 hrs. When LC/MS indicated the starting material was consumed, the reaction mixture was cooled down and diluted with ethyl acetate (1000 ml). The mixture was filtered through a pad of celite and the filtration was then washed with brine (×3), dried over anhydrous magnesium sulfate and filtered. The filtration was evaporated under reduced pressure to give the crude product, which was purified by column chromatography on silica gel eluting with 0˜5% ethyl acetate in hexanes to afford the title compound as a white solid.

LC-MS: Rt=1.24 mins; MS m/z [M+H]+ 391.1; Method 5-95AB 1.5minLC_v003

¹H NMR (400 MHz, DMSO-d₆) δ 8.36 (1H, d), 8.25 (1H, d), 8.21 (1H, d), 7.71 (1H, d), 7.35 (1H, d), 6.83 (1H, d), 4.29 (2H, d), 4.17 (2H, d), 2.07 (1H, m), 1.30 (3H, t), 0.98 (6H, d).

### Step 5: ethyl 1-(5-chloro-6-isobutoxypyridin-3-yl)-6-fluoro-3-(trifluoromethyl)-1H-indole-5-carboxylate and ethyl 1-(5-chloro-6-isobutoxypyridin-3-yl)-6-fluoro-2-(trifluoromethyl)-1H-indole-5-carboxylate

To a mixture of ethyl 1-(5-chloro-6-isobutoxypyridin-3-yl)-6-fluoro-1H-indole-5-carboxylate (step 4) (1.1 g, 2.8 mmol) in CH₃CN (20 ml) was added trimethyl(trifluoromethyl)silane (8 g, 56 mmol), K₃PO₄ (2.3 g, 11 mmol), PhI(OAc)₂ (1.8 g, 5.6 mmol), benzoquinone (28 mg, 0.26 mmol) and magnesium sulfate (182 mg, 1.3 mmol) at room temperature. Then the resulting mixture reaction was stirred at 80° C. overnight under nitrogen atmosphere. The reaction mixture was diluted with ethyl acetate and filtered over celite. The filtration was washed with NH₄Cl aqueous solution (×2) and brine (×2), dried over anhydrous magnesium sulfate and filtered. The filtration was concentrated under reduced pressure to give crude product, which was purified by flash column chromatography on silica gel eluting a gradient from 0-5% ethyl acetate in hexanes to afford the title compound as yellow solid.

LC-MS: Rt=1.24 mins; MS m/z [M+H]+ 459.0; Method 5-95AB 1.5minLC_v003

### Step 6: 1-(5-chloro-6-isobutoxypyridin-3-yl)-6-fluoro-3-(trifluoromethyl)-1H-indole-5-carboxylic acid and 1-(5-chloro-6-isobutoxypyridin-3-yl)-6-fluoro-2-(trifluoromethyl)-1H-indole-5-carboxylic acid

To a solution ethyl 1-(5-chloro-6-isobutoxypyridin-3-yl)-6-fluoro-3-(trifluoromethyl)-1H-indole-5-carboxylate and ethyl 1-(5-chloro-6-isobutoxypyridin-3-yl)-6-fluoro-2-(trifluoromethyl)-1H-indole-5-carboxylate (step 5) (900 mg, 1.9 mmol) in THF (50 ml) then was added LiOH aqueous solution (50 ml, 2N). The resulting mixture was stirred at 100° C. for 12 hrs. When TLC indicated the starting material was consumed, the reaction mixture was adjusted with HCl aqueous solution to pH=2-3. The mixture was extracted with ethyl acetate (×3). The combined organic phases were washed with brine (×3), and dried over anhydrous magnesium sulfate and filtered. The filtration was concentrated under reduced pressure to give the title compound.

LC-MS: Rt=1.04 mins; MS m/z [M+H]+ 430.9; Method 5-95AB 1.5minLC_v003

### Step 7: 1-(5-chloro-6-isobutoxypyridin-3-yl)-6-fluoro-3-(trifluoromethyl)-1H-indole-5-carboxamide and 1-(5-chloro-6-isobutoxypyridin-3-yl)-6-fluoro-2-(trifluoromethyl)-1H-indole-5-carboxamide

To a mixture of 1-(5-chloro-6-isobutoxypyridin-3-yl)-6-fluoro-3-(trifluoromethyl)-1H-indole-5-carboxylic acid and 1-(5-chloro-6-isobutoxypyridin-3-yl)-6-fluoro-2-(trifluoromethyl)-1H-indole-5-carboxylic acid (step 6) (675 mg, 1.5 mmol) in DCM (50 ml) was added DMAP (480 mg, 3.9 mmol) and EDCl (750 mg, 3.9 mmol) at 25° C. The resulting mixture was stirred at room temperature for 0.5 h and then added NH₄Cl (210 mg, 3.9 mmol). The mixture was stirred at 25° C. for additional 16 hrs. When TLC indicated the reaction was completed, the reaction mixture was washed with 0.5 N HCl aqueous solution (×3) and brine (×2), dried over anhydrous magnesium sulfate and filtered. The filtration was concentrated under reduced pressure to give crude product, which was purified by flash column chromatography on silica gel eluting a gradient from 0-20% ethyl acetate in hexanes to afford the title compound as white solid.

LC-MS: Rt=1.04 mins; MS m/z [M+H]+ 430.0; Method 5-95AB 1.5minLC_v003

### Step 8: 1-(5-chloro-6-isobutoxypyridin-3-yl)-6-fluoro-3-(trifluoromethyl)-1H-indole-5-carbonitrile and 1-(5-chloro-6-isobutoxypyridin-3-yl)-6-fluoro-2-(trifluoromethyl)-1H-indole-5-carbonitrile

To a mixture of 1-(5-chloro-6-isobutoxypyridin-3-yl)-6-fluoro-3-(trifluoromethyl)-1H-indole-5-carbonitrile and 1-(5-chloro-6-isobutoxypyridin-3-yl)-6-fluoro-2-(trifluoromethyl)-1H-indole-5-carbonitrile (step 7)(654 mg, 1.5 mmol) and triethylamine (1.5 g, 1.5 mmol) in DCM (10 ml) was added 2,2,2-trifluoroacetic anhydride (1.5 g, 15 mmol). The resulting mixture was stirred at 40° C. for 3 hrs. When TLC indicated the reaction was completed, the reaction mixture was wased with brine (×3), dried over anhydrous magnesium sulfate and filtered. The filtration was concentrated under reduced pressure to give the crude product, which was purified by flash column chromatography on silica gel eluting a gradient from 0-20% ethyl acetate in hexanes to afford the title compound as white solid.

LC-MS: Rt=1.19 mins; MS m/z [M+H]+ 452.9; Method 5-95AB 1.5minLC_v003

### Step 9: 7-(5-chloro-6-isobutoxypyridin-3-yl)-5-(trifluoromethyl)-7H-isoxazolo[4,5-f]indol-3-amine and 7-(5-chloro-6-isobutoxypyridin-3-yl)-6-(trifluoromethyl)-7H-isoxazolo[4,5-f]indol-3-amine

To a mixture of propan-2-one oxime (32 mg, 0.43 mmol) in anhydrous DMF (5 ml) was added t-BuOK (49 mg, 0.43 mmol) at room temperature. The resulting mixture was stirred at room temperature for 30 min and then a solution of 1-(5-chloro-6-isobutoxypyridin-3-yl)-6-fluoro-3-(trifluoromethyl)-1H-indole-5-carbonitrile and 1-(5-chloro-6-isobutoxypyridin-3-yl)-6-fluoro-2-(trifluoromethyl)-1H-indole-5-carbonitrile (step 8) (100 mg, 0.29 mmol) in DMF (1 ml) added. After stirring at room temperature for 5 hrs, the reaction was quenched with saturated ammonium chloride solution. The reaction mixture was extracted with ethyl acetate (×3). The combined organic phases were washed with brine (×2), dried over anhydrous magnesium sulfate and filtered. The filtration was concentrated under reduced pressure to give the crude product, which was treated with 10 ml of ethanol and 10 ml of 5% HCl aqueous solution. The resulting mixture was heated to reflux for 45 min. After removal of the organic solvent, the remaining aqueous phase was basified with saturated Na₂CO₃ aqueous solution. The mixture was extracted with ethyl acetate (×3), washed with brine (×2), dried over anhydrous magnesium sulfate and filtered. The filtration was concentrated under reduced pressure to give the crude product, which was purified by flash chromatography on silica gel eluting with 0˜20% ethyl acetate in hexanes to afford the title compound as a black solid.

LC-MS: Rt=0.99 mins; MS m/z [M+H]+ 424.9; Method 5-95AB 1.5minLC_v003

### Step 10: N-(7-(5-chloro-6-isobutoxypyridin-3-yl)-5-(trifluoromethyl)-7H-isoxazolo[4,5-f]indol-3-yl)methanesulfonamide and N-(7-(5-chloro-6-isobutoxypyridin-3-yl)-6-(trifluoromethyl)-7H-isoxazolo[4,5-f]indol-3-yl)methanesulfonamide

To a mixture of 7-(5-chloro-6-isobutoxypyridin-3-yl)-5-(trifluoromethyl)-7H-isoxazolo[4,5-f]indol-3-amine and 7-(5-chloro-6-isobutoxypyridin-3-yl)-6-(trifluoromethyl)-7H-isoxazolo[4,5-f]indol-3-amine (step 9) (120 mg, 0.28 mmol) in dry DCM (5 ml) was added TEA (141 mg, 1.4 mmol) and methanesulfonyl chloride (96 mg, 0.84 mmol) at room temperature. The resulting mixture was stirred at room temperature for 2 hrs and quenched with saturated ammonium chloride aqueous solution. The reaction mixture was washed with brine (×3), dried over anhydrous magnesium sulfate and filtered. The filtration was concentrated under reduced pressure to afford the crude product, which was dissovled in THF (5 ml). To the above mixture was added TBAF (147 mg, 0.56 mmol) and the resulting mixture was then stirred at room temperature for 2 hrs. After removal of the solvent, the residue was treated with ethyl acetate and water. The separated organic phase was washed with water (×2), dried over anhydrous magnesium sulfate and filtered. The filtration was concentrated under reduced pressure to give the crude product, which was purified by column chromatograph on silica gel eluting with 0˜20% ethyl acetate in hexanes to afford the title compound as a white solid. The two isomers were separated by SFC.

LC-MS: Rt=6.34, 6.38 mins; MS m/z [M+H]+ 503.0; Method 0-60AB 7minLC_v002

1H NMR (400 MHz, CDCl₃) δ 8.22 (2H, s), 7.82 (1H, d), 7.69 (1H, s), 7.43 (1H, s), 7.22 (1H, d), 4.26 (2H, d), 3.43 (3H, s), 2.22 (1H, m), 1.10 (6H, d).

1H NMR (400 MHz, CDCl₃) δ: 8.24 (1H, s), 8.14 (1H, s), 7.72 (1H, s), 7.27 (1H, s), 7.22 (1H, d), 7.10 (1H, s), 4.25 (2H, m), 3.38 (3H, s), 2.22 (1H, m), 1.11 (6H, d).

### Example 41

### N-(7-(5-chloro-6-isobutoxypyridin-3-yl)-5-(trifluoromethyl)-7H-isoxazolo[4,5-f]indol-3-yl)cyclopropanesulfonamide

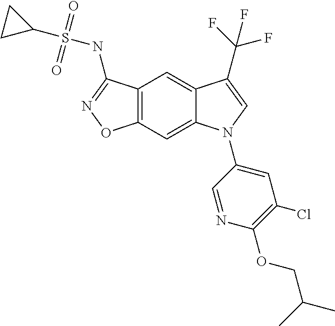 [SMILES: CC(C)COc1ncc(-n2cc(C(F)(F)F)c3cc4c(NS(=O)(=O)C5CC5)noc4cc32)cc1Cl]

The title compound was prepared by a method similar to that of Example 40 by replacing methanesulfonyl chloride (Example 40 step 10) with cyclopropanesulfonyl chloride;

LC-MS: Rt=4.64 mins; MS m/z [M+H]+ 529.1; Method 10-80AB 7minLC_v002

1H NMR (400 MHz, CDCl₃) δ 8.21 (1H, d), 8.19 (1H, s), 7.84 (1H, d), 7.70 (1H, s), 7.46 (1H, s), 7.24 (1H, s), 4.25 (2H, d), 2.22 (1H, m), 1.80 (1H, m), 1.73 (2H, m), 1.25 (2H, m), 1.08 (6H, d).

### Example 42

### N-(7-(5-chloro-6-isobutoxypyridin-3-yl)-5-methyl-7H-isoxazolo[4,54]indol-3-yl)cyclopropanesulfonamide

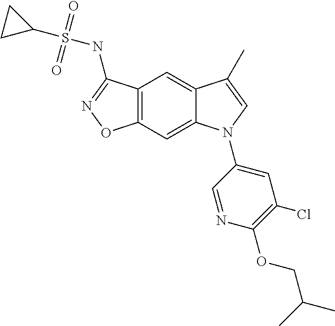 [SMILES: Cc1cn(-c2cnc(OCC(C)C)c(Cl)c2)c2cc3onc(NS(=O)(=O)C4CC4)c3cc12]

### Step 1: 1-(5-chloro-6-isobutoxypyridin-3-yl)-6-fluoro-3-methyl-1H-indole-5-carbonitrile

To a mixture of 3-bromo-1-(5-chloro-6-isobutoxypyridin-3-yl)-6-fluoro-1H-indole-5-carbonitrile (Example 48, step 6) (300 mg, 0.71 mmol) in dioxane (10 ml) was added Pd(dppf)Cl₂ (104 mg, 0.14 mmoL), Cs₂CO₃ (694 mg, 2.13 mmol) and 2,4,6-trimethyl-1,3,5,2,4,6-trioxatriborinane (267 mg, 2.13 mmol). The resulting mixture was stirred at 120° C. for 1 hr under N₂ atmosphere. After removal of the solvents, the residue was treated with water and ethyl acetate. The separated organic phase was washed with brine (×1). The combined organic layers were dried over anhydrous Na₂SO₄ and filtered. The filtration was concentrated under reduced pressure to give the crude product, which was purified by flash column chromatography on silica gel using a gradient 0-5% ethyl acetate in hexanes to afford the title compound as yellow solid.

LC-MS: Rt=1.46 mins; MS m/z [M+H]+ 358.1; Method 5-95AB 1.5minLC_v003

¹H NMR (400 MHz, CDCl₃) δ 8.11 (1H, d), 7.84 (1H, m), 7.69 (1H, d), 7.07 (2H, m), 4.20 (2H, d), 2.34 (3H, s), 2.17 (1H, m), 1.07 (6H, d).

### Step 2: 7-(5-chloro-6-isobutoxypyridin-3-yl)-5-methyl-7H-isoxazolo[4,5-f]indol-3-amine

To a mixture of propan-2-one oxime (55 mg, 0.75 mmol) in DMF (6 ml) was added t-BuOK (0.75 ml, 0.75 mmol, 1 M in THF) dropwise at room temperature. The resulting mixture was stirred at room temperature for 30 min and then added a solution of 1-(5-chloro-6-isobutoxypyridin-3-yl)-6-fluoro-3-methyl-1H-indole-5-carbonitrile (step 1) (180 mg, 0.5 mmol). After stirring at room temperature for 5 hrs, the reaction was quenched with saturated ammonium chloride solution. The reaction mixture was extracted with ethyl acetate (×3). The combined organic phases were washed with brine (×2), dried over anhydrous magnesium sulfate and filtered. The filtration was concentrated under reduced pressure to give the crude product, which was treated with 50 ml of ethanol and 5 ml of 5% HCl aqueous solution. The resulting mixture was heated to reflux for 45 min. After removal of the organic solvent, the remaining aqueous phase was basified with saturated Na₂CO₃ aqueous solution. The mixture was extracted with ethyl acetate (×3), washed with brine (×2), dried over anhydrous magnesium sulfate and filtered. The filtration was concentrated under reduced pressure to give the crude product, which was purified by flash chromatography on silica gel eluting with 0˜20% ethyl acetate in hexanes to afford the title compound as a white solid.

LC-MS: Rt=1.32 mins; MS m/z [M+H]+ 371.1; Method 5-95AB 1.5minLC_v003

### Step 3: N-(7-(5-chloro-6-isobutoxypyridin-3-yl)-5-methyl-7H-isoxazolo[4,5-f]indol-3-yl)cyclopropanesulfonamide

To a solution of 7-(5-chloro-6-isobutoxypyridin-3-yl)-5-methyl-7H-isoxazolo[4,5-f]indol-3-amine (step 2) (25 mg, 0.067 mmol) in pyridine (1 ml) was added DMAP (16 mg, 0.134 mmol) and cyclopropanesulfonyl chloride (95 mg, 0.674 mmol). The reaction solution was stirred at room temperature for 30 hrs. After removal of the solvent, the residue was treated with water and ethyl acetate. The separated organic phase was washed with water (×2), dried over anhydrous Na₂SO₄ and filtered. The filtration was concentrated under reduced pressure to give the crude product, which was purified by prep-HPLC to afford the title compound as white solid.

LC-MS: Rt=4.68 mins; MS m/z [M+H]+ 475.1; Method 10-80AB 7minLC_v002

1H NMR (400 MHz, CDCl₃) δ 8.17 (1H, s), 8.05 (1H, s), 7.76 (1H, d), 7.36 (1H, s), 7.28 (1H, s), 7.08 (1H, s), 4.20 (2H, d), 2.82 (1H, m), 2.40 (3H, s), 2.18 (1H, m), 1.28 (2H, m), 1.07 (8H, d).

### Example 43

### N-(7-(5-chloro-6-isobutoxypyridin-3-yl)-6-(trifluoromethyl)-7H-imidazo[4′,5′:4,5]benzo[1,2-d]isoxazol-3-yl)cyclopropanesulfonamide

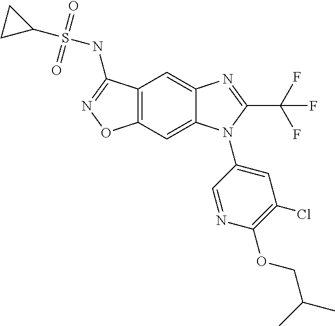 [SMILES: CC(C)COc1ncc(-n2c(C(F)(F)F)nc3cc4c(NS(=O)(=O)C5CC5)noc4cc32)cc1Cl]

### Step 1: 4-((5-chloro-6-isobutoxypyridin-3-yl)amino)-2-fluoro-5-nitrobenzonitrile

To a stirred solution of 2,4-difluoro-5-nitrobenzonitrile (Aldrich) (3 g, 16.3 mmol) in DMF (20 ml) was added 5-chloro-6-isobutoxypyridin-3-amine (intermediate V) (3.3 g, 16.3 mmol) and K₂CO₃ (2.7 g, 19.6 mmol). The resulting mixture was stirred at 60° C. for 15 hrs. When TLC indicated the starting material was consumed, the reaction was added 100 ml of water. The resulting mixture was extracted with ethyl acetate (×3). The combined organic phases were washed with brine, dried over anhydrous Magnesium sulfate and filtered. The filtration was concentrated under reduced pressure to give the crude product, which was purified by flash column chromatography on silica gel using a gradient 0-10% ethyl acetate in hexanes to afford the title compound as yellow solid.

LC-MS: Rt=1.16 mins; MS m/z [M+H]+ 365.1; Method 5-95AB 1.5minLC_v003

### Step 2: 5-amino-4-((5-chloro-6-isobutoxypyridin-3-yl)amino)-2-fluorobenzonitrile

To a solution of 4-((5-chloro-6-isobutoxypyridin-3-yl)amino)-2-fluoro-5-nitrobenzonitrile (step 1) (2.3 g, 6.3 mmol) in MeOH (100 ml) was added Zn (4.1 g, 63 mmol) and NH₄Cl (3.3 g, 63 mmol) at 25° C. This resulting mixture was stirred at 25° C. for 2 hrs. When TLC indicated the reaction was completed, the reaction mixture was filtered. The filtrate was concentrated under reduced pressure to give the residue, which was washed with water and dried to afford the title compound as pale yellow solid.

LC-MS: Rt=0.91 mins; MS m/z [M+H]+ 335.2; Method 5-95AB 1.5minLC_v003

### Step 3: N-(2-((5-chloro-6-isobutoxypyridin-3-yl)amino)-5-cyano-4-fluorophenyl)-2,2,2-trifluoroacetamide

A mixture of 5-amino-4-((5-chloro-6-isobutoxypyridin-3-yl)amino)-2-fluorobenzonitrile (step 2) (1.5 g, 4.5 mmol) in TFA (20 ml) was refluxed for 16 hrs. When LC/MS indicated the starting material was consumed, the reaction mixture was evaporated via vacuum to the residue. The crude product was purified by flash column chromatography on silica gel using a gradient 0-10% ethyl acetate in hexanes to afford the title compound as yellow solid.

LC-MS: Rt=1.17 mins; MS m/z [M+H]+ 431.1; Method 5-95AB 1.5minLC_v003

### Step 4: 1-(5-chloro-6-isobutoxypyridin-3-yl)-6-fluoro-2-(trifluoromethyl)-1H-benzo[d]imidazole-5-carbonitrile

A mixture of N-(2-((5-chloro-6-isobutoxypyridin-3-yl)amino)-5-cyano-4-fluorophenyl)-2,2,2-trifluoroacetamide (step 3) (650 mg, 1.5 mmol) and p-TsOH (130 mg, 0.75 mmol) in EtOH (20 ml) was heated to refulx. The resulting mixture was stirred at that temperature for 16 hrs. After removal of the solvent, the residue was treated with ethyl acetate and water. The separated organic phase was washed with brine (×2), dried over anhydrous magnesium sulfate and filtered. The filtration was evaporated under reduced pressure to afford the title compound as white solid.

LC-MS: Rt=1.21 mins; MS m/z [M+H]+ 413.1; Method 5-95AB 1.5minLC_v003

¹H NMR (400 MHz, CDCl₃) δ 8.24 (1H, d), 8.11 (1H, d), 7.69 (1H, d), 7.00 (1H, d), 4.24 (2H, d), 2.20 (1H, m), 1.08 (6H, d).

### Step 5: 7-(5-chloro-6-isobutoxypyridin-3-yl)-6-(trifluoromethyl)-7H-Imidazo-[4′,5′:4,5]benzo[1,2-d]isoxazol-3-amine

To a mixture of 1-(5-chloro-6-isobutoxypyridin-3-yl)-6-fluoro-2-(trifluoromethyl)-1H-benzo-[d]imidazole-5-carbonitrile (step 4) (590 mg, 1.4 mmol) and N-hydroxyacetamide (322 mg, 4.3 mmol) in DMF (30 ml) was added t-BuOK (4.3 ml, 1 M in THF, 4.3 mmol). The mixture was stirred at 25° C. for 16 hrs. When LC/MS indicated the reaction was completed, the mixture was evaporated via vacuum to the residue, which was treated with water and ethyl acetate. The separated organic phase was washed with brine (×2), dried over anhydrous magnesium sulfate and filtered. The filtration was concentrated under reduced pressure to give the crude product, which was purified by flash column chromatography on silica gel using a gradient 0-50% ethyl acetate in hexanes to afford the title compound as white solid.

LC-MS: Rt=0.76 mins; MS m/z [M+H]+ 426.1; Method 5-95AB 1.5minLC_v003

¹H NMR (400 MHz, CDCl₃) δ 8.17 (1H, d), 8.05 (1H, s), 7.74 (1H, d), 7.10 (1H, s), 4.52 (2H, s), 4.27 (2H, d), 2.22 (1H, m), 1.11 (6H, d).

### Step 6: N-(7-(5-chloro-6-isobutoxypyridin-3-yl)-6-(trifluoromethyl)-7H-imidazo[4′,5′:4,5]benzo[1,2-d]isoxazol-3-yl)cyclopropanesulfonamide

To a solution of 7-(5-chloro-6-isobutoxypyridin-3-yl)-6-(trifluoromethyl)-7H-imidazo[4′,5′:4,5]benzo[1,2-d]isoxazol-3-amine (step 5) (200 mg, 0.47 mmol) and DMAP (115 mg, 0.94 mmol) in pyridine (1.5 ml) was added cyclopropanesulfonyl chloride (660 mg, 4.7 mmol). The reaction mixture was stirred at room temperature for 16 hrs. When TLC indicated the starting material was consumed, the reaction mixture was diluted with ethyl acetate (50 ml), washed with brine (×2), dried over anhydrous magnesium sulfate and filtered. The filtration was concentrated under reduced pressure to give the residue, which was purified by preparative HPLC to afford the title compound as white solid.

LC-MS: Rt=3.29 mins; MS m/z [M+H]+ 530.1; Method 10-80AB 7minLC_v001

¹H NMR (400 MHz, CDCl₃) δ 8.45 (1H, s), 8.16 (1H, d), 7.75 (1H, d), 7.28 (1H, s), 7.21 (1H, s), 4.25 (2H, d), 2.85 (1H, m), 2.21 (1H, m), 1.33 (2H, m), 1.13 (2H, m), 1.09 (6H, d).

### Example 44

### N-(7-(5-chloro-6-isobutoxypyridin-3-yl)-5-methyl-7H-isoxazolo[4,54]indazol-3-yl)methanesulfonamide

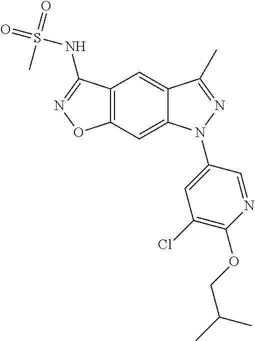 [SMILES: Cc1nn(-c2cnc(OCC(C)C)c(Cl)c2)c2cc3onc(NS(C)(=O)=O)c3cc12]

### Step 1: 5-bromo-6-fluoro-3-methyl-1H-indazole

To a stirred solution of 1-(5-bromo-2,4-difluorophenyl) ethanone (Apollo) (6 g, 25 mmol) in ethane-1,2-diol (50 ml) was added hadrazine monohydrate (2 ml). The resulting mixture was stirred at 130° C. for 15 hrs. When LC/MS indicated the reaction was completed, the reaction mixture was added 100 ml of water. The precipitate was collected and washed with DCM/hexanes (100 ml, 10:1) to afford the title compound as yellow solid.

LC-MS: Rt=1.09 mins; MS m/z [M+H]+ 228.9; Method 5-95AB 1.5minLC_v003

¹H NMR (400 MHz, CDCl₃) δ 12.41 (1H, s), 7.87 (1H, d), 7.18 (1H, d), 2.55 (3H, s).

### Step 2: 6-fluoro-3-methyl-1H-indazole-5-carbonitrile

To a mixture of 5-bromo-6-fluoro-3-methyl-1H-indazole (step 1) (2 g, 8.8 mmol), Zn(CN)₂ (2.0 g, 17.5 mmol) and DPPF (5.8 g, 10.5 mmol) in DMF (20 ml) was added Pd₂dba₃ (2.5 g, 4.4 mmol). The resulting mixture was degassed and charged with N₂ three times, and then heated to 120° C. The mixture was stirred at that temperature under N₂ atmosphere for 4 hrs. When LC/MS indicated the starting material was consumed, the reaction mixture was diluted with ethyl acetate (100 ml). The mixture was filtered over celite. The filtration was washed with brine (×3), dried over anhydrous magnesium sulfate, and evaporated to give a residue, which was purified by flash column chromatography on silica gel using a gradient 0-35% ethyl acetate in hexanes to afford the title compound as pale yellow solid.

LC-MS: Rt=1.01 mins; MS m/z [M+H]+ 176.1; Method 5-95AB 1.5minLC_v003

¹H NMR (400 MHz, CDCl₃) δ12.41 (1H, s), 8.01 (1H, d), 7.21 (1H, d), 2.58 (3H, s).

### Step 3: 1-(5-chloro-6-isobutoxypyridin-3-yl)-6-fluoro-3-methyl-1H-indazole-5-carbonitrile

To a mixture of 6-fluoro-3-methyl-1H-indazole-5-carbonitrile (step 2) (1.2 g, 6.9 mmol) in MeCN (50 ml) was added 5-bromo-3-chloro-2-isobutoxypyridine (intermediate A) (5.4 g, 21 mmol), CuI (640 mg, 3.5 mmol), K₂CO₃ (1.9 g, 13.8 mmol), and DMEDA (360 mg, 4.2 mmol). When the addition was completed, the mixture was degassed and charged with N₂ three times. Then the mixture was heated to 100° C. and stirred at that temperature under N₂ atmosphere for 16 hrs. When LC/MS indicated the starting material was consumed, the reaction mixture was diluted with DCM (200 ml). The mixture was filtered over celite. The filtration was washed with brine (×3), dried over anhydrous magnesium sulfate, and evaporated to give a residue, which was purified by flash column chromatography on silica gel using a gradient 0-10% ethyl acetate in hexanes to afford the title compound as pale yellow solid.

LC-MS: Rt=1.18 mins; MS m/z [M+H]+ 359.0; Method 5-95AB 1.5minLC_v003

¹H NMR (400 MHz, CDCl₃) δ 8.28 (1H, d), 8.05 (1H, d), 7.93 (1H, d), 7.31 (1H, d), 4.21 (2H, d), 2.64 (3H, s), 2.18 (1H, td), 1.06 (6H, d).

### Step 4: 7-(5-chloro-6-isobutoxypyridin-3-yl)-5-methyl-7H-isoxazolo[4,5-f]indazol-3-amine

To a stirred solution of propan-2-one oxime (125 mg, 1.7 mmol) in DMF (10 ml) was added t-BuOK (1.7 ml, 1.7 mmol). The mixture was stirred at 0° C. for 0.5 h. Then 1-(5-chloro-6-isobutoxypyridin-3-yl)-6-fluoro-3-methyl-1H-indazole-5-carbonitrile (step 3) (410 mg, 1.1 mmol) was added to the mixture. After stirring at room temperature for additional 0.5 h, LC/MS indicated the reaction was completed. Then the reaction mixture was evaporated under reduced pressure to give the residue, which was treated with water and ethyl acetate. The separated organic phase was washed with brine (×2) and concentrated to the crude product, which was added EtOH (8 ml) and 5% HCl aqueous solution (8 ml). The resulting mixture was stirred at 80° C. for 16 hrs. After removal of the organic solvent, the mixture was extracted with ethyl acetate (×3). The combined organic phases were washed with brine (×2), dried over anhydrous Magnesium sulfate and concentrated under reduced pressure to give the crude product, which was purified by flash column chromatography on silica gel using a gradient 0-35% ethyl acetate in hexanes to afford the title compound as brown solid.

LC-MS: Rt=0.88 mins; MS m/z [M+H]+ 372.0; Method 5-95AB 1.5minLC_v003

¹H NMR (400 MHz, DMSO-d₆) δ 8.38 (1H, d), 7.99 (1H, d), 7.83 (1H, s), 7.43 (1H, s), 4.47 (2H, s), 4.21 (2H, d), 2.68 (3H, s), 2.18 (1H, td), 1.06 (6H, d).

### Step 5: N-(7-(5-chloro-6-isobutoxypyridin-3-yl)-5-methyl-7H-isoxazolo[4,5-f]indazol-3-yl)methanesulfonamide

To a solution of 7-(5-chloro-6-isobutoxypyridin-3-yl)-5-methyl-7H-isoxazolo[4,5-f]indazol-3-amine (step 4) (200 mg, 0.54 mmol) in DCM (10 ml) was added methanesulfonyl chloride (184 mg, 1.6 mmol) and TEA (273 mg, 2.7 mmol). The reaction mixture was stirred at room temperature for 1 h. When TLC indicated the starting material was consumed, the reaction mixture was washed with water (×3), dried over anhydrous magnesium sulfate and concentrated under reduced pressure to give the crude product. To a solution of the above crude product N-(7-(5-chloro-6-isobutoxypyridin-3-yl)-5-methyl-7H-isoxazolo[4,5-f]indazol-3-yl)-N-(methylsulfonyl)methanesulfonamide (280 mg, 0.53 mmol) in THF (3 ml) was added TBAF (1.6 ml, 1.6 mmol). The reaction mixture was stirred at room temperature for 1 h. When TLC indicated the reaction was completed, the reaction mixture was concentrated under reduced pressure to give the crude product, which was purified by flash column chromatography on silica gel using a gradient 0-50% ethyl acetate in hexanes to afford the title compound as white solid.

LC-MS: Rt=3.46 mins; MS m/z [M+H]+ 450.0; Method 10-80AB 7minLC_v001

¹H NMR (400 MHz, CDCl₃) δ 8.37 (1H, d), 8.21 (1H, s), 7.99 (1H, d), 7.54 (1H, s), 7.24 (1H, s), 4.21 (2H, d), 3.35 (3H, s), 2.69 (3H, s), 2.18 (1H, td), 1.07 (6H, d).

### Example 45

### N-(7-(5-chloro-6-isobutoxypyridin-3-yl)-5-methyl-7H-isoxazolo[4,5-f]indazol-3-yl)cyclopropanesulfonamide

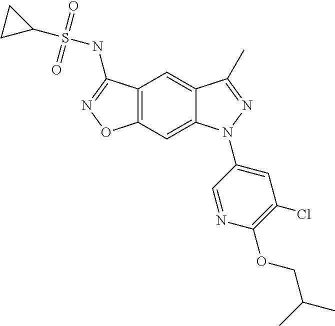 [SMILES: Cc1nn(-c2cnc(OCC(C)C)c(Cl)c2)c2cc3onc(NS(=O)(=O)C4CC4)c3cc12]

The title compound was prepared by a method similar to that of Example 44 by replacing methanesulfonyl chloride (Example 44 step 5) with cyclopropanesulfonyl chloride;

LCMS: Rt=4.67 mins; MS m/z: [M+H]+ 476.0; Method 10-80AB 7minLC_v002

¹H NMR (400 MHz, CDCl₃) δ 8.38 (1H, d), 8.30 (1H, s), 8.00 (1H, d), 7.54 (1H, s), 7.24 (1H, s), 4.21 (2H, d), 2.78 (1H, dt), 2.70 (3H, s), 2.19 (1H, td), 1.30 (2H, m), 1.10 (8H, m).

### Example 46

### N-(7-(3,4-dichlorophenyl)-5-methyl-7H-isoxazolo[4,5-f]indazol-3-yl)cyclopropanesulfonamide

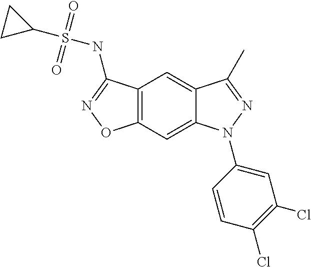 [SMILES: Cc1nn(-c2ccc(Cl)c(Cl)c2)c2cc3onc(NS(=O)(=O)C4CC4)c3cc12]

The title compound was prepared by a method similar to that of Example 44 by replacing 5-bromo-3-chloro-2-isobutoxypyridine (intermediate A Example 44 step 3) with 4-bromo-1,2-dichlorobenzene and replacing methanesulfonyl chloride (Example 46 step 5) with cyclopropanesulfonyl chloride;

LC-MS: Rt=4.09 mins; MS m/z [M+H]+ 437.0; Method 10-80AB 7minLC_v002

¹H NMR (400 MHz, CDCl₃) δ 8.28 (1H, s), 7.85 (1H, s), 7.61 (3H, m), 7.21 (1H, s), 2.78 (1H, m), 2.69 (3H, s), 1.28 (2H, m), 1.09 (2H, m).

### Example 47

### N-(7-(5-chloro-6-isobutoxypyridin-3-yl)-5-methyl-5H-isoxazolo[5,4-f]indazol-3-yl)cyclopropanesulfonamide

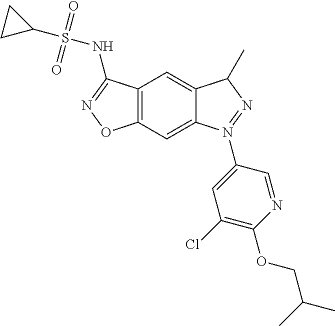

### Step 1: methyl 4-bromo-2,5-difluorobenzoate

To a mixture of 4-bromo-2,5-difluorobenzoic acid (Oakwood) (5 g, 21 mmol) in MeOH (50 ml) was added conc. H₂SO₄ (3 ml) at room temperature. The reaction mixture was stirred at 80° C. for 4 hrs. When TLC indicated the reaction was completed, the mixture was concentrated under reduced pressure to the residue, which was treated with ethyl acetate and water. The separated organic phase was washed with brine (×2), dried over anhydrous magnesium sulfate and filtered. The filtration was concentrated under reduced pressure to afford the title compound.

¹H NMR (400 MHz, CDCl₃) δ 7.69 (1H, dd), 7.39 (1H, dd), 3.93 (3H, s).

### Step 2: 6-bromo-5-fluoro-1-methyl-1H-indazol-3(2H)-one

To a solution of methyl 4-bromo-2,5-difluorobenzoate (step 1) (4 g, 16 mmol) in EtOH (40 ml) was added MeNH₂NH₂ (4.8 ml) and p-TsOH (200 mg). The reaction mixture was irritated under microwave condition at 150° C. for 1 h. The mixture was concentrated to give the crude product, which was purified by flash column chromatography on silica gel eluting a gradient from 0-35% ethyl acetate in hexanes to afford the title compound as pale yellow solid.

¹H NMR (400 MHz, DMSO-d₆) δ 7.90 (1H, d), 7.49 (1H, d), 3.73 (3H, s).

### Step 3: 5-fluoro-1-methyl-3-oxo-2,3-dihydro-1H-indazole-6-carbonitrile

To a mixture of 6-bromo-5-fluoro-1-methyl-1H-indazol-3(2H)-one (step 2) (1.5 g, 6.1 mmol), DPPF (681 mg, 1.2 mmol) and Zn(CN)₂ (1.43 g, 12 mmol) in DMF (25 ml) was added Pd₂(dba)₃ (1.42 g, 2.5 mmol) at room temperature. The resulting mixture was degassed and charged with nitrogen three times. After heating to 120° C., the reaction mixture was stirred at that temperature under nitrogen atmosphere for 4 hrs. When LC/MS indicated the starting material was consumed, the reaction mixture was cooled down and diluted with ethyl acetate (1000 ml). The mixture was filtered through a pad of celite and the filtration was then washed with brine (×3), dried over anhydrous magnesium sulfate and filtered. The filtration was evaporated under reduced pressure to give the crude product, which was purified by column chromatography on silica gel eluting with 0˜30% ethyl acetate in hexanes to afford the title compound as pale yellow solid.

LC-MS: Rt=0.58 mins; MS m/z [M+H]+ 192.1; Method 5-95AB 1.5minLC_v003

¹H NMR (400 MHz, DMSO-d₆) δ 8.26 (1H, d), 7.64 (1H, d), 3.83 (3H, s).

### Step 4: 6-cyano-5-fluoro-1-methyl-1H-indazol-3-yl trifluoromethanesulfonate

To a mixture of 5-fluoro-1-methyl-3-oxo-2,3-dihydro-1H-indazole-6-carbonitrile (step 3) (830 mg, 4.3 mmol) and DIEA (1.8 ml, 13 mmol) in 40 ml of DCM was added trifluoromethanesulfonic anhydride (950 uL, 5.6 mmol) at 0° C. under N₂. The mixture was stirred at 0° C. for 30 min. When LC/MS indicated the reaction was completed, the reaction mixture was diluted with DCM and washed with 5% HCl aqueous solution and brine (×2), dried over anhydrous magnesium sulfate and filtered. The filtration was evaporated under reduced pressure to afford the title compound as brown oil.

LC-MS: Rt=0.65 mins; MS m/z [M+H]+ 324.1; Method 5-95AB 1.5minLC_v003

¹H NMR (400 MHz, DMSO-d₆) δ 8.72 (1H, d), 8.04 (1H, d), 4.13 (3H, s).

### Step 5: 3-(5-chloro-6-isobutoxypyridin-3-yl)-5-fluoro-1-methyl-1H-indazole-6-carbonitrile

To a solution of 6-cyano-5-fluoro-1-methyl-1H-indazol-3-yl trifluoromethanesulfonate (step 4) (1.4 g, 4.3 mmol) in dioxane (100 ml) was added 3-chloro-2-isobutoxy-5-(4,4,5,5-tetramethyl-1,3,2-dioxaborolan-2-yl)pyridine (intermediate U) (2.7 g, 8.6 mmol), Pd(dppf)Cl₂ (317 mg, 0.43 mmol) and Cs₂CO₃ (2.1 g, 6.4 mmol). The resulting mixture was bubbled into N₂ for 10 min, then stirred under N₂ atmosphere at 100° C. for 16 h rs. When the reaction was completed as monitored by LC-MS, the reaction mixture was cooled to room temperature and filtered over celite. The filtration was concentrated under reduced pressure to the resulting mixture, which was treated with water (100 ml) and ethyl acetate (100 ml). The separated aqueous phase was extracted with ethyl acetate (×3). The combined organic phases were washed with brine (×2), and dried over anhydrous magnesium sulfate and filtered. The filtration was concentrated under reduced pressure to give the crude product, which was purified by flash column chromatography on silica gel using a gradient from 0-10% ethyl acetate in hexanes to afford the title compound as brown solid.

LC-MS: Rt=0.96 mins; MS m/z [M+H]+ 359.4; Method 5-95AB 1.5minLC_v003

¹H NMR (400 MHz, CDCl₃) δ 8.50 (1H, d), 8.16 (1H, d), 7.77 (1H, d), 7.70 (1H, d), 4.22 (2H, d), 4.17 (3H, s), 2.19 (1H, m), 1.08 (6H, d).

### Step 6: 3-(5-chloro-6-isobutoxypyridin-3-yl)-1-methyl-5-((propan-2-ylideneamino)oxy)-1H-indazole-6-carbonitrile

To a mixture of propan-2-one oxime (352 mg, 4.8 mmol) in anhydrous DMF (20 ml) was added t-BuOK (538 mg, 4.8 mmol) by portion at room temperature. The resulting mixture was stirred at room temperature for 30 min and then added a solution of 3-(5-chloro-6-isobutoxypyridin-3-yl)-5-fluoro-1-methyl-1H-indazole-6-carbonitrile (step 5) (1.1 g, 3.2 mmol) in DMF (5 ml). After stirring at room temperature for 5 hrs, the reaction was quenched with saturated ammonium chloride solution. The reaction mixture was extracted with ethyl acetate (×3). The combined organic phases were washed with brine (×2), dried over anhydrous magnesium sulfate and filtered. The filtration was concentrated under reduced pressure to give the crude product, which was treated with 20 ml of ethanol and 20 ml of 10% HCl aqueous solution. The resulting mixture was heated to reflux for 60 min. After removal of the organic solvent, the remaining aqueous phase was basified with saturated Na₂CO₃ aqueous solution. The mixture was extracted with ethyl acetate (×3), washed with brine (×2), dried over anhydrous magnesium sulfate and filtered. The filtration was concentrated under reduced pressure to give the crude product, which was purified by flash chromatography on silica gel eluting with 0˜20% ethyl acetate in hexanes to afford the title compound as a yellow solid.

LC-MS: Rt=0.75 mins; MS m/z [M+H]+ 372.1; Method 5-95AB 1.5minLC_v003

¹H NMR (400 MHz, MeOD) δ 8.66 (1H, d), 8.32 (1H, d), 7.95 (1H, s), 7.90 (1H, s), 4.29 (2H, m), 4.14 (3H, s), 2.18 (1H, m), 1.10 (6H, d).

### Step 7: N-(7-(5-chloro-6-isobutoxypyridin-3-yl)-5-methyl-5H-isoxazolo[5,4-f]indazol-3-yl)cyclopropanesulfonamide

To a solution of 3-(5-chloro-6-isobutoxypyridin-3-yl)-1-methyl-5-((propan-2-ylideneamino)oxy)-1H-indazole-6-carbonitrile (step 6) (200 mg, 0.27 mmol) in pyridine (4 ml) was added cyclopropanesulfonyl chloride (756 mg, 2.7 mmol) and DMAP (132 mg), the reaction mixture was stirred at rt. for 16 hrs. When LC/MS indicated the reaction was completed, the reaction mixture was diluted with DCM, washed with water (×2), dried over anhydrous magnesium sulfate and filtered. The filtration was concentrated to give the crude product, which was purified by preparative HPLC to afford the title compound as yellow solid.

LC-MS: Rt=5.68 mins; MS m/z [M+H]+ 476.4; Method 0-60AB 7minLC_v002

¹H NMR (400 MHz, MeOD) δ 8.68 (1H, d), 8.35 (1H, d), 8.11 (1H, s), 8.04 (1H, s), 4.29 (5H, m), 3.10 (1H, m), 2.19 (1H, m), 1.28 (2H, d), 1.16 (8H, m).

### Example 48

### N-(7-(5-chloro-6-isobutoxypyridin-3-yl)-5-cyano-7H-isoxazolo[4,54]indol-3-yl)cyclopropanesulfonamide

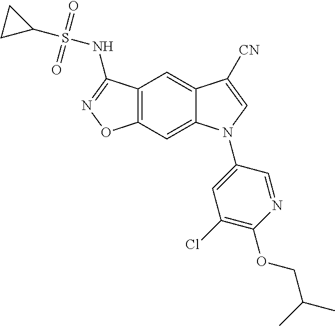 [SMILES: CC(C)COc1ncc(-n2cc(C#N)c3cc4c(NS(=O)(=O)C5CC5)noc4cc32)cc1Cl]

### Step 1: 4-bromo-5-fluoro-2-iodoaniline

To a mixture of 4-bromo-3-fluoroaniline (4 g, 21 mmol) in 50 ml of HOAc was added NIS (5.7 g, 25 mmol) by portion. This reaction mixture was stirred at 30° C. under N₂ for 1.5 hrs and then poured into ice-water. The solid was collected and dried to the title compound as brown solid.

LC-MS: Rt=1.29 mins; MS m/z [M+H]+ 315.8; Method 5-95AB 1.5minLC_v003

¹H NMR (400 MHz, CDCl₃) δ 7.73 (1H, d), 6.53 (1H, d), 4.22 (2H, s).

### Step 2: 4-bromo-5-fluoro-2-((trimethylsilyl)ethynyl)aniline

To a mixture of 4-bromo-5-fluoro-2-iodoaniline (step 1) (5.2 g, 16 mmol), Pd(PPh₃)₂Cl₂ (582 mg, 0.83 mmol) and CuI (313 mg, 1.6 mmol) in 50 ml of Et₃N was added ethynyltrimethylsilane (2.3 mL, 1.6 mmol) slowly at 0° C. The resulting mixture was degassed and charged with N₂ three times. After stirring at 30° C. for 2 hrs, the reaction mixture was filtered over celite. The filtrate was diluted with ethyl acetate (×2), washed with water (×2) and brine (×2). The combined organic layers were dried over anhydrous Na₂SO₄ and filtered. The filtration was evaporated under reduced pressure to afford the crude product, which was purified by column chromatography on silica gel eluting with 0˜5% ethyl acetate in hexanes to afford the title compound as yellow oil.

LC-MS: Rt=1.29 mins; MS m/z [M+H]+ 286.0; Method 5-95AB 1.5minLC_v003

¹H NMR (400 MHz, CDCl₃) δ 7.19 (1H, d), 6.21 (1H, d), 4.11 (2H, s), 0.00 (9H, s).

### Step 3: 5-bromo-6-fluoro-1H-indole

To a mixture of 4-bromo-5-fluoro-2-((trimethylsilyl)ethynyl)aniline (step 2)(3 g, 10 mmol) in 50 ml of DMF was added CuI (4.0 g, 21 mmol) by portion at 0° C. The resulting mixture was stirred at 100° C. under N₂ for 4 hrs. After poured into water, the mixture was extracted with EtOAc (×3). The combined organic layers were dried over anhydrous Na₂SO₄ and filtered. The filtration was evaporated under reduced pressure to afford the crude product, which was purified by column chromatography on silica gel eluting with 0˜10% ethyl acetate in hexanes to afford the title compound as yellow solid.

LC-MS: Rt=1.29 mins; MS m/z [M+H]+ 213.9; Method 5-95AB 1.5minLC_v003

¹H NMR (400 MHz, CDCl₃) δ 10.11 (1H, s), 7.77 (1H, d), 7.19 (2H, d), 6.48 (1H, s).

### Step 4: 6-fluoro-1H-indole-5-carbonitrile

To a mixture of 5-bromo-6-fluoro-1H-indole (step 3)(1.8 g, 8.4 mmol), Zn (1.6 g, 25 mmol), Zn(CN)₂ (2.0 g, 17 mmol) and Pd₂dba₃ (1.5 g, 1.7 mmol) in 60 ml of DMF was added Pd(dppf)Cl₂ (1.2 g, 1.7 mmol). The resulting mixture was degassed and charged with N₂ three times, and then stirred at 150° C. under N₂ for 2.5 hrs. After filtered over celite, the filtrate was diluted with EtOAc, washed with water (×2), dried over anhydrous Na₂SO₄ and filtered. The filtration was evaporated under reduced pressure to afford the crude product, which was purified by column chromatography on silica gel eluting with 0˜10% ethyl acetate in hexanes to afford the title compound as yellow solid.

LC-MS: Rt=1.29 mins; MS m/z [M+H]+ 161.0; Method 5-95AB 1.5minLC_v003

¹H NMR (400 MHz, CDCl₃) δ10.11 (1H, s), 7.89 (1H, d), 7.30 (1H, d), 7.20 (1H, d), 6.60 (1H, s).

### Step 5: 1-(5-chloro-6-isobutoxypyridin-3-yl)-6-fluoro-1H-indole-5-carbonitrile

To a mixture of 6-fluoro-1H-indole-5-carbonitrile (step 4) (500 mg, 3.1 mmol), 5-bromo-3-chloro-2-isobutoxypyridine (Intermediate A, 2.5 g, 9.4 mmol), DMEDA (668 ul, 6.2 mmol), K₂CO₃ (863 mg, 6.2 mmol) in 100 ml of MeCN was added CuI (595 mg, 3.1 mmol). The resulting mixture was degassed and charged with N₂, and then stirred at 100° C. under N₂ for 4 hrs. After filtered over celite, the mixture was evaporated under reduced pressure to the residue, which was treated with ethyl acetate and water. The separated layer was dried over anhydrous Na₂SO₄ and filtered. The filtration was evaporated under reduced pressure to afford the crude product, which was purified by column chromatography on silica gel eluting with 0˜10% ethyl acetate in hexanes to afford the title compound as white solid.

LC-MS: Rt=1.29 mins; MS m/z [M+H]+ 344.2; Method 5-95AB 1.5minLC_v003

¹H NMR (400 MHz, CDCl₃) δ 8.15 (1H, d), 7.94 (1H, d), 7.73 (1H, d), 7.30 (1H, d), 7.13 (1H, d), 6.74 (1H, d), 4.21 (2H, d), 2.19 (1H, m), 1.07 (6H, d).

### Step 6: 3-bromo-1-(5-chloro-6-isobutoxypyridin-3-yl)-6-fluoro-1H-indole-5-carbonitrile

To a solution of 1-(5-chloro-6-isobutoxypyridin-3-yl)-6-fluoro-1H-indole-5-carbonitrile (step 5) (800 mg, 2.3 mmol) in 30 ml of THF was added NBS (414 mg, 2.3 mmol) by portion. The resulting mixture was stirred at 0° C. under N₂ for 2 hrs, and then diluted with ethyl acetate. The mixture was washed with aqueous Na₂S₂O₃ solution (×2) and brine (×2), dried over anhydrous Na₂SO₄ and filtered. The filtration was evaporated under reduced pressure to afford the crude product, which was purified by column chromatography on silica gel eluting with 0˜10% ethyl acetate in hexanes to afford the title compound as white solid.

LC-MS: Rt=1.29 mins; MS m/z [M+H]+ 421.9; Method 5-95AB 1.5minLC_v003

¹H NMR (400 MHz, CDCl₃) δ 8.13 (1H, d), 7.91 (1H, d), 7.71 (1H, d), 7.34 (1H, s), 7.12 (1H, d), 4.21 (2H, d), 2.18 (1H, m), 1.07 (6H, d).

### Step 7: 1-(5-chloro-6-isobutoxypyridin-3-yl)-6-fluoro-1H-indole-3,5-dicarbonitrile

To a mixture of 3-bromo-1-(5-chloro-6-isobutoxypyridin-3-yl)-6-fluoro-1H-indole-5-carbonitrile (step 6) (310 mg, 0.12 mmol), Zn (23 mg, 0.35 mmol), Zn(CN)₂ (28 mg, 0.24 mmol) and Pd₂dba₃ (22 mg, 0.024 mmol) in 5 ml of DMF was added Pd(dppf)Cl₂ (17 mg 0.024 mmol). The resulting mixture was degassed and charged with N₂ three times, and then stirred at 150° C. for 2.5 hrs. When LCMS showed the reaction was completed, the reaction mixture was diluted with ethyl acetate and filtered over celite. The filtrate was washed with brine (×3), dried over anhydrous Na₂SO₄ and filtered. The filtration was evaporated under reduced pressure to afford the crude product, which was purified by column chromatography on silica gel eluting with 0˜10% ethyl acetate in hexanes to afford the title compound as yellow solid.

LC-MS: Rt=1.29 mins; MS m/z [M+H]+ 369.0; Method 5-95AB 1.5minLC_v003

¹H NMR (400 MHz, MeOD) δ 8.15 (1H, d), 8.12 (1H, d), 7.81 (1H, s), 7.73 (1H, d), 7.18 (1H, d), 4.23 (2H, d), 2.19 (1H, m), 1.07 (6H, d).

### Step 8: 3-amino-7-(5-chloro-6-isobutoxypyridin-3-yl)-7H-isoxazolo[4,5-f]indole-5-carbonitrile

To a mixture of N-hydroxyacetamide (110 mg, 1.5 mmol) and t-BuOK (1.5 ml, 1.5 mmol) in 10 ml of DMF was added 1-(5-chloro-6-isobutoxypyridin-3-yl)-6-fluoro-1H-indole-3,5-dicarbonitrile (step 7)(180 mg, 0.49 mmol). The resulting mixture was stirred at room temperature for 18 hrs, and then quenched with water. The mixture was extracted with EtOAc (×3). The combined organic layer was washed with brine (×3), dried over anhydrous Na₂SO₄ and filtered. The filtration was evaporated under reduced pressure to afford the crude product, which was purified by column chromatography on silica gel eluting with 0˜30% ethyl acetate in hexanes to afford the title compound as white solid.

LC-MS: Rt=1.29 mins; MS m/z [M+H]+ 382.1; Method 5-95AB 1.5minLC_v003

¹H NMR (400 MHz, CDCl₃) δ 8.20 (1H, d), 7.94 (1H, s), 7.79 (2H, m), 7.32 (1H, s), 4.24 (2H, d), 2.20 (1H, m), 1.08 (6H, d).

### Step 9: N-(7-(5-chloro-6-isobutoxypyridin-3-yl)-5-cyano-7H-isoxazolo[4,5-f]indol-3-yl)cyclopropanesulfonamide

To a mixture of 3-amino-7-(5-chloro-6-isobutoxypyridin-3-yl)-7H-isoxazolo[4,54]indole-5-carbonitrile (step 8)(100 mg, 0.25 mmol), DMAP (31 mg, 0.25 mmol) in 3 ml of pyridine was added cyclopropanesulfonyl chloride (254 ul, 2.50 mmol). This reaction was stirred at room temperature for 18 hrs, and then diluted with DCM, washed with water (×2). The organic layer was dried over anhydrous Na₂SO₄ and filtered. The filtration was evaporated under reduced pressure to afford the crude product, which was purified by preparative HPLC to afford the title compound as white solid.

LCMS: Rt=5.77 mins; MS m/z: [M+H]+ 486.0; Method 10-80AB 7minLC_v002

¹H NMR (400 MHz, CDCl₃) δ 8.20 (1H, s), 8.14 (1H, d), 7.83 (1H, s), 7.76 (1H, d), 7.35 (1H, s), 7.25 (1H, s), 4.17 (2H, d), 2.89 (1H, m), 2.12 (1H, m), 1.28 (2H, m), 0.99 (8H, m).

### Example 49

### 7-(5-chloro-6-isobutoxypyridin-3-yl)-3-(cyclopropanesulfonamido)-7H-isoxazolo[4,5-f]indole-5-carboxamide

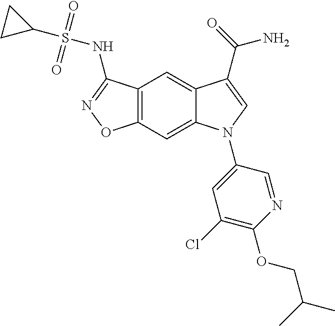 [SMILES: CC(C)COc1ncc(-n2cc(C(N)=O)c3cc4c(NS(=O)(=O)C5CC5)noc4cc32)cc1Cl]

To a solution of NaOH (165 mg, 4.12 mmol) in H₂O (1 ml) and MeOH (2 ml) was added N-(7-(5-chloro-6-isobutoxypyridin-3-yl)-5-cyano-7H-isoxazolo[4,5-f]indol-3-yl)cyclopropanesulfonamide (Example 48) (20 mg, 0.041 mmol). The reaction mixture was stirred at room temperature for 40 hrs. The reaction mixture was poured into ice-water, and extracted with ethyl acetate (×3). The combined organic layers were dried over anhydrous Na₂SO₄, and filtered. The filtration was concentrated under reduced pressure to give the crude product, which was purified by preparative HPLC to the title compound as white solid.

LC-MS: Rt=3.74 mins; MS m/z [M+H]+ 504.0; Method 10-80AB 7minLC_v002

¹HNMR (400 MHz, CDCl₃) δ 8.77 (1H, s), 8.26 (1H, s), 7.93 (1H, s), 7.70 (1H, s), 7.28 (1H, s), 7.20 (1H, s), 6.08 (2H, s), 4.24 (2H, d), 2.95 (1H, m), 2.23 (1H, m), 1.33 (2H, m), 1.09 (8H, m).

### Example 50

### N-(8-(5-chloro-6-isobutoxypyridin-3-yl)isoxazolo[4,5-g]isoquinolin-3-yl)cyclopropanesulfonamide

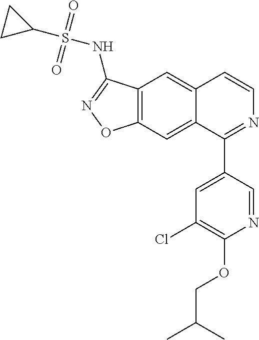 [SMILES: CC(C)COc1ncc(-c2nccc3cc4c(NS(=O)(=O)C5CC5)noc4cc23)cc1Cl]

### Step 1: (E)-3-(4-fluoro-3-methoxyphenyl)acrylic acid

To a mixture of 4-fluoro-3-methoxybenzaldehyde (Aldrich) (77 g, 0.5 mol) in EtOH (1000 ml) was added malonic acid (62 g, 0.6 mol) and pyridine (300 ml). The reaction mixture was stirred at 100° C. overnight. TLC showed the reaction was completed. After removal of the solvent, the resulting mixture was diluted with water and the pH was adjusted to pH=2 using 2 N HCl aqueous solution. The solid was collected and washed with water to afford the title compound as white solid.

LC-MS: Rt=1.06 mins; MS m/z [M+H]+ 197.4; Method 5-95AB 1.5minLC_v003

¹H NMR (400 MHz, DMSO-d₆) δ 13.11 (1H, br s), 7.51 (2H, m), 7.21 (2H, m), 6.52 (1H, d), 3.85 (3H, s).

### Step 2: (E)-3-(4-fluoro-3-methoxyphenyl)acryloyl azide

To a mixture of (E)-3-(4-fluoro-3-methoxyphenyl) acrylic acid (step 1) (80 g, 0.41 mol) in toluene (2000 ml) was added anhydrous triethylamine (81 g, 0.8 mol) and diphenyl phosphorazidate (220 g, 0.8 mol) at 0° C. The reaction mixture was stirred at room temperature for 16 h. After removal of the solvent, the resulting mixture was treated with water and ethyl acetate. The aqueous phase was extracted with ethyl acetate (×3). The combined organic phases were washed with brine (×2), dried over anhydrous magnesium sulfate and filtered. The filtration was concentrated under reduced pressure to give the title compound as white solid.

LC-MS: Rt=1.21 mins; MS m/z [M+H]+ 221.9; Method 5-95AB 1.5minLC_v003

¹H NMR (400 MHz, DMSO-d₆) δ 7.64 (2H, m), 7.25 (2H, m), 6.69 (1H, d), 3.85 (3H, s).

### Step 3: 7-fluoro-6-methoxyisoquinolin-1(2H)-one

A mixture of (E)-3-(4-fluoro-3-methoxyphenyl)acryloylazide (step 2) (66 g, 0.3 mol) in 1,2-dichlorobenzene (300 ml) was stirred at 140° C. for 1 h, and then a catalytic amount of iodine was added. The resulting mixture was stirred at 180° C. for additional 1.5 hrs. After removal of the solvent under reduced pressure, the resulting mixture was washed with DCM (×2) to afford the title compound as white solid.

LC-MS: Rt=1.29 mins; MS m/z [M+H]+ 193.7; Method 5-95AB 1.5minLC_v003

¹H NMR (400 MHz, DMSO-d₆) δ 11.20 (1H, br s), 7.78 (1H, d), 7.34 (1H, d), 7.12 (1H, t), 6.49 (1H, d), 3.92 (3H, s).

### Step 4: 7-fluoro-6-hydroxyisoquinolin-1(2H)-one

A mixture of 7-fluoro-6-methoxyisoquinolin-1(2H)-one (step 3) (40 g, 0.21 mol) in DCM (200 ml) was added BBr₃ (200 ml) at 0° C. The resulting mixture was then warmed to 20° C. and stirred at that temperature for 16 hrs. After the reaction was completed, the reaction mixture was treated with water and DCM. The aqueous phase was extracted with DCM (×3). The combined organic phases were washed with brine (×2), dried over anhydrous magnesium sulfate and filtered. The filtration was concentrated under reduced pressure to give the crude product by flash column chromatography on silica gel using a gradient from 0-30% ethyl acetate in hexanes to afford the title compound as white solid.

LC-MS: Rt=1.13 mins; MS m/z [M+H]+ 180.4; Method 5-95AB 1.5minLC_v003

¹H NMR (400 MHz, DMSO-d₆) δ 11.21 (1H, br s), 11.08 (1H, br s), 7.77 (1H, d), 7.53 (1H, br s), 6.90 (1H, br s), 6.41 (1H, d).

### Step 5: 7-fluoro-1-oxo-1,2-dihydroisoquinolin-6-yltrifluoromethanesulfonate

To a solution of 7-fluoro-6-hydroxy isoquinolin-1(2H)-one (step 4) (25 g, 0.14 mol) in DCM (500 ml) was added 1,1,1-trifluoro-N-phenyl-N-((trifluoromethyl)sulfonyl) methanesulfonamide (100 g, 0.28 mol) and pyridine (22 g, 0.28 mol). The resulting mixture was stirred at 0° C. for 2 hrs. When the reaction was completed as monitored by TLC, the reaction mixture was poured into stirred water. The separated aqueous phase was extracted with DCM (×3). The combined organic phases were washed with brine (×2), and dried over anhydrous magnesium sulfate and filtered. The filtration was concentrated under reduced pressure to give the crude product, which was purified by flash column chromatography on silica gel eluting a gradient from 0-10% ethyl acetate in hexanes to afford the title compound as white solid.

LC-MS: Rt=1.34 mins; MS m/z [M+H]+ 311.9; Method 5-95AB 1.5minLC_v003

¹H NMR (400 MHz, DMSO-d₆) δ 11.63 (1H, brs), 8.10 (2H, m), 7.27 (1H, t), 6.64 (1H, d).

### Step 6: 7-fluoro-1-oxo-1,2-dihydroisoquinoline-6-carbonitrile

To a mixture of 7-fluoro-1-oxo-1,2-dihydroisoquinolin-6-yltrifluoromethanesulfonate (step 5) (28 g, 90 mmol), DPPF (5 g, 9 mmol) and Zn(CN)₂ (21 g, 180 mmol) in DMF (200 ml) was added Pd₂(dba)₃ (8.2 g, 9 mmol) at room temperature. The resulting mixture was degassed and charged with nitrogen three times. After heating to 120° C., the reaction mixture was stirred at that temperature under nitrogen atmosphere for 4 hrs. When LC/MS indicated the starting material was consumed, the reaction mixture was cooled down and diluted with ethyl acetate (1000 ml). The mixture was filtered through a pad of celite and the filtration was then washed with brine (×3), dried over anhydrous magnesium sulfate and filtered. The filtration was evaporated under reduced pressure to give the crude product, which was purified by column chromatography on silica gel eluting with 0-30% ethyl acetate in hexanes to afford the title compound as black solid

LC-MS: Rt=1.33 mins; MS m/z [M+H]+ 189.4; Method 5-95AB 1.5minLC_v003

¹H NMR (400 MHz, DMSO-d₆) δ 11.68 (1H, brs), 8.39 (1H, d), 8.03 (1H, d), 7.28 (1H, m), 6.61 (1H, d).

### Step 7: 1-chloro-7-fluoroisoquinoline-6-carbonitrile

A mixture of 7-fluoro-1-oxo-1,2-dihydroisoquinoline-6-carbonitrile (step 6) (10 g, 53 mmol) in POCl₃ (150 ml) was stirred at 100° C. for 16 hrs. When the reaction mixture was completed as monitored by TLC, the mixture was poured into ice water carefully. The aqueous solution was extracted with DCM (×3) three times. The combined organic phases were washed with brine (×2), dried over anhydrous magnesium sulfate and filtered. The filtration was concentrated under reduced pressure to give the crude product, which was purified by column chromatography on silica gel eluting with 0˜10% ethyl acetate in hexanes to afford the title compound as white solid.

LC-MS: Rt=1.43 mins; MS m/z [M+H]+ 206.8; Method 5-95AB 1.5minLC_v003

¹H NMR (400 MHz, DMSO-d₆) δ 8.93 (1H, d), 8.47 (1H, d), 8.25 (1H, d), 8.02 (1H, d).

### Step 8: 1-(5-chloro-6-isobutoxypyridin-3-yl)-7-fluoroisoquinoline-6-carbonitrile

To a solution 1-chloro-7-fluoroisoquinoline-6-carbonitrile (step 7) (5 g, 24.3 mmol) in dioxane (100 ml) was added 3-chloro-2-isobutoxy-5-(4,4,5,5-tetramethyl-1,3,2-dioxaborolan-2-yl)pyridine (Intermediate U) (15 g, 48 mol), Pd(dppf)Cl₂ (3.7 g, 5 mmol) and Cs₂CO₃ (19 g, 97 mmol). The resulting mixture was bubbled into N₂ for 10 min, then stirred under N₂ atmosphere at 100° C. for 16 hrs. When the reaction was completed as monitored by LC-MS, the reaction mixture was cooled to room temperature and filtered over celite. The filtration was concentrated under reduced pressure to the resulting mixture, which was treated with water and ethyl acetate. The separated aqueous phase was extracted with ethyl acetate (×3). The combined organic phases were washed with brine (×2), and dried over anhydrous magnesium sulfate and filtered. The filtration was concentrated under reduced pressure to give the crude product, which was purified by flash column chromatography on silica gel eluting a gradient from 0-10% ethyl acetate in hexanes to afford the title compound as white solid.

LC-MS: Rt=1.25 mins; MS m/z [M+H]+ 356.4; Method 5-95AB 1.5minLC_v003

¹H NMR (400 MHz, DMSO-d₆) δ 8.89 (1H, d), 8.74 (1H, d), 8.41 (1H, br s.), 8.21 (1H, br s.), 7.95-8.09 (2H, m), 4.22 (2H, d), 2.12 (1H, m), 1.02 (6H, d).

### Step 9: 8-(5-chloro-6-isobutoxypyridin-3-yl)isoxazolo[4,5-g]isoquinolin-3-amine

A mixture of potassium 2-methylpropan-2-olate (1.7 g, 15 mmol) and propan-2-oneoxime (1.1 g, 15 mmol) was dissolved in DMF (100 ml) and stirred for 30 min at 0° C. Then a solution of 1-(5-chloro-6-isobutoxypyridin-3-yl)-7-fluoro isoquinoline-6-carbonitrile (step 8, 3.6 g, 10 mmol) in 10 ml of DMF was added to flask, and then stirred at 20° C. for 2 hrs. After removal of the solvent, the resulting mixture was treated with water and DCM. The separated aqueous phase was extracted with DCM (×3). The combined organic phases were washed with brine (×2), and dried over anhydrous magnesium sulfate and filtered. The filtration was concentrated under reduced pressure to afford the crude product, which was treated with in EtOH (100 ml) and 5% of HCl aqueous solution (100 ml). The resulting mixture was stirred at 100° C. for 1.5 hrs. After removal of the solvent, the mixture was treated with water and DCM. The separated aqueous phase was extracted with DCM (×3). The combined organic phases were washed with NaHCO₃ aqueous solution (×2), and dried over anhydrous magnesium sulfate and filtered. The filtration was concentrated under reduced pressure to afford the title compound as light yellow solid.

LC-MS: Rt=1.03 mins; MS m/z [M+H]+ 369.3; Method 5-95AB 1.5minLC_v003

¹H NMR (400 MHz, DMSO-d₆) δ 8.81 (1H, s), 8.67 (2H, br s), 8.57 (2H, m), 8.36 (2H, m), 8.15 (1H, s), 4.27 (2H, d), 2.15 (1H, m), 1.04 (6H, d).

### Step 10: N-(8-(5-chloro-6-isobutoxypyridin-3-yl)isoxazolo[4,5-g]isoquinolin-3-yl)cyclopropanesulfonamide

To a mixture of 8-(5-chloro-6-isobutoxypyridin-3-yl)isoxazolo[4,5-g]isoquinolin-3-amine (step 9) (369 mg, 1.0 mmol) in pyridine (2 ml) was added cyclopropanesulfonyl chloride (280 mg, 2.0 mmol) and DMAP (123 mg, 1.0 mmol). The resulting mixture was stirred at 50° C. under N₂ atmosphere for 16 hrs. When the reaction was completed as monitored by LC-MS, the reaction mixture was concentrated under reduced pressure to the resulting mixture, which was treated with water and ethyl acetate. The separated aqueous phase was extracted with ethyl acetate (×3). The combined organic phases were washed with brine (×2), and dried over anhydrous magnesium sulfate and filtered. The filtration was concentrated under reduced pressure to give the crude product, which was purified by preparative HPLC to afford the title compound as white solid.

LC-MS: Rt=6.38 mins; MS m/z [M+H]+ 473.4; Method 0-60AB 7minLC_v002

¹H NMR (400 MHz, DMSO-d₆) δ 11.90 (1H, br s), 8.80 (1H, s), 8.60 (1H, d), 8.50 (1H, d), 8.28 (2H, m), 8.12 (1H, d), 4.26 (2H, d), 3.12 (1H, m), 2.15 (1H, m), 1.18 (2H, d), 1.13 (2H, m), 1.05 (6H, d).

### Example 51

### N-(8-(5-chloro-6-isobutoxypyridin-3-yl)isoxazolo[4,5-g]isoquinolin-3-yl)methanesulfonamide

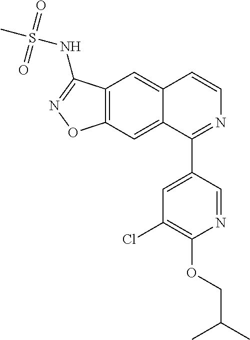 [SMILES: CC(C)COc1ncc(-c2nccc3cc4c(NS(C)(=O)=O)noc4cc23)cc1Cl]

The title compound was prepared by a method similar to that of Example 50 by replacing cyclopropanesulfonyl chloride (Example 50 step 10) with methanesulfonyl chloride;

LC-MS: Rt=4.96 mins; MS m/z 447.1 [M+H]+; Method 10-80AB 1.5minLC_v002

¹H NMR (400 MHz, DMSO-d6) δ 11.99 (1H, br s), 9.39 (1H, s), 8.84 (1H, s), 8.63 (1H, d), 8.41 (2H, s), 8.20 (1H, d), 4.27 (2H, d), 3.14 (3H, s), 2.15 (1H, m), 1.04 (6H, d).

### Example 52

### N-(8-(5,6-dichloropyridin-3-yl)isoxazolo[4,5-g]isoquinolin-3-yl)cyclopropanesulfonamide

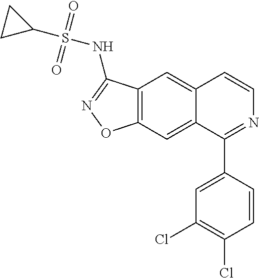 [SMILES: O=S(=O)(Nc1noc2cc3c(-c4ccc(Cl)c(Cl)c4)nccc3cc12)C1CC1]

The title compound was prepared by a method similar to that of Example 50 by replacing 3-chloro-2-isobutoxy-5-(4,4,5,5-tetramethyl-1,3,2-dioxaborolan-2-yl)pyridine (Intermediate U, Example 50 step 8) with (3,4-dichlorophenyl)boronic acid;

LC-MS: Rt=4.22 mins; MS m/z 433.9 [M+H]+; Method 10-80AB 1.5minLC_v002

¹H NMR (400 MHz, DMSO-d6) δ 11.75 (1H, br.s), 11.25 (1H, br.s), 8.57 (1H, d), 8.13 (1H, s), 8.09 (1H, d), 7.96 (1H, s), 7.82 (1H, d), 7.71 (1H, d), 3.06 (1H, d), 1.14 (2H, m), 1.09 (2H, m).

### Example 53

### N-(8-(5-chloro-6-isobutoxypyridin-3-yl)isoxazolo[4,5-g]isoquinolin-3-yl)N—N-dimethylsulfamide

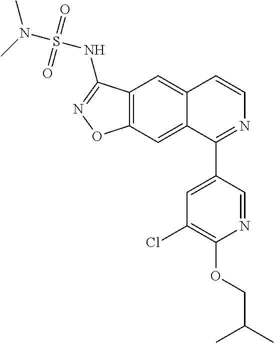 [SMILES: CC(C)COc1ncc(-c2nccc3cc4c(NS(=O)(=O)N(C)C)noc4cc23)cc1Cl]

The title compound was prepared by a method similar to that of Example 50 by replacing cyclopropanesulfonyl chloride (Example 50 step 10) with dimethylsulfamoyl chloride;

LC-MS: Rt=4.73 mins; MS m/z [M+H]+ 476.1; Method 10-80AB 7minLC_v002

¹H NMR (400 MHz, DMSO-d₆) δ11.31 (1H, s), 8.71 (1H, s), 8.63 (1H, s), 8.45 (1H, s), 8.21 (1H, s), 8.11 (1H, s), 7.97 (1H, br s), 4.26 (2H, d), 3.02 (6H, s), 2.12 (1H, m), 1.09 (6H, d).

### Example 54

### N-(8-(5-chloro-6-isobutoxypyridin-3-yl)isoxazolo[4,5-g]isoquinolin-3-yl)azetidine-1-sulfamide

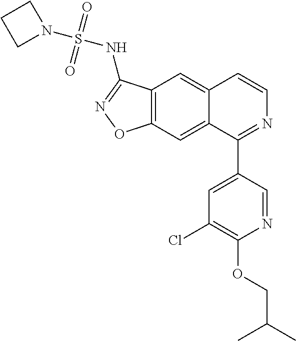 [SMILES: CC(C)COc1ncc(-c2nccc3cc4c(NS(=O)(=O)N5CCC5)noc4cc23)cc1Cl]

The title compound was prepared by a method similar to that of Example 50 by replacing cyclopropanesulfonyl chloride (Example 50 step 10) with azetidine-1-sulfonyl chloride;

LC-MS: Rt=4.85 mins; MS m/z [M+H]+ 488.2; Method 10-80AB 7minLC_v002

¹H NMR (400 MHz, DMSO-d₆) δ11.31 (1H, s), 8.67 (1H, s), 8.59 (1H, s), 8.46 (1H, s), 8.24 (1H, s), 8.10 (1H, m), 7.89 (1H, m), 4.35 (2H, m), 4.30 (2H, d), 3.82 (2H, m), 2.25 (1H, m), 2.16 (2H, m), 1.11 (6H, d).

### Example 55

### N-(8-(5-chloro-6-isobutoxypyridin-3-yl)isoxazolo[5,4-g]quinolin-3-yl)cyclopropanesulfonamide

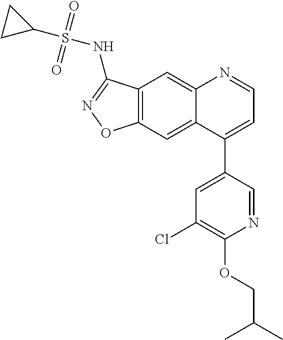 [SMILES: CC(C)COc1ncc(-c2ccnc3cc4c(NS(=O)(=O)C5CC5)noc4cc23)cc1Cl]

### Step 1: 5-(((3-bromo-4-fluorophenyl)amino)methylene)-2,2-dimethyl-1,3-dioxane-4,6-dione

A mixture of 2,2-dimethyl-1,3-dioxane-4,6-dione (7.6 g, 53 mmol) in trimethoxymethane (80 g, 371 mmol) was stirred at 100° C. for 1 h, then a solution of 3-bromo-4-fluoroaniline (10 g, 53 mmol) in trimethoxymethane (80 g, 371 mmol) was added. The resulting mixture was stirred at 90° C. for additional 16 hrs. After cooling down, the mixture was washed by a mixed solvent (hexanes: ethyl acetate=3:1) to give the title compound as white solid.

LC-MS: Rt=1.03 mins; MS m/z [M+H]+ 343.9; Method 5-95AB 1.5minLC_v003

¹H NMR (400 MHz, CDCl₃) δ11.19 (1H, d), 8.53 (1H, d), 7.50 (1H, m), 7.18 (2H, m), 1.76 (6H, s).

### Step 2: 7-bromo-6-fluoroquinolin-4-ol

5-(((3-bromo-4-fluorophenyl)amino)methylene)-2,2-dimethyl-1,3-dioxane-4,6-dione (step 1)(2 g, 5.8 mmol) was added to diphenylether (20 ml) by portions at 260° C. over 10 min. When the addition was completed, the mixture was stirred at 260° C. for 20 min under N₂. After cooling down to room temperature, the reaction mixture was added 20 ml of hexanes. The mixture was filtered to give the title compound.

LC-MS: Rt=1.03 mins; MS m/z [M+H]+ 241.9; Method 5-95AB 1.5minLC_v003

¹H NMR (400 MHz, MeOD) δ 7.93 (1H, d), 7.50 (1H, d), 6.29 (1H, d), 6.25 (1H, d).

### Step 3: 6-fluoro-4-hydroxyquinoline-7-carbonitrile

To a suspension of 7-bromo-6-fluoroquinolin-4-ol (step 2) (700 mg, 2.9 mmol) and Pd₂(dba)₃ (532 mg, 0.57 mmol), DPPF (637 mg, 1.15 mmol) in DMF (10 ml) was added Zn(CN)₂ (665 mg, 5.7 mmol) at 20° C. with stirring. The resulting mixture was degassed and charged with N₂ three times and then stirred at 120° C. for 4 hrs. When LC/MS indicated the reaction was completed, the mixture was portioned between ethyl acetate and brine. The organic phase was washed with brine (×3). The organic phase was dried over anhydrous Na₂SO₄ and filtered. The filtration was evaporated under reduced pressure to give the crude product, which was purified by flash column chromatography on silica gel using a gradient 0-30% ethyl acetate in hexanes to afford the title compound as yellow solid.

LC-MS: Rt=1.03 mins; MS m/z [M+H]+ 189.0; Method 5-95AB 1.5minLC_v003

¹H NMR (400 MHz, DMSO-d₆) δ 12.14 (1H, s), 8.11 (1H, d), 8.04 (1H, d), 7.93 (1H, d), 6.12 (1H, d).

### Step 4: 4-chloro-6-fluoroquinoline-7-carbonitrile

A mixture of 6-fluoro-4-hydroxyquinoline-7-carbonitrile (step 3) (250 mg, 1.3 mmol) in POCl₃ (2 ml) was stirred at 110° C. for 2 hrs. When TLC indicated the reaction was completed, the mixture was added to 20 ml of water and extracted by ethyl acetate (×3). The combined organic phases were dried over anhydrous Na₂SO₄ and filtered. The filtration was evaporated under reduced pressure to give the crude product, which was purified by flash column chromatography on silica gel using a gradient 0-5% ethyl acetate in hexanes to afford the title compound as yellow solid.

LC-MS: Rt=1.03 mins; MS m/z [M+H]+ 207.0; Method 5-95AB 1.5minLC_v003

¹H NMR (400 MHz, CDCl₃) δ 8.87 (1H, d), 8.52 (1H, d), 8.02 (1H, d), 7.65 (1H, d).

### Step 5: 4-(5-chloro-6-isobutoxypyridin-3-yl)-6-fluoroquinoline-7-carbonitrile

To a solution of 4-chloro-6-fluoroquinoline-7-carbonitrile (step 4) (120 mg, 0.58 mmol) in dioxane (3 ml) was added 3-chloro-2-isobutoxy-5-(4,4,5,5-tetramethyl-1,3,2-dioxaborolan-2-yl)pyridine (intermediate U) (271 g, 0.87 mmol), Pd(dppf)Cl₂ (42 mg, 0.058 mmol) and Cs₂CO₃ (283 g, 0.87 mmol). The mixture was degassed and charged with N₂ three times, and then stirred at 100° C. for 4 hrs. When LC/MS indicated the reaction was completed, the reaction mixture was cooled to room temperature and diluted with DCM. The mixture was filtered over celite and the filtration was washed with brine (×3) and dried over anhydrous Na₂SO₄. After removal of the solvent under reduced pressure, the crude product was purified by flash column chromatography on silica gel using a gradient 0-10% ethyl acetate in hexanes to afford the title compound as yellow solid.

LC-MS: Rt=1.03 mins; MS m/z [M+H]+ 356.1; Method 5-95AB 1.5minLC_v003

¹H NMR (400 MHz, CDCl₃) δ 9.00 (1H, d), 8.87 (1H, d), 8.52 (1H, d), 8.19 (1H, s), 8.02 (1H, d), 7.65 (1H, d), 4.21 (2H, d), 2.11 (1H, m), 1.01 (6H, d).

### Step 6: 8-(5-chloro-6-isobutoxypyridin-3-yl)isoxazolo[5,4-g]quinolin-3-amine

To the mixture of 4-(5-chloro-6-isobutoxypyridin-3-yl)-6-fluoroquinoline-7-carbonitrile (step 5)(90 mg, 0.25 mmol) in DMF (3 ml) was added t-BuOK (85 mg, 0.76 mmol) and N-hydroxyacetamide (57 mg, 0.76 mmol). The resulting mixture was stirred at 20° C. for 1 h. When TLC indicated the reaction was completed, the reaction mixture was treated with water and ether. The separated organic phase was washed with brine, dried over anhydrous magnesium sulfate and filtered. The filtration was concentrated under reduced pressure to give the crude product, which was purified by flash column chromatography on silica gel using a gradient 0-50% ethyl acetate in hexanes to afford the title compound as yellow solid.

LC-MS: Rt=1.03 mins; MS m/z [M+H]+ 369.1; Method 5-95AB 1.5minLC_v003

¹H NMR (400 MHz, DMSO-d₆) δ 9.00 (1H, d), 8.84 (1H, s), 8.32 (1H, s), 8.19 (1H, s), 7.99 (1H, s), 7.60 (1H, d), 5.60 (2H, br s), 4.21 (2H, d), 2.11 (1H, m), 1.01 (6H, d).

### Step 7: N-(8-(5-chloro-6-isobutoxypyridin-3-yl)isoxazolo[5,4-g]quinolin-3-yl)cyclopropanesulfonamide

To a mixture of 8-(5-chloro-6-isobutoxypyridin-3-yl)isoxazolo[5,4-g]quinolin-3-amine (step 6, 100 mg, 0.27 mmol) and DMAP (66 mg, 0.54 mmol) in pyridine (2 ml) was added cyclopropanesulfonyl chloride (2 ml). The mixture was stirred at 60° C. for 20 hrs. When TLC indicated the reaction was completed, the reaction mixture was treated with water and ethyl acetate. The separated organic phase was washed with brine (×3), dried over anhydrous magnesium sulfate and filtered. The filtration was concentrated under redcued pressure to give the crude product, which was dissovled in 2 ml of THF. To the mixture was added TBAF (45 mg, 0.18 mmol). The resulting mixture was stirred at 26° C. for 1 h. When LCMS showed the reaction was completed, the reaction mixture was concentrated under reduced pressure to give the crude product, which was purified by preparative HPLC to give the title compound.

LC-MS: Rt=3.77 mins; MS m/z [M+H]+ 473.0; Method 10-80AB 7minLC_v001

¹H NMR (400 MHz, DMSO-d₆) δ 11.84 (1H, s), 9.00 (1H, d), 8.84 (1H, s), 8.32 (1H, s), 8.19 (1H, s), 7.99 (1H, s), 7.60 (1H, d), 4.21 (2H, d), 3.09 (1H, m), 2.11 (1H, m), 1.11 (4H, m), 1.01 (6H, d).

### Example 56

### N-(8-(5-chloro-6-isobutoxypyridin-3-yl)isoxazolo[5,4-g]isoquinolin-3-yl)cyclopropanesulfonamide

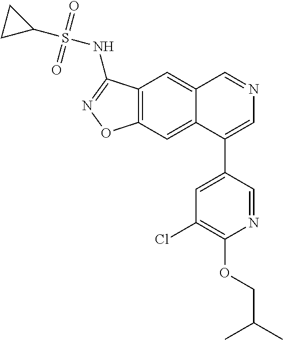 [SMILES: CC(C)COc1ncc(-c2cncc3cc4c(NS(=O)(=O)C5CC5)noc4cc23)cc1Cl]

### Step 1: Methyl 2-((4-fluoro-3-methoxybenzyl)amino)acetate

To a mixture of 4-fluoro-3-methoxybenzaldehyde (Aldrich) (21 g, 136 mmol) and methyl 2-aminoacetate hydrochloride (22 g, 176 mmol) in MeOH (300 ml) was added NaOAc (16 g, 204 mmol) and the mixture was stirred at 20° C. for 2 hrs. Then the mixture was stirred at 0° C., and NaBH₄ (8 g, 204 mmol) was added portionwise over 1 hr, then this solution was allowed to warm up slowly to room temperature for 16 hrs. The mixture was diluted with H₂O and the aqueous was extracted with DCM (×2), dried over anhydrous Na₂SO₄, filtered, and concentrated to afford crude, which was purified by flash column chromatography on silica gel using a gradient 0-20% ethyl acetate in hexanes afford the title compound as yellow oil.

LC-MS: Rt=1.23 mins; MS m/z [M+H]+ 228.2; Method 5-95AB 1.5minLC_v003

¹H NMR (400 MHz, CDCl₃) δ 6.99 (1H, m), 6.91 (1H, m), 6.71 (1H, m), 4.43 (2H, d), 4.13 (1H, m), 3.92 (2H, d), 3.84 (3H, s), 3.57 (3H, s).

### Step 2: Methyl 2-(N-(4-fluoro-3-methoxybenzyl)-4-methylphenylsulfonamido)acetate

To a solution of methyl 2-((4-fluoro-3-methoxybenzyl)amino)acetate (step 1) (16 g, 70 mmol) in DCM (250 ml) was added DMAP (1.72 g, 14 mmol), Et₃N (21 g, 211 mmol) and TsCl (27 g, 141 mmol). The mixture was stirred at 0° C. for 5 hrs, the mixture was diluted with H₂O and the aqueous was extracted with DCM (×3), and dried over anhydrous Na₂SO₄, filtered, and concentrated to afford crude, which was purified by flash column chromatography on silica gel using a gradient 0-20% ethyl acetate in hexanes afford the title compound as yellow oil.

LC-MS: Rt=1.35 mins; MS m/z [M+H]+ 382.2; Method 5-95AB 1.5minLC_v003

¹H NMR (400 MHz, CDCl₃) δ 7.78 (2H, d), 7.34 (2H, d), 6.99 (1H, dd), 6.91 (1H, dd), 6.71 (1H, dd), 4.43 (2H, s), 3.92 (2H, s), 3.84 (3H, s), 3.57 (3H, s), 2.45 (3H, s).

### Step 3: 2-(N-(4-fluoro-3-methoxybenzyl)-4-methylphenylsulfonamido)acetic acid

To a solution methyl 2-(N-(4-fluoro-3-methoxybenzyl)-4-methylphenylsulfonamido) acetate (step 2) (21 g, 55 mmol) in THF:H₂O (3:1, 200 ml) was added LiOH (11.5 g, 275 mmol), the reaction mixture was stirred for 1 hr. The mixture was diluted with water, extracted with DCM (×2). The combined organic layers were washed with brine (×1), and dried over anhydrous Na₂SO₄, filtered, and concentrated under reduced pressure to afford the title compound as yellow oil, which was used for the next step directly.

LC-MS: Rt=1.15 mins; MS m/z [M+H]+ 368.1; Method 5-95AB 1.5minLC_v003

¹H NMR (400 MHz, DMSO-d₆) δ 12.56 (1H, br s), 7.72 (2H, d), 7.37 (2H, d), 7.09 (1H, dd), 6.88 (1H, dd), 6.76 (1H, m), 4.33 (2H, s), 3.81 (2H, s), 3.57 (3H, s), 2.36 (3H, s).

### Step 4: 6-fluoro-7-hydroxy-2-tosyl-2,3-dihydroisoquinolin-4(1H)-one

To a solution methyl 2-(N-(4-fluoro-3-methoxybenzyl)-4-methylphenyl sulfonamido)acetic acid (step 3) (18 g, 49 mmol) in DCM (210 ml) was added oxalyl dichloride (62 g, 490 mmol) and DMF (Cat.), the reaction mixture was stirred at 0° C. for 1 hr. The reaction mixture was concentrated under reduced pressure to afford a residue, which was dissolved in DCE (210 ml), AlCl₃ (32 g, 245 mmol) was added. The reaction mixture was stirred at 100° C. for 1 hr, the mixture was poured into ice-water and then stirred for 0.5 hr, filtered, the aqueous was extracted with DCM (×3), dried over anhydrous Na₂SO₄, filtered, and concentrated under reduced pressure to afford crude, which was purified by flash column chromatography on silica gel using a gradient 0-20% ethyl acetate in hexanes afford the title compound as yellow solid.

LC-MS: Rt=1.02 mins; MS m/z [M+H]+ 336.1; Method 5-95AB 1.5minLC_v003

¹H NMR (400 MHz, DMSO-d₆) δ 11.27 (1H, s), 7.59 (2H, d), 7.33 (3H, m), 6.95 (1H, d), 4.48 (2H, s), 3.98 (2H, s), 2.34 (3H, s)

### Step 5: 6-fluoro-4-oxo-2-tosyl-1,2,3,4-tetrahydroisoquinolin-7-yltrifluoro methanesulfonate

To a solution of 6-fluoro-7-hydroxy-2-tosyl-2,3-dihydroisoquinolin-4(1H)-one (step 4) (3.35 g, 10 mmol) in CHCl₃ (30 ml) was added 1,1,1-trifluoro-N-phenyl-N-((trifluoromethyl) sulfonyl)methanesulfonamide (PhNTf2, 7.14 g, 20 mmol) and Et₃N (3 g, 30 mmol). The resulting mixture was stirred at 50° C. for 3 hrs. The mixture was evaporated to afford the crude product, which was purified by flash column chromatography on silica gel using a gradient 0-20% ethyl acetate in hexanes to afford the title compound as yellow solid.

LC-MS: Rt=1.16 mins; MS m/z [M+H]+ 467.1; Method 5-95AB 1.5minLC_v003

¹H NMR (400 MHz, DMSO-d₆) δ 7.65 (2H, d), 7.43 (3H, m), 6.87 (1H, d), 4.48 (2H, s), 3.98 (2H, s), 2.34 (3H, s)

### Step 6: 6-fluoro-4-hydroxyisoquinoline-7-carbonitrile

To a suspension of 6-fluoro-4-oxo-2-tosyl-1,2,3,4-tetrahydroisoquinolin-7-yltrifluoro methanesulfonate (step 5)(1.5 g, 3.2 mmol), dppf (710 mg, 1.28 mmol) and Pd₂(dba)₃ (585 mg, 0.64 mmol) in DMF (30 ml) was added Zn(CN)₂ (1.11 g, 9.6 mmol), the mixture was degassed stirred at 120° C. for 4 hrs under N₂. The mixture was poured into ethyl acetate (200 ml) and brine (400 ml). The organic phase was dried over anhydrous Na₂SO₄, filtered and the filtration was concentrated to afford a residue, which was purified by flash column chromatography on silica gel using a gradient 0-50% ethyl acetate in hexanes to afford the title compound as yellow solid.

LC-MS: Rt=0.75 mins; MS m/z [M+H]+ 189.1; Method 5-95AB 1.5minLC_v003

1H NMR (400 MHz, CDCl₃) δ 10.54 (1H, s), 9.15 (1H, s), 8.74 (1H, s), 8.55 (1H, d), 7.84 (1H, d).

### Step 7: 7-cyano-6-fluoroisoquinolin-4-yl trifluoromethanesulfonate

To a solution of 6-fluoro-4-hydroxyisoquinoline-7-carbonitrile (step 6) (270 mg, 1.38 mmol) in pyridine (15 ml) was added 1,1,1-trifluoro-N-phenyl-N-((trifluoromethyl)sulfonyl) methanesulfonamide (PhNTf2, 735 mg, 2.07 mmol) and DMAP (162 mg, 1.38 mmol). The resulting mixture was stirred at room temperature for 2 hrs. The mixture was diluted with H₂O and the aqueous was extracted with DCM (×3). The combined organic extracts were washed with brine, and dried over anhydrous Na₂SO₄, filtered, and the filtration was concentrated to afford the crude product, which was purified by flash column chromatography on silica gel using a gradient 0-30% ethyl acetate in hexanes to afford the title compound as white solid.

LC-MS: Rt=1.44 mins; MS m/z [M+H]+ 320.9; Method 5-95AB 1.5minLC_v003

1H NMR (400 MHz, CDCl₃) δ 9.34 (1H, s), 8.74 (1H, s), 8.55 (1H, d), 7.84 (1H, d).

### Step 8: 4-(5-chloro-6-isobutoxypyridin-3-yl)-6-fluoroisoquinoline-7-carbonitrile

To a solution of 7-cyano-6-fluoroisoquinolin-4-yl trifluoromethanesulfonate (step 7) (156 mg, 0.48 mmol) in dioxane (13 ml) was added 3-chloro-2-isobutoxy-5-(4,4,5,5-tetramethyl-1,3,2-dioxaborolan-2-yl)pyridine (intermediate U) (303 mg, 0.96 mmol), Pd(dppf)Cl₂ (34.8 mg, 0.048 mmol) and Cs₂CO₃ (315 mg, 0.96 mmol). The mixture was bubbled into N₂ for 10 mins, the resulting mixture was stirred at 100° C. for 4 hrs. The mixture was cooled to room temperature and diluted with H₂O and the aqueous was extracted with DCM (×3). The combined organic extracts were washed with brine, and dried over anhydrous Na₂SO₄, filtered, and the filtration was concentrated under reduced pressure to afford crude product, which was purified by flash column chromatography on silica gel using a gradient 0-50% ethyl acetate in hexanes to afford the title compound as yellow solid.

LC-MS: Rt=1.21 mins; MS m/z [M+H]+ 356.1; Method 5-95AB 1.5minLC_v003

1H NMR (400 MHz, CDCl₃) δ 9.33 (1H, s), 8.61 (1H, s), 8.50 (1H, d), 8.13 (1H, d), 7.76 (1H, d), 7.62 (1H, d), 4.25 (2H, d), 2.2 (1H, m), 1.10 (6H, d).

### Step 9: 8-(5-chloro-6-isobutoxypyridin-3-yl)isoxazolo[5,4-g]isoquinolin-3-amine

A mixture of 4-(5-chloro-6-isobutoxypyridin-3-yl)-6-fluoroisoquinoline-7-carbonitrile (step 8) (75 mg, 0.21 mmol), N-hydroxyacetamide (47.5 mg, 0.63 mmol) and t-BuOK (1M in THF) (0.63 ml, 0.63 mmol) in DMF (3 ml) was stirred for 16 hrs. The mixture was quenched with water (80 ml), extracted with ethyl acetate (×3), washed with brine (×3), dried over anhydrous Na₂SO₄, filtered and the filtration was concentrated to afford crude product, which was treated with 5 ml of ethanol and 5 ml of 5% HCl aqueous solution. The resulting mixture was heated to reflux for 45 min. After removal of the organic solvent, the remaining aqueous phase was basified with saturated Na₂CO₃ aqueous solution. The mixture was extracted with ethyl acetate (×3), washed with brine (×2), dried over anhydrous magnesium sulfate and filtered. The filtration was concentrated under reduced pressure to give the crude product, which was purified by flash chromatography on silica gel eluting with 0-50% ethyl acetate in hexanes to afford the title compound as a yellow solid.

LC-MS: Rt=1.43 mins; MS m/z [M+H]+ 369.1; Method 5-95AB 1.5minLC_v003

1H NMR (400 MHz, CDCl₃) δ 8.16 (1H, d), 8.07 (1H, d), 7.93 (1H, d), 7.71 (1H, d), 7.46 (1H, s), 7.22 (1H, s), 4.72 (2H, s), 4.25 (2H, d), 2.2 (1H, m), 1.10 (6H, d).

### Step 10: N-(8-(5-chloro-6-isobutoxypyridin-3-yl)isoxazolo[5,4-g]isoquinolin-3-yl)cyclopropanesulfonamide

To a solution of 8-(5-chloro-6-isobutoxypyridin-3-yl)isoxazolo[5,4-g]isoquinolin-3-amine (step 9) (0.01 g, 0.027 mmol) and DMAP (6.6 mg, 0.054 mmol) in pyridine (0.1 ml) was added cyclopropanesulfonyl chloride (0.2 ml), the mixture was stirred at 60° C. for 20 hrs. The mixture was extracted with ethyl acetate (×3), washed with brine, dried over anhydrous magnesium sulfate, filtered and concentrated to afford a residue, which was treated with TBAF (0.034 mmol) in THF (1 ml). The mixture was stirred at 25° C. for 2 hrs and then treated with water and ethyl acetate. The separated organic phase was washed with water, dried over anhydrous Na₂SO₄ and filtered. The filtration was concentrated to afford a crude product, which was purified by preparative HPLC to afford the title compound as yellow solid.

LC-MS: Rt=3.47 mins; MS m/z [M+H]+ 473.0; Method 10-80AB 7minLC_v001

1H NMR (400 MHz, CDCl₃) δ 8.16 (1H, d), 8.07 (1H, d), 7.93 (1H, d), 7.71 (1H, d), 7.46 (1H, s), 7.29 (1H, s), 7.22 (1H, s), 4.25 (2H, d), 3.09 (1H, m), 2.25 (1H, m), 1.41 (4H, m), 1.07 (6H, d).

### Example 57

### N-(8-(5-chloro-6-isobutoxypyridin-3-yl)isoxazolo[5,4-g]isoquinolin-3-yl)methanesulfonamide

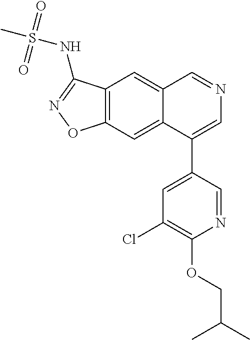 [SMILES: CC(C)COc1ncc(-c2cncc3cc4c(NS(C)(=O)=O)noc4cc23)cc1Cl]

The title compound was prepared by a method similar to that of Example 56 by replacing cyclopropanesulfonyl chloride (Example 56 step 10) with methanesulfonyl chloride;

LC-MS: Rt=3.85 mins; MS m/z [M+H]+ 447.0; Method 10-80AB 7minLC_v002

¹H NMR (400 MHz, CD₃OD) δ 9.42 (1H, s), 8.68 (1H, s), 8.32 (1H, s), 8.24 (1H, d), 8.02 (1H, d), 7.64 (1H, s), 4.28 (2H, d), 3.15 (3H, s), 2.20 (1H, m), 1.10 (6H, d).

### Example 58

### N-(8-(5-chloro-6-isobutoxypyridin-3-yl)naphtho[2,3-d]isoxazol-3-yl)cyclopropanesulfonamide

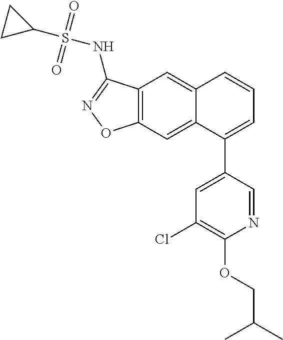 [SMILES: CC(C)COc1ncc(-c2cccc3cc4c(NS(=O)(=O)C5CC5)noc4cc23)cc1Cl]

### Step 1: 2-(3-bromo-4-fluorophenyl)ethanol

To a solution of 2-(3-bromo-4-fluorophenyl)acetic acid (Aldrich) (40 g, 172 mmol) in THF (300 ml) was added CDI (42 g, 258 mmol). The resulting mixture was stirred at room temperature for 2 hrs and then added a mixture of NaBH₄ (19 g, 517 mmol) in H₂O (100 ml). The mixture was stirred at room temperature for additional 16 hrs. After removal of the solvents, the residue was treated with water and ethyl acetate. The separated organic phase was washed with water (×3), dried over anhydrous Na₂SO₄ and filtered. The filtration was concentrated to afford the crude product, which was purified by flash column chromatography on silica gel using a gradient 0-20% ethyl acetate in hexanes to afford the title compound as colorless oil.

LC-MS: Rt=1.23 mins; MS m/z [M+H]+ 218.9; Method 5-95AB 1.5minLC_v003

¹H NMR (400 MHz, CDCl₃) δ7.43 (1H, m), 7.14 (1H, m), 7.06 (1H, m), 3.85 (2H, q), 2.83 (2H, t), 1.42 (1H, t)

### Step 2: (E)-methyl 4-(3-bromo-4-fluorophenyl)but-2-enoate

To a solution of 2-(3-bromo-4-fluorophenyl) ethanol (step 1) (15 g, 68 mmol) in DCM (300 ml) was added Dess-Martin Reagent (35 g, 82 mmol). The mixture was stirred at room temperature for 1 h. 75 ml of 1N NaOH was added to the reaction mixture, washed with brine (350 ml), dried and concentrated to afford the crude. The crude was dissolved in DCM (300 ml), Then methyl 2-(triphenylphosphoranylidene)acetate (34 g, 103 mmol) was added. This mixture reaction was stirred at room temperature for 3 hrs. This reaction mixture was evaporated to afford crude, which was purified by flash column chromatography on silica gel using a gradient 0-5% ethyl acetate in hexanes to afford the title compound as colorless oil.

LC-MS: Rt=1.33 mins; MS m/z [M+H]+ 272.9; Method 5-95AB 1.5minLC_v003

¹H NMR (400 MHz, CDCl₃) δ7.37 (1H, d), 7.05 (3H, m), 5.80 (1H, d), 3.73 (3H, s), 3.47 (2H, d).

### Step 3: methyl 4-(3-bromo-4-fluorophenyl)butanoate

To a solution of (E)-methyl 4-(3-bromo-4-fluorophenyl)but-2-enoate (step 2) (3 g, 11 mmol) and NiCl₂.6 H₂O (525 mg, 2.19 mmol) in MeOH (50 ml) was added NaBH₄ (2 g, 55 mmol) in MeOH (300 ml) at −5° C. slowly. After addition, the reaction mixture was stirred at 25° C. for 2 hrs. The reaction mixture was quenched with saturated NH₄Cl (100 ml), extracted with DCM (×3), washed with brine (×2). The organic layer was dried over anhydrous Na₂SO₄ and filtered. The filtration was concentrated to afford the title compound as colorless oil.

LC-MS: Rt=1.35 mins; MS m/z [M+H]+ 275.0; Method 5-95AB 1.5minLC_v003

¹H NMR (400 MHz, CDCl₃) δ7.35 (1H, m), 7.05 (2H, m), 3.67 (3H, s), 2.60 (2H, t), 3.32 (2H, t), 1.92 (2H, m).

### Step 4: 4-(3-bromo-4-fluorophenyl)butanoic acid

To a solution of methyl 4-(3-bromo-4-fluorophenyl)butanoate (step 3) (2.6 g, 9.45 mmol) in THF/H₂O (3:1, 40 ml) was added LiOH H₂O (595 mg, 14 mmol). The mixture was stirred at room temperature for 16 hrs. The mixture was adjusted with 1N HCl to pH=5 and extracted with EtOAc (×3). The combined organic layers was washed with brine, dried over anhydrous Na₂SO₄, filtered and concentrated to afford the title compound as a white solid.

LC-MS: Rt=1.18 mins; MS m/z [M+H]+ 260.9; Method 5-95AB 1.5minLC_v003

¹H NMR (400 MHz, CDCl₃) δ13.36 (1H, br s), 7.36 (1H, m), 7.05 (2H, m), 2.62 (2H, t), 2.36 (2H, t), 1.92 (2H, m).

### Step 5: 6-bromo-7-fluoro-3,4-dihydronaphthalen-1(2H)-one

4-(3-bromo-4-fluorophenyl)butanoic acid (step 4) (2 g, 8 mmol) in H₂SO₄ (20 ml) was stirred at 100° C. for 2 hrs. The mixture was poured into ice-water, extracted with DCM (×3), the organic layers was washed with brine, dried over anhydrous Na₂SO₄, filtered and concentrated to afford the title compound as a pale yellow solid.

LC-MS: Rt=1.09 mins; MS m/z [M+H]+ 242.9; Method 5-95AB 1.5minLC_v003

¹H NMR (400 MHz, CDCl₃) δ7.72 (1H, d), 7.49 (1H, d), 2.92 (2H, t), 2.64 (2H, t), 2.12 (2H, m).

### Step 6: 6-bromo-7-fluoronaphthalen-1-ol

To a solution of 6-bromo-7-fluoro-3,4-dihydronaphthalen-1(2H)-one (step 5) (1.7 g, 6.8 mmol) in HOAc (28 ml) and HBr (20 uL) was added Br₂ (1.2 g, 7.5 mmol) in HOAc (2 ml) in an ice bath. The mixture was stirred at room temperature for 3 hrs. The mixture was diluted with DCM (100 ml), washed with H₂O (×3) and sat. NaHCO₃ (100 ml). The organic layer was dried over anhydrous Na₂SO₄, filtered and concentrated to afford 1.8 g of crude as brown oil. This crude was dissolved in DMF (40 ml), LiBr (1 g, 12 mmol), Li₂CO₃ (834 mg, 11 mmol) was added. The reaction mixture was stirred at 160° C. for 3.5 hrs. The mixture was poured into ice water, extracted with hexanes/EtOAc (3:1, ×2). The organic layers were washed with H₂O (×2), brine (×2). The organic layer was dried over anhydrous Na₂SO₄, filtered and concentrated to afford the crude product, which was purified by flash column chromatography on silica gel using a gradient 0-10% ethyl acetate in hexanes to afford the title compound as white solid.

LC-MS: Rt=1.01 mins; MS m/z [M+H]+ 240.9; Method 5-95AB 1.5minLC_v003

¹H NMR (400 MHz, CDCl₃) δ8.03 (1H, d), 7.87 (1H, d), 7.31 (3H, m), 6.82 (1H, d).

### Step 7: 3-fluoro-5-hydroxy-2-naphthonitrile

To a mixture of 6-bromo-7-fluoronaphthalen-1-ol (step 6) (700 mg, 3 mmol), Zn (570 mg, 9 mmol), Zn(CN)₂ (682 mg, 6 mmol) and Pd₂(dba)₃ (456 mg, 0.6 mmol) in 20 ml of DMF was added Pd(dppf)Cl₂ (366 mg, 0.6 mmol). This reaction mixture was stirred at 130° C. under N₂ for 1.5 hrs. The mixture was cooled to room temperature, diluted with EtOAc and water, extracted with EtOAc (×3). The organic layer was washed with brine (×3). The organic layer was dried over anhydrous Na₂SO₄, filtered and evaporated to afford the crude product, which was purified by flash column chromatography on silica gel using a gradient 0-10% ethyl acetate in hexanes to afford the title compound as pale yellow solid.

LC-MS: Rt=0.98 mins; MS m/z [M+H]+ 188.0; Method 5-95AB 1.5minLC_v003

¹H NMR (400 MHz, CDCl₃) δ8.61 (1H, d), 7.92 (1H, d), 7.45 (3H, m), 7.07 (1H, d)

### Step 8: 6-cyano-7-fluoronaphthalen-1-yl trifluoromethanesulfonate

To a solution 3-fluoro-5-hydroxy-2-naphthonitrile (step 7) (300 mg, 1.6 mmol) in dry DCM (8 ml) was added pyridine (1 ml) and the solution was cooled to 0° C., Tf₂O (543 mg, 2 mmol) was added dropwise. After addition, the mixture was stirred at room temperature for 5 hrs. The mixture was diluted with DCM, and then washed with 1N HCl (×3), saturated NaHCO₃ (×1) and brine (×1). The organic layer was dried over anhydrous Na₂SO₄, filtered and concentrated to afford the crude product, which was purified by flash column chromatography on silica gel using a gradient 0-4% ethyl acetate in hexanes to afford the title compound as white yellow.

LC-MS: Rt=1.23 mins; MS m/z [M+H]+ 319.9; Method 5-95AB 1.5minLC_v003

¹H NMR (400 MHz, CDCl₃) δ7.92 (1H, d), 7.55 (1H, m), 7.45 (2H, m), 7.07 (1H, d)

### Step 9: 5-(5-chloro-6-isobutoxypyridin-3-yl)-3-fluoro-2-naphthonitrile

To a solution of 6-cyano-7-fluoronaphthalen-1-yl trifluoromethanesulfonate (step 8) (330 mg, 1 mmol) in dioxane (8 ml) was added 3-chloro-2-isobutoxy-5-(4,4,5,5-tetramethyl-1,3,2-dioxaborolan-2-yl)pyridine (intermediate U) (483 mg, 1.5 mmol), Pd(dppf)Cl₂ (151 mg, 0.2 mmol) and Cs₂CO₃ (505 mg, 1.5 mmol). The reaction mixture was stirred at 100° C. for 1.5 hrs under N₂. The mixture was cooled to room temperature and diluted with H₂O and extracted with DCM (×2). The combined organic layers were washed with brine (×2), and dried over anhydrous Na₂SO₄, filtered, and concentrated to afford the crude product, which was purified by flash column chromatography on silica gel using a gradient 0-10% ethyl acetate in hexanes to afford the title compound as white yellow.

LC-MS: Rt=1.15 mins; MS m/z [M+H]+ 355.0; Method 5-95AB 1.5minLC_v003

¹H NMR (400 MHz, CDCl₃) δ8.25 (2H, d), 8.02 (1H, d), 7.78 (2H, m), 7.46 (2H, m), 4.24 (2H, d), 2.21 (1H, m), 1.09 (6H, m).

### Step 10: 8-(5-chloro-6-isobutoxypyridin-3-yl)naphtho[2,3-d]isoxazol-3-amine

To a mixture of N-hydroxyacetamide (184 mg, 2.45 mmol) in dry DMF (10 ml) was added t-BuOK (2.45 ml, 2.45 mmol, 1M in THF) at room temperature. After addition, the mixture was stirred at room temperature for 5 min, then 5-(5-chloro-6-isobutoxy pyridin-3-yl)-3-fluoro-2-naphthonitrile (step 9) (290 mg, 0.82 mmol) in DMF (5 ml) was added. The reaction mixture was stirred at 40° C. for 16 hrs under N₂ protection. The mixture was poured into ice-water, extracted with EtOAc (×3). The combined organic layers were washed with brine (×3), dried over anhydrous Na₂SO₄, filtered, and concentrated to afford the crude product, which was purified by flash column chromatography on silica gel using a gradient 0-30% ethyl acetate in hexanes to afford the title compound as white yellow.

LC-MS: Rt=0.88 mins; MS m/z [M+H]+ 368.1; Method 5-95AB 1.5minLC_v003

¹H NMR (400 MHz, CDCl₃) δ8.15 (2H, d), 8.02 (1H, d), 7.78 (2H, m), 7.46 (2H, m), 4.58 (2H, br), 4.24 (2H, d), 2.21 (1H, m), 1.09 (6H, m).

### Step 11: N-(8-(5-chloro-6-isobutoxypyridin-3-yl)naphtho[2,3-d]isoxazol-3-yl)cyclopropanesulfonamide

To a solution of 8-(5-chloro-6-isobutoxypyridin-3-yl)naphtho[2,3-d]isoxazol-3-amine (step 10) (100 mg, 0.27 mmol) in pyridine (1 ml) was added cyclopropanesulfonyl chloride (1.15 g, 8.16 mmol) and DMAP (66 mg, 0.54 mmol). The mixture was stirred at 30° C. for 48 hrs. The mixture was concentrated to afford the crude product, which was purified by pre-HPLC to afford the title compound as white solid.

LC-MS: Rt=4.77 mins; MS m/z [M+H]+ 472.1; Method 10-80AB 7minLC_v001

¹H NMR (400 MHz, CDCl₃) δ8.61 (1H, s), 8.15 (1H, d), 8.09 (1H, d), 7.89 (1H, s), 7.78 (1H, d), 7.51 (2H, m), 7.21 (1H, m), 4.24 (2H, d), 2.83 (1H, m), 2.22 (1H, m), 1.31 (2H, m), 1.09 (8H, m).

### Example 59

### N-(8-(5-chloro-6-isobutoxypyridin-3-yl)isoxazolo[5,4-g]quinazolin-3-yl)cyclopropanesulfonamide

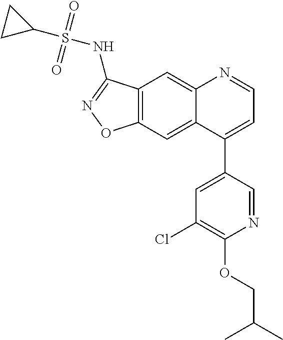 [SMILES: CC(C)COc1ncc(-c2ccnc3cc4c(NS(=O)(=O)C5CC5)noc4cc23)cc1Cl]

### Step 1: 4-bromo-5-fluoro-2-nitrobenzoic acid

To a mixture of 4-bromo-3-fluorobenzoic acid (Aldrich) (33 g, 150 mmol) in concentrated H₂SO₄ (250 ml) was added KNO₃ (16.6 g, 165 mmol) by portion. The resulting mixture was stirred at room temperature for 15 hrs. When TLC indicated the reaction was completed, the reaction mixture was pour to ice-water slowly. The solid was collected and washed with water and dried to afford the title compound as yellow solid.

¹H NMR (400 MHz, DMSO-d₆) δ 13.51 (1H, br s), 8.51 (1H, d), 7.90 (1H, d).

### Step 2: methyl 4-bromo-5-fluoro-2-nitrobenzoate

To a mixture of 4-bromo-5-fluoro-2-nitrobenzoic acid (step 1) (20 g, 76 mmol) in MeOH (200 ml) was added SOCl₂ (10 ml). The resulting mixture was stirred at 80° C. for 12 hrs. When TLC indicated the reaction was completed, the reaction mixture was evaporated under reduced pressure to give the residue, which was treated with NaHCO₃ aqueous solution and ethyl acetate. The separated organic phase was washed with brine (×2), dried over anhydrous magnesium sulfate and filtered. The filtration was concentrated under reduced pressure to give crude product, which was purified by column chromatography on silica gel eluting with 0-5% ethyl acetate in hexanes to afford the title compound as a yellow solid.

¹H NMR (400 MHz, CDCl₃) δ 8.20 (1H, d), 7.47 (1H, d), 3.94 (3H, s).

### Step 3: methyl 2-amino-4-bromo-5-fluorobenzoate

To a mixture of methyl 4-bromo-5-fluoro-2-nitrobenzoate (step 2) (9 g, 32 mmol) in MeOH (200 ml) was added SnCl₂ (31 g, 16 mmol). The reaction mixture was stirred at 70° C. for 5 hrs. When TLC indicated the reaction was completed, the reaction mixture was evaporated under reduced pressure to give the residue, which was treated with NaHCO₃ aqueous solution and ethyl acetate. The separated organic phase was washed with brine (×2), dried over anhydrous magnesium sulfate and filtered. The filtration was concentrated under reduced pressure to give crude product, which was purified by column chromatography on silica gel eluting with 0˜5% ethyl acetate in hexanes to afford the title compound as a yellow solid.

LC-MS: Rt=1.18 mins; MS m/z [M+H]+ 247.8; Method 5-95AB 1.5minLC_v003

¹H NMR (400 MHz, DMSO-d₆) δ 7.48 (1H, d), 7.09 (1H, d), 6.64 (2H, s), 3.76 (3H, s).

### Step 4: 7-bromo-6-fluoroquinazolin-4(3H)-one

A mixture of methyl 2-amino-4-bromo-5-fluorobenzoate (step 3) (4.6 g, 19 mmol) in formamide (20 ml) was stirred at 180° C. under microwave condition for 1 h. After cooling down, the reaction mixture was poured into water and stirred at room temperature overnight. Then the solid was collected, washed with water and dried to the title compound as white solid.

LC-MS: Rt=1.21 mins; MS m/z [M+H]+ 242.9; Method 5-95AB 1.5minLC_v003

¹H NMR (400 MHz, DMSO-d₆) δ12.66 (1H, br. s), 8.25 (1H, d), 8.17 (1H, s), 8.03 (1H, d).

### Step 5: 6-fluoro-4-oxo-3,4-dihydroquinazoline-7-carbonitrile

To a mixture of 7-bromo-6-fluoroquinazolin-4(3H)-one (step 4) (4.6 g, 19 mmol), DPPF (4.2 g, 7.6 mmol) and Zn(CN)₂ (4.4 g, 38 mmol) in DMF (20 ml) was added Pd₂(dba)₃ (3.5 g, 3.8 mmol) at room temperature. The resulting mixture was degassed and charged with nitrogen three times. After heating to 120° C., the reaction mixture was stirred at that temperature under nitrogen atmosphere for 4 hrs. When LC/MS indicated the starting material was consumed, the reaction mixture was cooled down and diluted with ethyl acetate. The mixture was filtered through a pad of celite and the filtration was then washed with brine (×3), dried over anhydrous magnesium sulfate and filtered. The filtration was evaporated under reduced pressure to give the crude product, which was purified by column chromatography on silica gel eluting with 0˜50% ethyl acetate in hexanes to afford the title compound as black solid

LC-MS: Rt=1.13 mins; MS m/z [M+H]+ 190.1; Method 5-95AB 1.5minLC_v003

¹H NMR (400 MHz, DMSO-d₆) δ12.66 (1H, br s), 8.35 (1H, d), 8.19 (1H, s), 8.03 (1H, d).

### Step 6: 4-chloro-6-fluoroquinazoline-7-carbonitrile

A mixture of 6-fluoro-4-oxo-3,4-dihydroquinazoline-7-carbonitrile (step 5) (700 mg, 3.7 mmol) in POCl₃ (20 g, 132 mmol) was stirred at 150° C. for 15 hrs. When TLC indicated the reaction was completed, the reaction mixture was poured into water and extrated with ethyl acetate (×3). The combined organic phases were washed with brine (×2), dried over anhydrous magnesium sulfate and filtered. The filtration was concentrated under reduced pressure to give a crude product, which was purified by column chromatography on silica gel eluting with 0˜50% ethyl acetate in hexanes to afford the title compound as black solid

LC-MS: Rt=1.36 mins; MS m/z [M+H]+ 208.1; Method 5-95AB 1.5minLC_v003

¹H NMR (400 MHz, DMSO-d₆) δ 8.33 (1H, d), 8.21 (1H, s), 8.03 (1H, d).

### Step 7: 4-(5-chloro-6-isobutoxypyridin-3-yl)-6-fluoroquinazoline-7-carbonitrile

To a solution 4-chloro-6-fluoroquinazoline-7-carbonitrile (step 6) (200 mg, 1.0 mmol) in dioxane (10 ml) was added 3-chloro-2-isobutoxy-5-(4,4,5,5-tetramethyl-1,3,2-dioxaborolan-2-yl)pyridine (Intermediate U) (778 mg, 2.5 mmol), Pd(dppf)Cl₂ (73.2 mg, 0.1 mmol) and Cs₂CO₃ (487 mg, 1.5 mmol). The resulting mixture was bubbled into N₂ for 10 min, then stirred under N₂ atmosphere at 100° C. for 16 hrs. When the reaction was completed as monitored by LC-MS, the reaction mixture was cooled to room temperature and filtered over celite. The filtration was concentrated under reduced pressure to the resulting mixture, which was treated with water and ethyl acetate. The separated aqueous phase was extracted with ethyl acetate (×3). The combined organic phases were washed with brine (×2), and dried over anhydrous magnesium sulfate and filtered. The filtration was concentrated under reduced pressure to give the crude product, which was purified by flash column chromatography on silica gel eluting a gradient from 0-20% ethyl acetate in hexanes to afford the title compound as white solid.

LC-MS: Rt=1.25 mins; MS m/z [M+H]+ 357.4; Method 5-95AB 1.5minLC_v003

¹H NMR (400 MHz, DMSO-d₆) δ 9.39 (1H, s), 8.84 (1H, s), 8.63 (1H, d), 8.53 (1H, d), 8.41 (1H, s), 4.28 (2H, d), 2.15 (1H, m), 1.05 (6H, d).

### Step 8: 8-(5-chloro-6-isobutoxypyridin-3-yl)isoxazolo[5,4-g]quinazolin-3-amine

To a mixture of 4-(5-chloro-6-isobutoxypyridin-3-yl)-6-fluoroquinazoline-7-carbonitrile (step 7) (200 mg, 0.56 mmol) in DMF (8 ml) was added t-BuOK (188 mg, 1.7 mmol) and N-hydroxyacetamide (126 mg, 1.7 mmol). The resulting mixture was stirred at 20° C. for 1 h. When LC/MS indicated the reaction was completed, the reaction mixture was diluted with ether, washed with water (×2), brine (×2), dried over anhydrous magnesium sulfate and filtered. The filtration was concentrated to give the crude product, which was purified by flash column chromatography on silica gel eluting a gradient from 0-20% ethyl acetate in hexanes to afford the title compound as white solid.

LC-MS: Rt=1.01 mins; MS m/z [M+H]+ 370.1; Method 5-95AB 1.5minLC_v003

¹H NMR (400 MHz, DMSO-d₆) δ 9.39 (1H, s), 8.84 (1H, s), 8.63 (1H, d), 8.53 (1H, d), 8.41 (1H, s), 5.76 (2H, s), 4.28 (2H, d), 2.15 (1H, m), 1.05 (6H, d).

### Step 9: N-(8-(5-chloro-6-isobutoxypyridin-3-yl)isoxazolo[5,4-q]quinazolin-3-yl)cyclopropanesulfonamide

To a mixture of 8-(5-chloro-6-isobutoxypyridin-3-yl)isoxazolo[5,4-g]quinazolin-3-amine (step 8) (170 mg, 0.4 mmol) in dry pyridine (6 ml) was added DMAP (195 mg, 1.6 mmol) and cyclopropanesulfonyl chloride (5.6 g, 40 mmol) at room temperature. The resulting mixture was stirred at room temperature for 2 hrs and quenched with saturated ammonium chloride aqueous solution. The reaction mixture was extrated with ethyl acetate (×3), washed with brine (×3), dried over anhydrous magnesium sulfate and filtered. The filtration was concentrated under reduced pressure to afford the crude product, which was dissovled in THF (10 ml). To the above mixture was added TBAF (477 mg, 1.8 mmol) and the resulting mixture was then stirred at room temperature for 2 hrs. After removal of the solvent, the residue was treated with ethyl acetate and water. The separated organic phase was washed with water (×2), dried over anhydrous magnesium sulfate and filtered. The filtration was concentrated under reduced pressure to give the crude product, which was purified by preparative HPLC to afford the title compound as a white solid.

LC-MS: Rt=4.56 mins; MS m/z 474.1 [M+H]+; Method 10-80AB 1.5minLC_v002

¹H NMR (400 MHz, DMSO-d₆) δ 11.99 (1H, br s), 9.39 (1H, s), 8.84 (1H, s), 8.63 (1H, d), 8.41 (2H, s), 4.28 (2H, d), 3.14 (1H, m), 2.15 (1H, m), 1.20 (2H, m), 1.14 (2H, m), 1.05 (6H, d).

### Example 60

### N-(8-(5-chloro-6-isobutoxypyridin-3-yl)-5-methylisoxazolo[4,5-g]isoquinolin-3-yl)cyclopropanesulfonamide

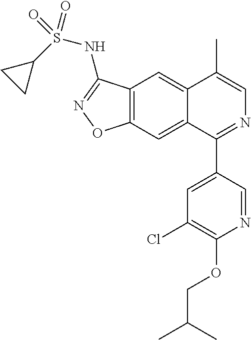 [SMILES: Cc1cnc(-c2cnc(OCC(C)C)c(Cl)c2)c2cc3onc(NS(=O)(=O)C4CC4)c3cc12]

### Step 1: 7-fluoro-1-hydroxy-4-iodoisoquinoline-6-carbonitrile

To a solution of 6-fluoro-4-hydroxyisoquinoline-7-carbonitrile (step 6 Example 56) (2 g, 11 mmol) in CH₂Cl₂ (70 ml) was added iodine monochloride (1.0 M in CH₂Cl₂, 2.24 g, 13.8 mmol) dropwise at 0° C. under nitrogen atmosphere. The resulting mixture was then allowed to warm to room temperature for 20 min and then heated to reflux overnight. The mixture was filtered and the cake was washed with DCM. The combined organic phases were concentrated to the title compound as a yellow solid.

LC-MS: Rt=1.25 mins; MS m/z [M+H]+ 314.9; Method 5-95AB 1.5minLC_v003

¹H NMR (400 MHz, DMSO-d₆) δ 11.92 (1H, s), 8.10 (2H, m), 7.70 (1H, d).

### Step 2: tert-butyl(6-cyano-7-fluoro-4-iodoisoquinolin-1-yl)carbonate

To a compound 6-fluoro-4-hydroxyisoquinoline-7-carbonitrile (step 1)(1.6 g, 5.1 mmol) in dry THF (200 ml) was added NaH (611 mg, 15.28 mmol) by portions at 0° C. under nitrogen atmosphere. The resulting mixture was stirred at 0° C. for 20 min, and then added a solution of Boc₂O (2.2 g, 10 mmol) in dry THF (2 ml). After stirring at room temperature for 1 h, the reaction mixture was quenched with 2 ml of NH₄Cl aqueous solution at 0° C. The mixture was extracted with ethyl acetate (×3). The combined organic phases were washed with water (×3), dried over anhydrous Na₂SO₄ and filtered. The filtration was concentrated to afford the title compound as a yellow solid.

LC-MS: Rt=1.38 mins; MS m/z [M+H]+ 414.8; Method 5-95AB 1.5minLC_v003

¹H NMR (400 MHz, CDCl₃) δ 8.22 (1H, d), 8.04 (1H, s), 8.01 (1H, d), 1.66 (9H, s).

### Step 3: 7-fluoro-1-hydroxy-4-methylisoquinoline-6-carbonitrile

To a mixture of tert-butyl(6-cyano-7-fluoro-4-iodoisoquinolin-1-yl) carbonate (step 2) (900 mg, 2.2 mmol) and Pd(dppf)Cl₂ (318 mg, 0.043 mmoL) in dioxane (30 ml) was added Zn(Me)₂ (4.3 ml, 1 M in toluene) dropwise at 0° C. under nitrogen atmosphere. The resulting mixture was stirred at 100° C. for 4 hrs before diluted with DCM. The reaction mixture was filtered over celite. The filtration was washed with water (×2), dried over anhydrous Na₂SO₄ and filtered. The filtration was concentrated under reduced pressure to afford the title compound as yellow solid.

LC-MS: Rt=1.29 mins; MS m/z [M+H]+ 203.1; Method 5-95AB 1.5minLC_v003

¹H NMR (400 MHz, DMSO-d₆) δ 11.10 (1H, s), 7.77 (1H, d), 7.58 (1H, d), 6.94 (1H, s), 2.38 (3H, s).

### Step 4: 1-chloro-7-fluoro-4-methylisoquinoline-6-carbonitrile

A mixture of 7-fluoro-1-hydroxy-4-methylisoquinoline-6-carbonitrile (step 3) (400 mg, 1.98 mmol) in POCl₃ (20 ml) was stirred at 100° C. for 4 hrs. The reaction mixture was poured into water (200 ml). The mixture was extracted with DCM (×3), dried over Na₂SO₄ and filtered. The filtration was concentrated under reduced pressure to give the crude product, which was by column chromatography on silica gel eluting with 0˜10% ethyl acetate in hexanes to afford the title compound as white solid.

LC-MS: Rt=1.45 mins; MS m/z [M+H]+ 221.0; Method 5-95AB 1.5minLC_v003

¹H NMR (400 MHz, CDCl₃) δ 8.36 (1H, d), 8.24 (1H, s), 8.13 (1H, d), 2.64 (3H, s).

### Step 5: 1-(5-chloro-6-isobutoxypyridin-3-yl)-7-fluoro-4-methylisoquinoline-6-carbonitrile

To a solution of 1-chloro-7-fluoro-4-methylisoquinoline-6-carbonitrile (150 mg, 0.68 mmol) in dioxane (12 ml) was added 3-chloro-2-isobutoxy-5-(4,4,5,5-tetramethyl-1,3,2-dioxaborolan-2-yl)pyridine (intermediate U) (318 mg, 1.0 mmol), Pd(dppf)Cl₂ (101 mg, 0.136 mmol) and Cs₂CO₃ (332 mg, 1.0 mmol). The resulting mixture was degassed and charged with N₂ three times and then stirred at 100° C. for 4 hrs. After cooling to room temperature, the reaction mixture was diluted with DCM, washed with brine (×2), dried over Na₂SO₄ and filtered. The filtration was concentrated under reduced pressure to give the crude product, which was purified by column chromatography on silica gel eluting with 0˜10% ethyl acetate in hexanes to afford the title compound as a white solid.

LC-MS: Rt=1.31 mins; MS m/z [M+H]+ 370.1; Method 5-95AB 1.5minLC_v003

¹H NMR (400 MHz, CDCl₃) δ 8.56 (1H, s), 8.43 (1H, d), 8.28 (1H, d), 7.98 (1H, d), 7.87 (1H, d), 4.24 (2H, d), 2.71 (3H, s), 2.20 (1H, m), 1.07 (6H, d).

### Step 6: 8-(5-chloro-6-isobutoxypyridin-3-yl)-5-methylisoxazolo[4,5-g]isoquinolin-3-amine

To a mixture of N-hydroxyacetamide (73 mg, 0.973 mmol) in dry DMF (2 ml) was added t-BuOK (109 mg, 0.97 mmoL) at room temperature. The resulting mixture was stirred at that temperature for 30 min and then added 1-(5-chloro-6-isobutoxypyridin-3-yl)-7-fluoro-4-methylisoquinoline-6-carbonitrile (step 5) (120 mg, 0.32 mmol) in DMF (2 ml). After stirring at room temperature for 16 hrs, the reaction mixture was poured into ice-water. The mixture was extracted with ethyl acetate, washed with water (×2), dried over anhydrous Na₂SO₄ and filtered. The filtration was concentrated under reduced pressure to give the crude product, which was purified by column chromatography on silica gel eluting with 0˜10% ethyl acetate in hexanes to afford the title compound as a white solid.

LC-MS: Rt=1.18 mins; MS m/z [M+H]+ 383.0; Method 5-95AB 1.5minLC_v003

¹H NMR (400 MHz, CDCl₃) δ 8.35 (1H, s), 8.26 (1H, s), 8.31 (1H, d), 8.03 (1H, s), 7.98 (1H, d), 4.85 (2H, br s), 4.25 (2H, d), 2.65 (3H, s), 2.21 (1H, m), 1.07 (6H, m).

### Step 7: N-(8-(5-chloro-6-isobutoxypyridin-3-yl)-5-methylisoxazolo[4,5-g]isoquinolin-3-yl)cyclopropanesulfonamide

To a solution of 8-(5-chloro-6-isobutoxypyridin-3-yl)-5-methylisoxazolo[4,5-g]iso quinolin-3-amine (step 6)(70 mg, 0.18 mmol) in pyridine (1 ml) was added cyclopropanesulfonyl chloride (1.83 ml, 1.82 mmol) and DMAP (45 mg, 0.36 mmol). The resulting mixture was stirred at room temperature for 16 hrs and then treated with water and ethyl acetate. The separated organic phase was washed with brine and concentrated under reduced pressure to the crude product, which was dissolve in THF (20 ml). To the mixture was added TBAF (0.2 mmol) at room temperature. The resulting mixture was stirred at room temperature for additional 1.5 hrs. After removal of the solvent, the residue was treated with water and ethyl acetate. The separated organic phase was concentrated under reduced pressure to the crude product, which was purified by preparative HPLC to afford the title compound as a white solid.

LC-MS: Rt=3.88 mins; MS m/z 487.0 [M+H]+; Method 10-80AB 7minLC_v002

¹H NMR (400 MHz, CDCl₃) δ 8.64 (1H, s), 8.38 (1H, s), 8.31 (1H, d), 8.05 (1H, s), 7.98 (1H, d), 7.28 (1H, s), 4.24 (2H, d), 3.17 (1H, m), 2.69 (3H, s), 2.20 (1H, m), 1.07 (8H, m), 0.89 (2H, m).

### Example 61

### N-(8-(5-chloro-6-isobutoxypyridin-3-yl)-6-methylisoxazolo[4,5-g]isoquinolin-3-yl)cyclopropanesulfonamide

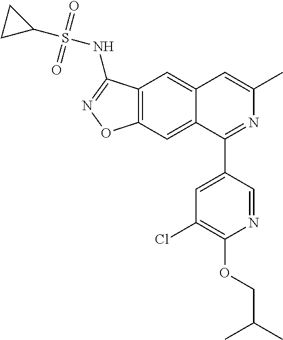 [SMILES: Cc1cc2cc3c(NS(=O)(=O)C4CC4)noc3cc2c(-c2cnc(OCC(C)C)c(Cl)c2)n1]

### Step 1: methyl 3-(4-fluoro-3-methoxyphenyl)-2-methylacrylate

To a solution of 4-bromo-1-fluoro-2-methoxybenzene (Aldrich) (20 g, 97.5 mmol), methyl methacrylate (Aldrich) (24 g, 244 mmol) in DMF (150 ml) was added Pd(OAc)₂ (2.2 g, 9.7 mmol), Bu₃N (45 g, 244 mmol), TOP (8.9 g, 29 mmol). The resulting mixture was degassed and charged with N₂ three times, and then stirred at 100° C. for 5 hrs. After cooling down, the mixture was filtered over celite. The filtration was evaporated under reduced pressure to the residue, which was treated with water and ethyl acetate. The separated organic layer was washed with brine (×3), dried over anhydrous Na₂SO₄ and filtered. The filtration was concentrated under reduced pressure to give the crude product, which was purified by column chromatography on silica gel eluting with 0˜10% ethyl acetate in hexanes to afford the title compound as a yellow solid.

LC-MS: Rt=1.48 mins; MS m/z [M+H]+ 225.0; Method 5-95AB 1.5minLC_v003

### Step 2: 3-(4-fluoro-3-methoxyphenyl)-2-methylacrylic acid

To a mixture of methyl 3-(4-fluoro-3-methoxyphenyl)-2-methylacrylate (step 1)(12 g, 53 mmol) in MeOH (50 ml) was added NaOH aqueous solution (50 ml, 2.0 N). The resulting mixture was stirred at 20° C. for 16 hrs. When TLC indicated the reaction was completed, the mixture was poured into HCl aqueous solution (2N) and extracted with ethyl acetate (×3). The combined organic phases were washed with brine, dried over anhydrous Na₂SO₄ and filtered. The filtration was concentrated under reduced pressure to afford the title compound as a white solid.

LC-MS: Rt=1.02 mins; MS m/z [M+H]+ 211.0; Method 5-95AB 1.5minLC_v003

### Step 3: methyl 3-(4-fluoro-3-methoxyphenyl)-2-methylacryloyl azide

To a solution of 3-(4-fluoro-3-methoxyphenyl)-2-methylacrylic acid (step 2) (10 g, 47 mmol) and Et₃N (14 g, 143 mmol) in toluene (450 ml) was added DPPA (13 g, 47 mmol). The mixture was stirred at 20° C. for 16 hrs and then concentrated to the residue, which was purified by column chromatography on silica gel eluting with 0˜10% ethyl acetate in hexanes to afford the title compound as a yellow solid.

LC-MS: Rt=1.12 mins; MS m/z [M−28]+235.9; Method 5-95AB 1.5minLC_v003

### Step 4: 7-fluoro-6-methoxy-3-methylisoquinolin-1(2H)-one

A mixture of methyl 3-(4-fluoro-3-methoxyphenyl)-2-methylacryloyl azide (step 3) (8 g, 34 mmol) in 1,2-dichlorobenzene (100 ml) was stirred 140° C. for 1 h and then added I₂ (100 mg, 0.4 mmol). After stirring at 200° C. for additional 2 hrs, the reaction mixture was cooled down. The solid was collected and washed with ethyl acetate to the title compound as a yellow solid.

LC-MS: Rt=0.86 mins; MS m/z [M+H]+ 208.0; Method 5-95AB 1.5minLC_v003

¹H NMR (400 MHz, DMSO-d₆) δ 8.73 (1H, s), 7.73 (1H, d), 7.21 (1H, d), 6.28 (1H, s), 3.93 (3H, s), 2.19 (3H, s).

### Step 5: 7-fluoro-6-hydroxy-3-methylisoquinolin-1(2H)-one

To a mixture of 7-fluoro-6-methoxy-3-methylisoquinolin-1(2H)-one (step 4) (4 g, 19 mmol) in DCM (40 ml) was added BBr₃ (24 g, 96 mmol) at 0° C. The resulting mixture was stirred at 20° C. for 15 hrs. Then the reaction mixture was poured into water and extracted with DCM (×3). The combined organic phases were washed with brine (×2), dried over Na₂SO₄ and filtered. The filtration was concentrated under reduced pressure to give the crude product, which was purified by column chromatography on silica gel eluting with 0˜10% ethyl acetate in hexanes to afford the title compound as a yellow solid.

LC-MS: Rt=0.42 mins; MS m/z [M+H]+ 194.0; Method 5-95AB 1.5minLC_v003

¹H NMR (400 MHz, CD₃OD-d₄) δ 7.78 (1H, d), 6.94 (1H, d), 6.28 (1H, s), 2.25 (3H, s).

### Step 6: 7-fluoro-3-methyl-1-oxo-1,2-dihydroisoquinolin-6-yl trifluoromethanesulfonate

To a solution of 7-fluoro-6-hydroxy-3-methylisoquinolin-1(2H)-one (step 5) (3 g, 15 mmol) in pyridine (100 ml) was added 1,1,1-trifluoro-N-phenyl-N-((trifluoromethyl) sulfonyl)methanesulfonamide (8.3 g, 23 mmol) and DMAP (1.9 g, 15 mmol). The resulting mixture was stirred at 25° C. for 2 hrs. After cooling to room temperature, the reaction mixture was diluted with water and extracted with DCM (×3). The combined organic extracts were washed with brine (×3), dried over Na₂SO₄ and filtered. The filtration was concentrated under reduced pressure to give the crude product, which was purified by column chromatography on silica gel eluting with 0˜10% ethyl acetate in hexanes to afford the title compound as a white solid.

LC-MS: Rt=1.12 mins; MS m/z [M+H]+ 326.0; Method 5-95AB 1.5minLC_v003

¹HNMR (400 MHz, CDCl₃) δ11.01 (1H, s), 8.19 (1H, d), 7.42 (1H, d), 6.30 (1H, s), 2.39 (3H, s).

### Step 7: 7-fluoro-3-methyl-1-oxo-1,2-dihydroisoquinoline-6-carbonitrile

To a suspension of 7-fluoro-3-methyl-1-oxo-1,2-dihydroisoquinolin-6-yltrifluoro methane sulfonate (step 6)(2.5 g, 7.7 mmol), DPPF (1.6 g, 3 mol) and Pd₂(dba)₃ (1.3 g, 1.5 mmol) in DMF (50 ml) was added Zn(CN)₂ (2.6 g, 23 mmol). The resulting mixture was degassed and charged with N₂ three times and then stirred at 120° C. for 4 hrs. After cooling down, the reaction mixture was diluted with ethyl acetate and filtered over celite. The filtration was washed with brine (×3), dried over anhydrous Na₂SO₄ and filtered. The filtration was evaporated under reduced pressure to give the crude product, which was purified by column chromatography on silica gel eluting with 0˜10% ethyl acetate in hexanes to afford the title compound as a yellow solid.

LC-MS: Rt=0.87 mins; MS m/z [M+H]+ 203.0; Method 5-95AB 1.5minLC_v003

¹H NMR (400 MHz, DMSO-d₆) δ 11.70 (1H, s), 8.24 (1H, d), 7.96 (1H, d), 6.39 (1H, s), 2.20 (3H, s).

### Step 8: 1-chloro-7-fluoro-3-methylisoquinoline-6-carbonitrile

A mixture of 7-fluoro-3-methyl-1-oxo-1,2-dihydroisoquinoline-6-carbonitrile (step 7) (0.7 g, 2.5 mmol) in POCl₃ (70 g) was stirred at 110° C. for 5 hrs. After cooling to room temperature, the reaction mixture was poured into ice-water and extracted with DCM (×3). The combined organic layers were washed with brine (×3), dried over Na₂SO₄ and filtered. The filtration was concentrated under reduced pressure to the crude product, which was purified by column chromatography on silica gel eluting with 0˜10% ethyl acetate in hexanes to afford the title compound as a white solid.

LC-MS: Rt=1.37 mins; MS m/z [M+H]+ 221.0; Method 5-95AB 1.5minLC_v003

¹HNMR (400 MHz, CDCl₃) δ 8.16 (1H, d), 8.06 (1H, d), 7.48 (1H, s), 2.69 (3H, s).

### Step 9: 1-(5-chloro-6-isobutoxypyridin-3-yl)-7-fluoro-3-methylisoquinoline-6-carbonitrile

To a solution of 1-chloro-7-fluoro-3-methylisoquinoline-6-carbonitrile (500 mg, 2.2 mmol) in dioxane (step 8) (25 ml) was added 3-chloro-2-isobutoxy-5-(4,4,5,5-tetramethyl-1,3,2-dioxaborolan-2-yl)pyridine (intermediate U)(1060 mg, 3.3 mmol), Pd(dppf)Cl₂ (140 mg, 0.2 mmol) and Cs₂CO₃ (1100 mg, 3.3 mmol). The resulting mixture was degassed and charged with N2 three times, and then stirred at 100° C. for 4 hrs. After cooling to room temperature, the reaction mixture was diluted with DCM. The mixture was filtered over celite and then washed with brine (×3), dried over Na₂SO₄ and filtered. The filtration was concentrated under reduced pressure to the crude product, which was purified by column chromatography on silica gel eluting with 0˜10% ethyl acetate in hexanes to afford the title compound as a white solid.

LC-MS: Rt=1.44 mins; MS m/z [M+H]+ 370.0; Method 5-95AB 1.5minLC_v003

¹HNMR (400 MHz, CDCl₃) δ 8.30 (1H, d), 8.20 (1H, d), 8.00 (1H, d), 7.82 (1H, d), 7.53 (1H, s), 4.25 (2H, d), 2.76 (3H, s), 2.20 (1H, m), 1.06 (6H, d).

### Step 10: 8-(5-chloro-6-isobutoxypyridin-3-yl)-6-methylisoxazolo[4,5-g]isoquinolin-3-amine

A mixture of 1-(5-chloro-6-isobutoxypyridin-3-yl)-7-fluoro-3-methylisoquinoline-6-carbonitrile (Step 9, 200 mg, 0.54 mmol), N-hydroxyacetamide (120 mg, 1.62 mmol) and t-BuOK in THF (1.6 ml, 1.6 mmol) in 3 ml of DMF was stirred at 25° C. under N₂ for 16 hrs. The reaction mixture was quenched with water, extracted with ethyl acetate (×3). The combined organic layers were washed with brine (×3), dried over anhydrous Na₂SO₄ and filtered. The filtration was evaporated under reduced pressure to afford the crude product, which was purified by column chromatography on silica gel eluting with 0˜10% ethyl acetate in hexanes to afford the title compound as a yellow solid.

LC-MS: Rt=1.04 mins; MS m/z [M+H]+ 383.0; Method 5-95AB 1.5minLC_v003

¹H NMR (400 MHz, CD₃OD-d₄) δ 8.30 (1H, d), 8.21 (1H, s), 7.96 (1H, d), 7.84 (1H, s), 7.64 (1H, s), 4.21 (2H, d), 2.69 (3H, s), 2.19 (1H, m), 1.05 (6H, m).

### Step 11: N-(8-(5-chloro-6-isobutoxypyridin-3-yl)-6-methylisoxazolo[4,5-g]isoquinolin-3-yl)cyclopropanesulfonamide

To a solution of 8-(5-chloro-6-isobutoxypyridin-3-yl)-6-methylisoxazolo[4,5-g]iso quinolin-3-amine (step 10) (0.1 g, 11 mmol) and DMAP (64 mg, 0.52 mmol) in pyridine (1 ml) was added cyclopropanesulfonyl chloride (2 ml). The mixture was stirred at 60° C. for 20 hrs, and then diluted with ethyl acetate, washed with brine (×3), dried over anhydrous magnesium sulfate and filtered. The filtration was concentrated under reduced pressure to give the residue, which was treated with TBAF in THF (0.6 ml, 0.6 mmol). After stirring at 25° C. for 2 hrs, the reaction mixture was diluted with ethyl acetate, washed with brine (×3), dried over anhydrous magnesium sulfate and filtered. The filtration was concentrated under reduced pressure to give the residue, which was purified by preparative HPLC to give the title compound as a yellow solid.

LC-MS: Rt=3.81 mins; MS m/z 487.0 [M+H]+; Method 10-80AB 1.5minLC_v002

¹H NMR (400 MHz, CD₃OD-d₄) δ 8.52 (1H, s), 8.40 (1H, d), 8.14 (1H, d), 8.05 (1H, s), 7.86 (1H, s), 4.32 (2H, d), 2.75 (3H, s), 2.22 (1H, m), 1.7 (1H, m), 1.28 (2H, m), 1.12 (8H, m).

### Example 62

### N-(8′-(5-chloro-6-isobutoxypyridin-3-yl)-7′-oxo-7′,8′-dihydrospiro[cyclopropane-1,6′-isoxazolo[5′,4′:4,5]benzo[1,2-b][1,4]oxazin]-3′-yl)cyclopropanesulfonamide

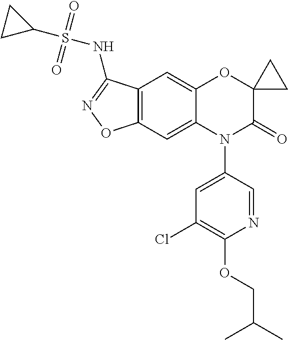 [SMILES: CC(C)COc1ncc(N2C(=O)C3(CC3)Oc3cc4c(NS(=O)(=O)C5CC5)noc4cc32)cc1Cl]

### Step 1: methyl 1-(5-bromo-4-fluoro-2-nitrophenoxy)cyclopropanecarboxylate

To a mixture of methyl 1-hydroxycyclopropanecarboxylate (Aldrich) (1.0 g, 8.8 mmol) and 15-crown-5 (cat. amount) in THF (30 ml) was added NaH (400 mg, 10 mmol) at room temperature. The resulting mixture was stirred at room temperature for 30 min and then added 1-bromo-2,5-difluoro-4-nitrobenzene (Aldrich) (2 g, 8.4 mmol). The mixture was stirred at room temperature for additional 16 hrs. When TLC indicated the reaction was completed, the reaction mixture was quenched with saturated NH₄Cl aqueous solution. After removal of the organic solvent, the mixture was extracted with ethyl acetate (×3). The combined organic phases were washed with brine (×3), dried over anhydrous Magnesium sulfate and filtered. The filtration was concentrated under reduced pressure to give the crude product, which was purified by flash column chromatography on silica gel using a gradient 0-10% ethyl acetate in hexanes to afford the title compound as yellow solid.

LC-MS: Rt=1.43 mins; MS m/z [M+H]+ 333.9; Method 5-95AB 1.5minLC_v003

¹H NMR (400 MHz, CDCl₃) δ 8.15 (1H, d), 6.98 (1H, d), 3.57 (3H, s), 1.38 (2H, m), 1.18 (2H, m).

### Step 2: 7-bromo-6-fluorospiro[benzo[b][1,4]oxazine-2,1′-cyclopropan]-3(4H)-one

To a solution of methyl 1-(5-bromo-4-fluoro-2-nitrophenoxy)cyclopropanecarboxylate (step 1) (1.5 g, 4.6 mmol) in HOAc (40 ml) was added Fe (2.5 g, 45.5 mmol). The resulting mixture was stirred at 60° C. for 3 hrs. When TLC indicated the reaction was completed, the reaction mixture was cooled down and filtered. The filtration was evaporated in vacuo to the residue, which was treated with ethyl acetate and water. The separated organic phase was washed with NaHCO₃ aqueous solution (×2), dried over anhydrous magnesium sulfate and filtered. The filtration was concentrated under reduced pressure to afford the title compound as pale white solid.

LC-MS: Rt=1.32 mins; MS m/z [M+H]+ 271.9; Method 5-95AB 1.5minLC_v003

¹H NMR (400 MHz, CDCl₃) δ 8.70 (1H, br. s.), 6.98 (1H, d), 6.57 (1H, d), 1.38 (2H, m), 1.18 (2H, m).

### Step 3: 6-fluoro-3-oxo-3,4-dihydrospiro[benzo[b][1,4]oxazine-2,1′-cyclopropane]-7-carbonitrile

A mixture of 7-bromo-6-fluorospiro[benzo[b][1,4]oxazine-2,1′-cyclopropan]-3(4H)-one (step 2) (500 mg, 1.8 mmol), Zn(CN)₂ (215 mg, 1.8 mmol) and Pd(PPh₃)₄ (65 mg, 0.055 mmol) in DMF (5 ml) was degassed and charged with N₂ three times. And then the resulting mixture was stirred at 120° C. under the irradiation of microwave for 0.5 h. The reaction mixture was diluted with ethyl acetate (50 ml) and filtered over celite. The filtration was washed with water (×2) and brine (×2), dried over magnesium sulfate and filtered. The filtration was concentrated under reduced pressure to give the crude product, which was purified by flash column chromatography on silica gel using a gradient 0-25% ethyl acetate in hexanes to afford the title compound as pale white solid.

LC-MS: Rt=1.29 mins; MS m/z [M+H]+ 219.2; Method 5-95AB 1.5minLC_v003

¹H NMR (400 MHz, CDCl₃) δ 8.70 (1H, br. s.), 7.06 (1H, d), 6.66 (1H, d), 1.48 (2H, m), 1.31 (2H, m).

### Step 4: 4-(5-chloro-6-isobutoxypyridin-3-yl)-6-fluoro-3-oxo-3,4-dihydrospiro[benzo[b][1,4]oxazine-2,1′-cyclopropane]-7-carbonitrile

A mixture of 6-fluoro-3-oxo-3,4-dihydrospiro[benzo[b][1,4]oxazine-2,1′-cyclopropane]-7-carbonitrile (step 3) (200 mg, 0.92 mmol), 5-bromo-3-chloro-2-isobutoxypyridine (intermediate A) (727 mg, 2.8 mmol), CuI (174 mg, 0.92 mmol), DMEDA (162 mg, 1.8 mmol) and K₂CO₃ (253 mg, 1.8 mmol) in CH₃CN (10 ml) was degassed and charged with N₂ three times. And then the resulting mixture was stirred at 120° C. under the irradiation of microwave for 1 h. The reaction mixture was diluted with ethyl acetate (50 ml) and filtered over celite. The filtration was washed with water (×2) and brine (×2), dried over Magnesium sulfate and filtered. The filtration was concentrated under reduced pressure to give the crude product, which was purified by flash column chromatography on silica gel using a gradient 0-25% ethyl acetate in hexanes to afford the title compound as yellow solid.

LC-MS: Rt=1.35 mins; MS m/z [M+H]+ 402.0; Method 5-95AB 1.5minLC_v003

¹H NMR (400 MHz, CDCl₃) δ 8.70 (1H, d), 8.01 (1H, d), 7.62 (1H, d), 7.06 (1H, d), 4.21 (2H, d), 2.68 (1H, m), 1.48 (2H, m), 1.31 (2H, m), 1.07 (6H, d).

### Step 5: 3′-amino-8′-(5-chloro-6-isobutoxypyridin-3-yl)spiro[cyclopropane-1,6′-isoxazolo[5′,4′:4,5]benzo[1,2-b][1,4]oxazin]-7′(8′H)-one

To a mixture of 4-(5-chloro-6-isobutoxypyridin-3-yl)-6-fluoro-3-oxo-3,4-dihydrospiro-[benzo[b][1,4]oxazine-2,1′-cyclopropane]-7-carbonitrile (step 4) (150 mg, 0.23 mmol) and N-hydroxyacetamide (51 mg, 0.67 mmol) in DMF (5 ml) was added t-BuOK (0.67 ml, 1 M in THF, 0.67 mmol). The mixture was stirred at 25° C. for 3 hrs. When LC/MS indicated the reaction was completed, the reaction mixture was evaporated under reduced pressure to the residue, which was treated with water and ethyl acetate. The separated organic phase was washed with water (×2), dried over anhydrous Magnesium sulfate and filtered. The filtration was concentrated to give the crude product, which was purified by flash column chromatography on silica gel using a gradient 0-50% ethyl acetate in hexanes to afford the title compound as white solid.

LC-MS: Rt=0.99 mins; MS m/z [M+H]+ 415.1; Method 5-95AB 1.5minLC_v003

¹H NMR (400 MHz, CDCl₃) δ 8.40 (1H, d), 8.11 (1H, d), 7.62 (1H, d), 7.06 (1H, d), 4.61 (2H, br s), 4.21 (2H, d), 2.68 (1H, m), 1.48 (2H, m), 1.31 (2H, m), 1.07 (6H, d).

### Step 6: N-(8′-(5-chloro-6-isobutoxypyridin-3-yl)-7-oxo-7′,8′-dihydrospiro[cyclopropane-1,6′-isoxazolo[5′,4′:4,5]benzo[1,2-b][1,4]oxazin]-3′-yl)cyclopropanesulfonamide

To a solution of 3′-amino-8′-(5-chloro-6-isobutoxypyridin-3-yl)spiro[cyclopropane-1,6′-isoxazolo[5′,4′:4,5]benzo[1,2-b][1,4]oxazin]-7′(8′H)-one (step 5) (80 mg, 0.19 mmol) and DMAP (48 mg, 0.38 mmol) in pyridine (1.0 ml) was added cyclopropanesulfonyl chloride (266 mg, 1.9 mmol). The resulting mixture was stirred at room temperature for 16 hrs. When LC/MS indicated the reaction was completed, the reaction mixture was diluted with ethyl acetate (10 ml), washed with brine (×3), dried over anhydrous Magnesium sulfate and filtered. The filtration was concentrated under reduced pressure to the crude product, which was purified by preparative HPLC to afford the title compound as white solid.

LC-MS: Rt=5.84 mins; MS m/z [M+H]+ 519.1; Method 0-60AB 7minLC_v002

¹H NMR (400 MHz, CDCl₃) δ 8.01 (1H, d), 7.62 (1H, d), 7.50 (1H, s), 7.20 (1H, s), 6.57 (1H, s), 4.21 (2H, d), 2.68 (1H, m), 2.19 (1H, m), 1.51 (2H, m), 1.39 (2H, m), 1.25 (2H, m), 1.09 (2H, m), 1.07 (6H, d).

### Example 63

### N-(8-(5-chloro-6-isobutoxypyridin-3-yl)-7,8-dihydro-6H-isoxazolo[5′,4′:4,5]benzo[1,2-b][1,4]oxazin-3-yl)methanesulfonamide

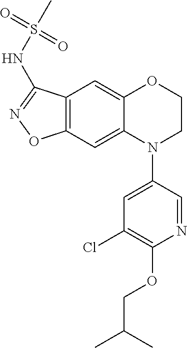 [SMILES: CC(C)COc1ncc(N2CCOc3cc4c(NS(C)(=O)=O)noc4cc32)cc1Cl]

### Step 1: 2-chloro-N-(5-fluoro-2-hydroxyphenyl)acetamide

To a stirred solution of 2-amino-4-fluorophenol (12.7 g, 100 mmol) in the mixture of 100 ml NaHCO₃ aqueous solution and chloroform (100 ml) was added 2-chloroacetyl chloride (10.0 ml, 120 mmol) at 0° C. The resulting mixture was stirred at 70° C. for 2 hrs. When the reaction was completed, the mixture was extracted with DCM (×2). The organic layer was washed with water and brine, dried over Na₂SO₄ and filtered. The filtrate was concentrated to afford the title compound as brown solid.

LC-MS: Rt=0.67 mins; MS m/z [M+H]+ 204.1; Method 5-95AB 1.5minLC_v003

¹H NMR (400 MHz, CDCl₃) δ 8.44 (1H, s), 7.30 (1H, d), 6.92 (2H, m), 6.82 (1H, m), 4.24 (2H, s).

### Step 2: 6-fluoro-2H-benzo[b][1,4]oxazin-3(4H)-one

To a mixture of 2-chloro-N-(5-fluoro-2-hydroxyphenyl)acetamide (step 1) (17 g, 86 mmol) in DMF (200 ml) was added K₂CO₃ (35 g, 258 mmol). The resulting mixture was heated to 70° C. and stirred at that temperature under N₂ atmosphere for 2 hrs. When LC/MS indicated the starting material was consumed, the reaction mixture was filtered and the filtrate was diluted with DCM (500 ml). The organic layer was washed with water (×2) and brine (×2), dried over anhydrous Na₂SO₄ and filtered. The filtration was evaporated to give a residue, which was purified by flash column chromatography on silica gel using a gradient 0-20% ethyl acetate in hexanes to afford the title compound as orange solid.

LC-MS: Rt=0.84 mins; MS m/z [M+H]+ 168.1; Method 5-95AB 1.5minLC_v003

¹H NMR (400 MHz, CDCl₃) δ 8.89 (1H, s), 6.92 (1H, m), 6.68 (1H, m), 6.59 (1H, m), 4.59 (2H, s).

### Step 3: 7-bromo-6-fluoro-2H-benzo[b][1,4]oxazin-3(4H)-one

To a mixture of 6-fluoro-2H-benzo[b][1,4]oxazin-3(4H)-one (step 2) (2.0 g, 12.0 mmol) in DMF (50 ml) was added NBS (3.2 g, 18 mmol) by portions at 0° C. Then the mixture was heated to 40° C. and stirred at that temperature under N₂ atmosphere for 16 hrs. When LC/MS indicated the starting material was consumed, the reaction mixture was diluted with EtOAc (200 ml). The organic layer was washed with water (×3) and brine (×2), dried over anhydrous Na₂SO₄ and filtered. The filtration was evaporated to give a residue, which was purified by flash column chromatography on silica gel using a gradient 0-20% ethyl acetate in hexanes to afford the title compound as orange solid.

LC-MS: Rt=0.85 mins; MS m/z [M+H]+ 245.9; Method 5-95AB 1.5minLC_v003

¹H NMR (400 MHz, CDCl₃) δ 8.89 (1H, s), 7.16 (1H, d), 6.62 (1H, d), 4.58 (2H, s).

### Step 4: 7-bromo-6-fluoro-3,4-dihydro-2H-benzo[b][1,4]oxazine

To a mixture of 7-bromo-6-fluoro-2H-benzo[b][1,4]oxazin-3(4H)-one (step 3) (880 mg, 3.58 mmol) in THF (20 ml) was added BH₃-Me₂S (1.7 ml, 18 mmol) at 0° C. The mixture was stirred at room temperature for 2 hrs. When LC/MS indicated the starting material was consumed, the reaction mixture was quenched with 1 N HCl aqueous solution and diluted with EtOAc (50 ml). The organic layer was washed with brine (×3), dried over anhydrous Na₂SO₄ and filtered. The filtration was evaporated to afford a residue, which was purified by flash column chromatography on silica gel using a gradient 0-20% ethyl acetate in hexanes to afford the title compound as pale yellow solid.

LC-MS: Rt=0.95 mins; MS m/z [M+H]+ 231.9; Method 5-95AB 1.5minLC_v003

¹H NMR (400 MHz, CDCl₃) δ 6.90 (1H, d), 6.37 (1H, d), 4.19 (2H, d), 3.87 (1H, s), 3.40 (2H, d).

### Step 5: 6-fluoro-3,4-dihydro-2H-benzo[b][1,4]oxazine-7-carbonitrile

To a mixture of 7-bromo-6-fluoro-3,4-dihydro-2H-benzo[b][1,4]oxazine (step 4) (400 mg, 1.7 mmol) in DMF (10 ml) was added Zn(CN)₂ (640 mg, 5.4 mmol), Pd₂(dba)₃ (600 mg, 0.65 mmol) and Pd(dppf)Cl₂ (600 mg, 0.82 mmol), Zn powder (100 mg, 1.52 mmol). After addition, the mixture was degassed and charged with N₂ three times. Then the mixture was heated to 120° C. and stirred at that temperature under N₂ atmosphere for 3 hrs. When LC/MS indicated the starting material was consumed, the reaction mixture was diluted with EtOAc. The mixture was filtered over celite. The filtration was washed with brine (×3), dried over anhydrous Na₂SO₄ and filtered. The filtration was evaporated to afford a residue, which was purified by flash column chromatography on silica gel using a gradient 0-50% ethyl acetate in hexanes to afford the title compound as pale yellow solid.

LC-MS: Rt=0.87 mins; MS m/z [M+H]+ 179.1; Method 5-95AB 1.5minLC_v003

¹H NMR (400 MHz, CDCl₃) δ 6.90 (1H, d), 6.28 (1H, d), 4.44 (1H, s), 4.18 (1H, m), 3.87 (1H, s), 3.48 (2H, m).

### Step 6: 4-(5-chloro-6-isobutoxypyridin-3-yl)-6-fluoro-3,4-dihydro-2H-benzo[b][1,4]oxazine-7-carbonitrile

To a mixture of 6-fluoro-3,4-dihydro-2H-benzo[b][1,4]oxazine-7-carbonitrile (step 5) (220 mg, 1.2 mmol) in dioxane (20 ml) was added 5-bromo-3-chloro-2-isobutoxypyridine (intermediate A) (980 mg, 3.7 mmol), CuI (470 mg, 2.5 mmol), K₂CO₃ (341 mg, 2.5 mmol), and DMEDA (260 mg, 2.5 mmol). After addition, the mixture was degassed and charged with N₂ three times. Then the mixture was heated to 110° C. and stirred at that temperature under N₂ atmosphere for 16 hrs. When LC/MS indicated the starting material was consumed, the reaction mixture was diluted with DCM (200 ml). The mixture was filtered over celite. The filtration was washed with brine (×3), dried over anhydrous Na₂SO₄ and filtered. The filtration was evaporated to afford a residue, which was purified by flash column chromatography on silica gel using a gradient 0-20% ethyl acetate in hexanes to afford the title compound as white solid.

LC-MS: Rt=1.27 mins; MS m/z [M+H]+ 362.0; Method 5-95AB 1.5minLC_v003

¹H NMR (400 MHz, CDCl₃) δ 7.96 (1H, d), 7.56 (1H, d), 6.98 (1H, d), 6.22 (1H, d), 4.31 (2H, m), 4.11 (2H, d), 3.68 (2H, m), 2.16 (1H, m), 1.05 (6H, d).

### Step 7: 8-(5-chloro-6-isobutoxypyridin-3-yl)-7,8-dihydro-6H-isoxazolo[5′,4′:4,5]benzo[1,2-b][1,4]oxazin-3-amine

To a mixture of N-hydroxyacetamide (105 mg, 1.41 mmol) in DMF (4 ml) was added t-BuOK solution in THF (1.4 ml, 1.4 mmol, 1 M). The mixture was stirred at room temperature for 0.5 h and then added the solution of 4-(5-chloro-6-isobutoxy pyridin-3-yl)-6-fluoro-3,4-dihydro-2H-benzo[b][1,4]oxazine-7-carbonitrile (step 6) (170 mg, 0.47 mmol) in DMF (3 ml). The mixture was stirred at 80° C. for 16 hrs and then diluted with ethyl acetate (×3). The combined organic phases were washed with brine (×3), dried over anhydrous Na₂SO₄ and filtered. The filtration was evaporated to afford the crude product, which was purified by flash column chromatography on silica gel using a gradient 0-20% ethyl acetate in hexanes to afford the title compound as yellow solid.

LC-MS: Rt=1.15 mins; MS m/z [M+H]+ 375.1; Method 5-95AB 1.5minLC_v003

¹H NMR (400 MHz, CDCl₃) δ 7.99 (1H, m), 7.62 (1H, d), 6.89 (1H, d), 6.43 (1H, s), 4.67 (2H, s), 4.37 (2H, m), 4.16 (2H, d), 3.68 (2H, m), 2.15 (1H, m), 1.06 (6H, d).

### Step 8: N-(8-(5-chloro-6-isobutoxypyridin-3-yl)-7,8-dihydro-6H-isoxazolo[5′,4′:4,5]benzo[1,2-b][1,4]oxazin-3-yl)methanesulfonamide

To a solution of 8-(5-chloro-6-isobutoxypyridin-3-yl)-7,8-dihydro-6H-isoxazolo[5′,4′:4,5]benzo[1,2-b][1,4]oxazin-3-amine (step 7) (12 mg, 0.03 mmol) in DCM (2 ml) was added MsCl (12 mg, 0.10 mmol) and TEA (12 mg, 0.10 mmol). The reaction mixture was stirred at room temperature for 1 h. When TLC indicated the starting material was consumed, the reaction mixture was washed with water (×3), dried over anhydrous Na₂SO₄ and filtered. The filtration was concentrated under reduced pressure to give the crude product. To a solution of the above crude product N-(8-(5-chloro-6-isobutoxypyridin-3-yl)-7,8-dihydro-6H-isoxazolo[5′,4′:4,5]benzo[1,2-b][1,4]oxazin-3-yl)-N-(methylsulfonyl)methanesulfonamide (20 mg, 0.025 mmol) in THF (0.5 ml) was added TBAF (0.5 ml, 0.5 mmol). The reaction mixture was stirred at room temperature for 1 h. When TLC indicated the reaction was completed, the reaction mixture was concentrated under reduced pressure to give the crude product, which purified by pre-HPLC to afford the title compound as white solid.

LC-MS: Rt=5.95 mins; MS m/z [M+H]+ 453.0; Method 0-60AB 7minLC_v002

¹H NMR (400 MHz, CDCl₃) δ 7.95 (1H, d), 7.56 (1H, d), 7.28 (1H, s), 7.16 (1H, s), 6.43 (1H, s), 4.29 (2H, m), 4.11 (2H, d), 3.65 (2H, m), 3.19 (3H, s), 2.15 (1H, m), 0.99 (6H, d).

### Intermediate A (Method 20)

### 5-bromo-3-chloro-2-isobutoxypyridine

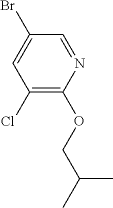

To a solution of 5-bromo-3-chloro-2-fluoropyridine (Aldrich) (105 g, 0.5 mol) and 2-methylpropan-1-ol (44 g, 0.6 mol) in dry THF (1 l) was added t-BuOK (112 g, 1 mol) dropwise with ice-bath, and then the mixture was stirred at room temperature for 16 hrs. The reaction was quenched with saturated NH₄Cl aqueous solution, extracted with EtOAc (×2). The separated organic phase was washed with brine (×2), dried over anhydrous Na₂SO₄ and filtered. The filtration solution was concentrated under reduced pressure to give the title compound.

LC-MS m/z Rt=1.16 mins; [M+H]+ 264, Method 5-95AB 1.5minLC_v003

¹H NMR (400 MHz, DMSO-d₆) δ 8.17 (1H, d), 7.88 (1H, d), 4.18 (2H, d), 2.06 (1H, m), 0.99 (6H, d).

Intermediate B to O were prepared by a similar method to that of intermediate A (Method 20) by replacing 2-methylpropan-1-ol with the appropriate compound (either commercially available or preparations described hereinafter).

### Intermediate B

### 5-bromo-3-chloro-2-(cyclohexylmethoxy)pyridine

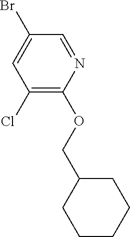 [SMILES: Clc1cc(Br)cnc1OCC1CCCCC1]

LC-MS m/z Rt=1.15 mins; [M+H]+ 304, Method 5-95AB 1.5minLC_v003

¹H NMR (400 MHz, CDCl₃) δ 8.06 (1H, d), 7.74 (1H, d), 4.14 (2H, d), 1.85 (1H, m), 1.73 (4H, m), 1.25 (4H, m), 1.05 (2H, m).

### Intermediate C

### 5-bromo-3-chloro-2-((tetrahydro-2H-pyran-4-yl)methoxy)pyridine

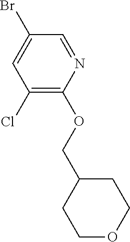 [SMILES: Clc1cc(Br)cnc1OCC1CCOCC1]

LC-MS m/z Rt=0.95 mins; [M+H]+ 305.9, Method 5-95AB 1.5minLC_v003

¹H NMR (400 MHz, CDCl₃) δ 8.39 (1H, d), 8.29 (1H, d), 3.90 (2H, d), 3.63 (2H, m), 3.53 (2H, m), 2.11 (1H, m), 1.69 (2H, m), 1.44 (2H, m).

### Intermediate D

### 5-bromo-3-chloro-2-((4,4-difluorocyclohexyl)methoxy)pyridine

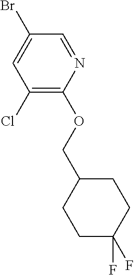 [SMILES: FC1(F)CCC(COc2ncc(Br)cc2Cl)CC1]

LC-MS m/z Rt=1.15 mins; [M+H]+ 340, Method 5-95AB 1.5minLC_v003

1H NMR (400 MHz, DMSO-d₆) δ 8.25 (2H, m), 4.20 (2H, d), 2.10 (7H, m), 1.30 (2H, m).

### Intermediate E

### 5-bromo-3-chloro-2-((4-(trifluoromethyl)cyclohexyl)methoxy)pyridine

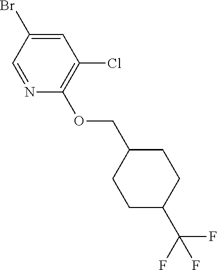 [SMILES: FC(F)(F)C1CCC(COc2ncc(Br)cc2Cl)CC1]

LC-MS m/z Rt=1.19 mins; [M+H]+ 372, Method 5-95AB 1.5minLC_v003

¹H NMR (400 MHz, DMSO-d₆) δ 8.30 (2H, m), 4.25 (2H, d), 2.25 (1H, m), 2.05 (8H, m), 1.35 (1H, m).

### Intermediate F

### 5-bromo-3-chloro-2-(4,4,4-trifluorobutoxyl)pyridine

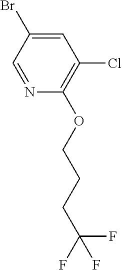 [SMILES: FC(F)(F)CCCOc1ncc(Br)cc1Cl]

LC-MS m/z Rt=1.15 mins; [M+H]+ 318, Method 5-95AB 1.5minLC_v003

¹H NMR (400 MHz, DMSO-d₆) δ 8.31 (2H, m), 4.25 (2H, d), 1.78 (4H, m).

### Intermediate G

### 5-bromo-3-chloro-2-(3,3-difluorocyclobutoxyl)pyridine

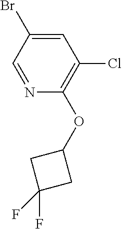 [SMILES: FC1(F)CC(Oc2ncc(Br)cc2Cl)C1]

LC-MS m/z Rt=1.21 mins; [M+H]+ 298, Method 5-95AB 1.5minLC_v003

1H NMR (400 MHz, CDCl₃) δ 8.08 (1H, d), 7.79 (1H, d), 5.10 (1H, m), 3.22 (2H, m), 2.70 (2H, m).

### Intermediate H

### (S)-5-bromo-3-chloro-2-(1-(4-fluorophenyl)ethoxy)pyridine

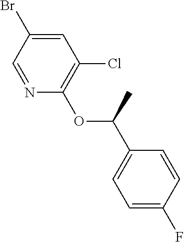 [SMILES: C[C@H](Oc1ncc(Br)cc1Cl)c1ccc(F)cc1]

LC-MS m/z Rt=1.09 mins; [M+H]+ 312, Method 5-95AB 1.5minLC_v003

¹H NMR (400 MHz, CDCl₃) δ 8.42 (1H, d), 7.70 (1H, d), 7.34 (2H, m), 7.20 (2H, m), 5.17 (1H, q), 1.75 (3H, d).

### Intermediate I

### 5-bromo-3-chloro-2-((1-methylcyclopropyl)methoxy)pyridine

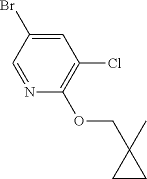

LC-MS m/z Rt=1.13 mins; [M+H]+ 276, Method 5-95AB 1.5minLC_v003

¹H NMR (400 MHz, CDCl₃) δ 8.05 (1H, d), 7.75 (1H, d), 4.14 (2H, s), 1.23 (3H, s), 0.58 (2H, m), 0.43 (2H, m).

### Intermediate J

### 5-bromo-3-chloro-2-(2,2,2-trifluoroethoxyl)pyridine

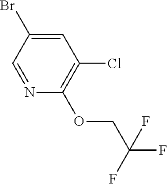 [SMILES: FC(F)(F)COc1ncc(Br)cc1Cl]

LC-MS m/z Rt=1.13 mins; [M+H]+ 289.9, Method 5-95AB 1.5minLC_v003

¹H NMR (400 MHz, CDCl₃) δ 8.08 (1H, d), 7.82 (1H, d), 4.78 (2H, q).

### Intermediate K

### 5-bromo-3-chloro-2-cyclobutoxypyridine

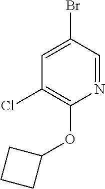 [SMILES: Clc1cc(Br)cnc1OC1CCC1]

LC-MS m/z Rt=1.33 mins; [M+H]+ 261.9, Method 5-95AB 1.5minLC_v003

¹H NMR (400 MHz, DMSO-d₆) δ: 8.25 (2H, m), 5.18 (1H, m), 2.40 (2H, m), 2.13 (2H, m), 1.79 (1H, m), 1.64 (1H, m)

### Intermediate L

### 5-bromo-3-fluoro-2-isobutoxypyridine

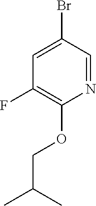

LC-MS m/z Rt=1.34 mins; [M+H]+ 248.2, Method 5-95AB 1.5minLC_v003

¹H NMR (400 MHz, DMSO-d₆) δ 8.19 (1H, m), 7.90 (1H, m), 4.28 (2H, d), 2.12 (1H, m), 0.99 (6H, d).

### Intermediate M

### 5-bromo-3-chloro-2-((4-methylcyclohexyl)methoxy)pyridine

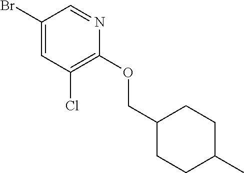 [SMILES: CC1CCC(COc2ncc(Br)cc2Cl)CC1]

LC-MS m/z Rt=1.44 mins; [M+H]+ 318.1, Method 5-95AB 1.5minLC_v003

¹H NMR (400 MHz, CDCl₃) δ 8.07 (1H, m), 7.74 (1H, m), 4.19 (2H, m), 2.02 (1H, m), 1.87 (1H, d), 1.69 (2H, m), 1.60 (1H, s), 1.57 (2H, d), 1.51 (1H, m), 1.29 (2H, m), 1.11 (1H, m), 0.92 (3H, m);

### Intermediate N

### 5-bromo-2-(sec-butoxy)-3-chloropyridine

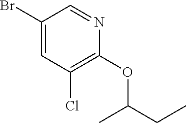

LC-MS m/z Rt=1.48 mins; [M+H]+ 263.9, Method 5-95AB 1.5minLC_v003

¹H NMR (400 MHz, CDCl₃) δ 8.04 (1H, d), 7.72 (1H, d), 5.09 (1H, m), 1.73 (2H, m), 1.32 (3H, d), 0.95 (3H, t).

### Intermediate O

### 5-bromo-3-chloro-2-(cyclobutylmethoxy)pyridine

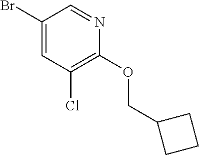 [SMILES: Clc1cc(Br)cnc1OCC1CCC1]

LC-MS m/z Rt=1.49 mins; [M+H]+ 275.9, Method 5-95AB 1.5minLC_v003

¹H NMR (400 MHz, CDCl₃) δ 8.07 (1H, d), 7.74 (1H, d), 4.31 (2H, d), 2.78 (1H, m), 2.14 (2H, m), 1.95 (4H, m).

### Intermediate P

### 5-bromo-3-chloro-2-(cyclohexyloxy)pyridine

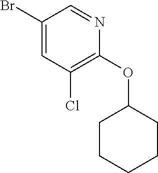 [SMILES: Clc1cc(Br)cnc1OC1CCCCC1]

LC-MS m/z Rt=1.43 mins; [M+H]+ 289.9, Method 5-95AB 1.5minLC_v003

¹H NMR (400 MHz, CDCl₃) δ 8.06 (1H, d), 7.74 (1H, d), 5.08 (1H, m), 1.96 (2H, m), 1.81 (2H, m), 1.60 (2H, m), 1.54 (1H, m), 1.41 (3H, m).

### Intermediate Q

### 5-bromo-3-chloro-2-(cyclopentylmethoxy)pyridine

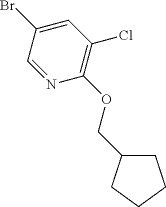 [SMILES: Clc1cc(Br)cnc1OCC1CCCC1]

LC-MS m/z Rt=1.43 mins; [M+H]+ 289.9, Method 5-95AB 1.5minLC_v003

¹H NMR (400 MHz, CDCl₃) δ 8.07 (1H, d), 7.75 (1H, d), 4.24 (2H, m), 2.41 (1H, m), 1.83 (2H, m), 1.65 (4H, m), 1.39 (2H, m).

### Intermediate R

### 5-bromo-3-chloro-2-(cyclopropylmethoxy)pyridine

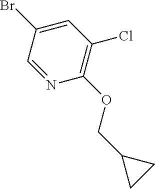 [SMILES: Clc1cc(Br)cnc1OCC1CC1]

LC-MS m/z Rt=1.41 mins; [M+H]+ 261.9, Method 5-95AB 1.5minLC_v003

¹H NMR (400 MHz, CDCl₃) δ 8.07 (1H, d), 7.75 (1H, d), 4.20 (2H, m), 1.30 (1H, m), 0.62 (2H, m), 0.40 (2H, m).

### Intermediate S

### 5-bromo-3-chloro-2-methoxypyridine

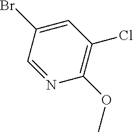

LC-MS m/z Rt=1.36 mins; [M+H]+ 221.9, Method 5-95AB 1.5minLC_v003

¹H NMR (400 MHz, CDCl₃) δ 8.10 (1H, d), 7.76 (1H, d), 4.01 (3H, s).

### Intermediate T

### 5-bromo-3-chloro-2-(difluoromethoxy)pyridine

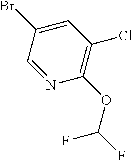 [SMILES: FC(F)Oc1ncc(Br)cc1Cl]

LC-MS m/z Rt=1.32 mins; [M+H]+ 257.9, Method 5-95AB 1.5minLC_v003

¹H NMR (400 MHz, CDCl₃) δ 8.51 (1H, d), 8.41 (1H, d), 7.69 (1H, t).

### Intermediate U (Method 21)

### 3-chloro-2-isobutoxy-5-(4,4,5,5-tetramethyl-1,3,2-dioxaborolan-2-yl)pyridine

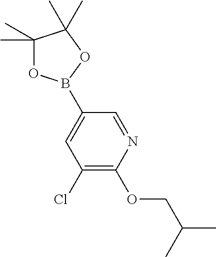 [SMILES: CC(C)COc1ncc(B2OC(C)(C)C(C)(C)O2)cc1Cl]

To a solution of 5-bromo-3-chloro-2-isobutoxypyridine (Intermediate A, 53 g, 0.2 mmol) in dioxane (800 ml) was added 4,4,4′,4′,5,5,5′,5′-octamethyl-2,2′-bi(1,3,2-dioxaborolane) (102 g, 0.4 mol), KOAc (39 g, 0.4 mol) and Pd(dppf)₂Cl₂ (7 g, 10 mmol). The mixture was degassed and charged with N₂ three times and stirred at 80° C. for 5 hrs. The reaction was cooled to room temperature and filtered through celite, washed with EtOAc (×2). The combined organic solvent was washed with brine (×5), dried over anhydrous Na₂SO₄ and filtered. The filtration solution was concentrated under reduced pressure to give the crude product, which was purified by flash column chromatography on silica gel eluting a gradient 0-5% ethyl acetate in hexanes to afford the title compound as colorless oil.

LC-MS m/z Rt=1.20 mins; [M+H]+ 312, Method 5-95AB 1.5minLC_v003

¹HNMR (400 MHz, CDCl₃) δ 8.36 (1H, d), 7.95 (1H, d), 4.16 (2H, d), 2.12 (1H, m), 1.33 (12H, s), 1.02 (6H, d).

### Intermediate V (Method 22)

### 5-chloro-6-isobutoxypyridin-3-amine

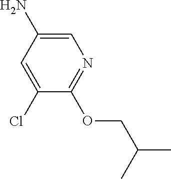

### Step 1: 3-chloro-2-isobutoxy-5-nitropyridine

To a mixture of 2,3-dichloro-5-nitropyridine (Aldrich) (192 g, 1 mol) and 2-methylpropan-1-ol (112 g, 1.5 mol) in THF (1 l) was added t-BuOK (168 g, 1.5 mol) by portion with ice-bath. After addition, the reaction mixture was stirred at room temperature for 16 hrs. When TLC indicated the reaction was completed, the reaction mixture was quenched with saturated NH₄Cl aqueous solution, extracted with EtOAc (×3). The combined organic phases were washed with brine (×2), dried over anhydrous magnesium sulfate and filtered. The filtration solution was concentrated under reduced pressure to give the title compound.

LC-MS m/z Rt=1.01 mins; [M+H]+ 231, Method 5-95AB 1.5minLC_v003

¹H NMR (400 MHz, CDCl₃) δ 8.95 (1H, d), 8.44 (1H, d), 4.27 (2H, d), 2.17 (1H, m), 1.06 (6H, d).

### Step 2: 5-chloro-6-isobutoxypyridin-3-amine

To a solution of 3-chloro-2-isobutoxy-5-nitropyridine (step 1) (23 g, 0.1 mol) in HOAc (100 ml) was added Zn (38 g, 0.6 mol) carefully. The mixture was stirred at room temperature for 3 hrs. The reaction was filtered through celite, washed with EtOAc (×2). The combined organic solvent was washed with brine (×2), dried over anhydrous Na₂SO₄ and filtered. The filtration solution was concentrated under reduced pressure to give the crude product, which was purified by flash column chromatography on silica gel eluting a gradient 30-60% ethyl acetate in hexanes to afford the title compound as purple oil.

LC-MS m/z Rt=0.840 mins; [M+H]+ 201, Method 5-95AB 1.5minLC_v003

¹H NMR (400 MHz, DMSO-d₆) δ 7.54 (1H, d), 7.11 (1H, d), 4.03 (2H, d), 3.40 (2H, s), 2.09 (1H, m), 1.03 (6H, d).

### Intermediate W (Method 23)

### 5-bromo-1-chloro-3-fluoro-2-isobutoxybenzene

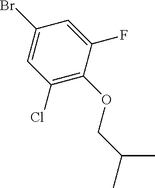

To a mixture of 4-bromo-2-chloro-6-fluorophenol (4 g, 18 mmol) in DMF (60 ml) was added K₂CO₃ (4.9 g, 35 mmol) and 1-bromo-2-methylpropane (3.6 g, 27 mmol). The resulting mixture was heated to 120° C. for 0.5 h. After cooling down, the reaction mixture was diluted with water and extracted with ethyl acetate (×3). The combined organic phases were washed with water (×3), dried over anhydrous magnesium and filtered. The filtration was concentrated to afford the title compound as colorless oil.

¹H NMR (400 MHz, CDCl₃) δ 7.29 (1H, s), 7.16 (1H, m), 3.82 (2H, d), 2.07 (1H, m), 1.03 (6H, d).

### Intermediate X (Method 24)

### 5-bromo-3-chloro-2-isobutoxybenzonitrile

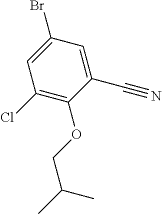

### Step 1: 5-bromo-3-chloro-2-hydroxybenzaldehyde

To a mixture of 4-bromo-2-chlorophenol (Alfa) (10 g, 48 mmol) in 40 ml of trifluoroacetic acid was added hexamethylenetetramine (14 g, 97 mmol). The resulting mixture was stirred at 90° C. for 20 hrs. After cooling to ambient temperature, the reaction mixture was water and followed by an 50% sulfuric acid aqueous solution (28 ml). The mixture was stirred at ambient temperature for an additional 2 hrs. The precipitate was collected and dried to afford the title compound as yellow solid.

¹H NMR (400 MHz, CDCl₃) δ 11.39 (1H, s), 9.84 (1H, s), 7.74 (1H, d), 7.62 (1H, d).

### Step 2: 5-bromo-3-chloro-2-hydroxybenzonitrile

To a mixture of the 5-bromo-3-chloro-2-hydroxybenzaldehyde (step 1) (5.5 g, 23 mmol) in HCOOH (100 ml) was added hydroxylamine hydrochloride (3.2 g, 47 mmol) and HCOONa (3.2 g, 47 mmol). The reaction mixture was stirred at 100° C. for 10 hrs before added 100 ml of water. The mixture was extracted with ethyl acetate (×3). The combined organic layers were washed with water (×3) and brine (300 ml), dried over anhydrous magnesium sulfate and filtered. The filtration was evaporated to afford the title compound as white solid.

¹H NMR (400 MHz, DMSO-d₆) δ 7.96 (1H, d), 7.91 (1H, d).

### Step 3: 5-bromo-3-chloro-2-isobutoxybenzonitrile

To a reaction mixture of 5-bromo-3-chloro-2-hydroxybenzonitrile (step 2) (2 g, 8.7 mmol) in 60 ml of DMF was added 1-iodo-2-methylpropane (4.8 g, 26 mmol) and K₂CO₃ (5.9 g, 43 mmol). The mixture was stirred at 50° C. for 16 hrs under N₂. When TLC indicated the reaction was completed, the reaction mixture was evaporated under reduced pressure to give the residue, which was treated with ethyl acetate and water. The separated organic pahse was washed with water (×3) and brine (×1), dried over anhydrous magnesium and filtered. The filtration was evaporated to give crude product, which was purified by flash column chromatography on silica gel using hexanes afforded the title compound as white solid.

¹H NMR (400 MHz, CDCl₃) δ 7.72 (1H, d), 7.58 (1H, d), 3.96 (2H, d), 2.17 (1H, td), 1.07 (6H, d).

### Biological Data

hNav1.7 Channel In Vitro Patch-Clamp Assay

Cells: Sable HEK293 (human embryonic kidney) cell line expressing hNav1.7 channels was used in the assay. The cell line was obtained from Millipore (Millipore Corporation, MA).

Patch-clamp recordings: The recordings were performed on QPatch HTX (Sophion Bioscience, Denmark) and/or conventional patch-clamp (Axon Multiclamp 700B systems, Molecular Devices, CA) platforms at room temperature, using whole cell patch-clamp techniques. The composition of extracellular solution was (in mM): HEPES 5, NaCl 40, KCl 3, CaCl₂ 1, MgCl₂ 1, Choline Chloride 100. Adjust pH to 7.2 with 1N NaOH, osmolarity to 300-310 mOsm with sucrose. The composition of intracellular solution was (in mM): HEPES 10, NaCl 10, Na₂ATP 1, MgCl₂ 2, KF 135, EGTA 10. Adjust pH to 7.2 with 1N NaOH, osmolarity to 290-300 mOsm with sucrose.

For conventional patch-clamp, 35 mm culture dishes containing hNav1.7 cells were placed on the stage of an inverted microscope and continuously perfused with extracellular solution from a perfusion system (Biologic Rapid solution changer, RSC-160, ˜1 ml/min). Micropipettes was filled with the intracellular solutions and had a resistance of 2˜4 MΩ. The recordings were controlled by pCLAMP10 software. Test compounds were dissolved in 100% DMSO to make stock solutions for each test concentration, and then diluted into extracellular solution to achieve final concentration for testing. Final DMSO concentration was less than 0.3%. Two voltage protocols were used in hNav1.7 assay: for non-inactivated protocol, the sodium currents were elicited by a 2 ms pulse to −10 mV from a holding potential of −110 mV. For inactivated protocol, the holding potential was set at V_{1/2}. The voltage was firstly stepped to −110 mV for 50 ms, then to −10 mV for 2 ms and then stepped back to holding potential. In both protocols the voltage command was given in every 10 s. Compound effect (% inhibition) was determined by difference in current amplitude before and after the application of test compounds. IC₅₀ values were determined from concentration-response curves that were obtained with Hill fitting.

For QPatch HTX assay, hNav1.7 cells grown to 50%-80% confluence were rinsed with DPBS and dissociated with Tryple (Invitrogen, 12605-010). The dissociated cells were then mixed with extracellular solution. After centrifuge, the cells were resuspended in extracellular solution and used in the QPatch experiment. The whole-cell protocols, voltage protocols and application protocols were established with QPatch Assay Software 3.4 (Sophion Bioscience, Denmark). Only the inactivated protocol was used in the QPatch assay.

TABLE 1
The following table gives the IC₅₀ values for exemplified
compounds as measured in the above assay.

| Example | Compound name | IC₅₀ (μM) |
|---|---|---|
| 1 | N-(7′-(5-chloro-6-isobutoxypyridin-3-yl)-6′- | 0.15 |
| | oxo-6′,7′-dihydrospiro [cyclopropane-1,5′- | |
| | isoxazolo[4,5-f]indol]-3′- | |
| | yl)methanesulfonamide | |
| 2 | N-(7′-(5-chloro-6-isobutoxypyridin-3-yl)-6′- | 0.08 |
| | oxo-6′,7′-dihydrospiro [cyclopropane-1,5′- | |
| | isoxazolo[4,5-f]indol]-3′- | |
| | yl)cyclopropanesulfonamide | |
| 3 | N-(7′-(5-chloro-6-isobutoxypyridin-3-yl)-6′- | 0.1 |
| | oxo-6′,7′-dihydrospiro [cyclopropane-1,5′- | |
| | isoxazolo[4,5-f]indol]-3′-yl)propane-2- | |
| | sulfonamide | |
| 4 | N-(7′-(5-chloro-6-isobutoxypyridin-3-yl)-6′- | 0.1 |
| | oxo-6′,7′-dihydrospiro [cyclopropane-1,5′- | |
| | isoxazolo[4,5-f]indol]-3′- | |
| | yl)ethanesulfonamide | |
| 5 | N-(7′-(5-chloro-6-isobutoxypyridin-3-yl)-6′- | 0.43 |
| | oxo-6′,7′-dihydrospiro [cyclopropane-1,5′- | |
| | isoxazolo[4,5-f]indol]-3′-yl)-1,1,1- | |
| | trifluoromethanesulfonamide | |
| 6 | N-(7′-(5-chloro-6-isobutoxypyridin-3-yl)-6′- | 3.00 |
| | oxo-6′,7′-dihydrospiro [cyclopropane-1,5′- | |
| | isoxazolo[4,5-f]indol]-3′- | |
| | yl)benzenesulfonamide | |
| 7 | N-(7′-(5-chloro-6-isobutoxypyridin-3-yl)-6′- | 0.50 |
| | oxo-6′,7′-dihydrospiro [cyclopropane-1,5′- | |
| | isoxazolo[4,5-f]indol]-3′-yl)thiophene-2- | |
| | sulfonamide | |
| 8 | N-(7′-(5-chloro-6-isobutoxypyridin-3-yl)-6′- | 2.00 |
| | oxo-6′,7′-dihydrospiro [cyclopropane-1,5′- | |
| | isoxazolo[4,5-f]indol]-3′- | |
| | yl)aminosulfonamide | |
| 9 | N-(7′-(5-chloro-6-isobutoxypyridin-3-yl)-6′- | 1.23 |
| | oxo-6′,7′-dihydrospiro [cyclopropane-1,5′- | |
| | isoxazolo[4,5-f]indol]-3′-yl)dimethylamino-1- | |
| | sulfonamide | |
| 10 | N-(7′-(5-chloro-6-isobutoxypyridin-3-yl)-6′- | 0.50 |
| | oxo-6′,7′-dihydrospiro [cyclopropane-1,5′- | |
| | isoxazolo[4,5-f]indol]-3′-yl)pyrrolidine-1- | |
| | sulfonamide | |
| 11 | N-(7′-(5-chloro-6- | 0.10 |
| | (cyclohexylmethoxy)pyridin-3-yl)-6′-oxo- | |
| | 6′,7′-dihydrospiro[cyclopropane-1,5′- | |
| | isoxazolo[4,5-f]indol]-3′- | |
| | yl)methanesulfonamide | |
| 12 | N-(7′-(5-chloro-6-((tetrahydro-2H-pyran-4- | 2.00 |
| | yl)methoxy)pyridin-3-yl)-6′-oxo-6′,7′- | |
| | dihydrospiro[cyclopropane-1,5′- | |
| | isoxazolo[4,5-f]indol]-3′- | |
| | yl)methanesulfonamide | |
| 13 | N-(7′-(5-chloro-6-((4,4- | 0.30 |
| | difluorocyclohexyl)methoxy)pyridin-3-yl)-6′- | |
| | oxo-6′,7′-dihydrospiro[cyclopropane-1,5′- | |
| | isoxazolo[4,5-f]indol]-3′- | |
| | yl)methanesulfonamide | |
| 14 | N-(7′-(5-chloro-6-((4- | 0.20 |
| | (trifluoromethyl)cyclohexyl)methoxy)pyridin- | |
| | 3-yl)-6′-oxo-6′,7′- | |
| | dihydrospiro[cyclopropane-1,5′- | |
| | isoxazolo[4,5-f]indol]-3′- | |
| | yl)methanesulfonamide | |
| 15 | N-(7′-(5-chloro-6-((1- | 0.07 |
| | methylcyclopropyl)methoxy)pyridin-3-yl)- | |
| | 6′-oxo-6′,7′-dihydrospiro[cyclopropane-1,5′- | |
| | isoxazolo[4,5-f]indol]-3′-yl) | |
| | methanesulfonamide | |
| 16 | N-(7′-(5-chloro-6-((1- | 0.06 |
| | methylcyclopropyl)methoxy)pyridin-3-yl)- | |
| | 6′-oxo-6′,7′-dihydrospiro[cyclopropane-1,5′- | |
| | isoxazolo[4,5-f]indol]-3′-yl) | |
| | cyclopropanesulfonamide | |
| 17 | N-(7′-(5-chloro-6-(4,4,4- | 0.20 |
| | trifluorobutoxy)pyridin-3-yl)-6′-oxo-6′,7′- | |
| | dihydrospiro[cyclopropane-1,5′- | |
| | isoxazolo[4,5-f]indol]-3′-yl) | |
| | cyclopropanesulfonamide | |
| 18 | (S)-N-(7′-(5-chloro-6-(1-(4- | 0.10 |
| | fluorophenyl)ethoxy)pyridin-3-yl)-6′-oxo- | |
| | 6′,7′-dihydrospiro[cyclopropane-1,5′- | |
| | isoxazolo[4,5-f]indol]-3′- | |
| | yl)cyclopropanesulfonamide | |
| 19 | N-(7′-(5-chloro-6-(3,3- | 0.20 |
| | difluorocyclobutoxy)pyridin-3-yl)-6′-oxo-6′,7′- | |
| | dihydrospiro[cyclopropane-1,5′- | |
| | isoxazolo[4,5-f]indol]-3′- | |
| | yl)cyclopropanesulfonamide | |
| 20 | N-(7′-(5-chloro-6-(2,2,2- | 0.50 |
| | trifluoroethoxy)pyridin-3-yl)-6′-oxo-6′,7′- | |
| | dihydrospiro[cyclopropane-1,5′- | |
| | isoxazolo[4,5-f]indol]-3′- | |
| | yl)cyclopropanesulfonamide | |
| 21 | N-(7′-(5-chloro-6-cyclobutoxypyridin-3-yl)- | 0.15 |
| | 6′-oxo-6′,7′-dihydrospiro [cyclopropane- | |
| | 1,5′-isoxazolo[4,5-f]indol]-3′- | |
| | yl)cyclopropanesulfonamide | |
| 22 | N-(7′-(5-fluoro-6-isobutoxypyridin-3-yl)-6′- | 0.30 |
| | oxo-6′,7′-dihydrospiro [cyclopropane-1,5′- | |
| | isoxazolo[4,5-f]indol]-3′- | |
| | yl)cyclopropanesulfonamide | |
| 23 | N-(7′-(5-chloro-6-((4- | 0.20 |
| | methylcyclohexyl)methoxy)pyridin-3-yl)-6′- | |
| | oxo-6′,7′-dihydrospiro[cyclopropane-1,5′- | |
| | isoxazolo[4,5-f]indol]-3′- | |
| | yl)cyclopropanesulfonamide | |
| 24 | N-(7′-(6-(sec-butoxy)-5-chloropyridin-3-yl)- | 0.07 |
| | 6′-oxo-6′,7′-dihydrospiro [cyclopropane- | |
| | 1,5′-isoxazolo[4,5-f]indol]-3′- | |
| | yl)cyclopropanesulfonamide | |
| 25 | N-(7′-(5-chloro-6- | 0.08 |
| | (cyclobutylmethoxy)pyridin-3-yl)-6′-oxo- | |
| | 6′,7′-dihydrospiro[cyclopropane-1,5′- | |
| | isoxazolo[4,5-f]indol]-3′-yl) | |
| | cyclopropanesulfonamide | |
| 26 | N-(7′-(5-chloro-6-(cyclohexyloxy)pyridin-3- | 0.11 |
| | yl)-6′-oxo-6′,7′-dihydro spiro[cyclopropane- | |
| | 1,5′-isoxazolo[4,5-f]indol]-3′- | |
| | yl)cyclopropanesulfonamide | |
| 27 | N-(7′-(5-chloro-6- | 0.09 |
| | (cyclopentylmethoxy)pyridin-3-yl)-6′-oxo- | |
| | 6′,7′-dihydrospiro[cyclopropane-1,5′- | |
| | isoxazolo[4,5-f]indol]-3′-yl) | |
| | cyclopropanesulfonamide | |
| 28 | N-(7′-(5-chloro-6- | 0.20 |
| | (cyclopropylmethoxy)pyridin-3-yl)-6′-oxo- | |
| | 6′,7′-dihydrospiro[cyclopropane-1,5′- | |
| | isoxazolo[4,5-f]indol]-3′- | |
| | yl)cyclopropanesulfonamide | |
| 29 | N-(7′-(5-chloro-6-methoxypyridin-3-yl)-6′- | 0.80 |
| | oxo-6′,7′-dihydrospiro [cyclopropane-1,5′- | |
| | isoxazolo[4,5-f]indol]-3′- | |
| | yl)cyclopropanesulfonamide | |
| 30 | N-(7′-(5-chloro-6-(difluoromethoxy)pyridin- | 0.63 |
| | 3-yl)-6′-oxo-6′,7′-dihydro | |
| | spiro[cyclopropane-1,5′-isoxazolo[4,5- | |
| | f]indol]-3′-yl)cyclopropanesulfonamide | |
| 31 | N-(7′-(3,4-dichlorophenyl)-6′-oxo-6′,7′- | 0.80 |
| | dihydrospiro[cyclopropane-1,5′- | |
| | isoxazolo[4,5-f]indol]-3′- | |
| | yl)methanesulfonamide | |
| 32 | N-(7′-(3-chloro-5-cyano-4- | 0.90 |
| | isobutoxyphenyl)-6′-oxo-6′,7′-dihydro | |
| | spiro[cyclopropane-1,5′-isoxazolo[4,5- | |
| | f]indol]-3′-yl)methanesulfonamide | |
| 33 | N-(7′-(3-chloro-5-fluoro-4-isobutoxyphenyl)- | 0.10 |
| | 6′-oxo-6′,7′-dihydro spiro[cyclopropane- | |
| | 1,5′-isoxazolo[4,5-f]indol]-3′- | |
| | yl)cyclopropanesulfonamide | |
| 34 | N-(7-(5-chloro-6-isobutoxypyridin-3-yl)-5- | 0.10 |
| | methyl-6-oxo-6,7-dihydro-5H- | |
| | imidazo[4′,5′:4,5]benzo[1,2-d]isoxazol-3- | |
| | yl)methanesulfonamide | |
| 35 | N-(7-(5-chloro-6-isobutoxypyridin-3-yl)-5- | 0.10 |
| | methyl-6-oxo-6,7-dihydro-5H- | |
| | imidazo[4′,5′:4,5]benzo[1,2-d]isoxazol-3- | |
| | yl)cyclopropanesulfonamide | |
| 36 | N-(7-(5-chloro-6-isobutoxypyridin-3-yl)-5,5- | 0.07 |
| | dimethyl-6-oxo-6,7-dihydro-5H- | |
| | isoxazolo[4,5-f]indol-3- | |
| | yl)cyclopropanesulfonamide | |
| 37 | N-(7′-(5-chloro-6-isobutoxypyridin-3-yl)-6′- | 0.04 |
| | oxo-6′,7′-dihydrospiro [cyclobutane-1,5′- | |
| | isoxazolo[4,5-f]indol]-3′- | |
| | yl)cyclopropanesulfonamide | |
| 38 | N-(7′-(5-chloro-6-isobutoxypyridin-3-yl)-6′- | 0.50 |
| | oxo-6′,7′-dihydrospiro [cyclopentane-1,5′- | |
| | isoxazolo[4,5-f]indol]-3′- | |
| | yl)cyclopropanesulfonamide | |
| 39 | N-(7′-(5-chloro-6-isobutoxypyridin-3-yl)-6′- | 0.70 |
| | oxo-6′,7′-dihydrospiro [cyclohexane-1,5′- | |
| | isoxazolo[4,5-f]indol]-3′- | |
| | yl)cyclopropanesulfonamide | |
| 40a | N-(7-(5-chloro-6-isobutoxypyridin-3-yl)-5- | 0.03 |
| | (trifluoromethyl)-7H-isoxazolo[4,5-f]indol-3- | |
| | yl)methanesulfonamid | |
| 40b | N-(7-(5-chloro-6-isobutoxypyridin-3-yl)-6- | 0.04 |
| | (trifluoromethyl)-7H-isoxazolo[4,5-f]indol-3- | |
| | yl)methanesulfonamide | |
| 41 | N-(7-(5-chloro-6-isobutoxypyridin-3-yl)-5- | 0.05 |
| | (trifluoromethyl)-7H-isoxazolo[4,5-f]indol-3- | |
| | yl)cyclopropanesulfonamide | |
| 42 | N-(7-(5-chloro-6-isobutoxypyridin-3-yl)-5- | 0.07 |
| | methyl-7H-isoxazolo[4,5-f] indol-3- | |
| | yl)cyclopropanesulfonamide | |
| 43 | N-(7-(5-chloro-6-isobutoxypyridin-3-yl)-6- | 0.10 |
| | (trifluoromethyl)-7H- | |
| | imidazo[4′,5′:4,5]benzo[1,2-d]isoxazol-3- | |
| | yl)cyclopropanesulfonamide | |
| 44 | N-(7-(5-chloro-6-isobutoxypyridin-3-yl)-5- | 0.10 |
| | methyl-7H-isoxazolo[4,5-f] indazol-3- | |
| | yl)methanesulfonamide | |
| 45 | N-(7-(5-chloro-6-isobutoxypyridin-3-yl)-5- | 0.03 |
| | methyl-7H-isoxazolo [4,5-f]indazol-3- | |
| | yl)cyclopropanesulfonamide | |
| 46 | N-(7-(3,4-dichlorophenyl)-5-methyl-7H- | 0.10 |
| | isoxazolo[4,5-f]indazol-3- | |
| | yl)cyclopropanesulfonamide | |
| 47 | N-(7-(5-chloro-6-isobutoxypyridin-3-yl)-5- | 0.40 |
| | methyl-5H-isoxazolo [5,4-f] indazol-3- | |
| | yl)cyclopropanesulfonamide | |
| 48 | N-(7-(5-chloro-6-isobutoxypyridin-3-yl)-5- | 0.22 |
| | cyano-7H-isoxazolo[4,5-f] indol-3- | |
| | yl)cyclopropanesulfonamide | |
| 49 | 7-(5-chloro-6-isobutoxypyridin-3-yl)-3- | 1.18 |
| | (cyclopropanesulfonamido)-7H- | |
| | isoxazolo[4,5-f]indole-5-carboxamide | |
| 50 | N-(8-(5-chloro-6-isobutoxypyridin-3- | 0.11 |
| | yl)isoxazolo[4,5-g]isoquinolin-3- | |
| | yl)cyclopropanesulfonamide | |
| 51 | N-(8-(5-chloro-6-isobutoxypyridin-3- | 0.30 |
| | yl)isoxazolo[4,5-g]isoquinolin-3- | |
| | yl)methanesulfonamide | |
| 52 | N-(8-(5,6-dichloropyridin-3-yl)isoxazolo[4,5- | 0.80 |
| | g]isoquinolin-3-yl) | |
| | cyclopropanesulfonamide | |
| 53 | N-(8-(5-chloro-6-isobutoxypyridin-3- | 1.07 |
| | yl)isoxazolo[4,5-g] isoquinolin-3-yl) N-N- | |
| | dimethylsulfamide | |
| 55 | N-(8-(5-chloro-6-isobutoxypyridin-3- | 0.85 |
| | yl)isoxazolo[5,4-g]quinolin-3- | |
| | yl)cyclopropanesulfonamide | |
| 57 | N-(8-(5-chloro-6-isobutoxypyridin-3- | 2.36 |
| | yl)isoxazolo[5,4-g]isoquinolin-3- | |
| | yl)methanesulfonamide | |
| 58 | N-(8-(5-chloro-6-isobutoxypyridin-3- | 0.01 |
| | yl)naphtho[2,3-d]isoxazol-3-yl) | |
| | cyclopropanesulfonamide | |
| 59 | N-(8-(5-chloro-6-isobutoxypyridin-3- | 1.80 |
| | yl)isoxazolo[5,4-g]quinazolin-3- | |
| | yl)cyclopropanesulfonamide | |
| 60 | N-(8-(5-chloro-6-isobutoxypyridin-3-yl)-5- | 1.87 |
| | methylisoxazolo[4,5-g] isoquinolin-3- | |
| | yl)cyclopropanesulfonamide | |
| 61 | N-(8-(5-chloro-6-isobutoxypyridin-3-yl)-6- | 0.51 |
| | methylisoxazolo[4,5-g] isoquinolin-3- | |
| | yl)cyclopropanesulfonamide | |
| 62 | N-(8′-(5-chloro-6-isobutoxypyridin-3-yl)-7′- | 0.10 |
| | oxo-7′,8′-dihydrospiro[cyclopropane- | |
| | 1,6′- | |
| | isoxazolo[5′,4′:4,5]benzo[1,2-b][1,4]oxazin]- | |
| | 3′-yl)cyclopropan esulfonamide | |
| 63 | N-(8-(5-chloro-6-isobutoxypyridin-3-yl)-7,8- | 0.53 |
| | dihydro-6H-isoxazolo [5′,4′:4,5]benzo[1,2- | |
| | b][1,4]oxazin-3-yl)methanesulfonamide | |

The following compound did not show any activity in the above assay: N-(7′-(5-chloro-6-isobutoxypyridin-3-yl)-6′-oxo-6′,7′-dihydrospiro[cyclopropane-1,5′-isoxazolo[4,5-f]indol]-3′-yl)azetidine-1-sulfonamide.

1. A compound of formula (I) 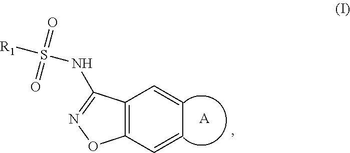
wherein
R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;
R¹¹ and R¹² are independently selected from the group consisting H and C₁₋₄ alkyl;
A is selected from the group consisting of 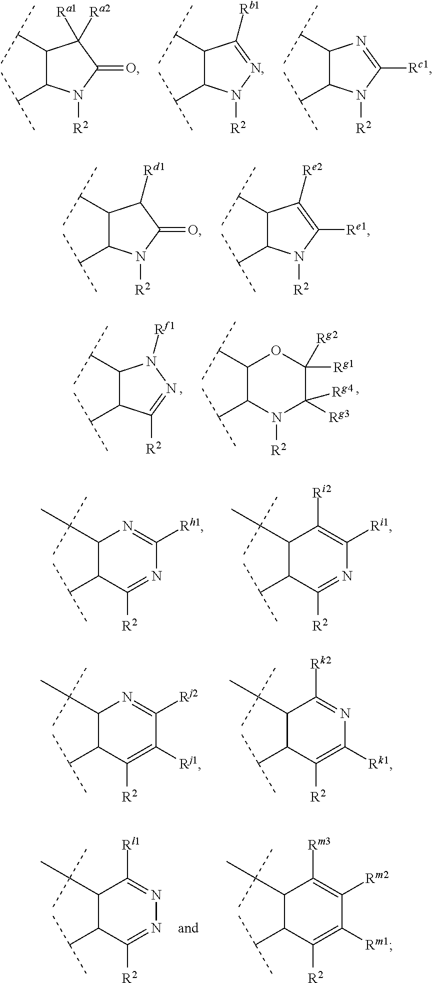
R^{a1} and R^{a2} are independently selected from the group consisting of H, C₁₋₄ alkyl, C₁₋₄ haloalkyl, halogen and CN;
or R^{a1} and R^{a2} form together with the carbon atom to which they are attached a C₃₋₆ cycloalkyl ring;
R^{b1} is selected from the group consisting of H, C₁₋₄ alkyl, C₁₋₄ haloalkyl, halogen and CN;
R^{c1} is selected from the group consisting of H, C₁₋₄ alkyl, C₁₋₄ haloalkyl, halogen and CN;
R^{d1} is selected from the group consisting of H, C₁₋₄ alkyl and C₁₋₄ haloalkyl;
R^{e1} is selected from the group consisting of H, C₁₋₄ alkyl, C₁₋₄ haloalkyl, halogen and CN;
R^{e2} is selected from the group consisting of H, C₁₋₄ alkyl, C₁₋₄ haloalkyl, halogen, CN and —C(═O)NR^{e21}R^{e22};
R^{e21} and R^{e22} are independently selected from H and C₁₋₄ alkyl;
R^{f1} selected from the group consisting of H, C₁₋₄ alkyl and C₁₋₄ haloalkyl;
R^{g1} and R^{g2} are independently selected from the group consisting of H, C₁₋₄ alkyl and C₁₋₄ haloalkyl;
or R^{g1} and R^{g2} form together with the carbon atom to which they are attached a C₃₋₆ cycloalkyl ring;
R^{g3} and R^{g4} are independently selected from the group consisting of H, C₁₋₄ alkyl and C₁₋₄ haloalkyl;
or R^{g3} and R^{g4} form together an oxo group;
R^{h1}, Rⁱ¹, Rⁱ², R^{j1}, R^{j2}, R^{k1}, R^{k2}, R^{l1}, R^{m1}, R^{m2} and R^{m3} are independently selected from the group consisting of H, C₁₋₄ alkyl, C₁₋₄ haloalkyl, halogen and CN;
R² is selected from the group consisting of 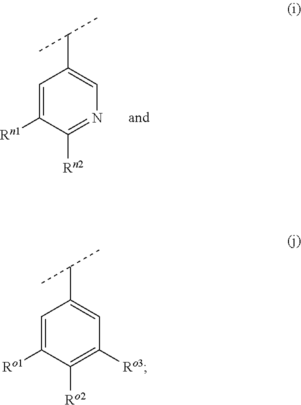
Rⁿ¹ is selected from the group consisting of H, halogen and CN;
Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;
Rⁿ²¹ and Rⁿ²² are independently selected from the group consisting of H, C₁₋₄ alkyl and halogen;
R^{o1} is selected from the group consisting of H and halogen;
R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;
R^{o21} and R^{o22} are independently selected from the group consisting of H, C₁₋₄ alkyl and halogen;
n is independently selected from the group consisting of 0, 1 and 2;
R^{o3} is selected from the group consisting of halogen and CN;
or a pharmaceutically acceptable salt thereof.

2. A compound according to claim 1 of formula (I) 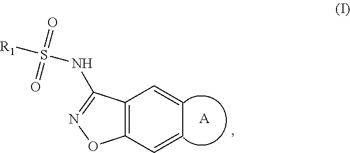
wherein
R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;
R¹¹ and R¹² are independently selected from the group consisting H and C₁₋₄ alkyl;
A is selected from the group consisting of 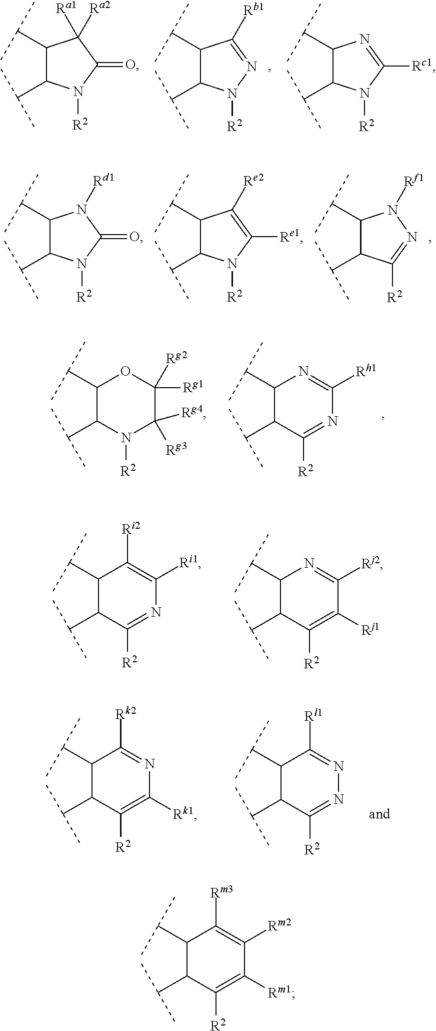
R^{a1} and R^{a2} are independently selected from the group consisting of H and C₁₋₄ alkyl;
or R^{a1} and R^{a2} form together with the carbon atom to which they are attached a C₃₋₆ cycloalkyl ring;
R^{b1} selected from the group consisting of H and C₁₋₄ alkyl;
R^{c1} selected from the group consisting of H and C₁₋₄ haloalkyl;
R^{d1} is selected from the group consisting of H and C₁₋₄ alkyl;
R^{e1} is selected from the group consisting of H, C₁₋₄ alkyl, C₁₋₄ haloalkyl and CN;
R^{e2} is selected from the group consisting of H, C₁₋₄ alkyl, C₁₋₄ haloalkyl, CN and —C(═O)NR^{e21}R^{e22};
R^{e21} and R^{e22} are independently selected from H and C₁₋₄ alkyl;
R^{f1} selected from the group consisting of H and C₁₋₄ alkyl;
R^{g1} and R^{g2} are independently selected from the group consisting of H and C₁₋₄ alkyl;
or R^{g1} and R^{g2} form together with the carbon atom to which they are attached a C₃₋₆ cycloalkyl ring;
R^{g3} and R^{g4} are independently selected from the group consisting of H, C₁₋₄ alkyl and C₁₋₄ haloalkyl;
or R^{g3} and R^{g4} form together an oxo group or together with the carbon atom to which they are attached a C₃₋₆ cycloalkyl ring;
R^{h1}, Rⁱ¹, Rⁱ², R^{j1}, R^{j2}, R^{k1}, R^{k2}, R^{l1}, R^{m1}, R^{m2} and R^{m3} are independently selected from the group consisting of H and C₁₋₄ alkyl;
R² is selected from the group consisting of 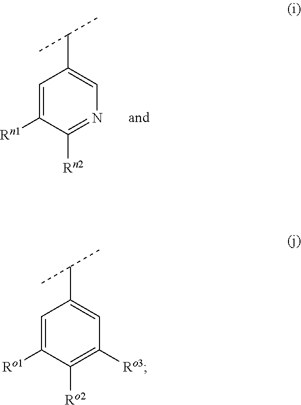
Rⁿ¹ is halogen;
Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;
Rⁿ²¹ and Rⁿ²² are independently selected from the group consisting of H, C₁₋₄ alkyl and halogen;
R^{o1} is selected from the group consisting of H and halogen;
R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;
R^{o21} and R^{o22} are independently selected from the group consisting of H, C₁₋₄ alkyl and halogen;
n is independently selected from the group consisting of 0, 1 and 2;
R^{o3} is selected from the group consisting of halogen and CN;
or a pharmaceutically acceptable salt thereof.

3. A compound or salt according to claim 1, wherein
R² is 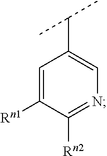
Rⁿ¹ is halogen;
Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;
n is independently selected from the group consisting of 0, 1 and 2;
Rⁿ²¹ and Rⁿ²² are independently selected from the group consisting of H, C₁₋₄ alkyl and halogen;
or a pharmaceutically acceptable salt thereof.

4. A compound or salt according to claim 3, wherein
Rⁿ¹ is chloro or fluoro and Rⁿ² is selected from the group consisting of 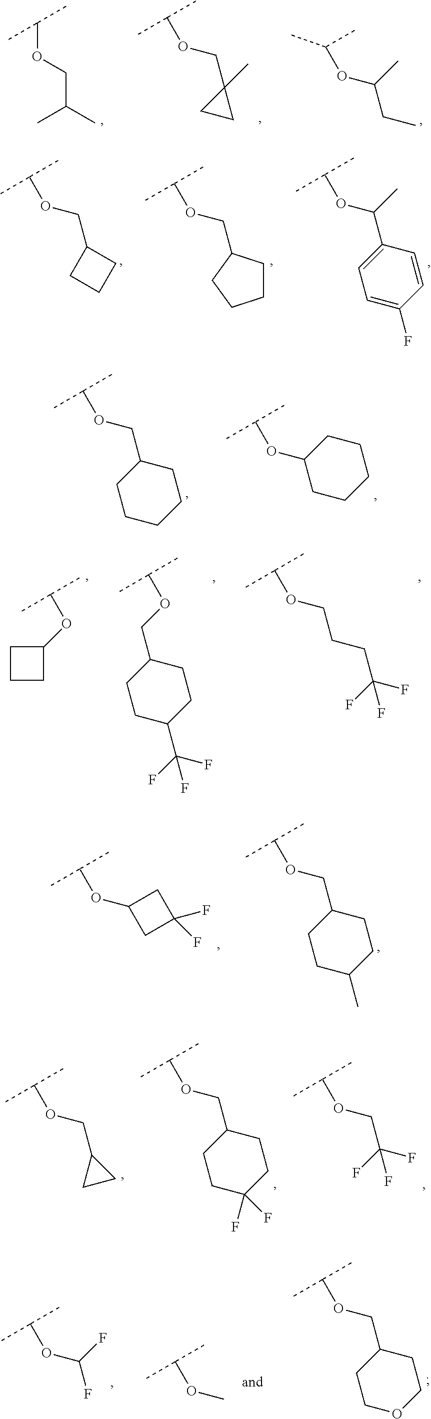
or a pharmaceutically acceptable salt thereof.

5. A compound according to claim 1 of formula (Ia) 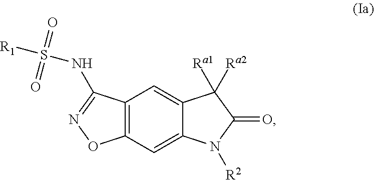
wherein
R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;
R¹¹ and R¹² are independently selected from the group consisting H and C₁₋₄ alkyl;
R^{a1} and R^{a2} are independently selected from the group consisting of H, C₁₋₄ alkyl, C₁₋₄ haloalkyl, halogen and CN;
or R^{a1} and R^{a2} form together with the carbon atom to which they are attached a C₃₋₆ cycloalkyl ring;
R² is selected from the group consisting of 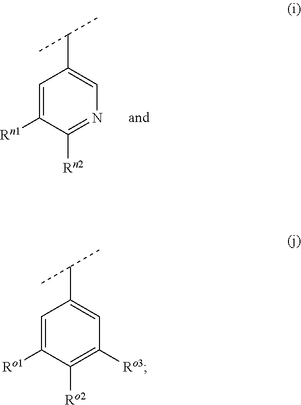
Rⁿ¹ is selected from the group consisting of halogen and CN;
Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;
Rⁿ²¹ and Rⁿ²² are independently selected from the group consisting of H, C₁₋₄ alkyl and halogen;
R^{o1} is selected from the group consisting of H and halogen;
R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹R²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;
R^{o21} and R^{o22} are independently selected from the group consisting of H, C₁₋₄ alkyl and halogen;
n is independently selected from the group consisting of 0, 1 and 2;
R^{o3} is selected from the group consisting of halogen and CN;
or a pharmaceutically acceptable salt thereof.

6. A compound according to claim 1 of formula (Ib) 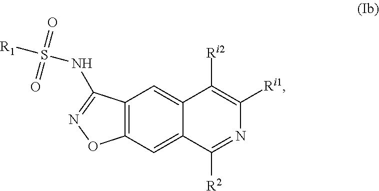
wherein
R₁ is selected from the group consisting of C₁₋₄ alkyl, C₁₋₄haloalkyl, C₃₋₆ cycloalkyl, NR¹¹R¹², 4 to 6-membered heterocyclyl, thienyl and phenyl, wherein the heterocyclyl, thienyl and phenyl is unsubstituted or substituted with 1 or 2 substituents independently selected from C₁₋₄ alkyl;
R¹¹ and R¹² are independently selected from the group consisting H and C₁₋₄ alkyl;
Rⁱ¹ and Rⁱ² are independently selected from the group consisting of H, C₁₋₄ alkyl, C₁₋₄ haloalkyl, halogen and CN;
R² is selected from the group consisting of 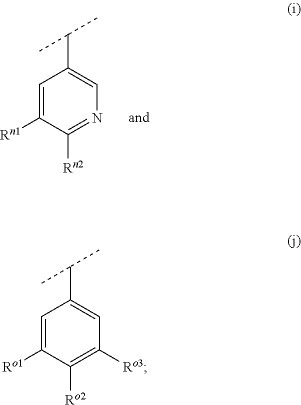
Rⁿ¹ is selected from the group consisting of halogen and CN;
Rⁿ² is selected from the group consisting of C₁₋₄ alkoxy, C₁₋₄ haloalkoxy, —O—(CRⁿ²¹Rⁿ²²)ₙ—C₃₋₆ cycloalkyl, —O—(CRⁿ²¹Rⁿ²²)ₙ-4 to 6-membered heterocyclyl and —O—(CRⁿ²¹Rⁿ²²)ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;
Rⁿ²¹ and Rⁿ²² are independently selected from the group consisting of H, C₁₋₄ alkyl and halogen;
R^{o1} is selected from the group consisting of H and halogen;
R^{o2} is selected from the group consisting of halogen, C₁₋₄ alkoxy, C₁₋₄ haloalkoxy, —O—(CR^{o21}R^{o22})ₙ—C₃₋₆ cycloalkyl, —O—(CRⁿ²¹Rⁿ²²)ₙ-4 to 6-membered heterocyclyl and —O—(CR^{o21}R^{o22})ₙ-phenyl, wherein the cycloalkyl, heterocyclyl and phenyl are unsubstituted or substituted by 1 to 3 substituents independently selected from C₁₋₄ alkyl, halogen and C₁₋₄ haloalkyl;
R^{o21} and R^{o22} are independently selected from the group consisting of H, C₁₋₄ alkyl and halogen;
n is independently selected from the group consisting of 0, 1 and 2;
R^{o3} is selected from the group consisting of halogen and CN;
or a pharmaceutically acceptable salt thereof.

7. A compound or salt according to claim 1 selected from the group consisting of
N-(8-(5-chloro-6-isobutoxypyridin-3-yl)naphtho[2,3-d]isoxazol-3-yl)cyclopropanesulfonamide;
N-(7-(5-chloro-6-isobutoxypyridin-3-yl)-5-(trifluoromethyl)-7H-isoxazolo[4,5-f]indol-3-yl)methanesulfonamide;
N-(7′-(5-chloro-6-isobutoxypyridin-3-yl)-6′-oxo-6′,7′-dihydrospiro[cyclobutane-1,5′-isoxazolo[4,5-f]indole]-3′-yl)cyclopropanesulfonamide;
N-(7′-(5-chloro-6-((1-methylcyclopropyl)methoxy)pyridin-3-yl)-6′-oxo-6′,7′-dihydrospiro[cyclopropane-1,5′-isoxazolo[4,5-f]indole]-3′-yl)methanesulfonamide;
N-(7-(5-chloro-6-isobutoxypyridin-3-yl)-5,5-dimethyl-6-oxo-6,7-dihydro-5H-isoxazolo[4,5-f]indol-3-yl)cyclopropanesulfonamide;
N-(7-(5-chloro-6-isobutoxypyridin-3-yl)-5-methyl-7H-isoxazolo[4,5-f]indol-3-yl)cyclopropanesulfonamide;
N-(7′-(6-sec-butoxy-5-chloropyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-(cyclobutylmethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7′-(5-chloro-6-(cyclopentylmethoxy)pyridin-3-yl)-6′-oxo-6′,7′-dihydrospiro[cyclopropane-1,5′-isoxazolo[4,5-f]indole]-3′-yl)cyclopropanesulfonamide;
N-(7-(5-chloro-6-isobutoxypyridin-3-yl)-5-methyl-7H-isoxazolo[4,5-f]indazol-3-yl)methanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)methanesulfonamide;
N-(7′-(5-chloro-6-isobutoxypyridin-3-yl)-6′-oxo-6′,7′-dihydrospiro[cyclopropane-1,5′-isoxazolo[4,5-f]indole]-3′-yl)propane-2-sulfonamide;
N-(7-(3,4-dichlorophenyl)-5-methyl-7H-isoxazolo[4,5-f]indazol-3-yl)cyclopropanesulfonamide;
N-(8-(5-chloro-6-isobutoxypyridin-3-yl)isoxazolo[4,5-g]isoquinolin-3-yl)cyclopropanesulfonamide;
N-(7′-(5-chloro-6-(cyclohexylmethoxy)pyridin-3-yl)-6′-oxo-6′,7′-dihydrospiro[cyclopropane-1,5′-isoxazolo[4,5-f]indole]-3′-yl)methanesulfonamide;
or a pharmaceutically acceptable salt thereof.

8. A pharmaceutical composition comprising a therapeutically effective amount of a compound according to claim 1 or a pharmaceutically acceptable salt thereof and one or more pharmaceutically acceptable carriers.

9. A combination comprising a therapeutically effective amount of a compound according to claim 1 or a pharmaceutically acceptable salt thereof and one or more therapeutically active co-agents.

10. A combination comprising a therapeutically effective amount of a compound according to claim 1 or a pharmaceutically acceptable salt thereof and one or more pain-relieving agent.

11. A combination according to claim 10 wherein the pain-relieving agent is selected from the group consisting of
a) opioid analgesics, for example morphine, ketobemidone or fentanyl; b) analgesics of the NSAID or COX-1/2 class, for example ibuprofen, naproxen, celecoxib or acetylsalicylic acid, and their analogues containing nitric oxide-donating groups; c) analgesic adjuvants such as amitriptyline, imipramine, duloxetine or mexiletine; d) NMDA antagonists for example ketamine or dextrometorfan; e) sodium channel blocking agents, for example lidocaine; f) anticonvulsants, for example carbamazepine, topiramate or lamotrigine; g) anticonvulsant/analgesic amino acids such as gabapentin or pregabalin; h) cannabinoids.

12. A method of treating pain, comprising administering to the subject a therapeutically effective amount of a compound according to claim 1 or a pharmaceutically acceptable salt thereof.
